SEQUENCE LISTING (114 sequences)

SEQ ID NO: 1 - DNA, 8905 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtacctagctaggtagcaattgaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcaagcttcccggcgcgccccggtccgtccggactacggcaagctgaccctgaagttcatcccaaaacttacgctgagtacttcgatctggtcaccccggatccgtgatagtaacctgatagtaacctgataatagcagatctcgccgccatgggagctgatgatgtggttgattcttcgaaatcttttgtcatggaaaacttttcttcgtaccacgggacgaaacctggttatgtggattccattcaaaaaggcatacaaaagccaaaatctggtacacaaggaaactatgacgatgattggaaagggttttatagtaccgacaacaaatatgacgctgcgggatactctgtggataatgaaaacccgctctctggaaaagctggaggcgtggtcaaagtgacgtatccaggactgacgaaggttctcgcactaaaggtggataatgccgaaactattaagaaagagttaggtttaagtctcactgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgaactagtatccggaatctagagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 2 - DNA, 12082 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccgctagctgataatagccgataccctgtcaccggatgtgctttccggtctgatgagtccgtgaggacgaaacaggactgtcgcagagacaaccgccgataccctgtcaccggatgtgctttccggtctgatgagtccgtgaggacgaaacaggactgtcgcagggcaccgtatcaactctgagatgcaggtacatccagctgatgagtcccaaataggacgaaacgcgcttcggtgcgtcctggattccactgctatccactattcatccaaagagactatcaactctgagatgcaggtacatccagctgatgagtcccaaataggacgaaacgcgcttcggtgcgtcctggattccactgctatccactattcatccgtctccgatcgtctagagcctgacctagctgacctagccgccaccatgcagaggctgcaggtagtgctgggccacctgaggggtccggccgattccggctggatgccgcaggcagcgccttgcctgagatctactagtggtgacctagctgacctagccgtcaccatggaccctgttgtgctgcaaaggagagactgggagaaccctggagtcacccagctcactgaacttaaccctagccctgccaccatggcttggaggaactccgaggaagccaggactgacagtaacagtaggcagcgccgccatggcaacggagagtggaggtttgcctggtttgacgctaaggatagtgtggccaccatggggctggagtgcgacctcccagaggcggctgacgttaaagttagcagcagccgccatgggcacggctacgacgcgcccatctacagtgacgttagcactaagatcgccaccatggccccttttgtgcccaccgagaacccgactaactgtagcagtgacacctgccgccatggcgagagctggctgcaagaaggacagactaagattagctgtgacggagccaccatggccttccacctctggtgcaacggcaggtgtaacggtagcggtgaagacagccgccatggctccgagttcgacctctctgccttccgtaacgctgaagataacagggccaccatggtggtgctcaggtggtccgacggcagctatagggctgaccgtaacatgtgccgccatggtggcatcttcagggacgtcagcctgcttagcactgagactaaccaggccaccatggtccacgttgccacgaggttcaacgacgatagcagtgaagctaagctgggccgccatgggcagatgtgtggagaactcagagagtctgacagtagcactaagagcgccaccatgggcgagacccaggtggcctctggcacagctgactttagagctaagatcagccgccatggaggaggctacgccgacagagccacccttgagcttagcgttaagaacgccaccatggggtctgccgagacccccaacctctacagtaacgttagggctgagcacagccgccatggcacgctcatcgaagccgaagcctgcgataacggtgacagtagagtcgccaccatggacggcctgctgctgctcaacggcaagcctaagcttgacagtagagtcagccgccatgggcaccatcctctgcacggacaagtcattaaggctgagactagggtggccaccatggtgctcacgaagcagaacaacttcaacgctaagagtgactctagctaccgccgccatggtctctggtacaccctgtgcgacaggaataaccttagggttgacgaggccaccatggtcgagacacacggcatggtgcccacgaataagcttagagctgaccccagccgccatggtgccatgtccgagagagtcaccaggattaagcatagagctgagaacgccaccatggtcatcatctggtctctgggcaacgagtctaagcatagagctgaccacggccgccatggcaggtggatcaagtctgccgaccccagtagacctgagcataacgaagccaccatggcagacaccacagccacagacatcatctgtagcattgaggctaaggtcggccgccatgggcccttccctgctgtgcccaagtggagtagcactgagtgtaactctgccaccatggaaacgagacctctcatcctgtgcgagtatagacctgaaagtaaacccggccgccatggctttgccaagtactggcaagccttcactgagtatagcactaagcaagccaccatggcccgcgactgggtggaccagtcactcattgagtatagcgataacggcagccgccatggtgcctacggaggagactttggcgacactgagaatagcactaagttcgccaccatgggcctggtctttgccgaccggactccgcctgaccctagcactaaggccagccgccatggacagttcttcccgttcacgctgtctggtgaaactaacgatagcacagccaccatggtcttcagacactccgacaacgagctgcttgactgtaaggctaggctgggccgccatggtctggcttctggcgaggtgcctctggctgaggctaatcatagaaaggccaccatggaactgcccgagctgcctcagccagagtctgacgataaccttaggctcagccgccatggggttcagcccaacgcaacagcttggtctgagcctagccataactctgccaccatggagtggaggctggccgagaacctctcggttgaccttagggctaactctcgccgccatggtcacctcacaacatccgaaatggagtttgacattaggcttaacaacgccaccatggagttcaacaggcagtctggcttcctgtctgagattaggactaaagacagccgccatggcctctctcctctccgagacctgttcactagggctgagcttaacactgccaccatggtgtcagaggccaccaggatcgacccaaatagttgtgaggataagtggagccgccatggacactaccaggccgaggctgccctgcttagctgtgaagctaaccagcgaatgcctggggctctcatcaccacagcccacgcttgtagccctgaagctaagacagcgaatgcctggggcaagacctacagaatcgacggccataggcctgaggctaaccagcgaatgcctggggctgcctccgacacacctcaccctgctaggattgagcttaactcagcgaatgcctggggcgcagagagggtcaactggctgggtgagggtaatcataggcagcgaatgcctggggccacagctgcctgcttcgacacctgtgagcttaaccttagcgcagcgaatgcctggggcgtgttcccttccgagaacggccttgagtgtaacactaggcagcgaatgcctggggctcaccagtggaggggagacttgcctgacagtaactctaggccagcgaatgcctggggcatggaaacctctcacagacagcttgaccataaggataggcagcgaatgcctggggccgacggcttccacatgggcattggtgaagataactctagctcagcgaatgcctggggcgagttcccgacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcaagcttcccggcgcgccccggtccgtccggactacggcaagctgaccctgaagttcatcccaaaacttacgctgagtacttcgatctggtcaccccggatctcgccgccatgggagctgatgatgtggttgattcttcgaaatcttttgtcatggaaaacttttcttcgtaccacgggacgaaacctggttatgtggattccattcaaaaaggcatacaaaagccaaaatctggtacacaaggaaactatgacgatgattggaaagggttttatagtaccgacaacaaatatgacgctgcgggatactctgtggataatgaaaacccgctctctggaaaagctggaggcgtggtcaaagtgacgtatccaggactgacgaaggttctcgcactaaaggtggataatgccgaaactattaagaaagagttaggtttaagtctcactgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgaactagtatccggaatctagagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 3 - DNA, 9536 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtacctagctaggtagcaattgaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcaagcttcccggcgcgccccggtccgtccggactacggcaagctgaccctgaagttcatcccaaaacttacgctgagtacttcgatctggtcaccccggatccgtgatagtaacctgatagtaacctgataatagcagatctgcagcttggggtatcagtcacattcggctggtacccctccggaagcgaatgggagccgacgatgtggtcgattcttcgaaatcttttgtcatggaaaacttttcttcgtaccacgggacgaaacctggttatgtggattccattcaaaaaggtaggtttaatgttcgttagatatagttgcagcttctaacaaacatcaaaactgattatgcttagggtttttctttttattttttaacaggcatacaaaagccaaaatctggtacacaaggaaactacgacgacgattggaaaggtgaggcactcagggtgcaggacttggactataaacccaatggagaagatagcccttcaacctctgtgacttttctaaagctactttcccccctttttgccttagggttttacagtaccgacaacaaatacgacgctgcgggatactctgtggacaacgaaaacccgctctctggaaaagctggaggcgtggtcaaagtcacgtatccaggtcaaaggaaataaatttttagaatccatttatttgtactgaagtaaaagttcacatatgcaacttctatttaataggttaacttcacaaacctattctgtaccatagggctcacgaaagttctcgcactcaaagtggacaatgccgaaactatcaagaaagagttgggtctctctctcaccgaaccgctcatggagcaagtcggaacggaagagtttatcaaaagattcggcgatggtgcttcgcgtgtcgtgctcagccttcccttcgccgaggggagttccagcgtcgagtacatcaacaactgggaacaggtatgaatgcaattgttggcatctttttttaaagttatgtttaagatatgaagttaaaattattttcaaatctgtagttaggctagtcattaaaactttttccaggtcagaacttacgacctgcttttatttccaaataggcgaaagcgctcagcgtcgaactcgagatcaacttcgaaacccgtggaaaacgtggccaagatgcgatgtacgagtatatggctcaagcctgtgcaggtgggcagctcatgagcccaggagattctgtcttgtttctgtgcctagtggagtttgttagtttgctgtgattagctggcaacggaaactggattcatgttgcagagggtttttctcatctgggtattcttggttttccacttacactttccccgtcttttctgtaggaaatcgtgtcaggcgatagtgagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 4 - DNA, 9174 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtacctagctaggtagcaattgaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcaagcttcccggcgcgccccggtccgtccggactacggcaagctgaccctgaagttcatcccaaaacttacgctgagtacttcgatctggtcaccggtaccatgggagccgacgatgtggtcgattcttcgaaatcttttgtcatggaaaacttttcttcgtaccacgggacgaaacctggttatgtggattccattcaaaaaggcatacaaaagccaaaatctggtacacaaggaaactacgacgacgattggaaagggttttacagtaccgacaacaaatacgacgctgcgggatactctgtggacaacgaaaacccgctctctggaaaagctggaggcgtggtcaaagtcacgtatccaggtgagtctctagccctgcctttgcctgtcctctcagcacttccattagccagctacctacttccatccactcccaaacttcagggctctgcctgcccccagaggcacaggacttagttctgggaccagggatcaggccgcagccctggcctgctgttgcttctgtcagggacttgcctttgaccccagcctctctgaccctcagggtctccttggggagctcttctgaatttgggctggcagataccccacccagaccaggtctgccggtgcggcagggccagtggggcaggttggctgtggctgctgtgccctagtctgccctttctgacttgcagggctcacgaaggttctcgcactcaaggtggacaatgccgaaactatcaagaaagagttgggtctcagcctcaccgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 5 - DNA, 10034 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtacctagctaggtagcaattgaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcaagcttcccggcgcgccctggctgagctgtacaagggtaagtcactgactgtctatgcctgggaaagggtgggcaggagatggggcagtgcaggaaaagtggcactatgaacccaactacacaaatcagcgatttcaacaacaactacacaaatcagcgatttcaattgtactaaccttcttctctttcctctcctgacaggaggagccatcatcgcccgatatcccaatcgcttaccgattcagaatctacggcaagctgaccctgaagttcatcaatcgcttaccgattcagaatccctacggcaagctgaccctgaagttcatcaatcgcttaccgattcagaatccctacggcaagctgaccctgaagttcatcaatcgcttaccgattcagaatccctacggcaagctgaccctgaagttcatcaatcgcttaccgattcagaatccctacggcaagctgaccctgaagttcatcaatcgcttaccgattcagaatccctacggcaagctgaccctgaagttcatcaatcgcttaccgattcagaatccctacggcaagctgaccctgaagttcatcaatcgcttaccgattcagaatccctacggcaagctgaccctgaagttcatcagatctgcagcttggggtatcagtcacattcggctggtacccctccggaagcgaatgggagccgacgatgtggtcgattcttcgaaatcttttgtcatggaaaacttttcttcgtaccacgggacgaaacctggttatgtggattccattcaaaaaggtaggtttaatgttcgttagatatagttgcagcttctaacaaacatcaaaactgattatgcttagggtttttctttttattttttaacaggcatacaaaagccaaaatctggtacacaaggaaactacgacgacgattggaaaggtgaggcactcagggtgcaggacttggactataaacccaatggagaagatagcccttcaacctctgtgacttttctaaagctactttcccccctttttgccttagggttttacagtaccgacaacaaatacgacgctgcgggatactctgtggacaacgaaaacccgctctctggaaaagctggaggcgtggtcaaagtcacgtatccaggtcaaaggaaataaatttttagaatccatttatttgtactgaagtaaaagttcacatatgcaacttctatttaataggttaacttcacaaacctattctgtaccatagggctcacgaaagttctcgcactcaaagtggacaatgccgaaactatcaagaaagagttgggtctctctctcaccgaaccgctcatggagcaagtcggaacggaagagtttatcaaaagattcggcgatggtgcttcgcgtgtcgtgctcagccttcccttcgccgaggggagttccagcgtcgagtacatcaacaactgggaacaggtatgaatgcaattgttggcatctttttttaaagttatgtttaagatatgaagttaaaattattttcaaatctgtagttaggctagtcattaaaactttttccaggtcagaacttacgacctgcttttatttccaaataggcgaaagcgctcagcgtcgaactcgagatcaacttcgaaacccgtggaaaacgtggccaagatgcgatgtacgagtatatggctcaagcctgtgcaggtgggcagctcatgagcccaggagattctgtcttgtttctgtgcctagtggagtttgttagtttgctgtgattagctggcaacggaaactggattcatgttgcagagggtttttctcatctgggtattcttggttttccacttacactttccccgtcttttctgtaggaaatcgtgtcaggcgatagtgagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 6 - DNA, 9441 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtacctagctaggtagcaattgaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcaagcttcccggcgcgtctacggcaagctgaccctgaagttcatccaaaactacacaaatcagcgatttcaacaaaactacacaaatcagcgatttcaacaacaaaactacacaaatcagcgatttcaacaaaatcgcttaccgattcagaatcgcccggggatctgtccactgctgttgctgttttgggcatccatcaggagaaggctcacggcaacaaagtgctcggtgcctttactacgggggggggggggggggggggggggccgaagttgtcagcccagaaccccacacgagttttgccactgggaagctgtgatccagtgcaggctgggacagccgacctccagcgcgcggtcaccggtaccatgggagccgacgatgtggtcgattcttcgaaatcttttgtcatggaaaacttttcttcgtaccacgggacgaaacctggttatgtggattccattcaaaaaggcatacaaaagccaaaatctggtacacaaggaaactacgacgacgattggaaagggttttacagtaccgacaacaaatacgacgctgcgggatactctgtggacaacgaaaacccgctctctggaaaagctggaggcgtggtcaaagtcacgtatccaggtgagtctctagccctgcctttgcctgtcctctcagcacttccattagccagctacctacttccatccactcccaaacttcagggctctgcctgcccccagaggcacaggacttagttctgggaccagggatcaggccgcagccctggcctgctgttgcttctgtcagggacttgcctttgaccccagcctctctgaccctcagggtctccttggggagctcttctgaatttgggctggcagataccccacccagaccaggtctgccggtgcggcagggccagtggggcaggttggctgtggctgctgtgccctagtctgccctttctgacttgcagggctcacgaaggttctcgcactcaaggtggacaatgccgaaactatcaagaaagagttgggtctcagcctcaccgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 7 - DNA, 9635 bp, Artificial Sequence
  Synthetic
agatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagacgcggtacctagctaggtagcaattgaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcgtgcgcacctacggcaagctgaccctgaagttcatccaacaaaactacacaaatcagcgatttccacaacaactacggcaagctgaccctgaagttcatccaacaaaactacacaaatcagcgatttccacaacaactacggcaagctgaccctgaagttcatccaacaaaactacacaaatcagcgatttccagcaaggcaaccaaaggctctttttagagccacctttcaacgcgcaaggcaacaaaaggcccttttcagggccacctttcaagagggcgcaaggcaaccaaaggctcttttcagagccacctttcaaggcgcaaggcaaccaaaggctcttttcagagccccctttattggacaaactacctacagagatttaaagctctaaggtaaatataaaatttttaagtgtataatgtgttaaactactgattctaattgtttgtgtattttagattccaacctatggaactgatcaatcggagcagtggtggaatccctttaaacatttgcgtctgacacaactgtgttcactagcaacctcaaacagacaccacggtgcatctgactcctgaggagaagtctgccgttactgccctgtggggcaaggtgaacgtggacgaagttggtgctgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactgctgaagctgttacgggcaaccctaagctgaaggctcctggcaagaaagtgctcggtgcctttagtgatcgcctggctcacctggacaacctcaagggcacctttgccacgctgagtgagctgcactgtgacaagctgcacgtgtatcctgagaacttcaggctcctgggcaacgtgctggtctgtgtgctggcccatcaccttggcaaagaattcaccccaccagtgcaggctgcctatcagaaagtggtggctggtgtggctaacgccctggcccacaagtatcactaagctcgctttcttgctgtccgatttctattagaggttcctttgttccctaagtccaactacgaaactgggggatattctgaagggccttgagcatctggattctgcctggcgcgccggtcaccccggatccgtgatagtaacctgatagtaacctgataatagcagatctcgccgccatgggagctgatgatgtggttgattcttcgaaatcttttgtcatggaaaacttttcttcgtaccacgggacgaaacctggttatgtggattccattcaaaaaggcatacaaaagccaaaatctggtacacaaggaaactatgacgatgattggaaagggttttatagtaccgacaacaaatatgacgctgcgggatactctgtggataatgaaaacccgctctctggaaaagctggaggcgtggtcaaagtgacgtatccaggactgacgaaggttctcgcactaaaggtggataatgccgaaactattaagaaagagttaggtttaagtctcactgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgaactagtatccggaatctagagcggccgctggccgcaataaaatatctttattttcattacatctgtgtgttggttttttgtgtgaggatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttggcattccggtactgttggtaaagccaccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 8 - DNA, 8866 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtacctagctaggtagcaattgaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcaagcttcccggcgcgccccggtccgtccggactacggcaagctgaccctgaagttcatcccaaaacttacgctgagtacttcgatctggtcaccccggatctcgccgccatgggagctgatgatgtggttgattcttcgaaatcttttgtcatggaaaacttttcttcgtaccacgggacgaaacctggttatgtggattccattcaaaaaggcatacaaaagccaaaatctggtacacaaggaaactatgacgatgattggaaagggttttatagtaccgacaacaaatatgacgctgcgggatactctgtggataatgaaaacccgctctctggaaaagctggaggcgtggtcaaagtgacgtatccaggactgacgaaggttctcgcactaaaggtggataatgccgaaactattaagaaagagttaggtttaagtctcactgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgaactagtatccggaatctagagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 9 - DNA, 9665 bp, Artificial Sequence
  Synthetic
agatctgcgcagcaccatggcctgaaataacctctgaaagaggaacttggttaggtacctaccggaaggaacccgcgctatgacggcaataaaaagacagaataaaacgcacggtgttcttataatggttacaaataaagcaatagcatcacaaatttcacaaataaagcatttttttcactgcattctagttgtggtaataaaatatctttattttcattacatctgtgtgttggttttttgtgtgtggcctcccaaagtgctgggattacaggcatgagccatcgagcccaacccaattttttttttttttaattttactttctgcaatcattcatccattcagccagtgcggtatttctgaggtgtgttcgatcgcggatccatgcctgccgcagtacagttgtgagccaaatgagactgagactagttcccgccctccaagagcttgcaagacccgcagtggcgtaaaaacactaacatcttttagtgatcgattctgcactccaggggttttcaatctactacaagagtgaataagagttcgcctttgtctgatatctgttgtcattctctctcgcttctttaactgattttttctcagctaataaaacatccacccacaaccccccgaacgcccgcaaacaccaggccactctagcaaaacctctctcactccgcctgcgcaatccagctgacttccggttacagataaccacgtgattgggaacccttgctgcgcatgtctagtaggaagtcggactataccactttccctacggaaggggtacttttttatgtttttaagtttaaaaccgatttctgatatttgacttttatcatttcaggcctatatggaggctatgagtgagtttagtgtggcagaagatgaaagaaccggacaggaatacggacgaaattggagcagggtttgggctctccccttcgcagataatcggaggagccgggcccgagcgagctctttcctttcgctgctgcggccgcagccgtgaggtgagggcgagctggtctccatcaggcgctgacgcgtgtcgacaagggactgtcggtcttgggaccgcagctggggttgggggagatgaaatggaggccgccctaaagcggccggtcccggggtttggggtaggccggagcactttcgtcccgggcctccggagtgagggggggcggggagcgtcgcagcaactgagaccaggaaaagtctgccccggctggtgccgcaccgcacacgtgtccggtcgacccacgcgagcagagcaaacggagcgaacaagaccaagccgtgggccctttcttgcttggcacacccggagcggagccgatctctgctttcacgtgatgtagggcaagcctagtgtaggccccaggcctccgactgccgagagaggtgatctctaactcttgactccattcactcctttggcctctcataaaggaaatctctgcgaatagccgaacgaggcttgttactgtgataaaacagggaaataagcccagaaaacagagtaacttgcctgcattcctagactagaaatcaggtctactcacctcgaatattctttaaacgctgagtaccagaaatggcataacccccctattcaatccaataagtccttggcttgactttccagaggagaaatgcgaacatgaggctccgagaggtgaaggcatagcgtgggttttgaagtcttaaacccaagggggccagctgcatagcccagagccttaaagatgatttagggaagagtcttatttcgcggctgtggtgtgggtcacaaagggcaggtcttgatggggacgttcattcttgcccaggattggctttcagagtctaatcatgttttctgtgtgtctagtatcctcaggcttcagaagaggctcgcctctagtgtcctccgctgtggcaagaagaaggtctggaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcaagcttcccggcgcgccccggtccgtccggtcccacgcgtcaattggaaaacttacgctgagtacttcgatctccctacggcaagctgaccctgaagttcaacagatctcgccgccatgggagctgatgatgtggttgattcttcgaaatcttttgtcatggaaaacttttcttcgtaccacgggacgaaacctggttatgtggattccattcaaaaaggcatacaaaagccaaaatctggtacacaaggaaactatgacgatgattggaaagggttttatagtaccgacaacaaatatgacgctgcgggatactctgtggataatgaaaacccgctctctggaaaagctggaggcgtggtcaaagtgacgtatccaggactgacgaaggttctcgcactaaaggtggataatgccgaaactattaagaaagagttaggtttaagtctcactgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgaactagtatccggaatctagagcggccgctggccgcaataaaatatctttattttcattacatctgtgtgttggttttttgtgtgaggatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttggcattccggtactgttggtaaagccaccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 10 - DNA, 8290 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtacctagctaggtagcaattgaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcaagcttcccggcgcgccccggtccgtccggactacggcaagctgaccctgaagttcatcccaaaacttacgctgagtacttcgatctggtcaccccggatccgtgatagtaacctgatagtaacctgataatagcagatctgcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 11 - DNA, 6441 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtacccccaagcttaccggtcccacgcgtcaattggaaaacttacgctgagtacttcgatctccctacggcaagctgaccctgaagttcaacagatctcgccgccatgggagctgatgatgtggttgattcttcgaaatcttttgtcatggaaaacttttcttcgtaccacgggacgaaacctggttatgtggattccattcaaaaaggcatacaaaagccaaaatctggtacacaaggaaactatgacgatgattggaaagggttttatagtaccgacaacaaatatgacgctgcgggatactctgtggataatgaaaacccgctctctggaaaagctggaggcgtggtcaaagtgacgtatccaggactgacgaaggttctcgcactaaaggtggataatgccgaaactattaagaaagagttaggtttaagtctcactgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgaactagtatccggaatctagagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 12 - DNA, 6368 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatctgctagcggcgcgcccccggtacctgataagcctagcagcgaatgcctggggcagacgatgtcgtcgacagtagcaagagctttgtgatggagaattttagtagctatcatggtactaagccgggatacgtagatagtatccagaagggaatccagaaacccaagagcggaactcagggcaactacgatgacgactggaagggtttctactcgaccgataacaaatatgatgcagccggttacagcgtggacaacgagaatcctttgagcggcaaggcaggcggggtcgtcaaggtcacctaccccggtttaaccaaagtgttagctttgaaggtggacaacgcggagacaatcaaaaaggaactcggactctcgctcacggagcctcttatggaacaggtcggcaccgaggaattcataaagcgttttggagatggagcaagtagggttgtcttatcattgccatttgcggaaggctcgagctcagtggagtacataaacaattgggagcaagccaaggcactctcagttgagctggagatcaacttcgagacaagaggcaagagagggcaggacgcgatgtacgagtacatggcacaggcgtgcgctggcaacagagtccgtaggtgaacataagcataggcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 13 - DNA, 6015 bp, Artificial Sequence
  Synthetic
agatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagacgcggtacctagctaggtagcaattgaccggtcccacgcgtcaattggaaaacttacgctgagtacttcgatctccctacggcaagctgaccctgaagttcaacagatctcgccgccatgggagctgatgatgtggttgattcttcgaaatcttttgtcatggaaaacttttcttcgtaccacgggacgaaacctggttatgtggattccattcaaaaaggcatacaaaagccaaaatctggtacacaaggaaactatgacgatgattggaaagggttttatagtaccgacaacaaatatgacgctgcgggatactctgtggataatgaaaacccgctctctggaaaagctggaggcgtggtcaaagtgacgtatccaggactgacgaaggttctcgcactaaaggtggataatgccgaaactattaagaaagagttaggtttaagtctcactgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgaactagtatccggaatctagagcggccgctggccgcaataaaatatctttattttcattacatctgtgtgttggttttttgtgtgaggatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttggcattccggtactgttggtaaagccaccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 14 - DNA, 6352 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatcttgtacattcgaacgccgccatgggcgctgatgatgttgttgattcttctaaatcttttgtcatggaaaacttttcttcgtaccacgggactaaacctggttatgtggattccattcaaaaaggtatacaaaagccaaaatctggtacacaaggaaattatgacgatgattggaaagggttttatagtaccgacaataaatacgacgctgcgggatactctgtggataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatagtgaactagttccggatctagagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 15 - DNA, 6658 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtaccatgggagccgacgatgtggtcgattcttcgaaatcttttgtcatggaaaacttttcttcgtaccacgggacgaaacctggttatgtggattccattcaaaaaggcatacaaaagccaaaatctggtacacaaggaaactacgacgacgattggaaagggttttacagtaccgacaacaaatacgacgctgcgggatactctgtggacaacgaaaacccgctctctggaaaagctggaggcgtggtcaaagtcacgtatccaggtgagtctctagccctgcctttgcctgtcctctcagcacttccattagccagctacctacttccatccactcccaaacttcagggctctgcctgcccccagaggcacaggacttagttctgggaccagggatcaggccgcagccctggcctgctgttgcttctgtcagggacttgcctttgaccccagcctctctgaccctcagggtctccttggggagctcttctgaatttgggctggcagataccccacccagaccaggtctgccggtgcggcagggccagtggggcaggttggctgtggctgctgtgccctagtctgccctttctgacttgcagggctcacgaaggttctcgcactcaaggtggacaatgccgaaactatcaagaaagagttgggtctcagcctcaccgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 16 - DNA, 6964 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtaccgaggagatctgccgccgcgatcgccggaagcgaatgggagccgacgatgtggtcgattcttcgaaatcttttgtcatggaaaacttttcttcgtaccacgggacgaaacctggttatgtggattccattcaaaaaggtaggtttaatgttcgttagatatagttgcagcttctaacaaacatcaaaactgattatgcttagggtttttctttttattttttaacaggcatacaaaagccaaaatctggtacacaaggaaactacgacgacgattggaaaggtgaggcactcagggtgcaggacttggactataaacccaatggagaagatagcccttcaacctctgtgacttttctaaagctactttcccccctttttgccttagggttttacagtaccgacaacaaatacgacgctgcgggatactctgtggacaacgaaaacccgctctctggaaaagctggaggcgtggtcaaagtcacgtatccaggtcaaaggaaataaatttttagaatccatttatttgtactgaagtaaaagttcacatatgcaacttctatttaataggttaacttcacaaacctattctgtaccatagggctcacgaaagttctcgcactcaaagtggacaatgccgaaactatcaagaaagagttgggtctctctctcaccgaaccgctcatggagcaagtcggaacggaagagtttatcaaaagattcggcgatggtgcttcgcgtgtcgtgctcagccttcccttcgccgaggggagttccagcgtcgagtacatcaacaactgggaacaggtatgaatgcaattgttggcatctttttttaaagttatgtttaagatatgaagttaaaattattttcaaatctgtagttaggctagtcattaaaactttttccaggtcagaacttacgacctgcttttatttccaaataggcgaaagcgctcagcgtcgaactcgagatcaacttcgaaacccgtggaaaacgtggccaagatgcgatgtacgagtatatggctcaagcctgtgcaggtgggcagctcatgagcccaggagattctgtcttgtttctgtgcctagtggagtttgttagtttgctgtgattagctggcaacggaaactggattcatgttgcagagggtttttctcatctgggtattcttggttttccacttacactttccccgtcttttctgtaggaaatcgtgtcaggcgatagtgagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 17 - DNA, 7733 bp, Artificial Sequence
  Synthetic
agatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagacgcggtacctagctaggtagcaattgaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgagcccgggaattcgtttaaacctagagcggccgctggccgcaataaaatatctttattttcattacatctgtgtgttggttttttgtgtgaggatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttggcattccggtactgttggtaaagccaccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 18 - DNA, 6679 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatcttgtacattcgaacgccgccatgggcgctgatgatgttgttgattcttctaaatcttttgtcatggaaaacttttcttcgtaccacgggactaaacctggttatgtggattccattcaaaaaggtatacaaaagccaaaatctggtacacaaggaaattatgacgatgattggaaagggttttatagtaccgacaataaatacgacgctgcgggatactctgtggataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatagtgaactagttccggatctagagcggccgcactcgaggtttaaacggccggccgcggtcatagctgtttcctgaacagatcccgggtggcatccctgtgacccctccccagtgcctctcctggccctggaagttgccactccagtgtccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatgaaaaagcctgaactcaccgcgacgtctgttgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcaaccggtcgcggaggccatggatgcaatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagctgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 19 - DNA, 870 bp, Artificial Sequence
  Synthetic
cgcggatccaccggtcaattgtatcaactctgagatgcaggtacatccagctgatgagtcccaaataggacgaaacgcgcttcggtgcgtcctggattccactgctatccactattcatctacttgcactgcacccgataccctgtcaccggatgtgctttccggtctgatgagtccgtgaggacgaaacaggactggaacgtactacgacaggaacttgtcctgagatgcaggtacatcccactgatgagtcccaaataggacgaaacgcgcttcggtgcgtctgggattccactgctatccacacgcgtcggtccgaagcttgtcgaccgccggtgcaaagatctgaattcacctgatagctgatagctgatagcccggggtctctgtggatagaccagagcggagcctgggagctctctggctatctacggaacccactgcttaagcctcaataccgcttgccttgagtgcttcaagtagtgtgtgccgaacacaagctcacgacccactacacaagctcacgacccactacacaagctcacgacccactacacgagcttggggcgcgtggtggcggctgcagccgccaccacgcgccccggatcggagattgtcaggagctaaggaagctaaacaacgactacagcaggcttttgcaaaaagctccaccacggcccaacgttgggccgtggtggagcttggattgtacttgcactgcatacacaacgagatcggaacgtactacgacaggaactacgaccctgcggtccaccacggccgatatcacggccgtggtggaccgcagggaagaacaacgtctccgatctttggtaccaacacatctagacaaagtactggcgcgcccaa

SEQ ID NO: 20 - DNA, 4950 bp, Artificial Sequence
  Synthetic polynucleotide
gcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggattcgaaagatctgccaccatacgttgccgcgcagcggactgcccgccaggatatggatcctgatgatgttgttgattcttctaaatcttttgttatggaaaacttttcttcgtaccacgggactaaacctggttatgtggattccattcaaaaaggtatacaaaagccaaaatctggtacccaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgccgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaatgactcgagccatgggacccacacttttctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctttagtcctgttcctcagctacaaaatggacacaatttccagcagggtctctgagggcaaattcccttccccaaggttgttcaccaatttctgtcatggctgggccagaggcatccctgaaatttgtgctgactacttctgaccattctgcataaagctcatctaggcctctgacccagacccaagcaagggtgttgtcagggacaacttggtcctgaactgctgagatgaagagggtgacatcatctctgacaacaccagcaaaatcatcttcaacaaagtctctggagaatcctaatctgtcagtccagaactctacagcccctgcaacatcccttgctgtgaggactgggactgcagaagtgagtttggccatgatggctcctcctgtcaggagaggaaagagaagaaggttagtacaattgctatagtgagttgtattatactatgcttatgattaattgtcaaactagtgggttcatagtgccacttttcctgcactgccccatctcctgcccaccctttcccaggcatagacagtcagtgacttacccttgtacagctcatccattcccagagtaattcctgctgctgtcacaaactccaggaggaccatgtggtctcttttctcattagggtctttggacagagcagattgagtgctgagatagtgattatctgggaggagaactgggccatcaccaataggggtgttctgctggtaatggtctgccagttggacagatccatcctcaatgttgtgtctaatcttgaaattagccttaattccattcctctgcttatctgccataatgtaaacattgtgagaattatagttgtactccagcttgtgacccagaatgtttccatcttccttaaaatcaatgcctttcagctcaattctgttaaccagtgtatcaccttcaaacttcacttctgcccttgtcttataatttccatcatccttaaagaagattgtcctctcctgaacataaccttctggcattgcagatttaaagaagtcatgctgcttcatgtggtcagggtatctgctgaaacattgaacaccataagtcagggtggtcaccagagttggccaaggcactggcagctttcctgttgtacaaatgaacttcagagtcagctttccataagttgcatctccttcaccttcaccagacacagagaatttgtggccattcacatcaccatccagctcaaccagaattgggacaacaccagtaaagagttcttctcccttgctcatggtggcttggatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagctgatctgacggttcactaaacgagctctgcttatatagacctcccaccgtacacgcctaccgcccatttgcgtcaatggggcggagttgttacgacattttggaaagtcccgttgatttactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatca

SEQ ID NO: 21 - DNA, 7241 bp, Artificial Sequence
  Synthetic
agatctgcgcagcaccatggcctgaaataacctctgaaagaggaacttggttaggtacctaccggaaggaacccgcgctatgacggcaataaaaagacagaataaaacgcacggtgttcttataatggttacaaataaagcaatagcatcacaaatttcacaaataaagcatttttttcactgcattctagttgtggtaataaaatatctttattttcattacatctgtgtgttggttttttgtgtgtggcctcccaaagtgctgggattacaggcatgagccatcgagcccaacccaattttttttttttttaattttactttctgcaatcattcatccattcagccagtgcggtatttctgaggtgtgttcgatcgcggatccatgcctgccgcagtacagttgtgagccaaatgagactgagactagttcccgccctccaagagcttgcaagacccgcagtggcgtaaaaacactaacatcttttagtgatcgattctgcactccaggggttttcaatctactacaagagtgaataagagttcgcctttgtctgatatctgttgtcattctctctcgcttctttaactgattttttctcagctaataaaacatccacccacaaccccccgaacgcccgcaaacaccaggccactctagcaaaacctctctcactccgcctgcgcaatccagctgacttccggttacagataaccacgtgattgggaacccttgctgcgcatgtctagtaggaagtcggactataccactttccctacggaaggggtacttttttatgtttttaagtttaaaaccgatttctgatatttgacttttatcatttcaggcctatatggaggctatgagtgagtttagtgtggcagaagatgaaagaaccggacaggaatacggacgaaattggagcagggtttgggctctccccttcgcagataatcggaggagccgggcccgagcgagctctttcctttcgctgctgcggccgcagccgtgaggtgagggcgagctggtctccatcaggcgctgacgcgtgtcgacaagggactgtcggtcttgggaccgcagctggggttgggggagatgaaatggaggccgccctaaagcggccggtcccggggtttggggtaggccggagcactttcgtcccgggcctccggagtgagggggggcggggagcgtcgcagcaactgagaccaggaaaagtctgccccggctggtgccgcaccgcacacgtgtccggtcgacccacgcgagcagagcaaacggagcgaacaagaccaagccgtgggccctttcttgcttggcacacccggagcggagccgatctctgctttcacgtgatgtagggcaagcctagtgtaggccccaggcctccgactgccgagagaggtgatctctaactcttgactccattcactcctttggcctctcataaaggaaatctctgcgaatagccgaacgaggcttgttactgtgataaaacagggaaataagcccagaaaacagagtaacttgcctgcattcctagactagaaatcaggtctactcacctcgaatattctttaaacgctgagtaccagaaatggcataacccccctattcaatccaataagtccttggcttgactttccagaggagaaatgcgaacatgaggctccgagaggtgaaggcatagcgtgggttttgaagtcttaaacccaagggggccagctgcatagcccagagccttaaagatgatttagggaagagtcttatttcgcggctgtggtgtgggtcacaaagggcaggtcttgatggggacgttcattcttgcccaggattggctttcagagtctaatcatgttttctgtgtgtctagtatcctcaggcttcagaagaggctcgcctctagtgtcctccgctgtggcaagaagaaggtctggaccggtcccacgcgtcaattggaaaacttacgctgagtacttcgatctccctacggcaagctgaccctgaagttcaacagatctcgccgccatgggagctgatgatgtggttgattcttcgaaatcttttgtcatggaaaacttttcttcgtaccacgggacgaaacctggttatgtggattccattcaaaaaggcatacaaaagccaaaatctggtacacaaggaaactatgacgatgattggaaagggttttatagtaccgacaacaaatatgacgctgcgggatactctgtggataatgaaaacccgctctctggaaaagctggaggcgtggtcaaagtgacgtatccaggactgacgaaggttctcgcactaaaggtggataatgccgaaactattaagaaagagttaggtttaagtctcactgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgaactagtatccggaatctagagcggccgctggccgcaataaaatatctttattttcattacatctgtgtgttggttttttgtgtgaggatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttggcattccggtactgttggtaaagccaccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 22 - DNA, 6326 bp, Artificial Sequence
  Synthetic
agatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcccgaccggtcaacaacgtctcagatcttcccccaaggcgcgccgaactctagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctaggcgatcgctcgagcccgggaattcgtttaaacctagagcggccgctggccgcaataaaatatctttattttcattacatctgtgtgttggttttttgtgtgaggatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttggcattccggtactgttggtaaagccaccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 23 - DNA, 9195 bp, Artificial Sequence
  Synthetic
agatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcccgaccggtcaacaacgtctcagatcccggtccgactagtcgtacgcaccggcggtcgaccacgtgcctgcaggaaagtgcgcacggtcaccgagctcaaaacgccaagaacctcatcatcttccctacggcaagctgaccctgaagttcatcaactacacaaatcagcgatttccagctcagcgggacccgcatccggaggcgcgccgaactctagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctaggcgatcgtctagagcctgacctagctgacctagccgccaccatgcagaggctgcaggtagtgctgggccacctgaggggtccggccgattccggctggatgccgcaggcagcgccttgcctgagatctactagtggtgacctagctgacctagccgtcaccatggaccctgttgtgctgcaaaggagagactgggagaaccctggagtcacccagctcactgaacttaaccctagccctgccaccatggcttggaggaactccgaggaagccaggactgacagtaacagtaggcagcgccgccatggcaacggagagtggaggtttgcctggtttgacgctaaggatagtgtggccaccatggggctggagtgcgacctcccagaggcggctgacgttaaagttagcagcagccgccatgggcacggctacgacgcgcccatctacagtgacgttagcactaagatcgccaccatggccccttttgtgcccaccgagaacccgactaactgtagcagtgacacctgccgccatggcgagagctggctgcaagaaggacagactaagattagctgtgacggagccaccatggccttccacctctggtgcaacggcaggtgtaacggtagcggtgaagacagccgccatggctccgagttcgacctctctgccttccgtaacgctgaagataacagggccaccatggtggtgctcaggtggtccgacggcagctatagggctgaccgtaacatgtgccgccatggtggcatcttcagggacgtcagcctgcttagcactgagactaaccaggccaccatggtccacgttgccacgaggttcaacgacgatagcagtgaagctaagctgggccgccatgggcagatgtgtggagaactcagagagtctgacagtagcactaagagcgccaccatgggcgagacccaggtggcctctggcacagctgactttagagctaagatcagccgccatggaggaggctacgccgacagagccacccttgagcttagcgttaagaacgccaccatggggtctgccgagacccccaacctctacagtaacgttagggctgagcacagccgccatggcacgctcatcgaagccgaagcctgcgataacggtgacagtagagtcgccaccatggacggcctgctgctgctcaacggcaagcctaagcttgacagtagagtcagccgccatgggcaccatcctctgcacggacaagtcattaaggctgagactagggtggccaccatggtgctcacgaagcagaacaacttcaacgctaagagtgactctagctaccgccgccatggtctctggtacaccctgtgcgacaggaataaccttagggttgacgaggccaccatggtcgagacacacggcatggtgcccacgaataagcttagagctgaccccagccgccatggtgccatgtccgagagagtcaccaggattaagcatagagctgagaacgccaccatggtcatcatctggtctctgggcaacgagtctaagcatagagctgaccacggccgccatggcaggtggatcaagtctgccgaccccagtagacctgagcataacgaagccaccatggcagacaccacagccacagacatcatctgtagcattgaggctaaggtcggccgccatgggcccttccctgctgtgcccaagtggagtagcactgagtgtaactctgccaccatggaaacgagacctctcatcctgtgcgagtatagacctgaaagtaaacccggccgccatggctttgccaagtactggcaagccttcactgagtatagcactaagcaagccaccatggcccgcgactgggtggaccagtcactcattgagtatagcgataacggcagccgccatggtgcctacggaggagactttggcgacactgagaatagcactaagttcgccaccatgggcctggtctttgccgaccggactccgcctgaccctagcactaaggccagccgccatggacagttcttcccgttcacgctgtctggtgaaactaacgatagcacagccaccatggtcttcagacactccgacaacgagctgcttgactgtaaggctaggctgggccgccatggtctggcttctggcgaggtgcctctggctgaggctaatcatagaaaggccaccatggaactgcccgagctgcctcagccagagtctgacgataaccttaggctcagccgccatggggttcagcccaacgcaacagcttggtctgagcctagccataactctgccaccatggagtggaggctggccgagaacctctcggttgaccttagggctaactctcgccgccatggtcacctcacaacatccgaaatggagtttgacattaggcttaacaacgccaccatggagttcaacaggcagtctggcttcctgtctgagattaggactaaagacagccgccatggcctctctcctctccgagacctgttcactagggctgagcttaacactgccaccatggtgtcagaggccaccaggatcgacccaaatagttgtgaggataagtggagccgccatggacactaccaggccgaggctgccctgcttagctgtgaagctaaccagcgaatgcctggggctctcatcaccacagcccacgcttgtagccctgaagctaagacagcgaatgcctggggcaagacctacagaatcgacggccataggaaacctagagcggccgctggccgcaataaaatatctttattttcattacatctgtgtgttggttttttgtgtgaggatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttggcattccggtactgttggtaaagccaccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 24 - RNA, 13 nt, Artificial Sequence
  Synthetic
gccgccrccaugg

SEQ ID NO: 25 - RNA, 7 nt, Homo sapiens
accaugg

SEQ ID NO: 26 - protein, 9 aa, Artificial Sequence
  Synthetic
XXXXXXXXX

SEQ ID NO: 27 - protein, 9 aa, Homo sapiens
RLRVLSGHL

SEQ ID NO: 28 - RNA, 31 nt, Artificial Sequence
  Synthetic
uggagcagaggcucuggcagcuuuugcagcg

SEQ ID NO: 29 - RNA, 21 nt, Artificial Sequence
  Synthetic
gccaaggagccagagagcaug

SEQ ID NO: 30 - RNA, 11 nt, Artificial Sequence
  Synthetic
gccaaggagcc

SEQ ID NO: 31 - RNA, 5 nt, Artificial Sequence
  Synthetic
auuua

SEQ ID NO: 32 - RNA, 9 nt, Artificial Sequence
  Synthetic
uuauuuann

SEQ ID NO: 33 - RNA, 4 nt, Artificial Sequence
  Synthetic
auuu

SEQ ID NO: 34 - RNA, 16 nt, Artificial Sequence
  Synthetic
ggcucuuuucagagcc

SEQ ID NO: 35 - RNA, 6 nt, Artificial Sequence
  Synthetic
uuuuau

SEQ ID NO: 36 - RNA, 7 nt, Artificial Sequence
  Synthetic
uuuuuau

SEQ ID NO: 37 - RNA, 7 nt, Artificial Sequence
  Synthetic
uuuuaau

SEQ ID NO: 38 - RNA, 9 nt, Artificial Sequence
  Synthetic
uuuuuuauu

SEQ ID NO: 39 - RNA, 7 nt, Artificial Sequence
  Synthetic
uuuuauu

SEQ ID NO: 40 - RNA, 11 nt, Artificial Sequence
  Synthetic
uuuuuauaaag

SEQ ID NO: 41 - RNA, 21 nt, Artificial Sequence
  Synthetic
uauuauuaucuuggccgcccg

SEQ ID NO: 42 - RNA, 36 nt, Artificial Sequence
  Synthetic
gcggcccgccguauuauuaucuuggccgcccgccac

SEQ ID NO: 43 - RNA, 49 nt, Artificial Sequence
  Synthetic
gauaauaauacggcgggccgccaagagcggcccgccguauuauuaucuu

SEQ ID NO: 44 - RNA, 21 nt, Artificial Sequence
  Synthetic
gauaauaauacggcgggccgc

SEQ ID NO: 45 - RNA, 27 nt, Artificial Sequence
  Synthetic
guggcgggcggccaagauaauaauauu

SEQ ID NO: 46 - RNA, 25 nt, Artificial Sequence
  Synthetic
uauuauuaucuuggccgcccgccac

SEQ ID NO: 47 - RNA, 21 nt, Artificial Sequence
  Synthetic
ggcggccaagauaauaauauu

SEQ ID NO: 48 - RNA, 21 nt, Artificial Sequence
  Synthetic
cgggcggccaagauaauaaua

SEQ ID NO: 49 - RNA, 725 nt, Artificial Sequence
  Synthetic
ccgcggcugagaugcagguacaucccacugaugagucccaaauaggacgaaacgcgcuucggugcgucugggauuccacugcuauccacgcggccgcgaccauggauggaaccggcgggcggccaagauaauaauacaccaugggcgcugaugauguuguugauucuucuaaaucuuuugucauggaaaacuuuucuucguaccacgggacuaaaccugguuauguggauuccauucaaaaagguauacaaaagccaaaaucugguacacaaggaaauuaugacgaugauuggaaaggguuuuauaguaccgacaauaaauacgacgcugcgggauacucuguggauaaugaaaacccgcucucuggaaaagcuggaggcguggucaaagugacguauccaggacugacgaagguucucgcacuaaaaguggauaaugccgaaacuauuaagaaagaguuagguuuaagucucacugaaccguugauggagcaagucggaacggaagaguuuaucaaaagguucggugauggugcuucgcguguagugcucagccuucccuucgcugaggggaguucuagcguugaauauauuaauaacugggaacaggcgaaagcguuaagcguagaacuugagauuaauuuugaaacccguggaaaacguggccaagaugcgauguaugaguauauggcucaagccugugcaggaaaucgugucaggcgauag

SEQ ID NO: 50 - RNA, 599 nt, Artificial Sequence
  Synthetic
gauaauauaucaccaugggcgcugaugauguuguugauucuucuaaaucuuuugucauggaaaacuuuucuucguaccacgggacuaaaccugguuauguggauuccauucaaaaagguauacaaaagccaaaaucugguacacaaggaaauuaugacgaugauuggaaaggguuuuauaguaccgacaauaaauacgacgcugcgggauacucuguggauaaugaaaacccgcucucuggaaaagcuggaggcguggucaaagugacguauccaggacugacgaagguucucgcacuaaaaguggauaaugccgaaacuauuaagaaagaguuagguuuaagucucacugaaccguugauggagcaagucggaacggaagaguuuaucaaaagguucggugauggugcuucgcguguagugcucagccuucccuucgcugaggggaguucuagcguugaauauauuaauaacugggaacaggcgaaagcguuaagcguagaacuugagauuaauuuugaaacccguggaaaacguggccaagaugcgauguaugaguauauggcucaagccugugcaggaaaucgugucaggcgauag

SEQ ID NO: 51 - RNA, 21 nt, Artificial Sequence
  Synthetic
cucuguccacuuggagcccuu

SEQ ID NO: 52 - RNA, 50 nt, Artificial Sequence
  Synthetic
ucagaagagaccuucucuguccacuuggagcccuuuguauacuccuacug

SEQ ID NO: 53 - RNA, 50 nt, Artificial Sequence
  Synthetic
ggaguauacaaagggcuccaagggcuccaaguggacagagaaggucucuu

SEQ ID NO: 54 - RNA, 21 nt, Artificial Sequence
  Synthetic
ggagcccuuuguauacuccuu

SEQ ID NO: 55 - RNA, 21 nt, Artificial Sequence
  Synthetic
ggaguauacaaagggcuccaa

SEQ ID NO: 56 - RNA, 29 nt, Artificial Sequence
  Synthetic
gggcuccaaguggacagagaaggucucuu

SEQ ID NO: 57 - RNA, 21 nt, Artificial Sequence
  Synthetic
gggcuccaaguggacagagaa

SEQ ID NO: 58 - RNA, 21 nt, Artificial Sequence
  Synthetic
aagggcuccaaguggacagag

SEQ ID NO: 59 - RNA, 722 nt, Artificial Sequence
  Synthetic
ccugucaccggauguguuuuccggucugaugaguccgugaggacgaaacaggaccaugggaaccauggaaagggcuccaaguggacagagagggguggggguugggggaaggggaugggcgcugaugauguuguugauucuucuaaaucuuuugucauggaaaacuuuucuucguaccacgggacuaaaccugguuauguggauuccauucaaaaagguauacaaaagccaaaaucugguacacaaggaaauuaugacgaugauuggaaaggguuuuauaguaccgacaauaaauacgacgcugcgggauacucuguggauaaugaaaacccgcucucuggaaaagcuggaggcguggucaaagugacguauccaggacugacgaagguucucgcacuaaaaguggauaaugccgaaacuauuaagaaagaguuagguuuaagucucacugaaccguugauggagcaagucggaacggaagaguuuaucaaaagguucggugauggugcuucgcguguagugcucagccuucccuucgcugaggggaguucuagcguugaauauauuaauaacugggaacaggcgaaagcguuaagcguagaacuugagauuaauuuugaaacccguggaaaacguggccaagaugcgauguaugaguauauggcucaagccugugcaggaaaucgugucaggcgauagccccuucccccaacccccacccc

SEQ ID NO: 60 - RNA, 631 nt, Artificial Sequence
  Synthetic
guggacagagagggguyaaggggaugggcgcugaugauguuguugauucuucuaaaucuuuugucauggaaaacuuuucuucguaccacgggacuaaaccugguuauguggauuccauucaaaaagguauacaaaagccaaaaucugguacacaaggaaauuaugacgaugauuggaaaggguuuuauaguaccgacaauaaauacgacgcugcgggauacucuguggauaaugaaaacccgcucucuggaaaagcuggaggcguggucaaagugacguauccaggacugacgaagguucucgcacuaaaaguggauaaugccgaaacuauuaagaaagaguuagguuuaagucucacugaaccguugauggagcaagucggaacggaagaguuuaucaaaagguucggugauggugcuucgcguguagugcucagccuucccuucgcugaggggaguucuagcguugaauauauuaauaacugggaacaggcgaaagcguuaagcguagaacuugagauuaauuuugaaacccguggaaaacguggccaagaugcgauguaugaguauauggcucaagccugugcaggaaaucgugucaggcgauagccccuucccccaacccccacccc

SEQ ID NO: 61 - RNA, 21 nt, Artificial Sequence
  Synthetic
uaccaaugcugcuugugccug

SEQ ID NO: 62 - RNA, 51 nt, Artificial Sequence
  Synthetic
uagcaauacagcagcuaccaaugcugcuugugccuggcuagaagcacaaga

SEQ ID NO: 63 - RNA, 21 nt, Artificial Sequence
  Synthetic
caggcacaagcagcauuggua

SEQ ID NO: 64 - RNA, 21 nt, Artificial Sequence
  Synthetic
agcauugguagcugcuguauu

SEQ ID NO: 65 - RNA, 21 nt, Artificial Sequence
  Synthetic
ugcuucuagccaggcacaagc

SEQ ID NO: 66 - RNA, 681 nt, Artificial Sequence
  Synthetic
augaccaugggaaccaugcgccuccgcguucucucaggccaccucggccaggcacaagcagcauugguagcaugggcgcugaugauguuguugauucuucuaaaucuuuugucauggaaaacuuuucuucguaccacgggacuaaaccugguuauguggauuccauucaaaaagguauacaaaagccaaaaucugguacacaaggaaauuaugacgaugauuggaaaggguuuuauaguaccgacaauaaauacgacgcugcgggauacucuguggauaaugaaaacccgcucucuggaaaagcuggaggcguggucaaagugacguauccaggacugacgaagguucucgcacuaaaaguggauaaugccgaaacuauuaagaaagaguuagguuuaagucucacugaaccguugauggagcaagucggaacggaagaguuuaucaaaagguucggugauggugcuucgcguguagugcucagccuucccuucgcugaggggaguucuagcguugaauauauuaauaacugggaacaggcgaaagcguuaagcguagaacuugagauuaauuuugaaacccguggaaaacguggccaagaugcgauguaugaguauauggcucaagccugugcaggaaaucgugucaggcgauaggccaggcacaagcagcauugguagc

SEQ ID NO: 67 - RNA, 21 nt, Artificial Sequence
  Synthetic
ggcacaagcagcauugguauu

SEQ ID NO: 68 - RNA, 610 nt, Artificial Sequence
  Synthetic
agcauugguagcaugggcgcugaugauguuguugauucuucuaaaucuuuugucauggaaaacuuuucuucguaccacgggacuaaaccugguuauguggauuccauucaaaaagguauacaaaagccaaaaucugguacacaaggaaauuaugacgaugauuggaaaggguuuuauaguaccgacaauaaauacgacgcugcgggauacucuguggauaaugaaaacccgcucucuggaaaagcuggaggcguggucaaagugacguauccaggacugacgaagguucucgcacuaaaaguggauaaugccgaaacuauuaagaaagaguuagguuuaagucucacugaaccguugauggagcaagucggaacggaagaguuuaucaaaagguucggugauggugcuucgcguguagugcucagccuucccuucgcugaggggaguucuagcguugaauauauuaauaacugggaacaggcgaaagcguuaagcguagaacuugagauuaauuuugaaacccguggaaaacguggccaagaugcgauguaugaguauauggcucaagccugugcaggaaaucgugucaggcgauaggccaggcacaagc

SEQ ID NO: 69 - RNA, 21 nt, Artificial Sequence
  Synthetic
aagcgccggccggccgcuggu

SEQ ID NO: 70 - RNA, 40 nt, Artificial Sequence
  Synthetic
ucccccuaagcgccggccggccgcuggucuguuuuuucgu

SEQ ID NO: 71 - RNA, 63 nt, Artificial Sequence
  Synthetic
gaaaaaacagaccagcggccgcagcggccggccggcgcuuaggccgcuggucuguuuuuucuu

SEQ ID NO: 72 - RNA, 21 nt, Artificial Sequence
  Synthetic
cagcggccggccggcgcuuag

SEQ ID NO: 73 - RNA, 21 nt, Artificial Sequence
  Synthetic
gaaaaaacagaccagcggccg

SEQ ID NO: 74 - RNA, 21 nt, Artificial Sequence
  Synthetic
gccgcuggucuguuuuuucuu

SEQ ID NO: 75 - RNA, 28 nt, Artificial Sequence
  Synthetic
ucccccuaagcgccggccggccgcuggu

SEQ ID NO: 76 - RNA, 21 nt, Artificial Sequence
  Synthetic
accagcggccggccggcgcuu

SEQ ID NO: 77 - RNA, 813 nt, Artificial Sequence
  Synthetic
augaccauggaaccauggaccagcggccggccggcgcuucaugggcgcugaugauguuguugauucuucuaaaucuuuugucauggaaaacuuuucuucguaccacgggacuaaaccugguuauguggauuccauucaaaaagguauacaaaagccaaaaucugguacacaaggaaauuaugacgaugauuggaaaggguuuuauaguaccgacaauaaauacgacgcugcgggauacucuguggauaaugaaaacccgcucucuggaaaagcuggaggcguggucaaagugacguauccaggacugacgaagguucucgcacuaaaaguggauaaugccgaaacuauuaagaaagaguuagguuuaagucucacugaaccguugauggagcaagucggaacggaagaguuuaucaaaagguucggugauggugcuucgcguguagugcucagccuucccuucgcugaggggaguucuagcguugaauauauuaauaacugggaacaggcgaaagcguuaagcguagaacuugagauuaauuuugaaacccguggaaaacguggccaagaugcgauguaugaguauauggcucaagccugugcaggaaaucgugucaggcgauaguuuuuuauunnnnnnnnnnnnnnnnnnnnnnnnnnnnnnnnnnnnnnnnnnnnnnnnnnnnnnnnnnnnnnnnnnnnnnnnnnnnnnnnnnuuuuauugaccagcggccggccggcgcuuaagaaccuggagcagaggcucuggcaagcuuuugcagcgaggaaaccgccaaggagccagagagcaug

SEQ ID NO: 78 - RNA, 706 nt, Artificial Sequence
  Synthetic
ccggcgaccagaugggcgcugaugauguuguugauucuucuaaaucuuuugucauggaaaacuuuucuucguaccacgggacuaaaccugguuauguggauuccauucaaaaagguauacaaaagccaaaaucugguacacaaggaaauuaugacgaugauuggaaaggguuuuauaguaccgacaauaaauacgacgcugcgggauacucuguggauaaugaaaacccgcucucuggaaaagcuggaggcguggucaaagugacguauccaggacugacgaagguucucgcacuaaaaguggauaaugccgaaacuauuaagaaagaguuagguuuaagucucacugaaccguugauggagcaagucggaacggaagaguuuaucaaaagguucggugauggugcuucgcguguagugcucagccuucccuucgcugaggggaguucuagcguugaauauauuaauaacugggaacaggcgaaagcguuaagcguagaacuugagauuaauuuugaaacccguggaaaacguggccaagaugcgauguaugaguauauggcucaagccugugcaggaaaucgugucaggcgauaguuuuuuauunnnnnnnnnnnnnnnnnnnnnnnnnnnnnnnnnnnnnnnnnnnnnnnnnnnnnnnnnnnnnnnnnnnnnnnnnnnnnnnnnnuuuuauuauucgcggccgg

SEQ ID NO: 79 - RNA, 21 nt, Artificial Sequence
  Synthetic
aauuaaguuuaugaacggguc

SEQ ID NO: 80 - RNA, 42 nt, Artificial Sequence
  Synthetic
cgggaccgguuaauuaaguuuaugaacgggucgauagccagc

SEQ ID NO: 81 - RNA, 36 nt, Artificial Sequence
  Synthetic
ggcuauccugaugaggccgaaaggccgaaacccguu

SEQ ID NO: 82 - RNA, 32 nt, Artificial Sequence
  Synthetic
cgggaccgguuaauuaaguuuaugaacggguc

SEQ ID NO: 83 - RNA, 27 nt, Artificial Sequence
  Synthetic
cccguucauaaacuuaauuaaccgguc

SEQ ID NO: 84 - RNA, 36 nt, Artificial Sequence
  Synthetic
gaacgggcugaugaggccgaaaggccgaaaccgguu

SEQ ID NO: 85 - RNA, 21 nt, Artificial Sequence
  Synthetic
cccguucauaaacuuaauuaa

SEQ ID NO: 86 - RNA, 21 nt, Artificial Sequence
  Synthetic
gacccguucauaaacuuaauu

SEQ ID NO: 87 - RNA, 657 nt, Artificial Sequence
  Synthetic
augaccauggaccauggucgacccguucauaaacuuaauuggcaaugggcgcugaugauguuguugauucuucuaaaucuuuugucauggaaaacuuuucuucguaccacgggacuaaaccugguuauguggauuccauucaaaaagguauacaaaagccaaaaucugguacacaaggaaauuaugacgaugauuggaaaggguuuuauaguaccgacaauaaauacgacgcugcgggauacucuguggauaaugaaaacccgcucucuggaaaagcuggaggcguggucaaagugacguauccaggacugacgaagguucucgcacuaaaaguggauaaugccgaaacuauuaagaaagaguuagguuuaagucucacugaaccguugauggagcaagucggaacggaagaguuuaucaaaagguucggugauggugcuucgcguguagugcucagccuucccuucgcugaggggaguucuagcguugaauauauuaauaacugggaacaggcgaaagcguuaagcguagaacuugagauuaauuuugaaacccguggaaaacguggccaagaugcgauguaugaguauauggcucaagccugugcaggaaaucgugucaggcgauagcccaaaggcucuuuucagagccccccua

SEQ ID NO: 88 - RNA, 627 nt, Artificial Sequence
  Synthetic
aaacuuaauuggcaaugggcgcugaugauguuguugauucuucuaaaucuuuugucauggaaaacuuuucuucguaccacgggacuaaaccugguuauguggauuccauucaaaaagguauacaaaagccaaaaucugguacacaaggaaauuaugacgaugauuggaaaggguuuuauaguaccgacaauaaauacgacgcugcgggauacucuguggauaaugaaaacccgcucucuggaaaagcuggaggcguggucaaagugacguauccaggacugacgaagguucucgcacuaaaaguggauaaugccgaaacuauuaagaaagaguuagguuuaagucucacugaaccguugauggagcaagucggaacggaagaguuuaucaaaagguucggugauggugcuucgcguguagugcucagccuucccuucgcugaggggaguucuagcguugaauauauuaauaacugggaacaggcgaaagcguuaagcguagaacuugagauuaauuuugaaacccguggaaaacguggccaagaugcgauguaugaguauauggcucaagccugugcaggaaaucgugucaggcgauagcccaaaggcucuuuucagagccccccua

SEQ ID NO: 89 - RNA, 23 nt, Artificial Sequence
  Synthetic
cugaugaggccgaaaggccgaaa

SEQ ID NO: 90 - RNA, 33 nt, Artificial Sequence
  Synthetic
accagagaaacacacguugugguauauuacugg

SEQ ID NO: 91 - RNA, 52 nt, Artificial Sequence
  Synthetic
ccugucaccggauguguuuuccggucugaugaguccgugaggacgaaacagg

SEQ ID NO: 92 - RNA, 97 nt, Artificial Sequence
  Synthetic
ccgcggcugagaugcagguacaucccacugaugagucccaaauaggacgaaacgcgcuucggugcgucugggauuccacugcuauccacgcggccgc

SEQ ID NO: 93 - RNA, 29 nt, Artificial Sequence
  Synthetic
ugggauuccacugcuauccacgcggccgc

SEQ ID NO: 94 - DNA, 21 bp, Artificial Sequence
  Synthetic RNA/DNA
aaacaugcagaaaaugcugtt

SEQ ID NO: 95 - DNA, 21 bp, Artificial Sequence
  Synthetic RNA/DNA
cagcauuuucugcauguuutt

SEQ ID NO: 96 - DNA, 23 bp, Artificial Sequence
  Synthetic RNA/DNA
aagcuaccuguuccauggccatt

SEQ ID NO: 97 - DNA, 23 bp, Artificial Sequence
  Synthetic RNA/DNA
tggccatggaacaggtagctttt

SEQ ID NO: 98 - RNA, 21 nt, Artificial Sequence
  Synthetic
gaguucccgacgcguccuagc

SEQ ID NO: 99 - RNA, 21 nt, Artificial Sequence
  Synthetic
uaggacgcgucgggaacucgc

SEQ ID NO: 100 - DNA, 21 bp, Artificial Sequence
  Synthetic
aactacacaaatcagcgattt

SEQ ID NO: 101 - RNA, 21 nt, Artificial Sequence
  Synthetic
cuacacaaaucagcgauuuuu

SEQ ID NO: 102 - RNA, 21 nt, Artificial Sequence
  Synthetic
aaaucgcugauuuguguaguu

SEQ ID NO: 103 - DNA, 26 bp, Artificial Sequence
  Synthetic
aaaacttacgctgagtacttcgatct

SEQ ID NO: 104 - DNA, 21 bp, Artificial Sequence
  Synthetic RNA/DNA
cuuacgcugaguacuucgatt

SEQ ID NO: 105 - DNA, 21 bp, Artificial Sequence
  Synthetic RNA/DNA
ucgaaguacucagcguaagtt

SEQ ID NO: 106 - DNA, 28 bp, Artificial Sequence
  Synthetic
ctacggcaagctgaccctgaagttcatc

SEQ ID NO: 107 - RNA, 22 nt, Artificial Sequence
  Synthetic
gcaagcugacccugaaguucau

SEQ ID NO: 108 - RNA, 22 nt, Artificial Sequence
  Synthetic
gaacuucagggucagcuugccg

SEQ ID NO: 109 - DNA, 24 bp, Artificial Sequence
  Synthetic
aatcgcttaccgattcagaatcgc

SEQ ID NO: 110 - DNA, 21 bp, Artificial Sequence
  Synthetic RNA/DNA
ucgcuuaccgauucagaautt

SEQ ID NO: 111 - DNA, 21 bp, Artificial Sequence
  Synthetic RNA/DNA
auucugaaucgguaagcgatt

SEQ ID NO: 112 - RNA, 7 nt, Artificial Sequence
  Synthetic
rnnaugg

SEQ ID NO: 113 - RNA, 47 nt, Artificial Sequence
  Synthetic
auuuauuuaauuauuuauuauuauuuaaauuauuuauauuauuuaau

SEQ ID NO: 114 - RNA, 4 nt, Artificial Sequence
  Synthetic
aaga

(19) United States
(12) Patent Application Publication
(10) Pub. No.: US 2013/0225660 A1
(43) Pub. Date: Aug. 29, 2013

(54) COMPOSITIONS AND METHODS FOR SPECIFIC CLEAVAGE OF EXOGENOUS RNA IN A CELL

(71) Applicant: Guy Abitbol, Rishon Lezion (IL)

(72) Inventor: Guy Abitbol, Rishon Lezion (IL)

(73) Assignee: NANODOC LTD., Rehovot (IL)

(21) Appl. No.: 13/881,356

(22) PCT Filed: Oct. 27, 2011
(86) PCT No.: PCT/IL11/00827

§ 371 Date: Apr. 24, 2013

(30) Foreign Application Priority Data
Oct. 28, 2010 (IL) PCT/IL2010/000895

(51) Int. Cl. C12N 15/113

(52) U.S. Cl. CPC C12N 15/113
USPC 514/44 A; 514/44 R

(57) ABSTRACT

There are provided compositions for cleaving an exogenous RNA of interest only in the presence of an endogenous signal RNA sequence, thereby activating expression of a polynucleotide of interest only in the presence of the endogenous signal RNA sequence. There are provided methods for the preparation of the composition and uses thereof in treatment and diagnosis of various conditions and disorders, for example by selectively activating expression of a toxin only in specific target cell populations.

### FIELD OF THE INVENTION

The present invention relates to compositions for cleaving an exogenous RNA of interest only in the presence of an endogenous signal RNA sequence, thereby activating expression of a polynucleotide of interest only in the presence of the endogenous signal RNA sequence. The invention further relates to uses of the compositions in treatment and diagnosis of various conditions and disorders, as exemplified by selectively activating expression of a toxin only in target cell populations.

### BACKGROUND OF THE INVENTION

RNA interference (RNAi) is a phenomenon in which dsRNA, composed of sense RNA and antisense RNA homologous to a certain region of a target gene effects cleavage of the homologous region of the target gene transcript, thereby inhibiting expression of the gene. In mammals, the dsRNA should be shorter than 31 base pairs to avoid induction of an interferon response that can cause cell death by apoptosis. RNAi technology is based on a natural mechanism that utilizes microRNAs (miRNAs) to regulate posttranscriptional gene expression [1]. miRNAs are very small RNA molecules of about 21 nucleotides in length that appear to be derived from 70-90 nucleotide precursors that form a predicted RNA stem-loop structure. miRNAs are expressed in organisms as diverse as nematodes, fruit flies, humans and plants.

In mammals, miRNAs are generally transcribed by RNA polymerase II and the resulting primary transcripts (pri-miRNAs) contain local stem-loop structures that are cleaved by the Drosha-DGCR8 complex. The product of this cleavage is one or more (in case of clusters) precursor miRNA (pre-miRNA). Pre-miRNAs are usually 70-90 nucleotides long with a strong stem-loop structure, and they usually contain 2 nucleotides overhang at the 3′ end [2]. The pre-miRNA is transported to the cytoplasm by Exportin-5. In the cytoplasm, the Dicer enzyme, which is an endoribonuclease of the RNase III family, recognizes the stem in the pre-miRNA as dsRNA and cleaves and releases a 21 bp dsRNA (miRNA duplex) from the 3′ and 5′ end of the pre-miRNA. The two strands of the duplex are separated from each other by the Dicer-TRBP complex and the strand that has thermodynamically weaker 5′ end is incorporated into the RNA induced silencing complex (RISC) [3]. This strand is the mature miRNA. The opposite strand, which is not incorporated into RISC is called miRNA* strand and it is degraded [1]. The mature miRNA guides RISC to a target site within mRNAs. If the target site is near perfect complementarity to the mature miRNA, the mRNA will be cleaved at a position that is located about 10 nucleotides upstream from the 3′ end of the target site [3]. After the cleavage, the RISC-mature miRNA strand complex is recycled for another round of activity [4]. If the target site has lower complementarity to the mature miRNA the mRNA will not be cleaved at the target site but the translation of the mRNA will be suppressed. Although about 530 miRNAs have been identified so far in humans, it is estimated that vertebrate genomes encode up to 1,000 unique miRNAs, which are predicted to regulate expression of at least 30% of the genes [5]. See FIG. 1.

The two portions of the mRNA cleaved by the RISC-mature miRNA strand complex in mammalian cells can be detected easily by Northern analysis [6]. Two RNA transcripts of about 23 nucleotides in length, which have a complementary region of about 19 nucleotides in length at the 5′ end, are hybridized with each other in the mammalian cell and are capable of directing target specific RNA interference [7]. Sequence and structural features of double stranded (ds)RNA molecules required to mediate target-specific nucleic acid modifications such as RNA-interference and/or DNA methylation are disclosed, for example, in U.S. Pat. No. 7,078,196 and U.S. Pat. No. 7,055,704. A dsRNA 52 nucleotides long that further comprises 20 nucleotides long ssRNA at one of the 3′ ends is a substrate for a Dicer only at the blunt end [8]. In mammals, Risc is coupled to Dicer [9]. While RNA polymerase III U6 promoter is a very strong promoter for transcribing small RNA (sRNA), RNA polymerase II CMV promoter is a strong promoter for transcribing protein-coding genes.

In mammalian cells, addition of a cap (7-methylguanosine cap) to the 5′ end of a mRNA, increases the translation of the mRNA by 35-50 fold. Further, addition of a poly(A) tail to the 3′ end of the mRNA increases the translation of the mRNA by 114-155-fold [10]. The poly(A) tail in mammalian cells increases the functional mRNA half-life by 2.6-fold and the cap increases the functional mRNA half-life by 1.7-fold [10]. The human HIST1H2AC (H2ac) gene encodes a member of the histone H2A family. Transcripts from this gene lack poly(A) tails but instead contain a palindromic termination element (5′-GGCUCUUUUCAGAGCC-3′) that forms a conserved stem-loop structure at the 3′-UTR, which plays an important role in mRNA processing and stability [11].

Ribosome inactivating proteins (RIPs) are protein toxins that are of plant or microbial origin. RIPs inhibit protein synthesis by inactivating ribosomes. Recent studies suggest that RIPs are also capable of inducing cell death by apoptosis. Type II RIPs contain a toxic A-chain and a lectin like subunit (B-chain) linked together by a disulfide bond. The B chain is catalytically inactive, but serves to mediate entry of the A-B protein complex into the cytosol. Ricin, Abrin and Diphtheria toxin are very potent Type II RIPs. It has been reported that a single molecule of Ricin or Abrin reaching the cytosol can kill the cell [12, 13]. In addition, a single molecule of Diphtheria toxin fragment A introduced into a cell can kill the cell [14].

According to the WHO (world health organization) in 2006 there were about 39.5 million people with HIV worldwide. Many viruses, including HIV exhibit a dormant or latent phase, during which little or no protein synthesis is conducted. The viral infection is essentially invisible to the immune system during such phases. Current antiviral treatment regimens are largely ineffective at eliminating cellular reservoirs of latent viruses [15]. Viruses may be oncogenic due to an oncogene in their genome. Retroviruses may also be oncogenic due to integration at a site which truncates a gene or which places a gene under control of the strong viral cis-acting regulatory element.

According to the American Cancer Society, 7.6 million people died from cancer in the world during 2007. The nature of and basic approaches to cancer treatment are constantly changing. Some approaches to cancer treatment, such as radiotherapy, surgery and inhibition of angiogenesis, are not useful against many small metastases. Other approaches to cancer treatment, such as inhibition of cell division and destroying dividing cells have no specificity and thus may cause harmful side effects that can even kill the patient. Further approaches for cancer treatment such as induction of differentiation of tumor tissues, inhibition of oncogenes, virus that contains ligands against membrane receptor protein that unique to cancer cells, manipulations of the immune system and immunotoxin therapy, have a narrow therapeutic index and usually are not sufficiently effective. Yet other approaches to cancer treatment using tumor suppressor genes and using toxins under a promoter that is uniquely activated in cancer cells have a narrow therapeutic index, a great potential for causing harmful side effects and usually are not sufficiently effective.

Many viruses that cause cancer are capable of causing latent infection. KSHV (Kaposi sarcoma-associated herpesvirus) causes Kaposi's sarcoma cancer; SV40 (Simian vacuolating virus 40) has the potential to cause tumors, but most often persists as a latent infection; and EBV (Epstein-Barr virus) causes Burkitt's lymphoma, nasopharyngeal carcinoma and EBV-associated gastric carcinomas.

On average, each tumor contains mutations in about 90 protein-coding genes [16]. Each tumor is initiated from a single founder cell [38]. It is most probable that at least one of these mutant genes is transcribed to mRNA. Therefore, it is highly probable that each cell of a specific tumor or each cell that is infected by a specific virus includes an RNA molecule, which comprises a specific RNA sequence (signal sequence) that is unique to the mutated or infected cell and that is not present in other normal cells of the same organism. The signal sequence can be from viral origin or from the mutated gene, that is unique to the specific tumor.

Various methods have been developed to identify any specific sequence that is unique to a specific tumor. These methods include, for example, DNA microarray, Tilling (Targeting Induced Local Lesions In Genomes) and large-scale sequencing of cancer genomes. Furthermore, the identification of this signal sequence is predicted to be even simpler thanks to the Cancer Genome Atlas (performed by the NIH), which was launched on Dec. 13, 2005 and has been cataloguing all the genetic mutations responsible for cancer.

Compositions for the selective killing of only those cells that contain a specific signal sequence, have been proposed. One approach, developed by Intronn Company, is to build an inactive Toxin that is activated by trans-splicing between the inactive Toxin to the signal sequence [17, 18 and 19]. However, this approach has several inherent problems: The first problem is that the RNA molecule that comprises the signal sequence must be present in the cell at very high copy number, since trans-splicing events are very rare. The second problem is that in most cases this approach is not suitable for a signal sequence that is of cancer origin, since in cancer, mutations spread over a short region. The third problem is that the trans-splicing events can also occur at random and may thus cause harmful side effects. The fourth problem is that the RNA molecule that comprises the signal sequence must include an intron at a very specific site. Another approach, which won the 2004 World Technology Award in Biotechnology, suggested using small dsDNA, ssDNA, hairpin DNA and restriction enzyme, however this approach can work only in cell extracts under very unique and not under physiological conditions in living cells [20]. Other approach, such as disclosed, for example, in WO 07/00068 are directed to a gene vector and comprising a miRNA sequence target and its use to prevent or reduce expression of transgene in a cell which comprises a corresponding miRNA. Also disclosed, for example, in WO 2010/055413, is a gene vector adapted for transient expression of a transgene in a peripheral organ cell comprising a regulatory sequence operably linked to a transgene wherein the regulatory sequence prevents or reduces expression of said transgene in hematopoietic lineage cells.

There is therefore a need for developing new compositions that are capable of selectively kill only cells that contain a signal sequence, wherein the compositions should be potent, reliable and specific as compared to compositions used in the prior are. Since that the development of these compositions can be a very complex multi-step process there is also a need for developing compositions for activating genes of interest in cells, only in the presence of a signal sequence, and for cleaving exogenous RNA of interest only in the presence of a signal sequence.

### SUMMARY OF THE INVENTION

The present invention provides compositions and methods for selectively cleaving an exogenous RNA of interest in response to the presence of an endogenous signal RNA in a cell. The exogenous RNA of interest is encoded by the composition. The endogenous signal RNA is an RNA molecule which comprises a predetermined signal sequence that is a sequence of 18-25 nucleotides long. Subsequent to specific cleavage of the exogenous RNA in the presence of the endogenous signal sequence transcription of a polynucleotide of interest may be activated. The polynucleotide activated may encode a toxin thereby providing means to kill target cell populations selectively.

The compositions of the invention comprise or, encode:
(a) an exogenous RNA of interest which is an RNA sequence that comprises a specific sequence that is of sufficient complementarity to the predetermined signal sequence.
(b) a functional RNA that is capable of effecting the cleavage of the endogenous signal RNA at the 5′ or 3′ end of the predetermined signal sequence; and
(c) a carrier RNA that is an RNA molecule that is capable of binding to the cleaved signal RNA portion that comprises the predetermined signal sequence at the predetermined signal sequence end, in such a way that the RNA duplex that formed is 14-31 nucleotides long and it comprises 3′ or 5′ overhang of 0-5 nucleotides, such that the RNA duplex is a substrate for a Dicer.

Thus, following introduction of the composition into a cell comprising the endogenous signal RNA, the functional RNA effects the cleavage of the endogenous signal RNA at the 5′ or 3′ end of the predetermined signal sequence and then the carrier RNA is hybridized to the cleaved signal RNA portion comprising the predetermined signal sequence at the predetermined signal sequence end and directs the processing of the predetermined signal sequence by Dicer and Risc and then the Risc-signal sequence complex directs the cleavage of the exogenous RNA of interest at a specific target/cleavage site.

The Dicer or Risc processing may involve additional proteins. In another embodiment of the invention, the carrier RNA may also be generated from a second exogenous RNA molecule. The predetermined signal sequence may be selected from, but is not limited to: a viral RNA sequence, and a sequence that is unique to neoplastic cells. The functional RNA may be selected from, but is not limited to: microRNA (miRNA), lariat-form RNA, short-hairpin RNA (shRNA), siRNA expression domain, ribozyme, or the like. In another embodiment of the invention, the composition of the invention may also comprise or encode an additional functional RNA that is capable of effecting the cleavage of the endogenous signal RNA at the opposite end of the predetermined signal sequence to that cleaved by the first functional RNA. In specific embodiments, the carrier RNA that is encoded by the composition may be driven by a polymerase I based promoter or polymerase III based promoter.

In one embodiment of the invention, the exogenous RNA of interest may further comprise:
(a) a sequence encoding an exogenous protein of interest; and
(b) an inhibitory sequence that is capable of inhibiting the expression of the exogenous protein of interest;
   such that the specific target/cleavage site is located between the inhibitory sequence and the sequence encoding the exogenous protein of interest, whereby, following introduction of the composition into a cell comprising the endogenous signal RNA, the exogenous RNA of interest may be transcribed and cleaved at the specific cleavage/target site so that the inhibitory sequence is detached from the sequence encoding the exogenous protein of interest and the exogenous protein of interest is capable of being expressed.

The exogenous protein of interest may be selected from, but is not limited to: the protein toxins Ricin, Abrin, Diphtheria toxin, fusion protein comprising protein toxins, and the like, or combinations thereof A single molecule of any one of these may be sufficient to kill the cell in which any of these molecules is expressed. The inhibitory sequence can be located downstream or upstream from the specific target/cleavage site. The inhibitory sequence that is located upstream from the specific target/cleavage site may be, but is not limited to a plurality of initiation codons, wherein each of the initiation codons is located within a Kozak consensus sequence, or any other translation initiation motif, wherein each of the initiation codons and the sequence encoding the protein of interest are not in the same reading frame. Thus these initiation codons will cause suppression of the expression of the protein of interest prior to cleavage.

In other embodiments, the predetermined signal sequence may be located at the 5′ or 3′ end of the endogenous signal RNA and the composition does not necessarily encode the functional RNA.

According to some embodiments, the components of the composition may be encoded by the same or different polynucleotide molecules. In some embodiments, one or more components of the composition may be on the same RNA molecule.

In additional specific embodiments, the present invention provides a composition for expressing an exogenous protein of interest only in the presence of an endogenous signal RNA in a cell, the exogenous protein of interest being encoded from the composition, the endogenous signal RNA being an RNA molecule which comprises a predetermined signal sequence, the predetermined signal sequence being a predetermined sequence that is at least 18 nucleotides in length and the composition comprising one or more polynucleotide molecules that comprise:
(a) one or more polynucleotide sequence(s) encoding a functional RNA that is capable of effecting the cleavage, directly or indirectly, of the endogenous signal RNA at a predetermined cleavage site, wherein the predetermined cleavage site is the 3′ end of the predetermined signal sequence; and
(b) a polynucleotide sequence encoding an exogenous RNA of interest molecule which consists essentially of:
   (1) a first sequence which is of sufficient complementarity to an edge sequence to hybridize therewith, the edge sequence being located 0-5 nucleotides upstream from the predetermined cleavage site and extending upstream in the signal RNA, wherein the first sequence comprises one or more initiation codon(s), wherein each of the initiation codons consists essentially of 5′-AUG-3′; and
   (2) a second sequence upstream to the first sequence, wherein the second sequence is a predetermined sequence that is 0-5 nucleotides in length; and
   (3) a third sequence downstream from the first sequence, wherein the third sequence is 0-7000 nucleotides in length; and
      wherein the exogenous RNA of interest molecule comprises a sequence encoding an exogenous protein of interest at least 21 nucleotides downstream from the 5′ end of said exogenous RNA of interest molecule; whereby following introduction of the composition into a cell comprising the endogenous signal RNA, the functional RNA effects the cleavage, directly or indirectly, of the endogenous signal RNA at the 3′ end of the predetermined signal sequence and thereby the exogenous RNA of interest molecule is hybridized to the edge sequence at the cleaved endogenous signal RNA and may direct the predetermined signal sequence to a Dicer processing that may cleave the exogenous RNA of interest molecule, whereby each of the initiation codon(s) is detached from the sequence encoding the exogenous protein of interest and the exogenous protein of interest is capable of being expressed.

In another embodiment, the edge sequence may be 25-30 nucleotides in length and may be located 2 nucleotides upstream from the predetermined cleavage site and extends upstream in the endogenous signal RNA, wherein the second sequences is 0 nucleotides in length. In yet another embodiment of the invention, each of the initiation codon(s) may be located 0-21 nucleotides downstream from the 5′ end of the exogenous RNA of interest molecule, such that each of the initiation codon(s) and the sequence encoding the exogenous protein of interest are not in the same reading frame. In further embodiment of the invention, at least one of the initiation codon(s) may be located within a Kozak consensus sequence or any other translation initiation motif/element. The functional RNA may be selected from, but is not limited to: microRNA (miRNA), short-hairpin RNA (shRNA), small-interfering RNA (siRNA) and/or ribozyme. The exogenous protein of interest may be, for example, but is not limited to Diphtheria toxin A chain, RIP protein, and any other protein toxin.

According to some embodiments, the compositions of the invention may be used in various methods and applications, such as, for example, but not limited to: regulation of gene expression, targeted cell death, treatment of a disease or a condition including, for example, proliferative disorders (such as cancer), infectious diseases, and the like, diagnosis of a disease or a condition, formation of transgenic organisms, suicide gene therapy, and the like.

According to some embodiments, there is provided a composition comprising one or more polynucleotides for directing specific cleavage of an exogenous RNA of interest at a specific target site, the cleavage taking place only in the presence of an endogenous signal RNA in a cell, the endogenous signal RNA being an RNA molecule which comprises a signal sequence, the signal sequence being any predetermined sequence of from 18 to 25 nucleotides in length, whereby introduction of said composition into a cell comprising said endogenous signal RNA, directs the cleavage of said exogenous RNA of interest at the specific target site that is located within a specific sequence, which is of sufficient complementarity to hybridize with the predetermined signal sequence.

In some embodiments, the one or more polynucleotides may comprise a first polynucleotide sequence encoding said exogenous RNA of interest; a second polynucleotide sequence encoding a functional RNA capable of mediating the cleavage of the endogenous signal RNA at a predetermined cleavage site; and a third polynucleotide sequence encoding a carrier RNA.

In some embodiments, the carrier RNA is an RNA molecule that is at least about 18 nucleotides in length and is consisting essentially of: a first sequence of from 14 to 31 nucleotides in length which is of sufficient complementarity to an edge sequence to hybridize therewith, said edge sequence is 14-31 nucleotides in length and is located 0-5 nucleotides downstream from said predetermined cleavage site and extends downstream in said endogenous signal RNA; a second sequence downstream from said first sequence, wherein said second sequence is a random sequence that is 0-5 nucleotides in length; a third sequence upstream from said first sequence, wherein said third sequence is 0-7000 nucleotides in length; and the predetermined cleavage site is the 5′ end of said predetermined signal sequence. In some embodiments, the edge sequence is 23-28 nucleotides in length and is located starting from the predetermined cleavage site to about 23-28 nucleotides downstream, wherein the second sequence is 2 nucleotides in length and wherein said third sequence is 0 nucleotides in length.

In some embodiments, the carrier RNA is an RNA molecule that is at least about 18 nucleotides in length and is consisting essentially of: a first sequence of from 14 to 31 nucleotides in length which is of sufficient complementarity to an edge sequence to hybridize therewith, said edge sequence is 14-31 nucleotides in length and is located 0-5 nucleotides upstream from said predetermined cleavage site and extends upstream in said endogenous signal RNA; a second sequence upstream from the first sequence, wherein said second sequence is a random sequence that is 0-5 nucleotides in length; a third sequence downstream from the first sequence, wherein said third sequence is 0-7000 nucleotides in length; and the predetermined cleavage site is the 3′ end of said predetermined signal sequence. In some embodiments, the edge sequence is 25-30 nucleotides in length and is located 2 nucleotides upstream from the predetermined cleavage site and extends upstream in said endogenous signal RNA, wherein said second sequence is 0 nucleotides in length and wherein said third sequence is 0 nucleotides in length.

In some embodiments, the carrier RNA may be processed from a polynucleotide sequence comprising a carrier sequence that is at least about 18 nucleotides in length, said carrier sequence consisting essentially of: a first sequence of from 14 to 31 nucleotides in length which is of sufficient complementarity to an edge sequence to hybridize therewith, said edge sequence is 14-31 nucleotides in length and is located 0-5 nucleotides downstream from said predetermined cleavage site and extends downstream in said endogenous signal RNA; a second sequence downstream from said first sequence, wherein said second sequence is a random sequence that is 0-5 nucleotides in length; and a third sequence upstream from said first sequence, wherein said third sequence is 0-7000 nucleotides in length; wherein the polynucleotide sequence is cleaved within the cell at a carrier cleavage site that is a 3′ end of said carrier sequence; wherein the cleavage at the carrier cleavage site is effected by a functional nucleic acid which is encoded by a fourth polynucleotide sequence of the composition; and wherein the predetermined cleavage site is the 5′ end of said predetermined signal sequence.

In additional embodiments, the carrier RNA may be processed from a polynucleotide sequence comprising a carrier sequence that is at least about 18 nucleotides in length, said carrier sequence consisting essentially of: a first sequence of from 14 to 31 nucleotides in length which is of sufficient complementarity to an edge sequence to hybridize therewith, said edge sequence is 14-31 nucleotides in length and is located 0-5 nucleotides upstream from said predetermined cleavage site and extends upstream in said endogenous signal RNA; a second sequence upstream from the first sequence, wherein said second sequence is a random sequence that is 0-5 nucleotides in length; and a third sequence downstream from said first sequence, wherein said third sequence is 0-7000 nucleotides in length; wherein the polynucleotide sequence is cleaved within the cell at a carrier cleavage site that is 5′ end of said carrier sequence; the cleavage at the carrier cleavage site is effected by a functional nucleic acid which is encoded by a fourth polynucleotide sequence of the composition; and the predetermined cleavage site is the 3′ end of said predetermined signal sequence.

According to some embodiments, the endogenous signal RNA is a cellular mRNA, viral RNA, or both. In further embodiments, the predetermined signal sequence is unique to neoplastic cells, viral infected cells, or both.

According to some embodiments, sufficient complementarity is at least 30% complementarity. In further embodiments, sufficient complementarity is at least 90%.

According to some embodiments, the one or more polynucleotide may comprise one or more DNA molecules, one or more RNA molecules or combinations thereof.

In some embodiments, the functional RNA may be selected from the group consisting of: microRNA (miRNA), lariat-form RNA, short-hairpin RNA (shRNA), siRNA expression domain, antisense RNA, double-stranded RNA (dsRNA), small-interfering RNA (siRNA) and ribozyme.

According to further embodiments, the exogenous RNA of interest may further comprise a sequence encoding an exogenous protein of interest; and an inhibitory sequence that is capable of inhibiting the expression of the exogenous protein of interest; wherein the specific target site is located between the inhibitory sequence and the sequence encoding the exogenous protein of interest, whereby following introduction of said composition into a cell comprising the endogenous signal RNA, the exogenous RNA of interest is transcribed and cleaved at the specific target site, whereby the inhibitory sequence is detached from the sequence encoding the exogenous protein of interest and the exogenous protein of interest is capable of being expressed.

In some embodiments, the exogenous protein of interest is a toxin. In some embodiments, the exogenous protein of interest is selected from the group consisting of: Ricin, Ricin A chain, Abrin, Abrin A chain, Diphtheria toxin A chain and modified forms thereof. In further embodiments, the exogenous protein of interest is selected from the group consisting of: alpha toxin, saporin, maize RIP, barley RIP, wheat RIP, corn RIP, rye RIP, flax RIP, Shiga toxin, Shiga-like RIP, momordin, thymidine kinase, pokeweed antiviral protein, gelonin, Pseudomonas exotoxin, Pseudomonas exotoxin A, Escherichia coli cytosine deaminase and modified forms thereof.

According to additional embodiments, the inhibitory sequence in the exogenous RNA of interest sequence is located upstream from the specific target site. In some embodiments, the inhibitory sequence comprise one or more initiation codons, wherein each of the initiation codons and the sequence encoding the exogenous protein of interest are not in the same reading frame, and wherein said inhibitory sequence, directly or indirectly, reduces the efficiency of translation of said exogenous protein of interest. In some embodiments the one or more initiation codons is consisting essentially of 5′-AUG-3′

In further embodiments, the exogenous RNA of interest may further comprise a stop codon that is located between the initiation codon and the start codon of the sequence encoding the exogenous protein of interest, wherein the stop codon and the initiation codon are in the same reading frame. The stop codon may be selected from the group consisting of: 5′-UAA-3′,5′-UAG-3′ and 5′-UGA-3′.

According to additional embodiments, the inhibitory sequence may further comprise a nucleotide sequence downstream from the initiation codon, wherein said nucleotide sequence and said initiation codon are in the same reading frame, and wherein the nucleotide sequence encodes a sorting signal for subcellular localization. The subcellular localization may be selected from the group consisting of: mitochondria, nucleus, endosome, lysosome, peroxisome and endoplastic reticulum (ER).

According to further embodiments, the inhibitory sequence may further comprise a nucleotide sequence downstream from the initiation codon, wherein the nucleotide sequence and the initiation codon are in the same reading frame; and wherein said nucleotide sequence encodes a protein degradation signal.

According to additional embodiments, the inhibitory sequence may further comprise a nucleotide sequence downstream from the initiation codon, wherein the nucleotide sequence and the initiation codon are in the same reading frame; wherein said nucleotide sequence and said sequence encoding the exogenous protein of interest are in the same reading frame; and wherein said nucleotide sequence encodes an amino acid sequence; whereby when the amino acid sequence is fused to the exogenous protein of interest the biological function of the exogenous protein of interest is inhibited.

According to further embodiments, the RNA of interest may further comprise a stop codon downstream from the initiation codon, wherein the stop codon and the initiation codon are in the same reading frame and wherein the exogenous RNA of interest further comprises an intron downstream from the stop codon, whereby the exogenous RNA of interest is a target for nonsense-mediated decay (NMD).

In further embodiments, the inhibitory sequence may be located downstream from the sequence encoding the exogenous protein of interest and the inhibitory sequence comprises an RNA localization signal for subcellular localization or an endogenous miRNA binding site.

In some embodiments, the inhibitory sequence may be located upstream from the sequence encoding the exogenous protein of interest, wherein the inhibitory sequence is capable of forming a secondary structure, having a folding free energy of lower than −30 kcal/mol, whereby said secondary structure is sufficient to block scanning ribosomes from reaching the start codon of said exogenous protein of interest.

In some embodiments, the exogenous RNA of interest may further comprise an internal ribosome entry site (IRES) sequence downstream from the specific cleavage site and upstream from the sequence encoding the exogenous protein of interest, wherein the IRES sequence is more functional within the cleaved exogenous RNA of interest than within the intact exogenous RNA of interest.

In some embodiments, the exogenous RNA of interest may comprise a nucleotide sequence immediately upstream from the sequence encoding the exogenous protein of interest, wherein the nucleotide sequence comprises an internal ribosome entry site (IRES) sequence, which increases the efficiency of translation of said exogenous protein of interest in the cleaved exogenous RNA of interest.

In some embodiments, the RNA of interest may further comprise a nucleotide sequence comprising a cytoplasmic polyadenylation element, located immediately downstream from said sequence encoding the exogenous protein of interest, wherein said cytoplasmic polyadenylation element increases the efficiency of translation of said exogenous protein of interest in the cleaved exogenous RNA of interest.

According to some embodiments, the composition may further comprise an additional polynucleotide sequence that encodes for an additional RNA molecule, said additional RNA molecule comprises at the 3′ end a nucleotide sequence that is capable of binding to a sequence that is located upstream of said specific target site and downstream from the sequence encoding the exogenous protein of interest, wherein the additional RNA molecule, directly or indirectly, increases the efficiency of translation of said exogenous protein of interest in the cleaved exogenous RNA of interest.

According to some embodiments, the composition may further comprise an additional polynucleotide sequence that encodes a cleaving component that is capable of effecting the cleavage, of said exogenous RNA of interest at a position that is located upstream from the inhibitory sequence, wherein said cleaving component(s) is selected from the group consisting of: a) a nucleic acid sequence that is located within said exogenous RNA of interest, wherein said nucleic acid sequence is selected from the group consisting of: a) endonuclease recognition site, endogenous miRNA binding site, cis acting ribozyme and miRNA sequence, wherein said nucleic acid sequence, directly or indirectly, reduces the efficiency of translation of said exogenous protein of interest in the exogenous RNA of interest; and b) an inhibitory RNA, wherein said inhibitory RNA is selected from the group consisting of: microRNA (miRNA), lariat-form RNA, short-hairpin RNA (shRNA), siRNA expression domain, antisense RNA, double-stranded RNA (dsRNA), small-interfering RNA (siRNA) and ribozyme; wherein said inhibitory RNA, directly or indirectly, reduces the efficiency of translation of said exogenous protein of interest in said exogenous RNA of interest.

In further embodiments, the specific sequence is a plurality of specific sequences and the specific target site is a plurality of specific target sites.

In some embodiments, the exogenous RNA of interest and the functional RNA are capable of being located on the same or different polynucleotide molecules. In some embodiments, the exogenous RNA of interest, the functional RNA and the functional nucleic acid are capable of being located on one or more polynucleotide molecules.

According to further embodiments, the one or more polynucleotides of the composition may be integrated into the cell genome.

In some embodiments, the cell may be selected from a group consisting of: human cell, animal cell, cultured cell and plant cell. In some embodiments, the cell may be present in an organism.

According to some embodiments, there is further provided a composition comprising one or more polynucleotides for directing specific expression of an exogenous protein of interest in a cell, wherein the exogenous protein of interest is expressed only in the presence of an endogenous signal RNA in a cell, the endogenous signal RNA being an RNA molecule which comprises a signal sequence, the signal sequence being any predetermined sequence of from 18 to 25 nucleotides in length, whereby introduction of said composition into a cell comprising said endogenous signal RNA directs the cleavage of an exogenous RNA of interest at a specific target site that is located within a specific sequence, which is of sufficient complementarity to hybridize with the predetermined signal sequence, wherein only after the cleavage of said exogenous RNA of interest in the cell, the exogenous protein of interest, which is encoded by said cleaved exogenous RNA of interest is capable of being expressed in the cell. In some embodiments, the one or more polynucleotides includes a first polynucleotide sequence encoding said exogenous RNA of interest; a second polynucleotide sequence encoding a functional RNA capable of mediating the cleavage of the endogenous signal RNA at a predetermined cleavage site; and a third polynucleotide sequence encoding a carrier RNA.

According to some embodiments, there is provided a method for killing a specific cell population, which comprises an endogenous signal RNA, the method comprises: introducing the cell with a composition comprising one or more polynucleotides for directing specific cleavage of an exogenous RNA of interest at a specific target site that is located within a specific sequence, which is of sufficient complementarity to hybridize with the endogenous signal RNA, the endogenous signal RNA being an RNA molecule which comprises a signal sequence, the signal sequence being any predetermined sequence of from 18 to 25 nucleotides in length; and wherein the cleavage of the exogenous RNA of interest in the cell, enables the expression of an exogenous protein of interest, capable of killing the cell population. The one or more polynucleotides comprises: a first polynucleotide sequence encoding said exogenous RNA of interest; a second polynucleotide sequence encoding a functional RNA capable of mediating the cleavage of the endogenous signal RNA at a predetermined cleavage site; and a third polynucleotide sequence encoding a carrier RNA. In some embodiments, the endogenous signal RNA is a cellular mRNA, viral RNA, or both. The cell population may be is selected from a group consisting of: human cell, animal cell, cultured cell and plant cell. In some embodiments, the cell population is a neoplastic cell population. In some embodiments, the cell population is present in an organism.

Objects and advantages of the present invention will be clear from the description that follows.

### BRIEF DESCRIPTION OF THE FIGURES

The following figures are offered by way of illustration and not by way of limitation.

FIG. 1 is a general scheme of a model for biogenesis and activity of microRNAs (miRNAs) in a cell.

FIG. 2 is a schematic drawing showing an example for cleaving exogenous RNA of interest in response to the presence of an endogenous signal RNA in a cell, according to some embodiments. In this exemplary embodiment, the composition encodes for: a carrier RNA of 27 nucleotides; an exogenous RNA of interest that comprises a specific sequence which is complementary to a predetermined signal sequence of the endogenous signal RNA; and a functional RNA which is shRNA that is capable of effecting the cleavage of the endogenous signal RNA at the 5′ end of the predetermined signal sequence.

FIG. 3 is a schematic drawing showing an example for cleaving exogenous RNA of interest in response to the presence of an endogenous signal RNA in a cell, according to some embodiments. In this example, the composition of the invention encodes for: a carrier RNA of 27 nucleotides, an exogenous RNA of interest that comprises a specific sequence which is complementary to the predetermined signal sequence of the endogenous signal RNA; and a functional RNA which is shRNA that is capable of effecting the cleavage of the endogenous signal RNA at the 3′ end of the predetermined signal sequence.

FIG. 4 is a schematic drawing showing an example for cleaving exogenous RNA of interest in response to the presence of an endogenous signal RNA in a cell, according to some embodiments. In this example, the composition of the invention encodes for: an exogenous RNA of interest that comprises a specific sequence which is complementary to the predetermined signal sequence of the endogenous signal RNA, a functional RNA which is shRNA that is capable of effecting the cleavage of the endogenous signal RNA at the 5′ end of the predetermined signal sequence, a carrier sequence that is of 27 nucleotides long and a functional nucleic acid which is cis acting ribozyme that is capable of effecting the cleavage of the carrier RNA at the 3′ end of the carrier sequence.

FIG. 5 is a schematic drawing showing an example for cleaving exogenous RNA of interest in response to the presence of an endogenous signal RNA in a cell, according to some embodiments. In this example, the composition of the invention encodes for: an exogenous RNA of interest that comprises a specific sequence which is complementary to the predetermined signal sequence of the endogenous signal RNA, a functional RNA which is shRNA that is capable of effecting the cleavage of the endogenous signal RNA at the 3′ end of the predetermined signal sequence, a carrier sequence that is of 27 nucleotides long and a functional nucleic acid which is cis acting ribozyme that is capable of effecting the cleavage of the carrier RNA at the 5′ end of the carrier sequence.

FIG. 6A is a schematic drawing showing an example for inhibitory RNA that is capable of effecting the cleavage of the endogenous signal RNA at the 5′ end of the predetermined signal sequence, according to some embodiments.

FIG. 6B is a schematic drawing showing an example for inhibitory RNA that is capable of effecting the cleavage of the endogenous signal RNA at the 3′ end of the predetermined signal sequence, according to some embodiments.

FIG. 7A is a schematic drawing showing an example for inhibitory RNA that is capable of effecting the cleavage of the carrier RNA at the 3′ end of the carrier sequence, according to some embodiments.

FIG. 7B is a schematic drawing showing an example for inhibitory RNA that is capable of effecting the cleavage of the carrier RNA at the 5′ end of the carrier sequence, according to some embodiments.

FIG. 8A is a schematic drawing showing an example for inhibitory RNA which, according to some embodiments, is an RNA duplex that may be a substrate for Dicer.

FIG. 8B is a schematic drawing showing an example for inhibitory RNA which, according to some embodiments, is an RNA duplex that may be a substrate for Dicer.

FIG. 9A is a schematic drawing showing an example, according to some embodiments, for hammerhead-type ribozyme (SEQ ID NO. 89) that is capable of effecting the cleavage of the endogenous signal RNA or the carrier RNA at the predetermined cleavage site or at the carrier cleavage site of, respectively.

FIG. 9B is a schematic drawing showing an example, according to some embodiments, for hairpin-type ribozyme that is capable of effecting the cleavage of the endogenous signal RNA or the carrier RNA at the predetermined cleavage site or at the carrier cleavage site, respectively. The exemplary hairpin-type ribozyme is composed of SEQ ID NO. 90, preceded by a sequence complementary to the target RNA, the tetra-nucleotide AAGA (SEQ ID NO. 114) and an additional sequence complementary to the target RNA (at the 5′ end of the ribozyme).

FIG. 10 is a schematic drawing showing an example, according to some embodiments, of a functional nucleic acid that is the very efficient cis-acting hammerhead ribozyme-snorbozyme (SEQ ID NO. 91) [22], which is capable of effecting the cleavage of the carrier RNA at the 3′ end of the carrier sequence.

FIG. 11 is a schematic drawing showing an example, according to some embodiments, of a functional nucleic acid that is the very efficient cis-acting hammerhead ribozyme—N117 (SEQ ID NO. 92) [23] which is capable of effecting the cleavage of the carrier RNA (SEQ ID NO. 93) at the 5′ end of the carrier sequence.

FIG. 12A is a schematic drawing showing an example, according to some embodiments, of a functional nucleic acid that is an endonuclease recognition site or an endogenous miRNA binding site, such that the functional nucleic acid is capable of effecting the cleavage of the carrier RNA at the 3′ end of the carrier sequence.

FIG. 12B is a schematic drawing showing an example, according to some embodiments, of a functional nucleic acid that is an endonuclease recognition site or an endogenous miRNA binding site, such that the functional nucleic acid is capable of effecting the cleavage of the carrier RNA at the 5′ end of the carrier sequence.

FIG. 12C is a schematic drawing showing an example, according to some embodiments, of a functional nucleic acid that is a miRNA sequence, such that the miRNA sequence is capable of effecting the cleavage of the carrier RNA at the 3′ end of the carrier sequence.

FIG. 12D is a schematic drawing showing an example, according to some embodiments, of a functional nucleic acid that is a miRNA sequence, such that the miRNA sequence is capable of affecting the cleavage of the carrier RNA at the 5′ end of the carrier sequence.

FIG. 13A is a schematic drawing showing an example, according to some embodiments, of a functional nucleic acid which is capable of forming stem loop structure with the carrier sequence, such that the stem loop structure is capable of effecting the cleavage of the carrier RNA at the 3′ end of the carrier sequence.

FIG. 13B is a schematic drawing showing an example, according to some embodiments, for functional nucleic acid which is capable of forming stem loop structure with the carrier sequence, such that the stem loop structure is capable of effecting the cleavage of the carrier RNA at the 5′ end of the carrier sequence.

FIG. 14A is a schematic drawing showing an example, according to some embodiments, for functional nucleic acid that has a stem loop structure, such that the loop comprises the carrier sequence and such that when the stem loop structure is processed by Drosha and Dicer, the carrier sequence is detached from the stem loop structure and the siRNA duplex thus formed is the functional RNA, which is then capable of effecting the cleavage of the endogenous signal RNA at the predetermined cleavage site.

FIG. 14B is a schematic drawing showing an example, according to some embodiments, of a functional nucleic acid that has a stem loop structure, such that the loop comprises the carrier sequence and such that the expression of the stem loop structure is driven by polymerase I or III based promoter and such that when the stem loop structure is processed by Dicer the carrier sequence is detached from the stem loop structure and the siRNA duplex thus formed is the functional RNA which is capable of effecting the cleavage of the endogenous signal RNA at the predetermined cleavage site.

FIG. 15A is a schematic drawing showing an example, according to some embodiments, of a carrier sequence that is located in the same RNA duplex with the functional RNA, such that the double strand region is located downstream from the carrier sequence and such that when the double strand region is processed by Dicer, the carrier sequence is detached from the RNA duplex and the siRNA duplex thus formed is the functional RNA ans is capable of effecting the cleavage of the endogenous signal RNA at the predetermined cleavage site.

FIG. 15B is a schematic drawing showing an example, according to some embodiments, for a carrier sequence that is located in the same RNA duplex with the functional RNA, such that the double strand region is located upstream from the carrier sequence and such that when the double strand region is processed by Dicer the carrier sequence is detached from the RNA duplex and the siRNA duplex thus formed is the functional RNA which is capable of effecting the cleavage of the endogenous signal RNA at the predetermined cleavage site.

FIG. 16A is a schematic drawing showing an example, according to some embodiments, for a carrier RNA that is located in the same RNA duplex with the functional RNA, such that the double strand region is located at the 5′ end of the carrier RNA and such that when the double strand region is processed by Dicer, the sequence that is located at the 3′ end of the carrier RNA is detached from the RNA duplex and the siRNA duplex thus formed is the functional RNA, which is capable of effecting the cleavage of the endogenous signal RNA at the predetermined cleavage site.

FIG. 16B is a schematic drawing showing an example, according to some embodiments, of a carrier RNA that is located in the same RNA duplex with the functional RNA, such that the double strand region is located at the 3′ end of the carrier RNA and such that when the double strand region is processed by Dicer, the sequence that is located at the 5′ end of the carrier RNA is detached from the RNA duplex and the siRNA duplex thus formed is the functional RNA, which is capable of effecting the cleavage of the endogenous signal RNA at the predetermined cleavage site.

FIG. 17A is a schematic drawing showing an example, according to some embodiments, for the carrier sequence that is located in the same RNA duplex with the functional RNA, such that the double strand region is located upstream from the carrier sequence and such that when the double strand region is processed by Dicer, the siRNA duplex that is formed is the functional RNA, which is capable of effecting the cleavage of the endogenous signal RNA at the predetermined cleavage site.

FIG. 17B is a schematic drawing showing an example, according to some embodiments, of a carrier sequence that is located in the same RNA duplex with the functional RNA, such that the double strand region is located downstream from the carrier sequence and such that when the double strand region is processed by Dicer, the siRNA duplex that is formed is the functional RNA, which is capable of effecting the cleavage of the endogenous signal RNA at the predetermined cleavage site.

FIG. 18A is a schematic drawing showing an example, according to some embodiments, of a carrier sequence that is located in the same RNA duplex with the functional nucleic acid, such that the double strand region is located upstream from the carrier sequence and such that when the double strand region is processed by Dicer, the siRNA duplex that is formed is the functional nucleic acid which is capable of effecting the cleavage of the carrier RNA at the carrier cleavage site.

FIG. 18B is a schematic drawing showing an example, according to some embodiments, for carrier sequence that is located in the same RNA duplex with the functional nucleic acid, such that the double strand region is located downstream from the carrier sequence and such that when the double strand region is processed by Dicer, the siRNA duplex that is formed is the functional nucleic acid, which and is capable of effecting the cleavage of the carrier RNA at the carrier cleavage site.

FIG. 19A is a schematic drawing illustrating an example, according to some embodiments, of a carrier sequence that is located in the same RNA duplex with the functional nucleic acid and with the functional RNA, such that the double strand region is located upstream from the carrier sequence, and such that when the double strand region is processed by Dicer, the siRNA duplexes that are formed are the functional nucleic acid and the functional RNA.

FIG. 19B is a schematic drawing illustrating an example, according to some embodiments, of a carrier sequence that is located in the same RNA duplex with the functional nucleic acid and with the functional RNA, such that the double strand region is located downstream from the carrier sequence and such that when the double strand region is processed by Dicer the siRNA duplexes that are formed are the functional nucleic acid and the functional RNA.

FIG. 20A is a schematic drawing showing an example, according to some embodiments, of a carrier RNA that comprises 3 contiguous carrier sequences downstream from the carrier sequence, such that the functional nucleic acid is capable of effecting the cleavage of the carrier RNA at the 3′ end of the carrier sequence.

FIG. 20B is a schematic drawing showing an example, according to some embodiments, for carrier RNA that comprises 3 contiguous carrier sequences upstream from the carrier sequence, such that the functional nucleic acid is capable of effecting the cleavage of the carrier RNA at the 5′ end of the carrier sequence.

FIG. 21A is a schematic drawing showing an example, according to some embodiments, for polynucleotide molecule(s) of the composition that, in addition to the functional RNA that cleaves the 5′ end of the predetermined signal sequence, further transcribes an additional functional RNA that cleaves the 3′ end of the predetermined signal sequence.

FIG. 21B is a schematic drawing showing an example, according to some embodiments, for polynucleotide molecule(s) of the composition that, in addition to the functional RNA that cleaves the 3′ end of the predetermined signal sequence, further transcribes an additional functional RNA that cleaves the 5′ end of the predetermined signal sequence.

FIG. 22A is a schematic drawing showing an example, according to some embodiments, of the schematic structure of an exogenous RNA of interest that is activated by its cleavage, such that the specific sequence is located downstream from the inhibitory sequence and upstream from the sequence encoding the exogenous protein of interest.

FIG. 22B is a schematic drawing showing an example, according to some embodiments, of the schematic structure of an exogenous RNA of interest that is activated by its cleavage, such that the specific sequence is located upstream from the inhibitory sequence and downstream from the sequence encoding the exogenous protein of interest.

FIG. 23A is a schematic drawing showing an example, according to some embodiments, for inhibitory sequence that is located upstream from the specific target/cleavage site of the exogenous RNA of interest, and comprises an AUG that is not in the same reading frame with the sequence encoding exogenous protein of interest.

FIG. 23B is a schematic drawing showing an example, according to some embodiments, for inhibitory sequence that is located upstream from the specific target/cleavage site of the exogenous RNA of interest, and comprises a Kozak consensus sequence (5′-ACCAUGG-3′—SEQ ID NO. 25) that is not in the same reading frame with the sequence encoding exogenous protein of interest.

FIG. 23C is a schematic drawing showing an example, according to some embodiments, for inhibitory sequence that is located upstream from the specific target/cleavage site of the exogenous RNA of interest, and comprises 2 Kozak consensus sequences that are not in the same reading frame with the sequence encoding exogenous protein of interest.

FIG. 24A is a schematic drawing showing an example, according to some embodiments, for inhibitory sequence that is located upstream from the specific target/cleavage site of the exogenous RNA of interest, and comprises an AUG and a downstream stop codon that are in the same reading frame.

FIG. 24B is a schematic drawing showing an example, according to some embodiments, for inhibitory sequence that is located upstream from the specific target/cleavage site of the exogenous RNA of interest, and comprises an AUG and a downstream: sorting signal for subcellular localization or protein degradation signal.

FIG. 24C is a schematic drawing showing an example, according to some embodiments, for inhibitory sequence that is located upstream from the specific target/cleavage site of the exogenous RNA of interest, and comprises an AUG and a downstream sequence encoding amino acids that are capable of inhibiting the biological function of the downstream protein of interest.

FIG. 24D is a schematic drawing showing an example, according to some embodiments, for inhibitory sequence that is located upstream from the specific target/cleavage site of the exogenous RNA of interest, and comprises an AUG, a downstream stop codon that is in the same reading frame with the AUG and a downstream intron, such that the exogenous RNA of interest is a target for nonsense-mediated decay (NMD).

FIG. 25A is a schematic drawing showing an example, according to some embodiments, for inhibitory sequence that is located upstream from the specific target/cleavage site of the exogenous RNA of interest and comprises a binding site for translation repressor.

FIG. 25B is a schematic drawing showing an example, according to some embodiments, for inhibitory sequence that is located upstream from the specific target/cleavage site of the exogenous RNA of interest and comprises an RNA localization signal for subcellular localization.

FIG. 25C is a schematic drawing showing an example, according to some embodiments, for inhibitory sequence that is located upstream from the specific target/cleavage site of the exogenous RNA of interest and comprises an RNA destabilizing element that is an AU-rich element or an endonuclease recognition site.

FIG. 25D is a schematic drawing showing an example, according to some embodiments, for inhibitory sequence that is located upstream from the specific target/cleavage site of the exogenous RNA of interest, and comprises a secondary structure.

FIG. 26 is a schematic drawing showing an example, according to some embodiments, for the activation of the exogenous RNA of interest by its cleavage, such that the inhibitory sequence creates a secondary structure that blocks translation and such that the cleavage creates an IRES (Internal ribosome entry site).

FIG. 27A is a schematic drawing showing an example, according to some embodiments, of additional structure that may increase the efficiency of translation of the exogenous RNA of interest, that is cleaved at the 5′ end, wherein the additional structure is an IRES (Internal ribosome entry site).

FIG. 27B is a schematic drawing showing an example, according to some embodiments, of additional structure that may increase the efficiency of translation of the exogenous RNA of interest that is cleaved at the 5′ end, wherein the additional structure is a stem loop structure.

FIG. 27C is a schematic drawing showing an example, according to some embodiments, of additional structure that may increase the efficiency of translation of the exogenous RNA of interest, that is cleaved at the 5′ end, wherein the additional structure is a cytoplasmic polyadenylation element.

FIG. 27D is a schematic drawing showing an example, according to some embodiments, of additional structures that may increase the efficiency of translation of the exogenous RNA of interest, which is cleaved at the 5′ end, wherein the additional structures are nucleotide sequences that are capable of binding to each other and by this force the exogenous RNA of interest to form a circular structure, particularly when the exogenous RNA of interest is cleaved at the specific target/cleavage site.

FIG. 28A is a schematic drawing showing an example, according to some embodiments, of additional structure that may increase the efficiency of translation of the exogenous RNA of interest, that is cleaved at the 5′ end, such that the additional structure is a polypeptide that is encoded from the composition of the invention, wherein the polypeptide is capable of binding to the poly-A tail of the exogenous RNA of interest, and to a sequence within the exogenous RNA of interest of the invention and by this force the exogenous RNA of interest to form a circular structure, particularly when the exogenous RNA of interest is cleaved at the specific target/cleavage site.

FIG. 28B is a schematic drawing illustrating an example, according to some embodiments, of additional structure that may reduce the efficiency of translation of the intact exogenous RNA of interest, such that the additional structure is a cis acting ribozyme that removes the CAP structure from the intact exogenous RNA of interest.

FIG. 29A is a schematic drawing illustrating an example, according to some embodiments, of inhibitory sequence that is located downstream from the specific target/cleavage site and comprises an intron, such that the exogenous RNA of interest is a target for nonsense-mediated decay (NMD).

FIG. 29B is a schematic drawing illustrating an example, according to some embodiments, of inhibitory sequence that is located downstream from the specific target/cleavage site and comprises a binding site for translation repressor.

FIG. 29C is a schematic drawing illustrating an example, according to some embodiments, of inhibitory sequence that is located downstream from the specific target/cleavage site and comprises an RNA localization signal for subcellular localization.

FIG. 29D is a schematic drawing illustrating an example, according to some embodiments, of an inhibitory sequence that is located downstream from the specific target/cleavage site and comprises an RNA destabilizing element that is an AU-rich element or an endonuclease recognition site.

FIG. 29E is a schematic drawing illustrating an example, according to some embodiments, of an inhibitory sequence that is located downstream from the specific target/cleavage site and comprises a secondary structure.

FIG. 30A is a schematic drawing illustrating an example, according to some embodiments, for inhibitory sequence that is located downstream from the sequence encoding exogenous protein of interest, such that the inhibitory sequence creates a secondary structure that may block translation.

FIG. 30B is a schematic drawing illustrating an example, according to some embodiments, of additional structure that may increase the efficiency of translation of the exogenous RNA of interest that is cleaved at the 3′ end, such that the additional structure is IRES (Internal ribosome entry site).

FIG. 30C is a schematic drawing illustrating an example, according to some embodiments, of additional structure that may increase the efficiency of translation of the exogenous RNA of interest, that is cleaved at the 3′ end, such that the additional structure is a stem loop structure.

FIG. 30D is a schematic drawing illustrating an example, according to some embodiments, of additional structure that may increase the efficiency of translation of the exogenous RNA of interest that is cleaved at the 3′ end, such that the additional structure is a cytoplasmic polyadenylation element.

FIG. 31A is a schematic drawing illustrating an example, according to some embodiments, of additional structures that may increase the efficiency of translation of the exogenous RNA of interest, that is cleaved at the 3′ end, such that the additional structures are nucleotide sequences that are capable of binding to each other and consequently force the exogenous RNA of interest to form a circular structure, particularly when the exogenous RNA of interest is cleaved at the specific target/cleavage site.

FIG. 31B is a schematic drawing illustrating an example, according to some embodiments, of additional structure that may increase the efficiency of translation of the exogenous RNA of interest, that is cleaved at the 3′ end, such that the additional structure is a polypeptide that is encoded from the composition, wherein the polypeptide is capable of binding to the CAP and to a sequence within the exogenous RNA of interest and consequently force the exogenous RNA of interest to form a circular structure, particularly when the exogenous RNA of interest is cleaved at the specific target/cleavage site.

FIG. 31C is a schematic drawing illustrating an example, according to some embodiments, of additional structure that may increase the efficiency of translation of the exogenous RNA of interest that is cleaved at the 3′ end, such that the additional structure is an additional RNA molecule that is encoded from the composition and is capable of binding to the exogenous RNA of interest an consequently provide it with a poly-A tail, particularly when the exogenous RNA of interest is cleaved at the specific target/cleavage site.

FIG. 31D is a schematic drawing illustrating an example, according to some embodiments, of additional structure that may reduce the efficiency of translation of the intact exogenous RNA of interest, such that the additional structure is a cis acting ribozyme that removes the poly-A from the intact exogenous RNA of interest.

FIG. 32A is a schematic drawing illustrating an example, according to some embodiments, of the structure of the exogenous RNA of interest, which comprises 2 specific sequences, such that the inhibitory sequence is located upstream from the specific target/cleavage sites.

FIG. 32B is a schematic drawing illustrating an example, according to some embodiments, of the structure of the exogenous RNA of interest that comprises 2 specific sequences, such that the inhibitory sequence is located downstream from the specific target/cleavage sites.

FIG. 32C is a schematic drawing illustrating an example, according to some embodiments, of an exogenous RNA of interest, which comprises a sequence encoding an exogenous protein of interest, between 2 sequences that are complementary to the predetermined signal sequence and 2 inhibitory sequences, one at the 5′ end and other at the 3′ end of the exogenous RNA of interest.

FIG. 33 is a schematic drawing illustrating an example, according to some embodiments, for expressing exogenous protein of interest in response to the presence of an endogenous signal RNA in a cell. The composition includes polynucleotide molecule/s that encode for an exogenous RNA of interest molecule that comprises a first sequence of 27 nucleotides at the 5′ end that is 100% complementary to the predetermined signal sequence and to a sequence that is upstream from the predetermined signal sequence, the first sequence also comprises an 5′-AUG-3′ sequence that is not in the same reading frame with the downstream sequence encoding the exogenous protein of interest and the composition further encodes a functional RNA which is shRNA that is capable of effecting the cleavage of the endogenous signal RNA at the 3′ end of the predetermined signal sequence.

FIG. 34 is a schematic drawing illustrating an example for expressing an exogenous protein of interest in response to the presence of an endogenous signal RNA in a cell, according to some embodiments. The composition includes polynucleotide molecule/s that encode for an exogenous RNA of interest molecule that comprises at the 5′ end a miRNA that is capable of effecting the cleavage of the endogenous signal RNA at the 3′ end of the predetermined signal sequence and a first sequence of 27 nucleotides that is complementary to the predetermined signal sequence and to the sequence that is upstream from the predetermined signal sequence, the first sequence also comprises an 5′-AUG-3′ sequence that is not in the same reading frame with the downstream sequence encoding the exogenous protein of interest and such that the 5′-AUG-3′ is located within a Kozak consensus sequence.

FIG. 35 is a schematic drawing illustrating an example, according to some embodiments, for expressing an exogenous protein of interest in response to the presence of an endogenous signal RNA in a cell. The composition encodes for an exogenous RNA of interest molecule that comprises at the 5′ end a first strand of siRNA, such that composition further transcribes the second strand of the siRNA by polymerase I or III based promoter. The first sequence of the exogenous RNA of interest molecule is 27 nucleotides in length and is complementary to the predetermined signal sequence and to the sequence that is upstream from the predetermined signal sequence, the first sequence also comprises an 5′-AUG-3′ sequence that is not in the same reading frame with the downstream sequence encoding the exogenous protein of interest, such that the 5′-AUG-3′ is located within a Kozak consensus sequence.

FIG. 36A is a schematic drawing illustrating an example, according to some embodiments, of an exogenous RNA of interest that comprises a cis acting ribozyme at the 5′ end, which removes the CAP structure from the exogenous RNA of interest. This removal reduces the efficiency of translation of the exogenous protein of interest in the intact exogenous RNA of interest molecule.

FIG. 36B is a schematic drawing illustrating an exemplary exogenous RNA of interest that comprises two nucleotide sequences that are capable of binding to each other and by this force the exogenous RNA of interest to form a circular structure that increases the efficiency of translation of the protein of interest particularly in the cleaved RNA of interest.

FIG. 37A is a schematic drawing illustrating an example, according to some embodiments, for cleaving exogenous RNA of interest in the presence of an endogenous signal RNA in a cell. The composition encodes for: a carrier RNA of 27 nucleotides and an exogenous RNA of interest that comprises a specific sequence which is complementary to the predetermined signal sequence.

FIG. 37B is a schematic drawing illustrating an example, according to some embodiments, for cleaving exogenous RNA of interest in the presence of an endogenous signal RNA in a cell. The composition encodes for: an exogenous RNA of interest that comprises a specific sequence which is complementary to the predetermined signal sequence; a carrier sequence that is of 27 nucleotides long and a functional nucleic acid which is a cis acting ribozyme that is capable of effecting the cleavage of the carrier RNA sequence at the 3′ end of the carrier sequence.

FIG. 38A is a schematic drawing illustrating an example, according to some embodiments, for cleaving exogenous RNA of interest in the presence of an endogenous signal RNA in a cell. The composition of the invention encodes for a carrier RNA of 27 nucleotides and an exogenous RNA of interest that comprises a specific sequence which is complementary to the predetermined signal sequence.

FIG. 38B is a schematic drawing illustrating an example, according to some embodiments, for cleaving exogenous RNA of interest in the presence of an endogenous signal RNA in a cell. The composition encodes for: an exogenous RNA of interest that includes a specific sequence which is 100% complementary to the predetermined signal sequence, a carrier sequence that is of 27 nucleotides long and a functional nucleic acid which is a cis acting ribozyme that is capable of effecting the cleavage of the carrier RNA sequence at the 5′ end of the carrier sequence.

FIG. 39A is a schematic drawing illustrating an example, according to some embodiments, of an exogenous RNA of interest having its inhibitory sequence located downstream from the specific target/cleavage site and theinhibitory sequence is capable of inhibiting the function of an RNA localization signal for subcellular localization.

FIG. 39B is a schematic drawing illustrating an example, according to some embodiments, of an exogenous RNA of interest having its inhibitory sequence located upstream from the specific target/cleavage site and it's the inhibitory sequence is capable of inhibiting the function of an RNA localization signal for subcellular localization.

FIG. 39C is a schematic drawing illustrating an example, according to some embodiments, of an exogenous RNA of interest, having its inhibitory sequence located upstream from the specific target/cleavage site, and comprises an AUG and a downstream sequence that encodes for amino acids that are capable of inhibiting the function of the sorting signal for subcellular localization of the exogenous protein of interest, encoded from the exogenous protein of interest.

FIG. 39D is a schematic drawing illustrating an example, according to some embodiments, of inhibitory sequence that is located downstream from the specific sequence, such that the exogenous RNA of interest does not comprise a stop codon downstream from the start codon of the sequence encoding the exogenous protein of interest, and such that the inhibitory sequence encodes an amino acid sequence that is capable of inhibiting the cleavage of a peptide sequence that is encoded upstream, wherein the peptide sequence is capable of being cleaved by a protease in a mammalian cell.

FIG. 40 is a schematic drawing illustrating an example for using the composition of the invention to kill cancer cells of a specific patient, according to some embodiments.

FIG. 41 is a schematic drawing illustrating an example for using the composition of the invention to kill cancer cells of Burkitt's lymphomas, Hodgkin's lymphomas, gastric carcinoma and nasopharyngeal carcinoma, which are latently infected with EBV by using the LMP1 mRNA as the endogenous signal RNA, according to some embodiments.

FIG. 42 is a schematic drawing illustrating an example for using the composition of the invention to kill HIV-1 infected cells, according to some embodiments.

FIG. 43 is a schematic drawing illustrating an example for using the composition of the invention to kill HSV-1 infected cells, according to some embodiments.

FIG. 44 is a schematic drawing illustrating an example for using the composition of the invention to kill cancer cells of a specific patient, according to some embodiments.

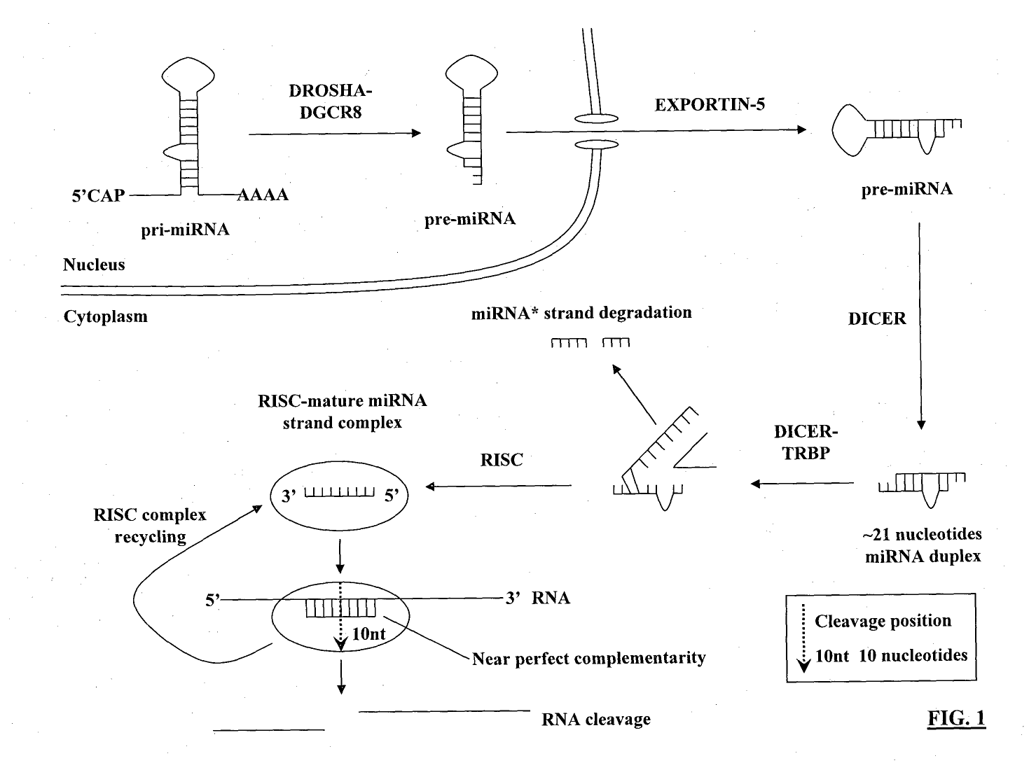

### DETAILED DESCRIPTION OF THE INVENTION

In the following detailed description of the invention when a reference term, such as: said, the, the last, the previous and the former; is used it refers to the exact term that is mentioned above (e.g. wherein said “The nucleic acid sequence” it refers to the nucleic acid sequence that is mentioned above and does not refer to the nucleotide sequence that is mentioned above). Furthermore, in the following detailed description of the invention each embodiment that refers to other embodiments is defined with them as a separate unit.

The following are terms which are used throughout the description and which should be understood in accordance with the various embodiments to mean as follows:

As referred to herein, the terms “polynucleotide molecules”, “oligonucleotide”, “polynucleotide”, “nucleic acid” and “nucleotide” sequences may interchangeably be used herein. The terms are directed to polymers of deoxyribonucleotides (DNA), ribonucleotides (RNA), and modified forms thereof in the form of a separate fragment or as a component of a larger construct, linear or branched, single stranded, double stranded, triple stranded, or hybrids thereof. The term also encompasses RNA/DNA hybrids. The polynucleotides may comprise sense and antisense oligonucleotide or polynucleotide sequences of DNA or RNA. The DNA or RNA molecules may be, for example, but are not limited to: complementary DNA (cDNA), genomic DNA, synthesized DNA, recombinant DNA, or a hybrid thereof or an RNA molecule such as, for example, mRNA, shRNA, siRNA, miRNA, and the like. Accordingly, as used herein, the terms “polynucleotide molecules”, “oligonucleotide”, “polynucleotide”, “nucleic acid” and “nucleotide” sequences are meant to refer to both DNA and RNA molecules. The terms further include oligonucleotides composed of naturally occurring bases, sugars, and covalent internucleoside linkages, as well as oligonucleotides having non-naturally occurring portions, which function similarly to respective naturally occurring portions.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As referred to herein, the term “complementarity” is directed to base pairing between strands of nucleic acids. As known in the art, each strand of a nucleic acid may be complementary to another strand in that the base pairs between the strands are non-covalently connected via two or three hydrogen bonds. Two nucleotides on opposite complementary nucleic acid strands that are connected by hydrogen bonds are called a base pair. According to the Watson-Crick DNA base pairing, adenine (A) forms a base pair with thymine (T) and guanine (G) with cytosine (C). In RNA, thymine is replaced by uracil (U). The degree of complementarity between two strands of nucleic acid may vary, according to the number (or percentage) of nucleotides that form base pairs between the strands. For example, “100% complementarity” indicates that all the nucleotides in each strand form base pairs with the complement strand. For example, “95% complementarity” indicates that 95% of the nucleotides in each strand from base pair with the complement strand. The term sufficient complementarity may include any percentage of complementarity from about 30% to about 100%.

The term “construct”, as used herein refers to an artificially assembled or isolated nucleic acid molecule which may be one or more nucleic acid sequences, wherein the nucleic acid sequences may comprise coding sequences (that is, sequence which encodes an end product), regulatory sequences, non-coding sequences, or any combination thereof. The term construct encompases, for example, vector but should not be seen as being limited thereto.

“Expression vector” refers to vectors that have the ability to incorporate and express heterologous nucleic acid fragments (such as, for example, DNA), in a foreign cell. In other words, an expression vector comprises nucleic acid sequences/fragments (such as DNA, mRNA, tRNA, rRNA), capable of being transcribed. Many prokaryotic and eukaryotic expression vectors are known and/or commercially available. Selection of appropriate expression vectors is within the knowledge of those having skill in the art.

The terms “Upstream” and “Downstream”, as used herein refers to a relative position in a nucleotide sequence, such as, for example, a DNA sequence or an RNA sequence. As well known, a nucleotide sequence has a 5′ end and a 3′ end, so called for the carbons on the sugar (deoxyribose or ribose) ring of the nucleotide backbone. Hence, relative to the position on the nucleotide sequence, the term downstream relates to the region towards the 3′ end of the sequence. The term upstream relates to the region towards the 5′ end of the strand.

The terms “promoter element”, “promoter” or “promoter sequence” as used herein, refer to a nucleotide sequence that is generally located at the 5′ end (that is, precedes, located upstream) of the coding sequence and functions as a switch, activating the expression of a coding sequence. If the coding sequence is activated, it is said to be transcribed. Transcription generally involves the synthesis of an RNA molecule (such as, for example, a mRNA) from a coding sequence. The promoter, therefore, serves as a transcriptional regulatory element and also provides a site for initiation of transcription of the coding sequence into mRNA. Promoters may be derived in their entirety from a native source, or be composed of different elements derived from different promoters found in nature, or even comprise synthetic nucleotide segments. It is understood by those skilled in the art that different promoters may direct the expression of a gene in different tissues or cell types, or at different stages of development, or in response to different environmental conditions, or at various expression levels. Promoters which cause a gene to be expressed in most cell types at most times are commonly referred to as “constitutive promoters”. Promoters that control gene expression in a specific tissue are called “tissue specific promoters”.

As referred to herein, the terms “RNA of interest”, “exogenous RNA of interest”, and “ROI” may interchangeably be used. The terms refer to a nucleotide sequence which is introduced into a target cell and may encode for an RNA molecule within the target cell.

As referred to herein, the terms “protein of interest”, “exogenous protein of interest”, and “POI” may interchangeably be used. The terms refer to a peptide sequence which is translated from the exogenous RNA of interest. In some embodiments, the peptide sequence can be one or more separate proteins or a fusion protein.

As referred to herein, the terms “signal RNA” and “endogenous signal RNA” may interchangeably be used. The terms refer to an intracellular RNA molecule/sequence which comprises a predetermined signal sequence. The endogenous signal RNA molecule may be encoded by the genome of the cell, and/or from a foreign genome residing within the cell, such as, for example, from a virus residing within the cell. In some embodiments, the endogenous signal RNA is a mature mRNA molecule. In some embodiments, the endogenous signal RNA is a viral RNA. The signal RNA is present within the target cell prior to introduction of an exogenous RNA of interest into the cell.

As referred to herein, the terms “predetermined signal sequence” and “signal sequence” may interchangeably be used.

As referred to herein, the terms “predetermined cleavage site” and “an additional cleavage site”, refer to a cleavage site within the sequence of the endogenous signal RNA.

As referred to herein, the terms “specific target site”, “specific cleavage site” and “specific target/cleavage sites” may interchangeably be used. The terms relate to one or more cleavage sites within the sequence of the exogenous RNA of interest.

The term “expression”, as used herein, refers to the production of a desired end-product molecule in a target cell. The end-product molecule may be, for example an RNA molecule (such as, for example, a mRNA molecule, siRNA molecule, and the like); a peptide or a protein; and the like; or combinations thereof.

As referred to herein, the term, “Open Reading Frame” (“ORF”) is directed to a coding region which contains a start codon and a stop codon.

As referred to herein, the term “Kozak sequence” is well known in the art and is directed to a sequence on an mRNA molecule that is recognized by the ribosome as the translational start site. The terms “Kozak consensus sequence”, “Kozak consensus” or “Kozak sequence”, is a sequence which occurs on eukaryotic mRNA and has the consensus (gcc)gccRccAUGG (SEQ ID NO. 24), where R is a purine (adenine or guanine), three bases upstream of the start codon (AUG), which is followed by another ‘G’. In some embodiments, the Kozak sequence has the sequence RNNAUGG, wherein N is any nucleotide of A, G, C or U (SEQ ID NO. 112).

As used herein, the terms “introducing” and “transfection” may interchangeably be used and refer to the transfer of molecules, such as, for example, nucleic acids, polynucleotide molecules, vectors, and the like into a target cell(s), and more specifically into the interior of a membrane-enclosed space of a target cell(s). The molecules can be “introduced” into the target cell(s) by any means known to those of skill in the art, for example as taught by Sambrook et al. Molecular Cloning: A Laboratory Manual, Cold Spring Harbor Laboratory Press, New York (2001), the contents of which are incorporated by reference herein. Means of “introducing” molecules into a cell include, for example, but are not limited to: heat shock, calcium phosphate transfection, PEI transfection, electroporation, lipofection, transfection reagent(s), viral-mediated transfer, and the like, or combinations thereof. The transfection of the cell may be performed on any type of cell, of any origin, such as, for example, human cells, animal cells, plant cells, and the like. The cells may be, for example, but not limited to: isolated cells, tissue cultured cells, cell lines, cells present within an organism body, and the like.

The term “Kill” with respect to a cell/cell population is directed to include any type of manipulation that will lead to the death of that cell/cell population.

As referred to herein, the term “Treating a disease” or “treating a condition” is directed to administering a composition, which includes at least one reagent (which may be, for example, one or more polynucleotide molecules, one or more expression vectors, one or more substance/ingredient, and the like), effective to ameliorate symptoms associated with a disease, to lessen the severity or cure the disease, or to prevent the disease from occurring. Administration may be any administration route.

The terms “Detection, “Diagnosis” refer to methods of detection of a disease, symptom, disorder, pathological or normal condition; classifying a disease, symptom, disorder, pathological condition; determining a severity of a disease, symptom, disorder, pathological condition; monitoring disease, symptom, disorder, pathological condition progression; forecasting an outcome and/or prospects of recovery thereof.

### 1. STRUCTURE OF A COMPOSITION OF THE INVENTION, ACCORDING TO SOME EMBODIMENTS

According to some embodiments of the present invention, there is provided a composition for directing cleavage of exogenous RNA of interest in response to the presence of an endogenous signal RNA in a cell. The exogenous RNA of interest is encoded from the composition. The endogenous signal RNA is an RNA molecule which comprises a predetermined signal sequence, such that the predetermined signal sequence is a random sequence of from 18 to 25 nucleotides in length.

In one embodiment of the invention, the composition comprises one or more polynucleotide molecules that comprise:
(a) a polynucleotide sequence encoding the exogenous RNA of interest, such that the exogenous RNA of interest is an RNA sequence that comprises a specific sequence which is of sufficient complementarity to the predetermined signal sequence to direct target-specific RNA interference;
(b) one or more polynucleotide sequence(s) encoding a functional RNA that is capable of effecting the cleavage, directly or indirectly, of the endogenous signal RNA at a predetermined cleavage site, such that the predetermined cleavage site is the 5′ end of the predetermined signal sequence; and
(c) a polynucleotide sequence encoding a carrier RNA which is an RNA molecule that is at least about 18 nucleotides in length and is consisting essentially of:
   (1) a first sequence of from 14 to 31 nucleotides in length which is of sufficient complementarity to an edge sequence to hybridize therewith, the edge sequence is 14-31 nucleotides in length and is located 0-5 nucleotides downstream from the predetermined cleavage site and extends downstream in the endogenous signal RNA;
   (2) a second sequence downstream from the first sequence, such that the second sequence is a random sequence that is 0-5 nucleotides in length; and
   (3) a third sequence upstream from the first sequence, such that the third sequence is 0-7000 nucleotides in length.

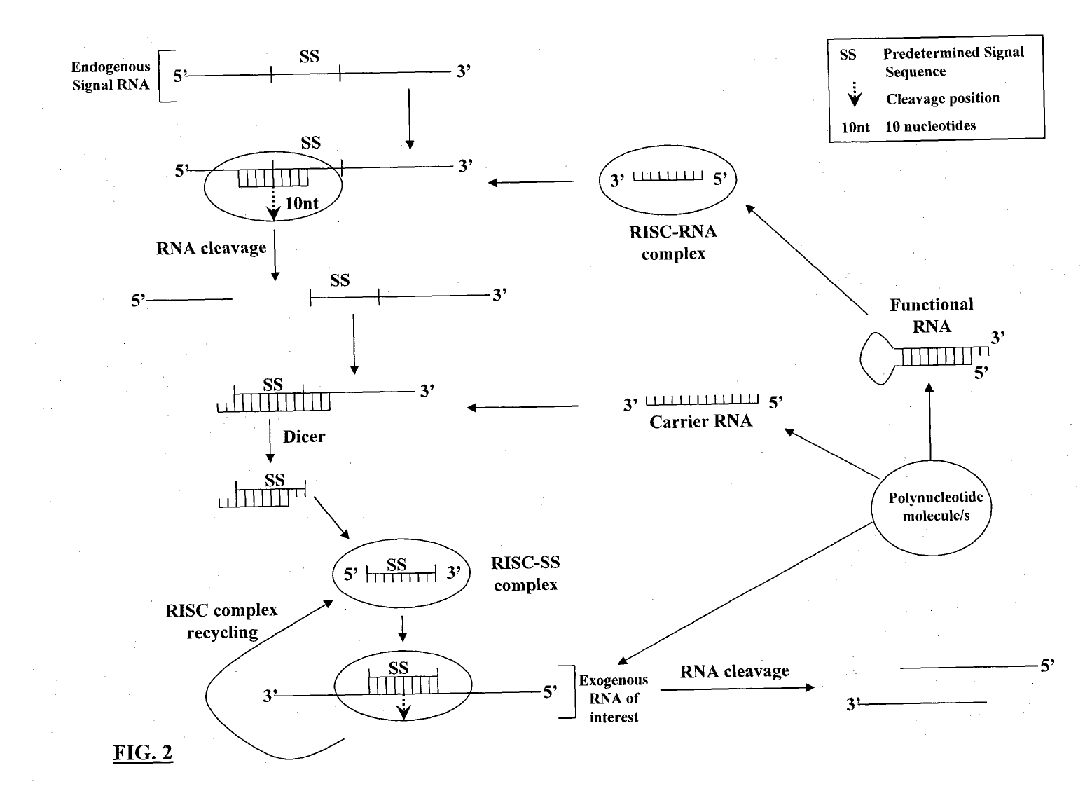

Thus, following introduction of the composition into a cell comprising the endogenous signal RNA, the functional RNA effects the cleavage, directly or indirectly, of the endogenous signal RNA at the 5′ end of the predetermined signal sequence and then the carrier RNA is hybridized to the edge sequence at the cleaved endogenous signal RNA and directs the processing of the predetermined signal sequence and then the processed predetermined signal sequence directs the cleavage of the exogenous RNA of interest at a specific target/cleavage site that is located within the specific sequence. For example, see FIG. 2.

In some embodiments, the composition comprises one or more polynucleotide molecules that comprise:
(a) a polynucleotide sequence encoding the exogenous RNA of interest, such that the exogenous RNA of interest is an RNA sequence that comprises a specific sequence which is of sufficient complementarity to the predetermined signal sequence to direct target-specific RNA interference;
(b) one or more polynucleotide sequence(s) encoding a functional RNA that is capable of effecting the cleavage, directly or indirectly, of the endogenous signal RNA at a predetermined cleavage site, such that the predetermined cleavage site is the 3′ end of the predetermined signal sequence; and
(c) a polynucleotide sequence encoding a carrier RNA which is an RNA molecule that is at least about 18 nucleotides in length and is consisting essentially of:
   (1) a first sequence of from 14 to 31 nucleotides in length which is of sufficient complementarity to an edge sequence to hybridize therewith, the edge sequence is 14-31 nucleotides in length and is located 0-5 nucleotides upstream from the predetermined cleavage site and extends upstream in the endogenous signal RNA;
   (2) a second sequence upstream from the first sequence, such that the second sequence is a random sequence that is 0-5 nucleotides in length; and
   (3) a third sequence downstream from the first sequence, such that the third sequence is 0-7000 nucleotides in length.

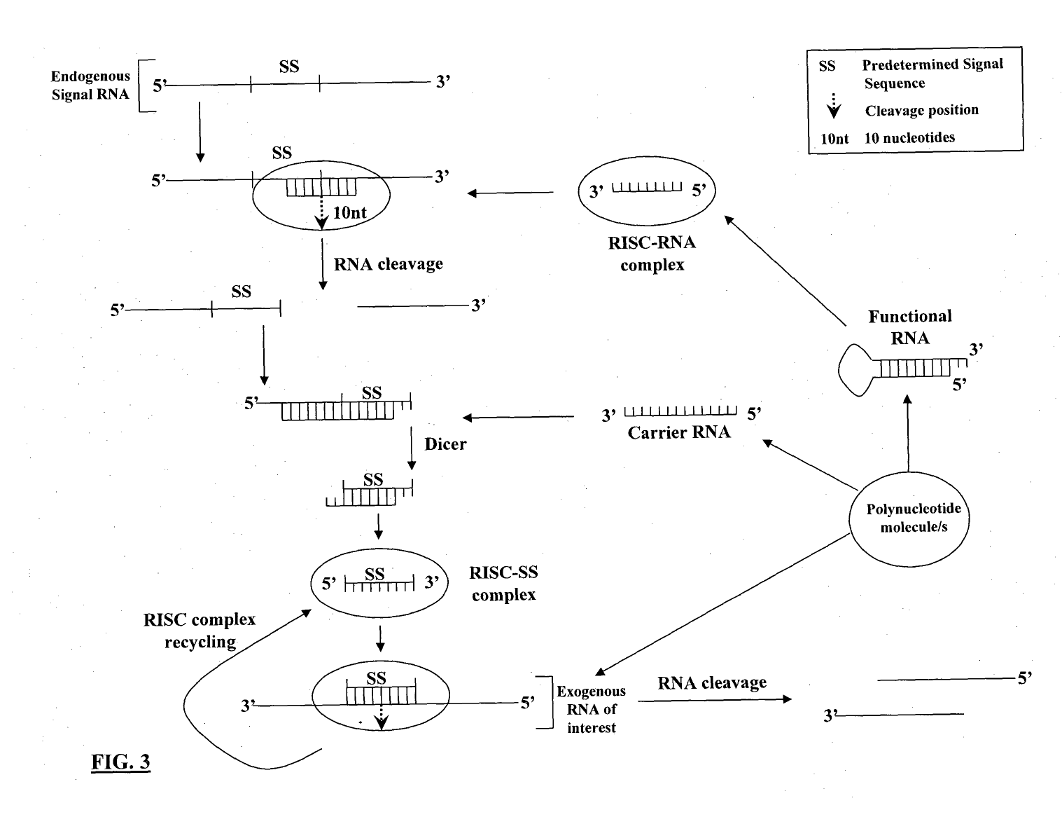

Thus, following introduction of the composition into a cell comprising the endogenous signal RNA, the functional RNA effects the cleavage, directly or indirectly, of the endogenous signal RNA at the 3′ end of the predetermined signal sequence and then the carrier RNA is hybridized to the edge sequence at the cleaved endogenous signal RNA and directs the processing of the predetermined signal sequence and then the processed signal sequence directs the cleavage of the exogenous RNA of interest at a specific target/cleavage site that is located within the specific sequence. For example, see FIG. 3.

In additional embodiment of the invention, the composition comprises one or more polynucleotide molecules that comprise:
(a) a polynucleotide sequence encoding the exogenous RNA of interest, such that the exogenous RNA of interest is an RNA sequence that comprises a specific sequence which is of sufficient complementarity to the predetermined signal sequence to direct target-specific RNA interference;
(b) one or more polynucleotide sequence(s) encoding a functional RNA that is capable of effecting the cleavage, directly or indirectly, of the endogenous signal RNA at a predetermined cleavage site, such that the predetermined cleavage site is the 5′ end of the predetermined signal sequence;
(c) a polynucleotide sequence encoding an RNA carrier sequence comprising a carrier sequence that is at least about 18 nucleotides in length and is consisting essentially of:
   (1) a first sequence of from 14 to 31 nucleotides in length which is of sufficient complementarity to an edge sequence to hybridize therewith, the edge sequence is 14-31 nucleotides in length and is located 0-5 nucleotides downstream from the predetermined cleavage site and extends downstream in the endogenous signal RNA;
   (2) a second sequence downstream from the first sequence, such that the second sequence is a random sequence that is 0-5 nucleotides in length; and
   (3) a third sequence upstream from the first sequence, such that the third sequence is 0-7000 nucleotides in length; and
(d) one or more polynucleotide sequence(s) encoding a functional nucleic acid that is capable of effecting the cleavage, directly or indirectly, of the carrier RNA sequence a carrier cleavage site, such that the carrier cleavage site is the 3′ end of the carrier sequence.

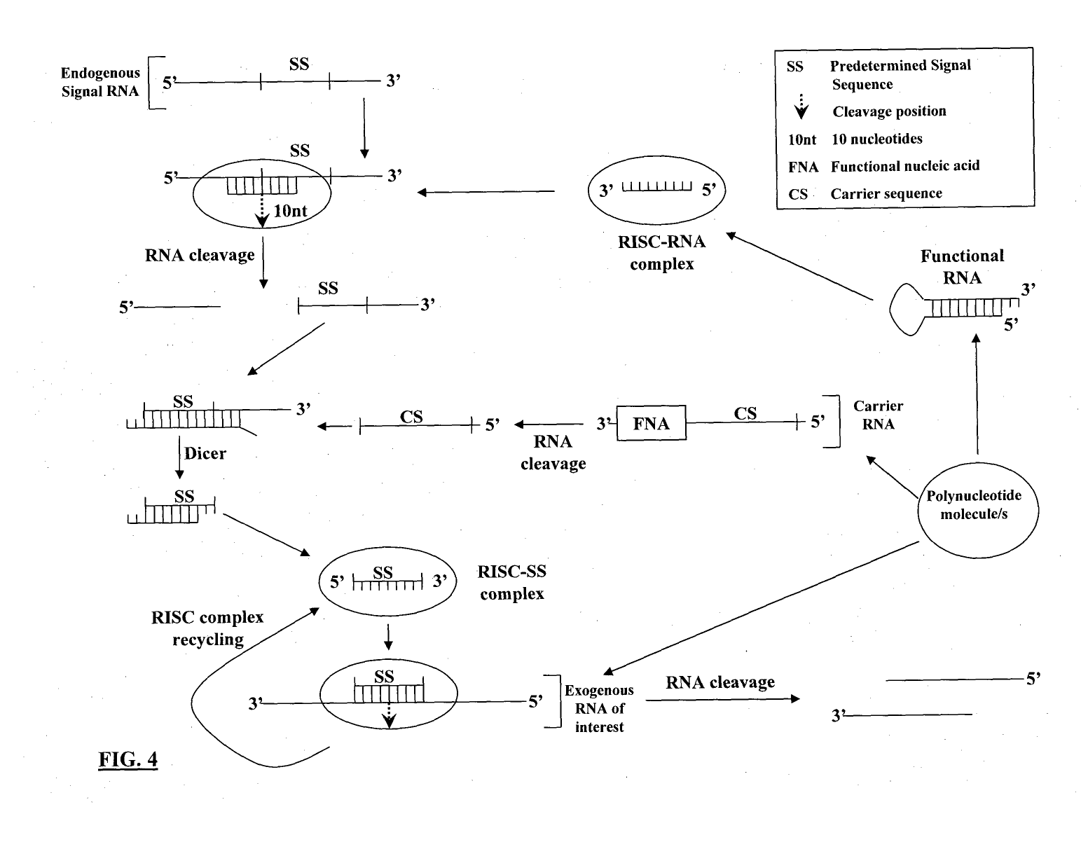

Thus, following introduction of the composition into a cell comprising the endogenous signal RNA, the functional RNA affects the cleavage, directly or indirectly, of the endogenous signal RNA at the 5′ end of the predetermined signal sequence and the functional nucleic acid effects the cleavage, directly or indirectly, of the carrier RNA at the 3′ end of the carrier sequence. The cleaved carrier RNA sequence is hybridized to the edge sequence at the cleaved endogenous signal RNA and directs the processing of the predetermined signal sequence. Then, the processed signal sequence may direct the cleavage of the exogenous RNA of interest at a specific target/cleavage site that is located within the specific sequence. For example, see FIG. 4.

In some embodiments, the composition comprises one or more polynucleotide molecules that comprise:
(a) a polynucleotide sequence encoding the exogenous RNA of interest, such that the exogenous RNA of interest is an RNA sequence that comprises a specific sequence which is of sufficient complementarity to the predetermined signal sequence to direct target-specific RNA interference;
(b) one or more polynucleotide sequence(s) encoding a functional RNA that is capable of effecting the cleavage, directly or indirectly, of the endogenous signal RNA at a predetermined cleavage site, such that the predetermined cleavage site is the 3′ end of the predetermined signal sequence;
(c) a polynucleotide sequence encoding a carrier RNA sequence comprising a carrier sequence that is at least about 18 nucleotides in length and is consisting essentially of:
   (1) a first sequence of from 14 to 31 nucleotides in length which is of sufficient complementarity to an edge sequence to hybridize therewith, the edge sequence is 14-31 nucleotides in length and is located 0-5 nucleotides upstream from the predetermined cleavage site and extends upstream in the endogenous signal RNA;
   (2) a second sequence upstream from the first sequence, such that the second sequence is a random sequence that is 0-5 nucleotides in length; and
   (3) a third sequence downstream from the first sequence, such that the third sequence is 0-7000 nucleotides in length; and
(d) one or more polynucleotide sequence(s) encoding a functional nucleic acid that is capable of effecting the cleavage, directly or indirectly, of the carrier RNA sequence at a carrier cleavage site, such that the carrier cleavage site is the 5′ end of the carrier sequence.

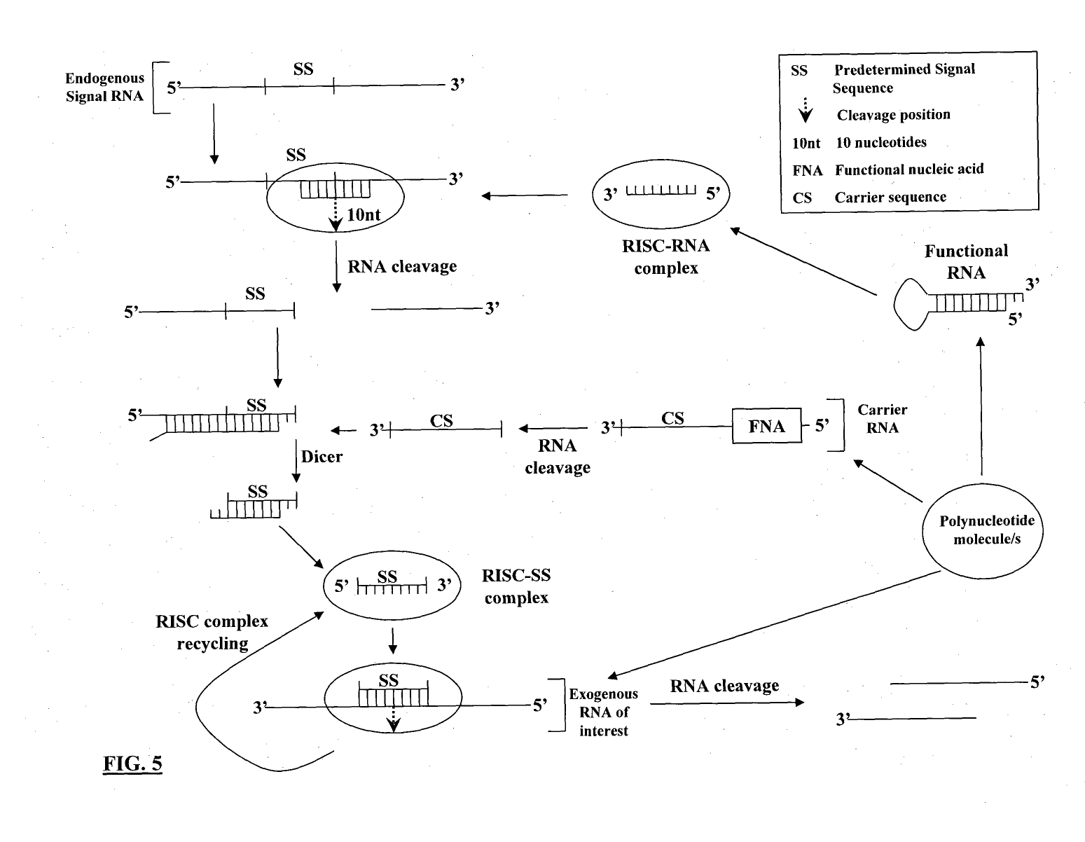

Thus, following introduction of the composition into a cell comprising the endogenous signal RNA, the functional RNA effects the cleavage, directly or indirectly, of the endogenous signal RNA at the 3′ end of the predetermined signal sequence and the functional nucleic acid effects the cleavage, directly or indirectly, of the carrier RNA sequence at the 5′ end of the carrier sequence. The cleaved carrier RNA sequence may then hybridize to the edge sequence at the cleaved endogenous signal RNA and direct the processing of the predetermined signal sequence. Then, the processed predetermined signal sequence may direct the cleavage of the exogenous RNA of interest at a specific target/cleavage site that is located within the specific sequence. For example, see FIG. 5.

According to some embodiments, the predetermined signal sequence may be chosen due to its presence within specific target cells, thereby providing a mechanism for targeting the cleavage of the exogenous RNA of interest in selected cells. The specific target cells may be any type of cells. For example, the specific target cells may be such cells as, but not limited to: benign or malignant neoplasms. On average, each tumor comprises mutations in 90 protein-coding genes [16]. Each tumor is initiated from a single founder cell [38], thus it is most probable that at least one of these mutant genes is transcribed into mRNA. The specific cells may also include, but are not limited to viral infected cells. Specificity may be achieved by modification of the sequences that encode the functional RNA, carrier RNA and/or the specific sequence in the exogenous RNA of interest.

In a co-pending application, which is directed to the activation of gene of interest in a cell expressing a specific miRNA, the predetermined signal sequence may comprise an endogenous miRNA.

According to some embodiments, the predetermined signal sequence of the present invention does not include an endogenous cellular miRNA molecule or any other type of endogenous RNA molecule (such as, for example, shRNA, ribozyme, stRNA, and the like), that is able to direct or effect cleavage of an RNA molecule within the cell.

In some embodiments, the predetermined signal sequence cannot induce/effect cleavage in the absence of one or more components of the composition of the invention.

Various methods have been developed to identify a predetermined signal sequence that is unique to specific cells. These methods include DNA microarray, Tilling (Targeting Induced Local Lesions In Genomes) and large-scale sequencing of cancer cells genomes. Furthermore the identification of the predetermined signal sequence is predicted to be even simpler thanks to the Cancer Genome Atlas (NIH project), which was launched at Dec. 13, 2005 and has been cataloguing all the genetic mutations responsible for cancer.

It has been reported that in mammal cells, the removal of the poly(A) tail reduces the functional mRNA half-life only by 2.6-fold and the removal of the cap reduces the functional mRNA half-life only by 1.7-fold [10]. It has also been reported that two portions of mRNA that has been cleaved by RISC-RNA complex in a cell can be easily detected by Northern analysis [6].

According to some embodiments, the carrier RNA/sequence of embodiments of the may be hybridized to the cleaved endogenous signal RNA portion that includes the predetermined signal sequence. It has been reported that in a cell, two RNA transcripts of about 23 nucleotides in length that have a complementary region of about 19 nucleotides in length at the 5′ end are hybridized to each other and are capable of directing target specific RNA interference [7].

According to further embodiments, the duplex that comprises the carrier RNA and the cleaved endogenous signal RNA portion that includes the predetermined signal sequence may be a substrate for Dicer and thereafter for Risc. It has been reported that a dsRNA of 52 nucleotides long that further comprises 20 nucleotides long ssRNA at one of the 3′ ends is a substrate for a Dicer at the blunt end [8]. It has also been reported that in mammalian cells, Risc is coupled to Dicer [9].

### 2. STRUCTURE OF THE FUNCTIONAL RNA AND THE FUNCTIONAL NUCLEIC ACID

This section describes various embodiments of the structure of the functional RNA and functional nucleic acid of the composition of the invention. This is illustrated, for example, in FIGS. 2, 3, 4, 5.

In another embodiment of the invention, the functional RNA, described in previous embodiments above (section 1) is:
(i) an inhibitory RNA comprising a sequence of from 18 to 25 nucleotides in length which is of sufficient complementarity to a target sequence for the inhibitory RNA to direct cleavage of the endogenous signal RNA at the predetermined cleavage site via, for example, RNA interference, such that the target sequence is a sequence of from 18 to 25 nucleotides in length that is located in a region within the endogenous signal RNA, such that the region is located from about 25 nucleotides downstream from the predetermined cleavage site to about 25 nucleotides upstream from the predetermined cleavage site; or a ribozyme capable of binding to the region of (i) and effecting the cleavage of the endogenous signal RNA at the predetermined cleavage site).

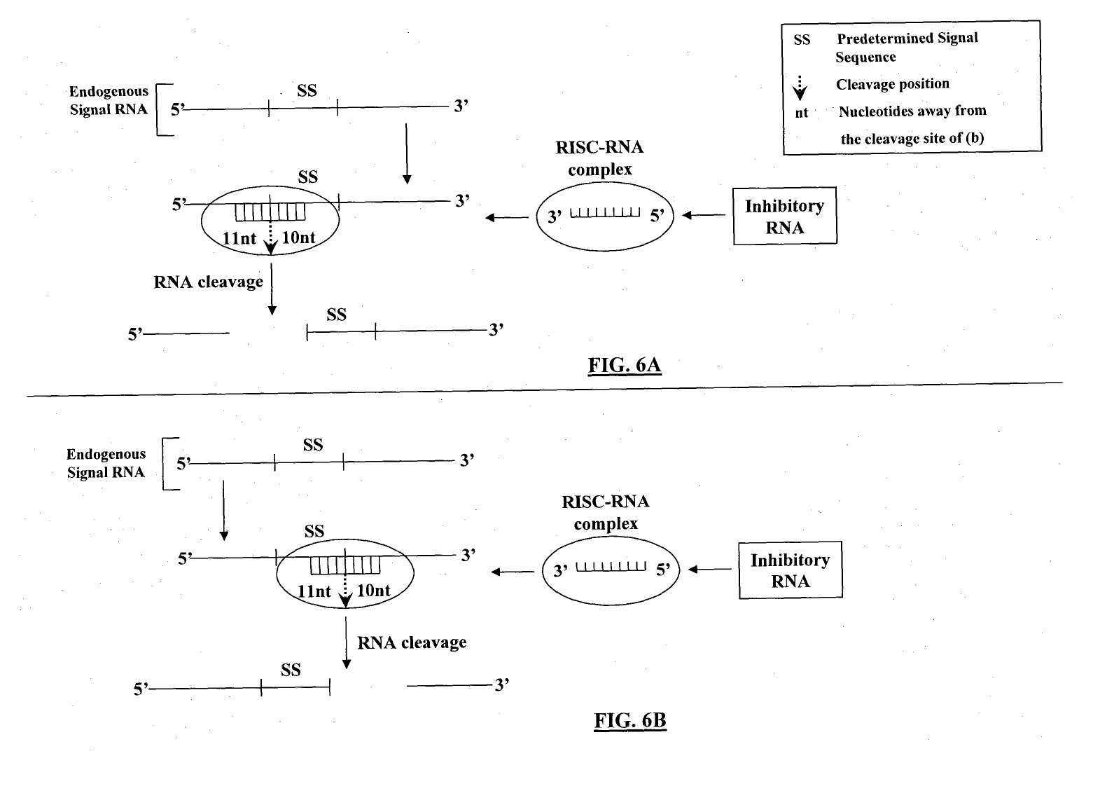

In one embodiment, the region of (i) that is described in the former embodiment may be located from about 11 nucleotides downstream from the predetermined cleavage site to about 12 nucleotides upstream from the predetermined cleavage site. In one embodiment, the region of (i) that is located from about 10 nucleotides downstream from the predetermined cleavage site to about 11 nucleotides upstream from the predetermined cleavage site. For example, see FIG. 6A, 6B.

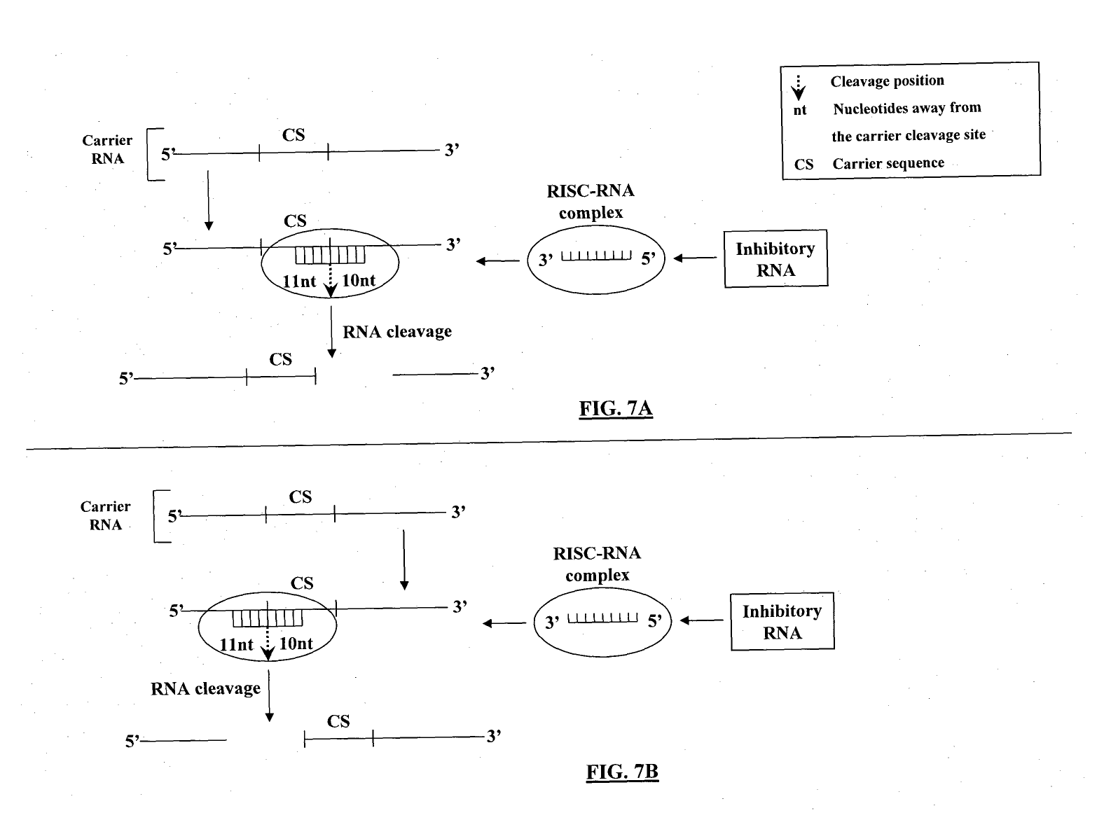

In another embodiment of the invention, the functional nucleic acid, described above (section 1) is:
(i) an inhibitory RNA comprising a sequence of from 18 to 25 nucleotides in length which is of sufficient complementarity to a target sequence for the inhibitory RNA to direct cleavage of the carrier RNA sequence at the carrier cleavage site via, for example, RNA interference, whereby the target sequence is a sequence of from 18 to 25 nucleotides in length that is located in a region within the carrier RNA and wherein the region is located from about 25 nucleotides downstream from the carrier cleavage site to about 25 nucleotides upstream from the carrier cleavage site; or
(ii) a ribozyme capable of binding to the region of (i) and effecting the cleavage of the carrier RNA at the carrier cleavage site. In one embodiment, the region of (i) that is described in the former embodiment is located from about 11 nucleotides downstream from the carrier cleavage site to about 12 nucleotides upstream from the carrier cleavage site. In another embodiment, the region of (i) is located from about 10 nucleotides downstream from the carrier cleavage site to about 11 nucleotides upstream from the carrier cleavage site. For example, see FIG. 7A, 7B.

According to some embodiments, the inhibitory RNA of (i), described above may be, for example, but is not limited to: antisense RNA, double-stranded RNA (dsRNA) and/or small-interfering RNA (siRNA). In some embodiments, the inhibitory RNA of (i), may be, for example, but not limited to: microRNA (miRNA), lariat-form RNA, short-hairpin RNA (shRNA) and/or siRNA expression domain.

According to additional embodiments, the inhibitory RNA of (i) that comprises:
(a) a first RNA molecule that comprises a nucleic acid sequence at the 5′ or 3′ end, such that the nucleic acid sequence is of sufficient complementarity to the target sequence of (i) to direct target-specific RNA interference; and
(b) a second RNA molecule that comprises a nucleotide sequence that is capable of binding to the nucleic acid sequence, such that the nucleotide sequence is 18-25 nucleotides in length and such that the nucleotide sequence is located at the 5′ or 3′ end of the second RNA molecule.

Thus, the first and second RNA molecule form 3′-overhang or 5′-overhang of 0-5 nucleotides on the active end of the duplex formed when each of the first and second RNA molecules is hybridized with the other whereby such that the active end of the duplex formed is the end that comprises the nucleic acid sequence and the nucleotide sequence.

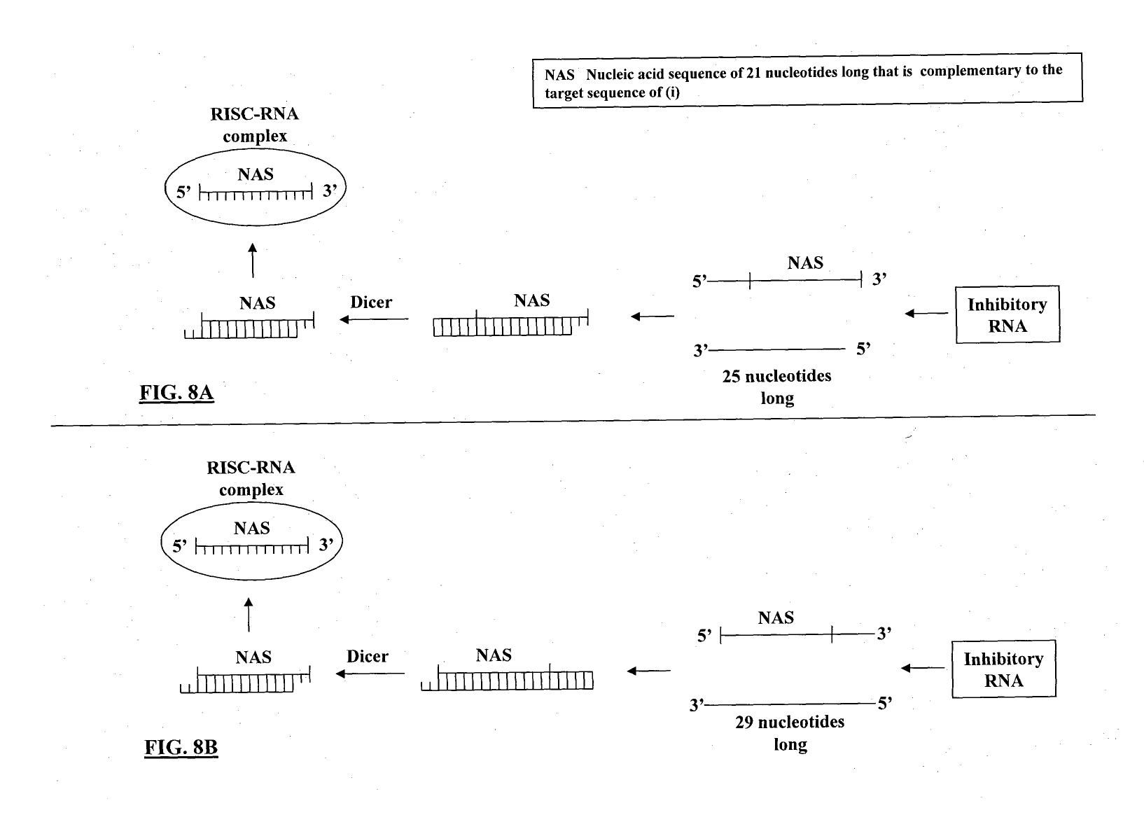

In another embodiment, the first RNA molecule that is described in the former embodiment is about 25 to 30 nucleotides long and the second RNA molecule is about 25 to 30 nucleotides long, such that the first and second RNA molecules form 3′-overhang of 2 nucleotides on the active end of the duplex formed when each of the first and second RNA molecules is hybridized with the other and such that the duplex may be a substrate for a Dicer. For example, see FIG. 8A, 8B.

In some embodiments, the ribozyme of (ii) that is described above, may be, for example, but is not limited to: hammerhead-type ribozyme, hairpin ribozyme and/or tetrahymena-type ribozyme.

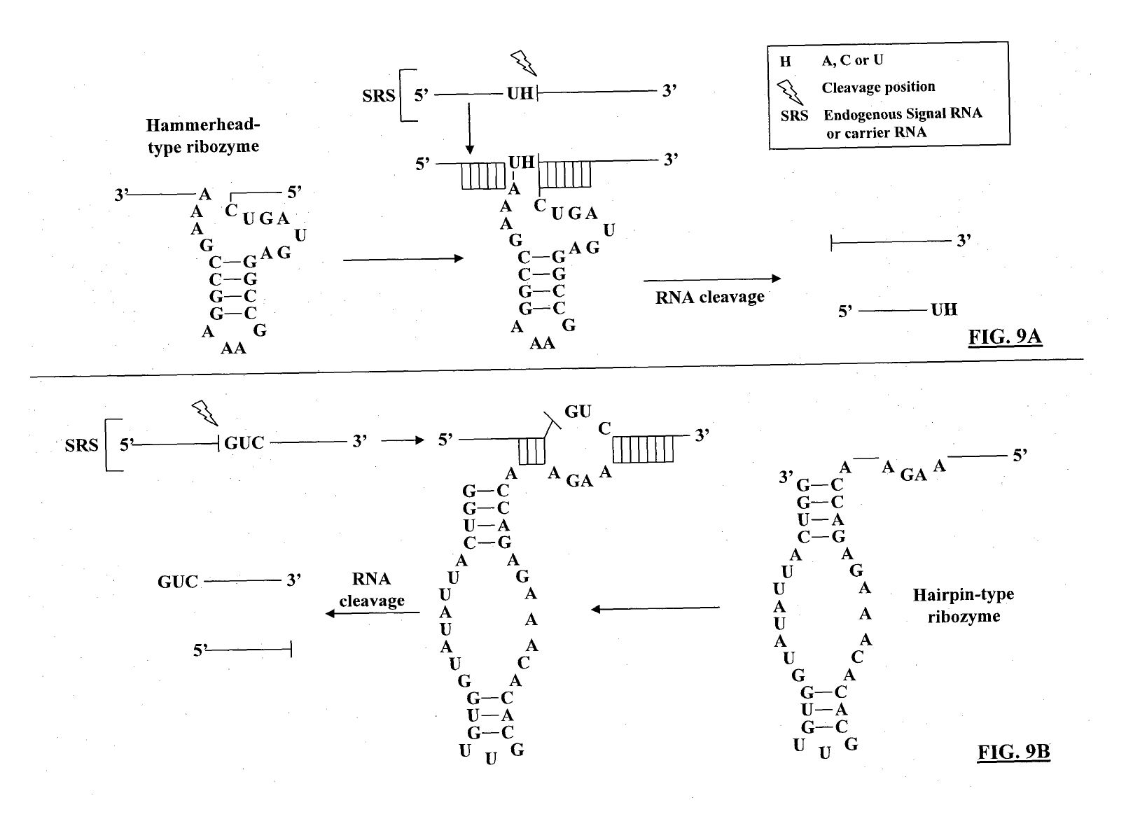

In additional embodiments, the ribozyme of (ii) is a hammerhead-type ribozyme [21] that comprises at the 3′ end a first sequence of 7 nucleotides in length that is complementary to a sequence that is located 26 nucleotides upstream from the 3′ end of the region of (i) and extends upstream in the region of (i), furthermore the hammerhead-type ribozyme comprises at the 5′ end a second sequence of 7 nucleotides in length that is complementary to a sequence that is located 18 nucleotides upstream from the 3′ end of the region of (i) and extends upstream in the region of (i) [21]. For example, see FIG. 9A.

In another embodiment, the ribozyme of (ii), is a hairpin ribozyme [21] that comprises at the 5′ end a nucleic acid sequence of 16 nucleotides in length, such that the nucleic acid sequence comprises at the 5′ end a sequence of 8 nucleotides in length that is complementary to a sequence that is located 28 nucleotides downstream from the 5′ end of the region of (i) and extends downstream in the region of (i) and such that the nucleic acid sequence comprises at the 3′ end a sequence of 4 nucleotides in length that is complementary to a sequence that is located 26 nucleotides upstream from the 3′ end of the region of (i) and extends upstream in the region of (i) [21]. For example, see FIG. 9B.

According to some embodiments, and without wishing to bound to theory or mechanism, the use of a ribozyme will not retain/use up, and consequently dilute, the cellular the components of the RNA interference pathway.

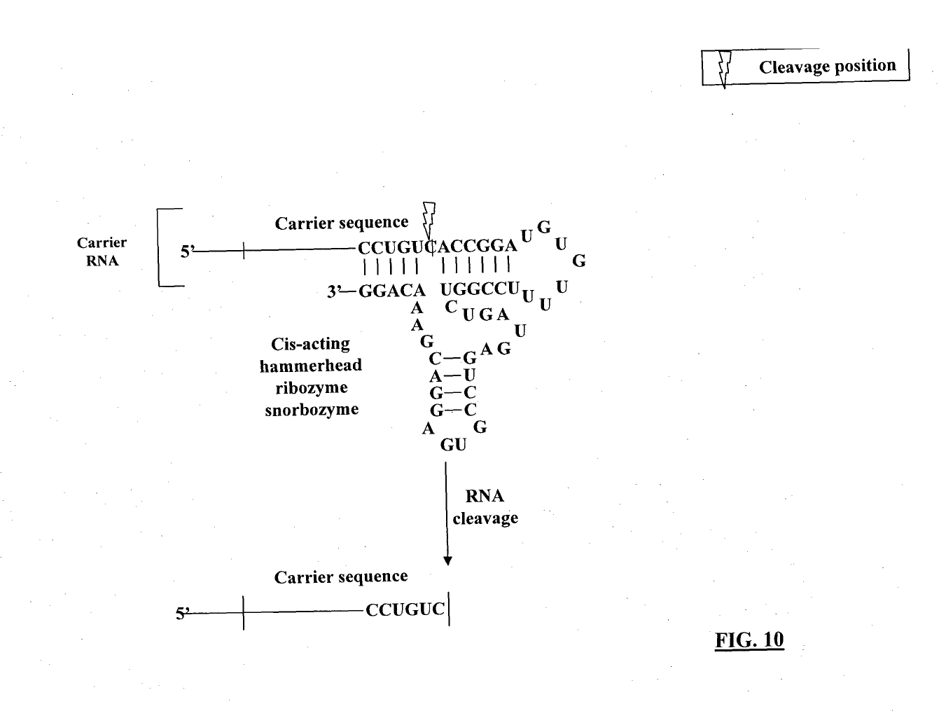

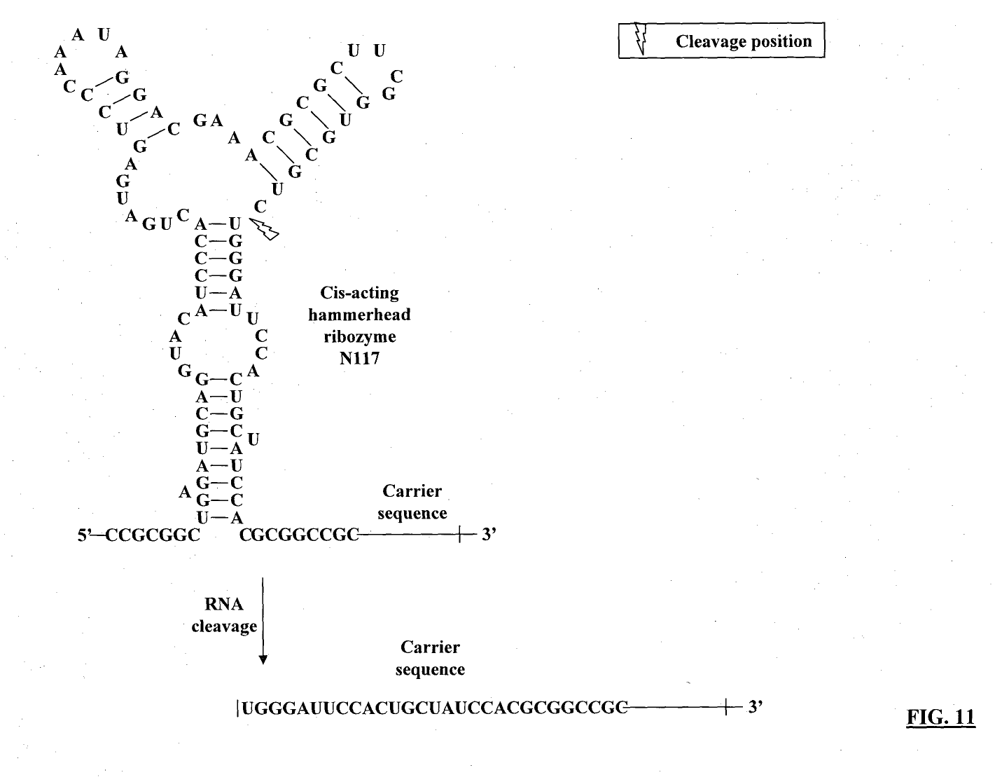

In another embodiment, the functional nucleic acid, described in embodiments in section 1 is a cis acting ribozyme that is located within the carrier RNA sequence and effects the cleavage of the carrier RNA at the carrier cleavage site. In some embodiments, the cis acting ribozyme may be, for example, but is not limited to the very efficient cis-acting hammerhead ribozyme: snorbozyme [22] and/or N117 [23]. For example, see FIG. 10, 11.

According to some embodiments, and without wishing to bound to theory or mechanism, in by using a cis acting ribozyme, the carrier sequence that comprises it may be cleaved by itself [22], which may yield preferred results.

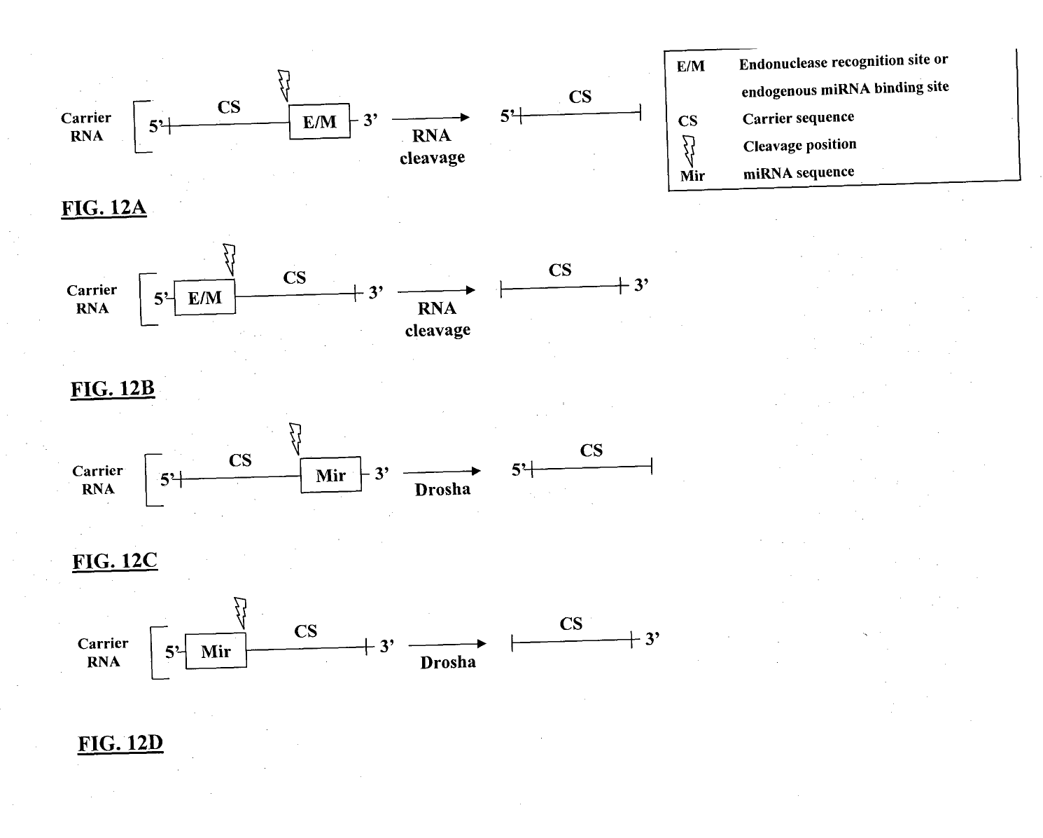

In another embodiment, the functional nucleic acid described in the embodiments in section 1 is an endonuclease recognition site or an endogenous miRNA binding site, such that the functional nucleic acid is located within the carrier RNA and is capable of effecting the cleavage, directly or indirectly, of the carrier RNA at the carrier cleavage site. For example, see FIG. 12A, 12B.

### 3. STRUCTURE OF A FUNCTIONAL NUCLEIC ACID THAT HAS A STEM LOOP STRUCTURE

The functional nucleic acid that is described in embodiments of section 1 may be, for example, but is not limited to, a stem loop structure or miRNA structure, whereby the functional nucleic acid directs the cleavage of the carrier sequence at the carrier cleavage site. This section describes embodiments of the structure of this functional nucleic acid that has a stem loop structure or miRNA structure.

In some embodiments, the functional nucleic acid described in embodiments in section 1 is a miRNA sequence that is located within the carrier RNA sequence, such that following introduction of the composition into a cell, the miRNA sequence is processed, such that the processing of the miRNA sequence is capable of effecting the cleavage, directly or indirectly, of the carrier RNA at the carrier cleavage site and such that the processing of the miRNA sequence comprises Drosha processing. In one embodiment, the miRNA sequence that is described in the former embodiment comprises a sequence corresponding to a naturally occurring miRNA, or a sequence substantially identical thereto. For example, see FIG. 12C, 12D.

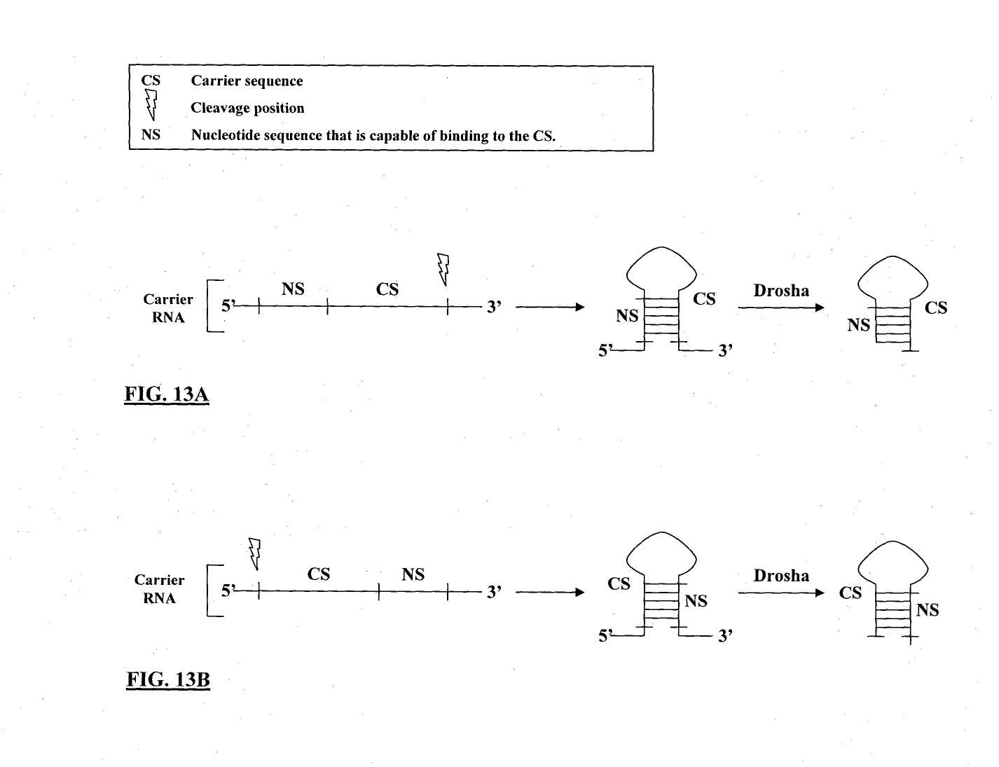

In another embodiment, the functional nucleic acid, described in section 1 has a nucleotide sequence that is located immediately upstream from the 5′ end of the carrier sequence in the carrier RNA sequence, such that the third sequence is 0 nucleotides in length and such that the nucleotide sequence is capable of binding to the carrier sequence, whereby the carrier sequence and the nucleotide sequence are capable of forming a stem loop structure that is a substrate for a Drosha. Following introduction of the composition into a cell, the stem loop structure may be processed and the processing of the stem loop structure is capable of affecting the cleavage, directly or indirectly, of the carrier RNA at the 3′ end of the carrier sequence. In another embodiment, the stem loop structure that is described in the former embodiment is a maximum of about 150 nucleotides long and the processed stem loop structure is not a substrate for Dicer. For example, see FIG. 13A.

In another embodiment, the functional nucleic acid, described in section 1 has a nucleotide sequence that is located immediately downstream from the 3′ end of the carrier sequence in the carrier RNA, such that the third sequence is 0 nucleotides in length and such that the nucleotide sequence is capable of binding to the carrier sequence. The carrier sequence and the nucleotide sequence are capable of forming a stem loop structure that is a substrate for Drosha. Following introduction of the composition into a cell, the stem loop structure may be processed, and the processing of the stem loop structure is capable of effecting the cleavage, directly or indirectly, of the carrier RNA at the 5′ end of the carrier sequence. In another embodiment, the stem loop structure that is described in the former embodiment has a maximum of about 150 nucleotides long and the processed stem loop structure is not a substrate for Dicer. For example, see FIG. 13B.

According to some embodiments, and without wishing to bound to theory or mechanism, the use of miRNA sequence or stem loop structure may provide enhanced results since the carrier sequence may be cleaved independently by Drosha. In another embodiment, the functional nucleic acid described in embodiments of section 1 comprises:
(i) a first nucleotide sequence that is located immediately upstream from the 5′ end of the carrier sequence in the carrier RNA, such that the third sequence is 0-50 nucleotides in length; and
(ii) a second nucleotide sequence that is located immediately downstream from the 3′ end of the carrier sequence, such that the second nucleotide sequence is capable of binding to the first nucleotide sequence, such that the second nucleotide sequence and the first nucleotide sequence and the carrier sequence are capable of forming a stem loop structure.

Whereby, following introduction of the composition into a cell, the stem loop structure is processed, wherein the processing of the stem loop structure is capable of effecting the cleavage, directly or indirectly, of the carrier RNA at the carrier cleavage site and such that the processing of the stem loop structure is capable of forming one or more RNA duplex(es). The processing of the stem loop structure may include, for example, Dicer processing and the RNA duplex(es) may be siRNA duplex(es) and/or miRNA duplex(es).

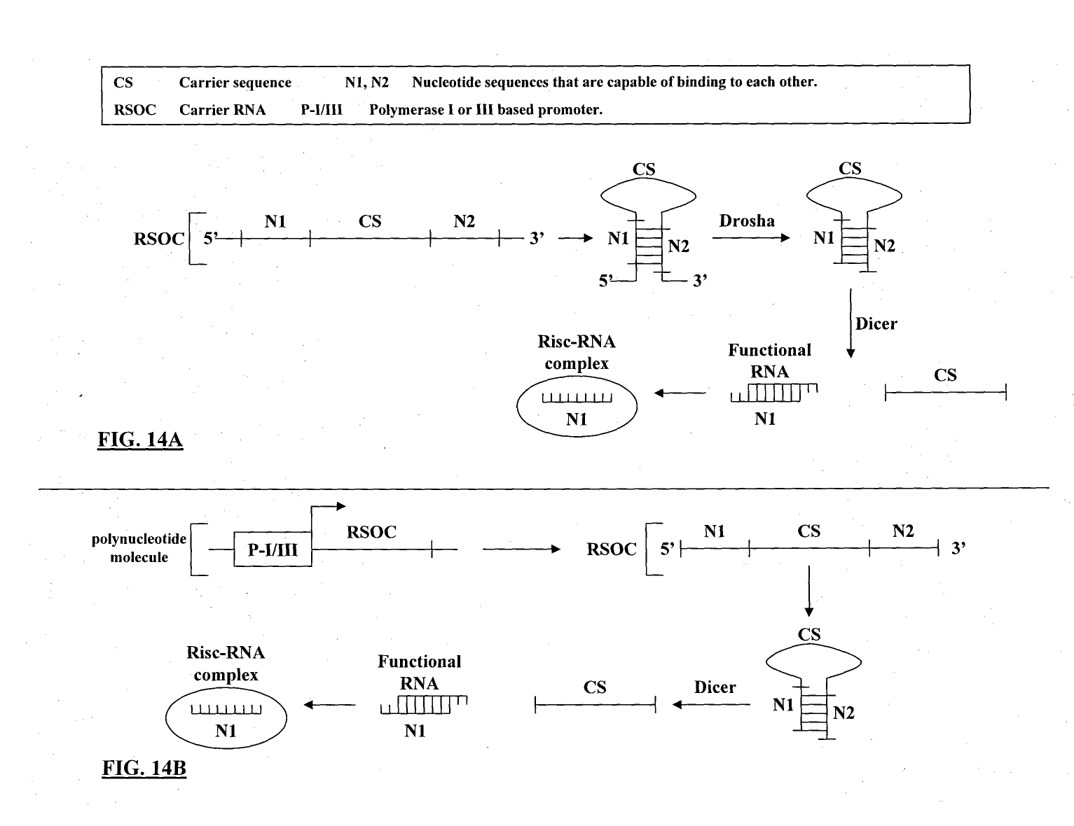

In additional embodiments, the functional RNA described in the former embodiment is a nucleic acid sequence of from 18 to 25 nucleotides in length which is of sufficient complementarity to a target sequence to direct target-specific RNA interference, such that the target sequence is a sequence of from 18 to 25 nucleotides in length that is located in a region within the endogenous signal RNA. The region is located from about 25 nucleotides downstream from the predetermined cleavage site to about 25 nucleotides upstream from the predetermined cleavage site, such that the nucleic acid sequence is located within the first nucleotide sequence or within the second nucleotide sequence. Following introduction of the composition into a cell, at least one RNA duplex from the one or more RNA duplex(es) comprises the nucleic acid sequence and the RNA duplex that comprises the nucleic acid sequence directs the cleavage of the endogenous signal RNA at the predetermined cleavage site via RNA interference. For example, see FIG. 14A.

In another embodiment, the first nucleotide sequence or the second nucleotide sequence described in the former embodiment is the nucleic acid sequence, such that the carrier RNA sequence is consisting essentially of: the first nucleotide sequence and the second nucleotide sequence and the carrier sequence. The first nucleotide sequence is 18-25 nucleotides in length and the second nucleotide sequence is 18-25 nucleotides in length. The stem loop structure forms 3′-overhang of 2 nucleotides and may be a substrate for a Dicer and such that the expression of the carrier RNA polynucleotide sequence is driven by polymerase I based promoter or polymerase III based promoter. For example, see FIG. 14B.

In some embodiments, the region described in any of the previous 2 embodiments is located from about 11 nucleotides downstream from the predetermined cleavage site to about 12 nucleotides upstream from the predetermined cleavage site. In another embodiment, the region described in the former embodiment is located from about 10 nucleotides downstream from the predetermined cleavage site to about 11 nucleotides upstream from the predetermined cleavage site.

According to some embodiments, and without wishing to bound to theory or mechanism, the use of functional RNA and a carrier sequence that are located in the same RNA molecule may require less transcriptional units, which may yield advantageous results. Additional advantage of the proximity of the functional RNA and the carrier sequence is that they are synthesized in the same location in the cell at the same time and at a constant ratio.

### 4. STRUCTURE OF A CARRIER SEQUENCE/RNA AND FUNCTIONAL RNA/NUCLEIC ACID THAT ARE LOCATED IN THE SAME RNA DUPLEX

This section describes various embodiments for the structure of the composition of the invention, described in section 1, wherein the carrier RNA and/or carrier sequence are located in the same RNA duplex together with the functional RNA or with the functional nucleic acid.

In another embodiment of the invention, the functional nucleic acid described in embodiments of section 1 may comprise:
(i) a nucleotide sequence that is located immediately downstream from the 3′ end of the carrier sequence in carrier RNA, such that the carrier RNA is consisting essentially of the carrier sequence and the nucleotide sequence; and (ii) one or more RNA molecule(s) that are capable of binding to the nucleotide sequence; such that the nucleotide sequence and at least one of the RNA molecules form 3′-overhang or 5′-overhang of 0-5 nucleotides on one end of the duplex, which is formed when each of the nucleotide sequence and the at least one RNA molecule is hybridized to the other. Following introduction of the composition into a cell, the nucleotide sequence and the one or more RNA molecule(s) are hybridized with each other and the nucleotide sequence is processed, such that the processing of the nucleotide sequence is capable of effecting the cleavage, directly or indirectly, carrier RNA at the carrier cleavage site and such that the processing of the nucleotide sequence is capable of forming one or more RNA duplex(es). The processing of the nucleotide sequence may include, for example, Dicer processing and the RNA duplex(es) may be siRNA duplex(es) and/or miRNA duplex(es).

In another embodiment of the invention, the functional nucleic acid, described in embodiments in section 1 may comprise:
(i) a nucleotide sequence that is located immediately upstream from the 5′ end of the carrier sequence in the carrier RNA, such that the carrier RNA is consisting essentially of: the carrier sequence and the nucleotide sequence; and (ii) one or more RNA molecule(s) that are capable of binding to the nucleotide sequence; such that the nucleotide sequence and at least one of the RNA molecules from the one or more RNA molecule(s) form 3′-overhang or 5′-overhang of 0-5 nucleotides on one end of the duplex formed when each of the nucleotide sequence and the one RNA molecule is hybridized with the other. Following introduction of the composition into a cell, the nucleotide sequence and the one or more RNA molecule(s) are hybridized with the other and the nucleotide sequence is processed. The processing of the nucleotide sequence may be capable of effecting the cleavage, directly or indirectly, of the carrier RNA at the carrier cleavage site and the processing of the nucleotide sequence is capable of forming one or more RNA duplex(es), such that the processing of the nucleotide sequence may include, for example, Dicer processing and the RNA duplex(es) may be siRNA duplex(es) and/or miRNA duplex(es).

In some embodiments, the functional RNA described in any of the previous 2 embodiments is a nucleic acid sequence of from 18 to 25 nucleotides in length which is of sufficient complementarity to a target sequence to direct target-specific RNA interference. The target sequence is a sequence of from 18 to 25 nucleotides in length that is located in a region within the endogenous signal RNA, such that the region is located from about 25 nucleotides downstream from the predetermined cleavage site to about 25 nucleotides upstream from the predetermined cleavage site, such that the nucleic acid sequence is located within the nucleotide sequence or within at least one RNA molecule from the one or more RNA molecule(s). Following introduction of the composition into a cell, at least one RNA duplex of the one or more RNA duplex(es) comprises the nucleic acid sequence, and the RNA duplex that comprises the nucleic acid sequence directs the cleavage of the endogenous signal RNA at the predetermined cleavage site via, for example, RNA interference.

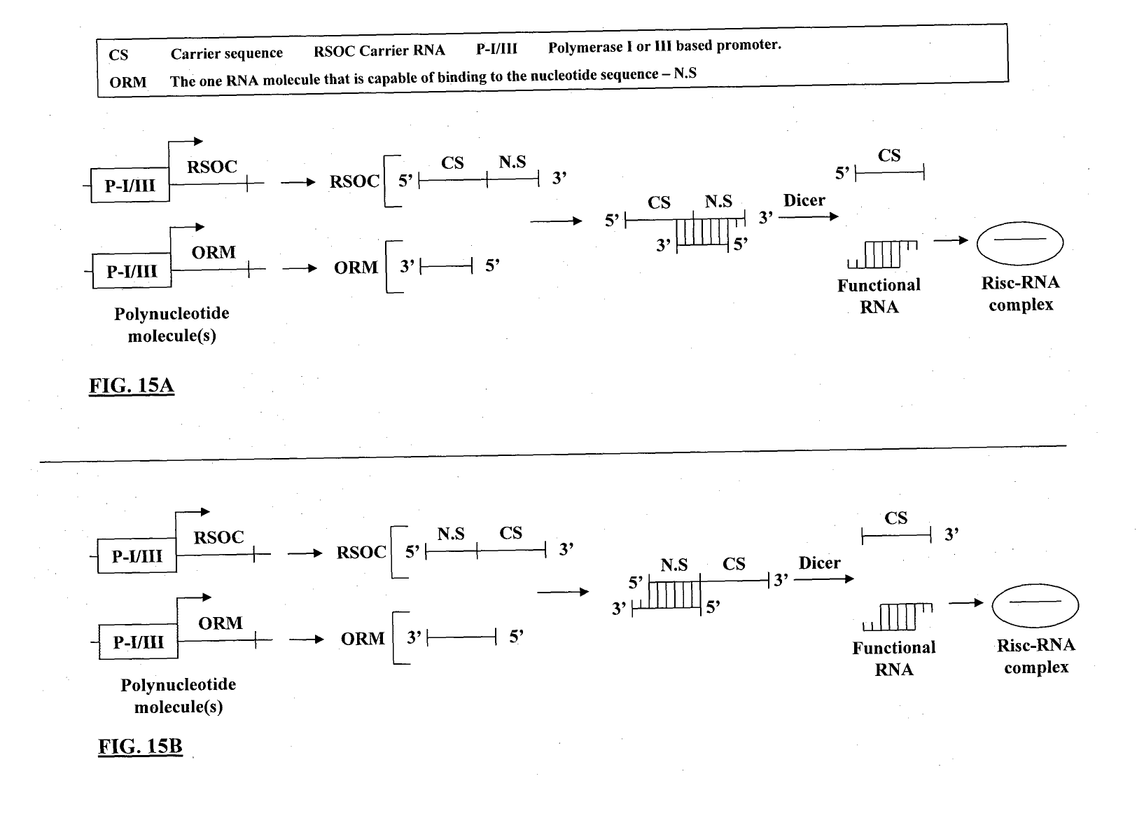

In one embodiment, the region described in the former embodiment is located from about 11 nucleotides downstream from the predetermined cleavage site to about 12 nucleotides upstream from the predetermined cleavage site. In another embodiment, the one or more RNA molecule(s) that are described in any of the previous 2 embodiments is one RNA molecule, consisting essentially of the nucleic acid sequence, such that the nucleotide sequence is 18-25 nucleotides in length and the one RNA molecule is 18-25 nucleotides in length. The nucleotide sequence and the one RNA molecule form 3′-overhang of 2 nucleotides on one end of the duplex formed when each of the nucleotide sequence and the one RNA molecule is hybridized with the other, the expressions of the carrier RNA and the one RNA molecule are driven by polymerase I or III based promoter. For example, see FIGS. 15A and 15B.

According to some embodiments, and without wishing to bound to theory or mechanism, when the functional RNA and the carrier sequence are located in the same RNA duplex, the carrier sequence may bring the functional RNA into proximity with the predetermined signal sequence of the endogenous signal RNA and may further bring also the components of the RNA interference pathway (for example, Dicer and Risc) into proximity with the predetermined signal sequence.

In another embodiment of the invention, the functional RNA described in embodiments in section 1 comprises:
(i) a nucleotide sequence that is located at the 5′ end of the carrier RNA; and (ii) one or more RNA molecule(s) that are capable of binding to the nucleotide sequence; such that the nucleotide sequence and at least one RNA molecule form 3′-overhang or 5′-overhang of 0-5 nucleotides on one end of the duplex, which is formed when each of the nucleotide sequence and the at least one RNA molecule is hybridized with the other. The nucleotide sequence or at least one RNA molecule include a nucleic acid sequence of from 18 to 25 nucleotides in length that is of sufficient complementarity to a target sequence to direct target-specific RNA interference, such that the target sequence is a sequence of from 18 to 25 nucleotides in length that is located in a region within the endogenous signal RNA, such that the region is located from about 25 nucleotides downstream from the predetermined cleavage site to about 25 nucleotides upstream from the predetermined cleavage site. Following introduction of the composition into a cell, the nucleotide sequence and the one or more RNA molecule(s) may be hybridized with the other and the nucleotide sequence may be processed, such that the processing of the nucleotide sequence is capable of forming one or more RNA duplex(es). The processing of the nucleotide sequence may include Dicer processing and the RNA duplex(es) may be siRNA duplex(es) and/or miRNA duplex(es), such that at least one RNA duplex from the one or more RNA duplex(es) comprises the nucleic acid sequence and such that the RNA duplex that comprises the nucleic acid sequence directs the cleavage of the endogenous signal RNA at the predetermined cleavage site via RNA interference.

In another embodiment of the invention, the functional RNA described in embodiments in section 1 may comprise:
(i) a nucleotide sequence that is located at the 3′ end of the carrier RNA; and (ii) one or more RNA molecule(s) that are capable of binding to the nucleotide sequence; such that the nucleotide sequence and at least one RNA molecule form 3′-overhang or 5′-overhang of 0-5 nucleotides on one end of the duplex which is formed when each of the nucleotide sequence and the one RNA molecule is hybridized with the other. The nucleotide sequence or at least one RNA molecule from the one or more RNA molecule(s) comprises a nucleic acid sequence of from 18 to 25 nucleotides in length that is of sufficient complementarity to a target sequence to direct target-specific RNA interference, such that the target sequence is a sequence of from 18 to 25 nucleotides in length that is located in a region within the endogenous signal RNA, wherein the region is located from about 25 nucleotides downstream from the predetermined cleavage site to about 25 nucleotides upstream from the predetermined cleavage site. Following introduction of the composition into a cell, the nucleotide sequence and the one or more RNA molecule(s) are hybridized with each other and the nucleotide sequence is processed, such that the processing of the nucleotide sequence is capable of forming one or more RNA duplex(es). The processing of the nucleotide sequence may include, for example, Dicer processing and the RNA duplex(es) may be siRNA duplex(es) and/or miRNA duplex(es), such that at least one RNA duplex from the one or more RNA duplex(es) comprises the nucleic acid sequence and such that the RNA duplex that comprises the nucleic acid sequence directs the cleavage of the endogenous signal RNA at the predetermined cleavage site via RNA interference.

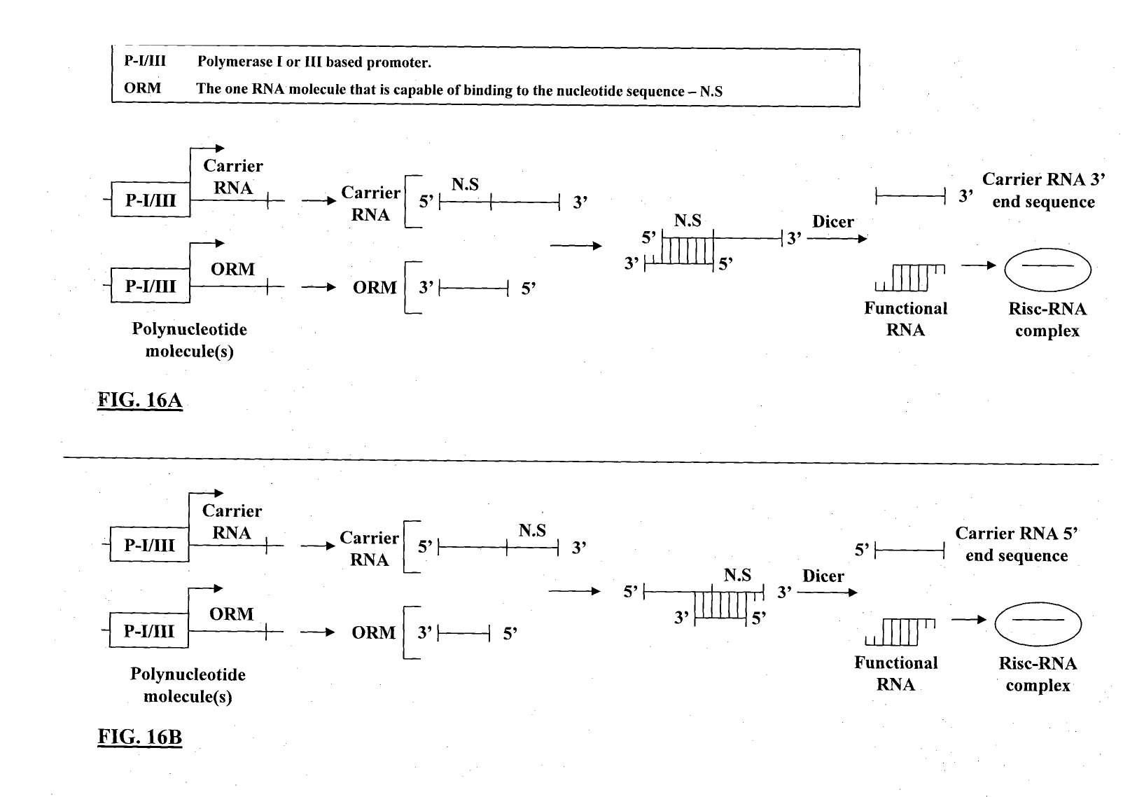

In additional embodiment, the region that is described in any of the previous 2 embodiments is located from about 11 nucleotides downstream from the predetermined cleavage site to about 12 nucleotides upstream from the predetermined cleavage site. In another embodiment, the one or more RNA molecule(s) that is described in any of the previous 3 embodiments is one RNA molecule, such that the nucleotide sequence or the one RNA molecule is consisting essentially of the nucleic acid sequence. The nucleotide sequence is 18-25 nucleotides in length and the one RNA molecule is 18-25 nucleotides in length, such that the nucleotide sequence and the RNA molecule form 3′-overhang of 2 nucleotides on one end of the duplex, which is formed when each of the nucleotide sequence and the one RNA molecule is hybridized with the other. In some embodiments, the expression of the carrier RNA and the one RNA molecule are driven by polymerase I or III based promoter. For example, see FIGS. 16A and 16B.

According to some embodiments, and without wishing to bound to theory or mechanism, when the functional RNA and the carrier RNA are located in the same RNA duplex, the carrier RNA may bring the functional RNA into proximity with the predetermined signal sequence of the endogenous signal RNA and by this may also bring the components of the RNA interference pathway (for example, Dicer and Rise) into proximity with the predetermined signal sequence.

In another embodiment of the invention, the functional RNA described in embodiments in section 1 may comprise:
(i) a nucleotide sequence that is located at the 5′ end of the carrier RNA; and; (ii) one or more RNA molecule(s) that are capable of binding to the nucleotide sequence; such that the nucleotide sequence and at least one RNA molecule form 3′-overhang or 5′-overhang of 0-5 nucleotides on one end of the duplex formed, when each of the nucleotide sequence and the RNA molecule is hybridized with each other. The nucleotide sequence or at least one RNA molecule may comprise a nucleic acid sequence of from 18 to 25 nucleotides in length, such that the nucleic acid sequence is of sufficient complementarity to a target sequence to direct target-specific RNA interference. The target sequence is a sequence of from 18 to 25 nucleotides in length that is located in a region within the endogenous signal RNA, such that the region is located from about 25 nucleotides downstream from the predetermined cleavage site to about 25 nucleotides upstream from the predetermined cleavage site. Following introduction of the composition into a cell, the nucleotide sequence and the RNA molecule are hybridized with each other and the nucleotide sequence is processed, such that the processing of the nucleotide sequence is capable of forming one or more RNA duplex(es). The processing of the nucleotide sequence may include, for example, Dicer processing and the RNA duplex(es) may be siRNA duplex(es) and/or miRNA duplex(es), such that at least one RNA duplex from the one or more RNA duplex(es) includes the nucleic acid sequence and such that the RNA duplex that comprises the nucleic acid sequence directs the cleavage of the endogenous signal RNA at the predetermined cleavage site via RNA interference.

In another embodiment of the invention, the functional RNA described in embodiments in section 1 may comprise:
(i) a nucleotide sequence that is located at the 3′ end of the carrier RNA sequence; and;
(ii) one or more RNA molecule(s) that are capable of binding to the nucleotide sequence; such that the nucleotide sequence and at least one RNA molecule form 3′-overhang or 5′-overhang of 0-5 nucleotides on one end of the duplex, which is formed when each of the nucleotide sequence and the one RNA molecule is hybridized with each other. The nucleotide sequence or the at least one RNA molecule may comprise a nucleic acid sequence of from 18 to 25 nucleotides in length, such that the nucleic acid sequence is of sufficient complementarity to a target sequence to direct target-specific RNA interference. The target sequence is a sequence of from 18 to 25 nucleotides in length that is located in a region within the endogenous signal RNA, such that the region is located from about 25 nucleotides downstream from the predetermined cleavage site to about 25 nucleotides upstream from the predetermined cleavage site. Following introduction of the composition into a cell, the nucleotide sequence and the one or more RNA molecule(s) are hybridized with each other and the nucleotide sequence is processed, such that the processing of the nucleotide sequence is capable of forming one or more RNA duplex(es). The processing of the nucleotide sequence may include, for example, Dicer processing and the RNA duplex(es) may be siRNA duplex(es) and/or miRNA duplex(es), such that at least one RNA duplex from the one or more RNA duplex(es) may comprise the nucleic acid sequence and such that the RNA duplex that comprises the nucleic acid sequence may direct the cleavage of the endogenous signal RNA at the predetermined cleavage site via RNA interference.

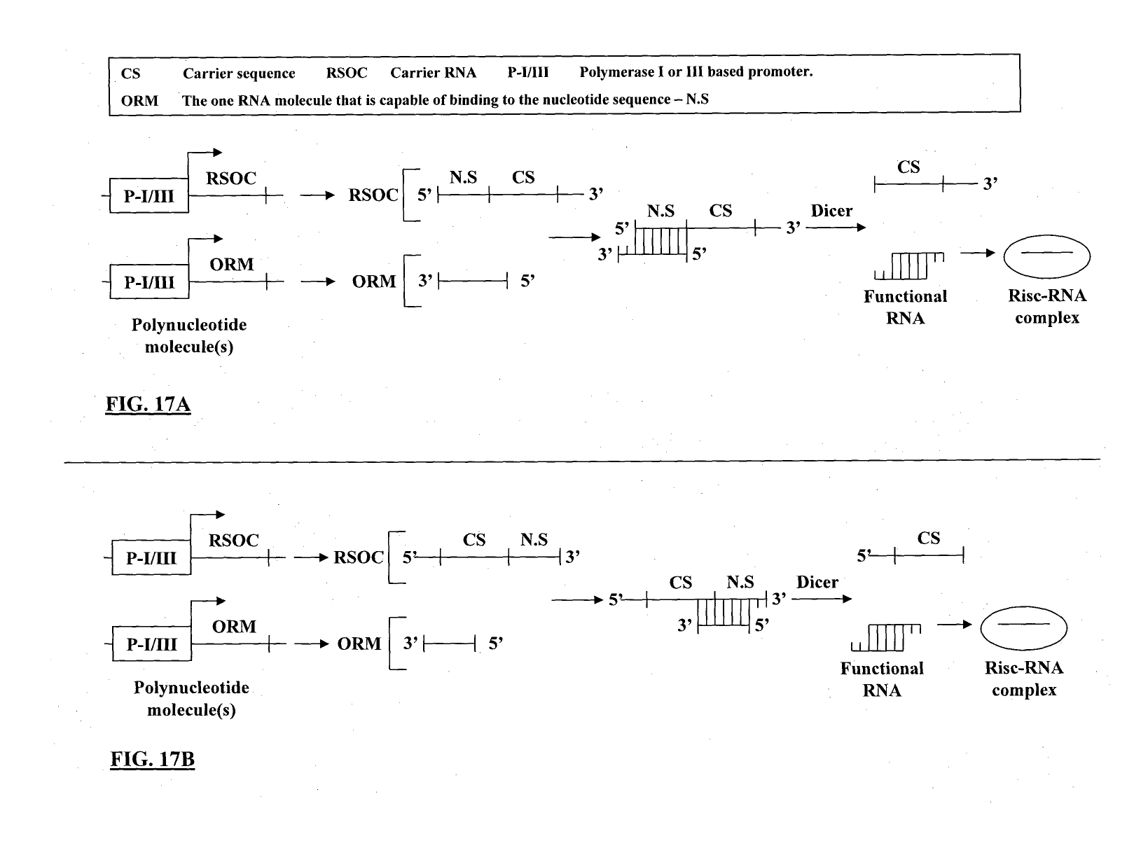

In another embodiment, the region that described in any of the previous two embodiments may be located from about 11 nucleotides downstream from the predetermined cleavage site to about 12 nucleotides upstream from the predetermined cleavage site. In another embodiment, the one or more RNA molecule(s) that are described in any of the previous 3 embodiments is one RNA molecule, such that the nucleotide sequence or the one RNA molecule is consisting essentially of the nucleic acid sequence. The nucleotide sequence may be 18-25 nucleotides in length and the one RNA molecule is 18-25 nucleotides in length. The nucleotide sequence and the one RNA molecule form 3′-overhang of 2 nucleotides on one end of the duplex, which is formed when each of the nucleotide sequence and the one RNA molecule is hybridized with each other. The expression of the carrier RNA and the one RNA molecule are driven by polymerase I or III based promoter. For example, see FIGS. 17A and 17B.

In some embodiments, the functional nucleic acid described in embodiments in section 1 may comprise:
(i) a nucleotide sequence that is located at the 5′ end of the carrier RNA sequence; and;
(ii) one or more RNA molecule(s) that are capable of binding to the nucleotide sequence; such that the nucleotide sequence and one RNA molecule from the one or more RNA molecule(s) form 3′-overhang or 5′-overhang of 0-5 nucleotides on one end of the duplex, which is formed when each of the nucleotide sequence and the one RNA molecule is hybridized with the other. Such that the nucleotide sequence or at least one RNA molecule from the one or more RNA molecule(s) comprises a nucleic acid sequence of from 18 to 25 nucleotides in length, such that the nucleic acid sequence is of sufficient complementarity to a target sequence to direct target-specific RNA interference, such that the target sequence is a sequence of from 18 to 25 nucleotides in length that is located in a region within the carrier RNA and such that the region is located from about 25 nucleotides downstream from the carrier cleavage site to about 25 nucleotides upstream from the carrier cleavage site. Such that following introduction of the composition into a cell, the nucleotide sequence and the one or more RNA molecule(s) are hybridized with the other and the nucleotide sequence is processed, such that the processing of the nucleotide sequence is capable of forming one or more RNA duplex(es). The processing of the nucleotide sequence may include, for example, Dicer processing and the RNA duplex(es) may be siRNA duplex(es) and/or miRNA duplex(es), such that at least one RNA duplex from the one or more RNA duplex(es) comprises the nucleic acid sequence and such that the RNA duplex that comprises the nucleic acid sequence directs the cleavage of the carrier RNA at the carrier cleavage site via RNA interference.

In another embodiment of the invention, the functional nucleic acid described in embodiments in section 1 comprises:
(i) a nucleotide sequence that is located at the 3′ end of the carrier RNA sequence); and
(ii) one or more RNA molecule(s) that are capable of binding to the nucleotide sequence, such that the nucleotide sequence and one RNA molecule from the one or more RNA molecule(s) form 3′-overhang or 5′-overhang of 0-5 nucleotides on one end of the duplex formed when each of the nucleotide sequence and the one RNA molecule is hybridized with the other.

Such that the nucleotide sequence or at least one RNA molecule from the one or more RNA molecule(s) comprises a nucleic acid sequence of from 18 to 25 nucleotides in length, wherein the nucleic acid sequence is of sufficient complementarity to a target sequence to direct target-specific RNA interference, such that the target sequence is a sequence of from 18 to 25 nucleotides in length that is located in a region within the carrier RNA sequence and such that the region is located from about 25 nucleotides downstream from the carrier cleavage site to about 25 nucleotides upstream from the carrier cleavage site. Following introduction of the composition into a cell, the nucleotide sequence and the one or more RNA molecule(s) are hybridized with each other and the nucleotide sequence is processed, such that the processing of the nucleotide sequence is capable of forming one or more RNA duplex(es). The processing of the nucleotide sequence may include, for example, Dicer processing and the RNA duplex(es) may comprise siRNA duplex(es) and/or miRNA duplex(es), such that at least one RNA duplex from the one or more RNA duplex(es) comprises the nucleic acid sequence and such that the RNA duplex that comprises the nucleic acid sequence directs the cleavage of the carrier RNA at the carrier cleavage site via RNA interference.

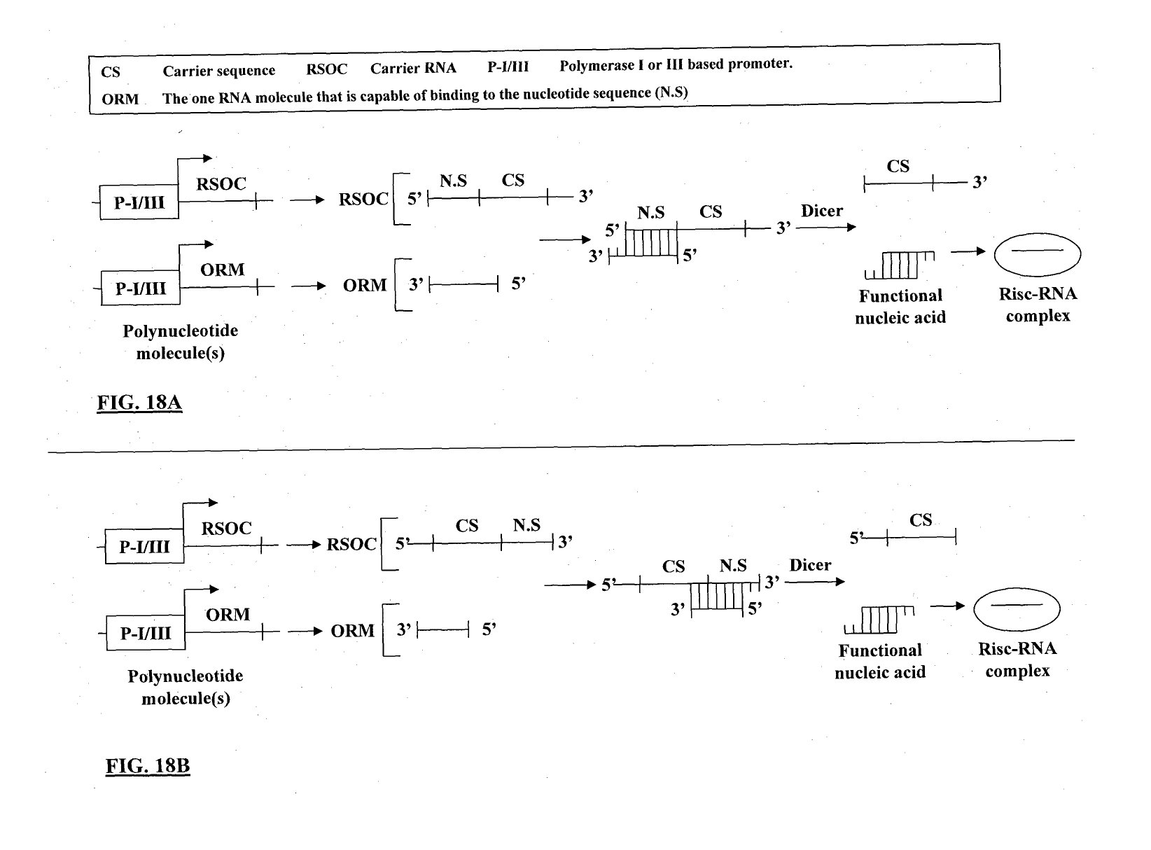

In another embodiment, the region described in any of the previous 2 embodiments may be located from about 11 nucleotides downstream from the carrier cleavage site to about 12 nucleotides upstream from the carrier cleavage site. In another embodiment, the one or more RNA molecule(s) described in any of the previous 3 embodiments is one RNA molecule, such that the nucleotide sequence or the one RNA molecule is consisting essentially of the nucleic acid sequence. The nucleotide sequence is 18-25 nucleotides in length, such that the one RNA molecule is 18-25 nucleotides in length, and the nucleotide sequence and the one RNA molecule form 3′-overhang of 2 nucleotides on one end of the duplex formed, when each of the nucleotide sequence and the one RNA molecule is hybridized with the other. The expression of the carrier RNA and the one RNA molecule may be driven by polymerase I or III based promoter. For example, see FIGS. 18A and 18B.

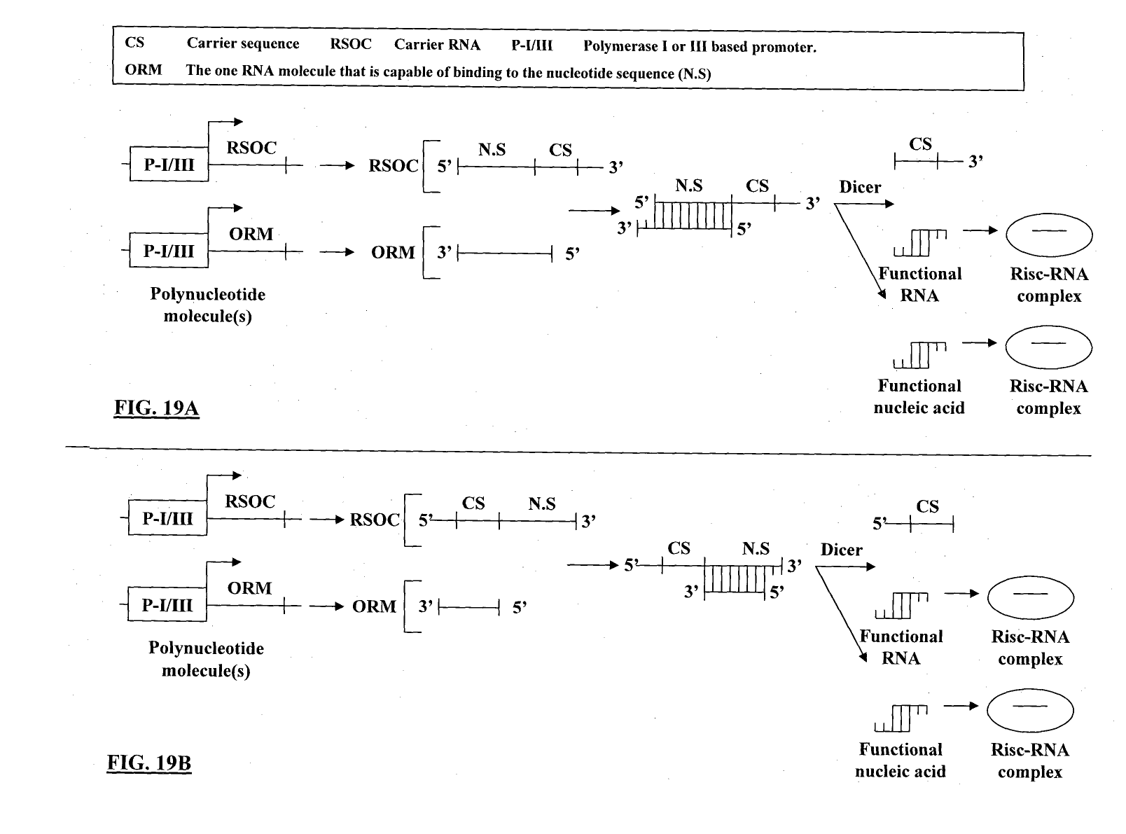

In another embodiment, the functional RNA is a specific nucleotide sequence of from 18 to 25 nucleotides in length which is of sufficient complementarity to a specific target sequence to direct target-specific RNA interference. The specific target sequence is a sequence of from 18 to 25 nucleotides in length that is located in a specific region within the endogenous signal RNA, such that the specific region is located from about 25 nucleotides downstream from the predetermined cleavage site to about 25 nucleotides upstream from the predetermined cleavage site. The specific nucleotide sequence is located within the nucleotide sequence or within at least one RNA molecule from the one or more RNA molecule(s). Following introduction of the composition into a cell, at least one RNA duplex from the one or more RNA duplex(es) include the specific nucleotide sequence and such that the RNA duplex that comprises the specific nucleotide sequence directs the cleavage of the endogenous signal RNA at the predetermined cleavage site via RNA interference. For example, see FIGS. 19A and 19B.

### 5 STRUCTURE OF A CARRIER RNA SEQUENCE THAT COMPRISES AT LEAST 3 CONTIGUOUS CARRIER SEQUENCES

This section describes the structure of the carrier RNA described in section 1, such that the carrier RNA comprises at least 3 contiguous carrier sequences.

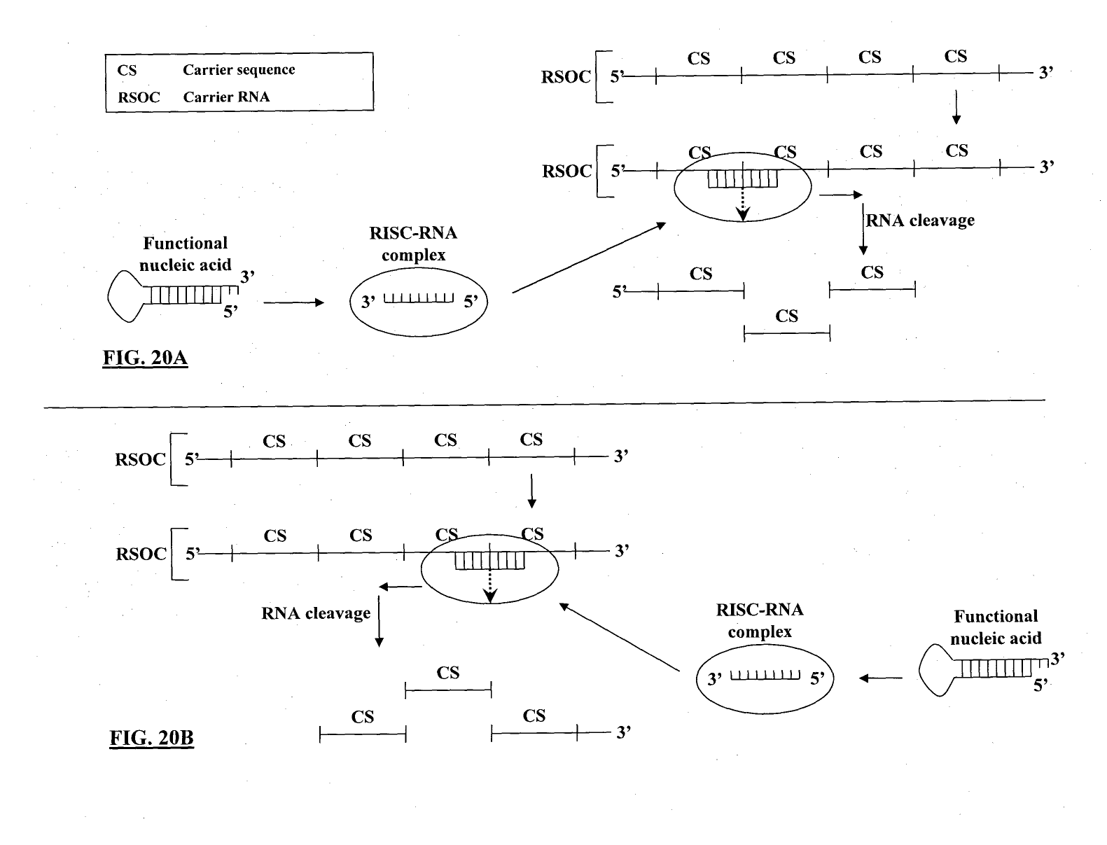

In some embodiments of the invention, the carrier RNA described in the embodiments in section 1 may further comprise at least 2 contiguous carrier sequences immediately downstream from the described carrier sequence. For example, see FIG. 20A.

In an additional embodiment, the carrier RNA described in embodiments of section 1 may further comprise 100 contiguous carrier sequences (that may be identical or different) immediately downstream from the carrier sequence described therein, such that the edge sequence is 23-28 nucleotides in length and is located from the predetermined cleavage site to about 23-28 nucleotides downstream; the second sequence is 2 nucleotides in length; the third sequence is 0 nucleotides in length and the expression of the polynucleotide sequence the carrier RNA is driven by CMV-IE promoter.

In further embodiments, the carrier RNA described in the embodiments in section 1 may further comprise at least 2 contiguous carrier sequences immediately upstream from the carrier sequence. For example, see FIG. 20B.

In another embodiment, the carrier RNA, described in embodiments in section 1 may further comprise 100 contiguous carrier sequences, that may be identical or different, immediately upstream from the carrier sequence, such that the edge sequence is 25-30 nucleotides in length and is located 2 nucleotides upstream from the predetermined cleavage site and extends upstream in the endogenous signal RNA; the second sequence is 0 nucleotides in length; the third sequence is 0 nucleotides in length; and such that the expression of the polynucleotide sequence the carrier RNA is driven by CMV-IE promoter.

According to some embodiments, and without wishing to bound to theory or mechanism, advantageous results may be obtained when the functional nucleic acid is, for example, microRNA (miRNA), lariat-form RNA, short-hairpin RNA (smRNA), siRNA expression domain, small-interfering RNA (siRNA) and/or trans acting ribozyme, since with such functional nucleic acids, many carrier sequences can be generated from one carrier RNA and from one functional nucleic acid.

### 6. STRUCTURE OF POLYNUCLEOTIDE MOLECULE(S) THAT MAY FURTHER TRANSCRIBE AN ADDITIONAL FUNCTIONAL RNA THAT MAY CLEAVE THE PREDETERMINED SIGNAL SEQUENCE AT THE OPPOSITE SIDE, WHICH IS NOT CLEAVED

This section describes embodiments of the structure of the polynucleotide molecule(s) (such as, for example, DNA molecules), described in embodiments of section 1, such that the polynucleotide molecule(s) together further transcribe an additional functional RNA that is capable of effecting the cleavage of the endogenous signal RNA at the opposite end of the predetermined signal sequence, which is not cleaved.

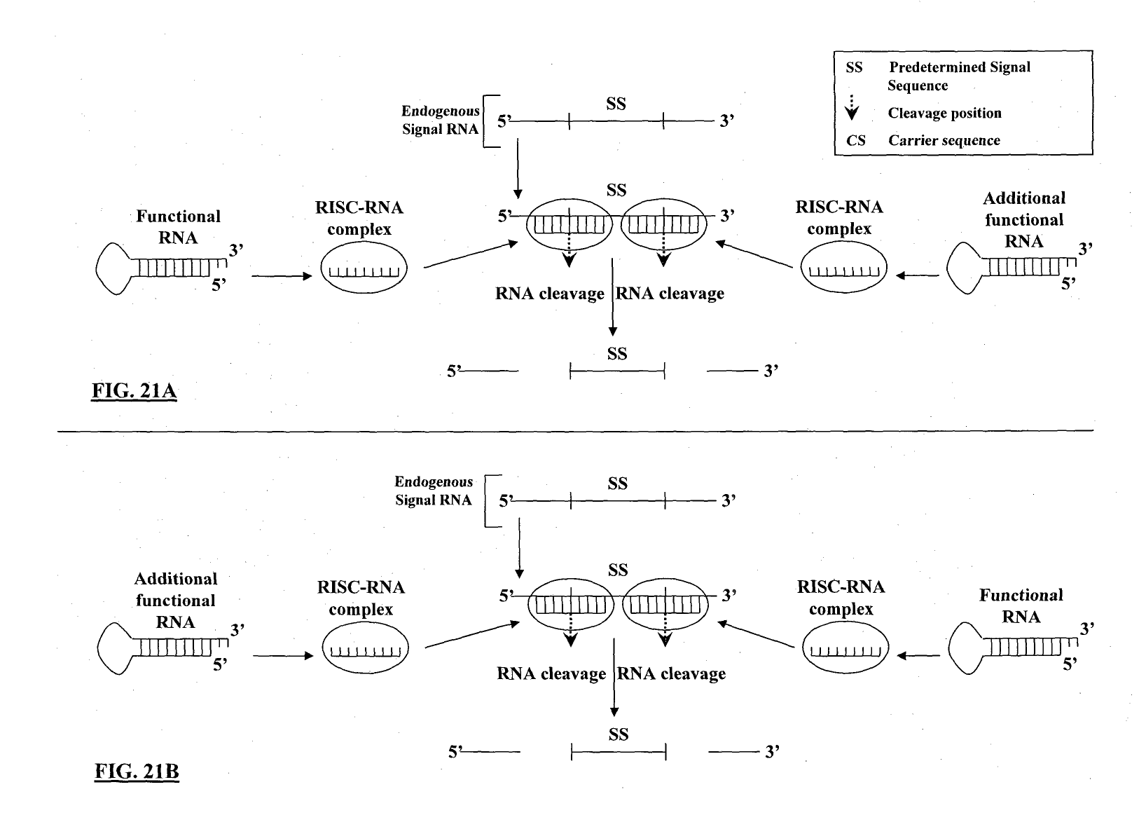

In some embodiments of the invention, the polynucleotide molecule(s) described in some embodiments of section 1 further comprise a polynucleotide sequence encoding an additional functional RNA that is capable of effecting the cleavage, directly or indirectly, of the endogenous signal RNA at an additional cleavage site, such that the additional cleavage site may be located 0-1000 nucleotides downstream from the 3′ end of the predetermined signal sequence. In one embodiment, the additional cleavage site may be located 0-5 nucleotides downstream from the 3′ end of the predetermined signal sequence. For example, see FIG. 21A.

In additional embodiments, the polynucleotide molecule(s) described in some embodiments in section 1 may further comprise a polynucleotide sequence encoding an additional functional RNA that is capable of effecting the cleavage, directly or indirectly, of the endogenous signal RNA at an additional cleavage site, such that the additional cleavage site may be located 0-1000 nucleotides upstream from the 5′ end of the predetermined signal sequence. In one embodiment, the additional cleavage site may be located 0-5 nucleotides upstream from the 5′ end of the predetermined signal sequence. For example, see FIG. 21B.

According to some embodiments, and without wishing to bound to theory or mechanism, the previous 4 embodiments may be advantageous since in these embodiments the predetermined signal sequence may be cleaved at both of its ends and thus with the carrier RNA/sequence it may be a better substrate for endogenous enzymes, such as, for example, Dicer and/or Risc.

### 7. STRUCTURE OF AN EXOGENOUS RNA OF INTEREST

In some embodiments of the invention, the exogenous RNA of interest described in embodiments in section 1 may further comprise:

a sequence encoding an exogenous protein of interest; and

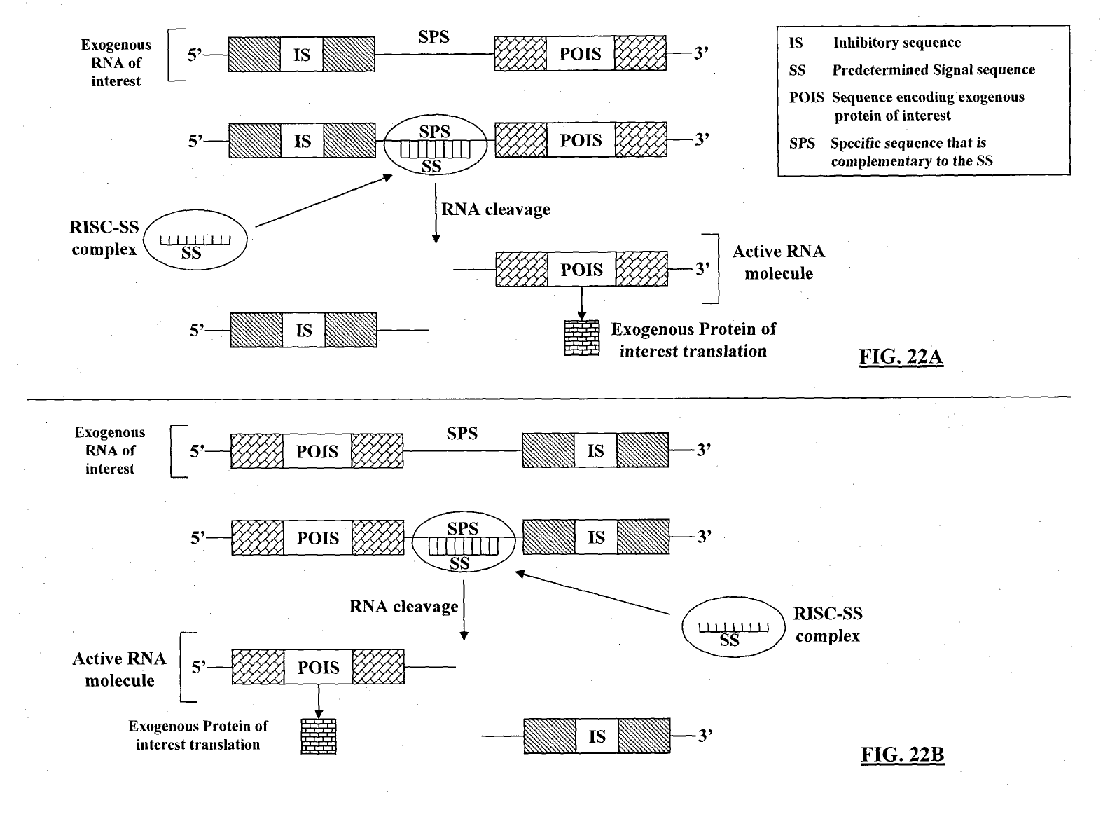

an inhibitory sequence that is capable of inhibiting the expression of the exogenous protein of interest;

whereby the specific target/cleavage site is located between the inhibitory sequence and the sequence encoding the exogenous protein of interest. Following introduction of the composition into a cell comprising the endogenous signal RNA, the exogenous RNA of interest is transcribed and cleaved at the specific target/cleavage site whereby the inhibitory sequence is detached from the sequence encoding the exogenous protein of interest and the exogenous protein of interest is capable of being expressed. For example, see FIGS. 22A and 22B. Accordingly, cleaving of the exogenous RNA of interest may lead to the expression of an active exogenous protein of interest within the cell.

In some embodiments, the exogenous RNA of interest molecule may further comprise a carrier RNA sequence and/or a Functional RNA sequence.

As known in the art, mRNAs without cap or poly A tail are still capable of translating proteins. In mammalian cells, an addition of a cap increases the translation of an mRNA by 35-50 fold and an addition of a poly(A) tail increases the translation of an mRNA by 114-155-fold [10]. The poly(A) tail in mammalian cells increases the functional mRNA half-life only by 2.6-fold and the cap increases the functional mRNA half-life only by 1.7-fold [10].

Some proteins may be biologically active even at a concentration of one protein per cell. It has been reported that a single protein of Ricin or Abrin reaching the cytosol can kill that cell [12, 13]. In addition, a single protein of Diphtheria toxin fragment A introduced into a cell can kill the cell [14]. The exogenous protein of interest of the invention can be any protein or peptide. For example, in some embodiments, the exogenous protein of interest may be any type of toxin (such as, for example, Ricin, Abrin, Diphtheria toxin (DTA), botulinium toxin); an enzyme; a reporter gene; a structural gene, and the like. In some embodiments, the exogenous protein of interest may be a polypeptide which is a fusion product of two proteins, that may be have a cleavage site there between, allowing the separation of the two proteins within the cell. For example, the exogenous protein of interest may be a fusion protein of Ricin and DTA, whereby cleavage of the fusion protein by, for example, a specific protease, can result in the formation of separate DTA and Ricin proteins in the cell. In some embodiment, the exogenous protein of interest may include two separate proteins, that may be expressed by the composition. For example, the exogenous RNA of interest may encode for two separate exogenous proteins of interest, such as, for example, Ricin and DTA.

7.1. Structure of an Exogenous RNA of Interest Having an Inhibitory Sequence Located Upstream from the Specific Target/Cleavage Site
7.1.1. Structure of the Inhibitory Sequence that is Located Upstream from the Specific Target/Cleavage Site

The inhibitory sequence in the exogenous RNA of interest described in embodiments of Section 7 may be located upstream or downstream from the specific target/cleavage site. This section describes the structure of the inhibitory sequence that is located upstream from the specific target/cleavage site in the exogenous RNA of interest, according to some embodiments. For example, see FIG. 22A.

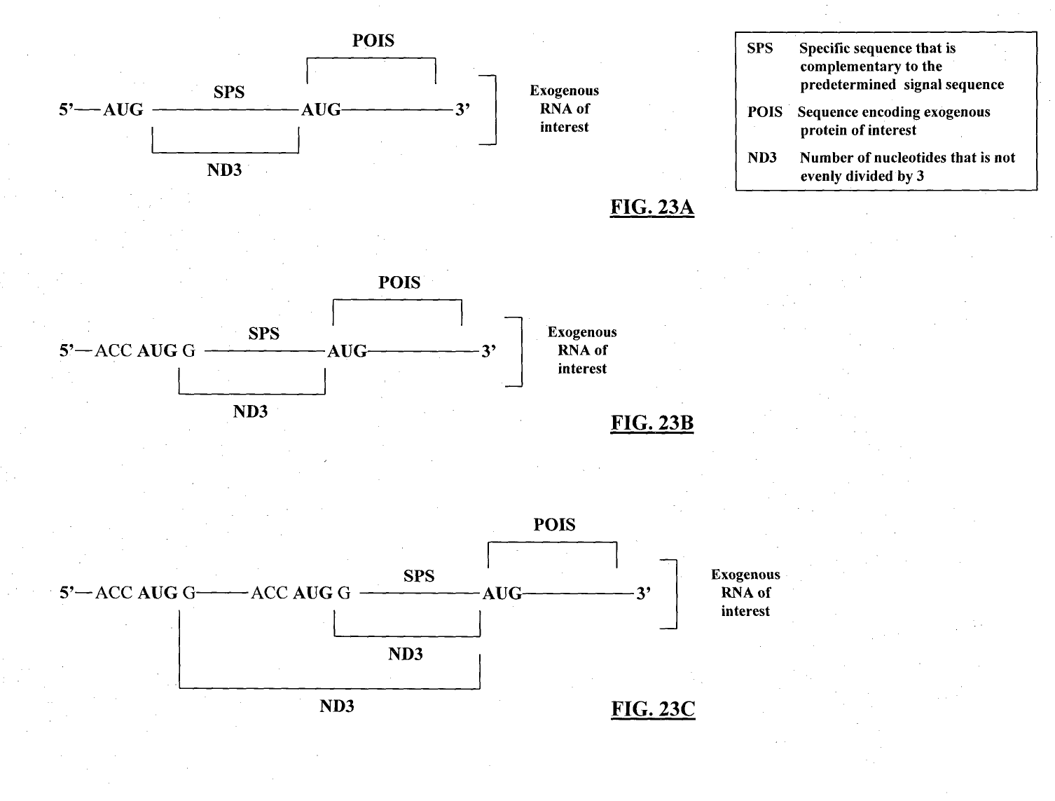

In some embodiments of the invention, the inhibitory sequence that is located upstream from the specific target/cleavage site and that is described in embodiments in section 7 may comprise, for example, but is not limited to an initiation codon, whereby the initiation codon and the sequence encoding for the exogenous protein of interest are not in the same reading frame, such that the initiation codon causes a frameshift mutation to the protein of interest that is encoded downstream. For example, see FIG. 23A. In one embodiment, the initiation codon is located within a Kozak consensus sequence. In addition, modified Kozak consensus sequences that maintain the ability to function as initiator of translation may be also used. In some embodiments, any initiator of translation element may be used. For example, see FIG. 23B.

For example, the Kozak consensus sequence in human is 5′-ACCAUGG-3′ (SEQ ID NO. 25) and the initiation codon is 5′-AUG-3′.

In another embodiment of the invention, the inhibitory sequence that is located upstream from the specific target/cleavage site and that is described in section 7 comprises a plurality of initiation codons, whereby each of the initiation codons and the sequence encoding exogenous protein of interest are not in the same reading frame, such that the initiation codons cause a frameshift mutation to the exogenous protein of interest that is encoded downstream. In addition, each of the initiation codons is located within a Kozak consensus sequence or a modified Kozak consensus sequences that maintain the ability to function as initiator of translation. For example, see FIG. 23C.

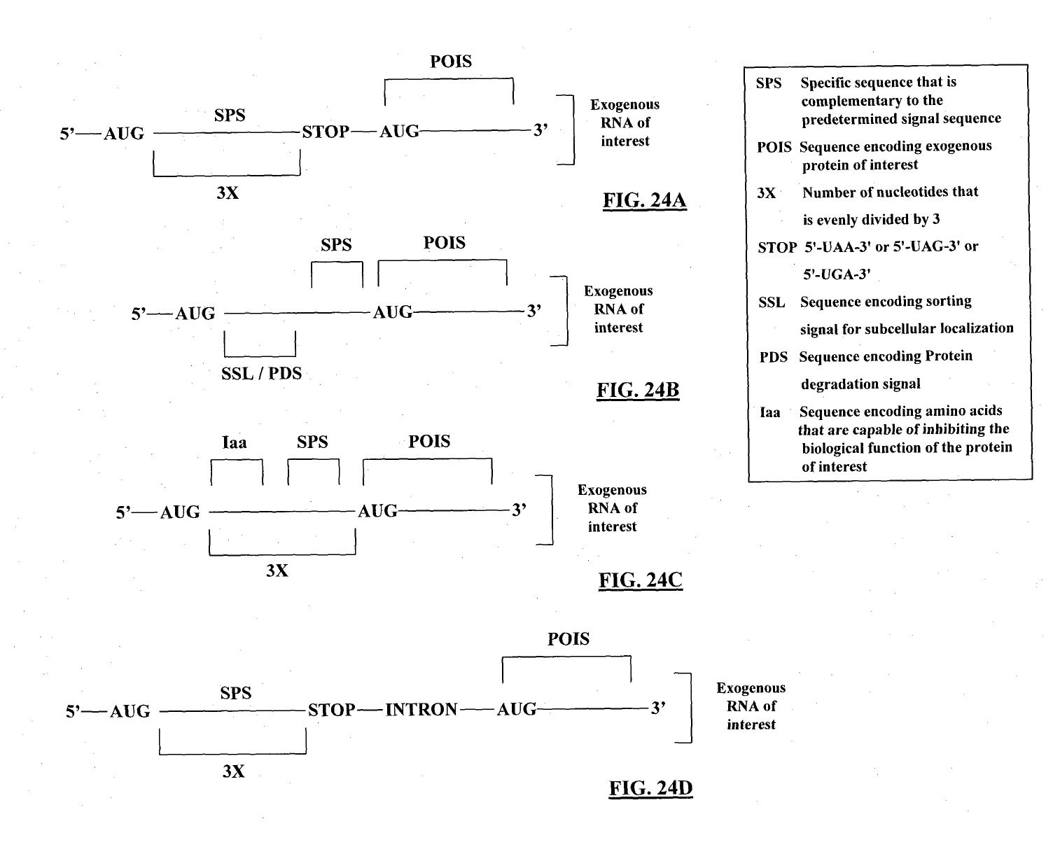

In some embodiments, the initiation codon may be located within or may comprise one or more TISU motifs. A TISU (Translation Initiator of Short 5′UTR) motif is distinguished from a Kozak consensus in its unique ability to direct efficient and accurate translation initiation from mRNAs with a very short 5′UTR. [39]. In other embodiment of the invention, the inhibitory sequence that is located upstream from the specific target/cleavage site and that is described in the specific embodiment in section 7 comprises an initiation codon and the exogenous RNA of interest further comprises a stop codon located between the initiation codon and the start codon of the sequence encoding the exogenous protein of interest, such that the stop codon and the initiation codon are in the same reading frame. Such a structure creates an upstream open reading frame (uORF) that reduces the efficiency of translation of the downstream sequence encoding protein of interest. For example, see FIG. 24A. In some embodiments, the stop codon may be, for example, 5′-UAA-3′ or 5′-UAG-3′ or 5′-UGA-3′.

In some embodiments, strong stems and loops may be located downstream to upstream ORF(s) at a location that is upstream or downstream to the target sequence for the miRNA (cleavage site). The creation of such stems and loops may aid in conditions, wherein despite having reached a stop codon, the small subunit of the ribosome does not detach from the mRNA continue to scan the mRNA. The small subunit of the ribosome is not capable of opening strong RNA secondary structures. Additionally, when these stems and loops are located downstream to the target sequence they may also block the degradation of the cleaved mRNA which may be performed, for example, by XRN1 exorinonuclease.

In another embodiment of the invention, the inhibitory sequence that is located upstream from the specific target/cleavage site and that is described in the specific embodiment in section 7 comprises an initiation codon and a nucleotide sequence downstream from the initiation codon that encodes a sorting/localization/targeting signal for subcellular localization, such that the nucleotide sequence and the initiation codon are in the same reading frame and such that the subcellular localization of the protein of interest inhibits its biological function. The sorting/localization signal for the subcellular localization includes, but is not limited to sorting localization signal for mitochondria, nucleus, endosome, lysosome, peroxisome, ER, or any subcellular localization or organelle. The sorting signal for the subcellular localization may be selected from, for example, but is not limited to: a peroxisomal targeting signal 2 [(R/K)(L/V/I)X5(Q/H)(L/A)] (SEQ ID NO. 26) or H₂N - - - RLRVLSGHL (SEQ ID NO. 27) (of human alkyl dihydroxyacetonephosphate synthase) [30]. For example, see FIG. 24B.

In another embodiment of the invention, the inhibitory sequence that is located upstream from the specific target/cleavage site and that is described in the specific embodiment in section 7 comprises an initiation codon and a nucleotide sequence downstream from the initiation codon that encodes a protein degradation signal, such that the nucleotide sequence and the initiation codon are in the same reading frame. The protein degradation signal includes, but is not limited to ubiquitin degradation signal. For example, see FIG. 24B.

In other embodiments of the invention, the inhibitory sequence that is located upstream from the specific target/cleavage site and that is described in section 7 is designed to comprise an initiation codon and a nucleotide sequence downstream from the initiation codon that is in the same reading frame with the initiation codon and with the sequence encoding the exogenous protein of interest, such that when the amino acid sequence, which is encoded by the nucleotide sequence, is fused to the protein of interest the biological function of the protein of interest is inhibited. For example, see FIG. 24C.

In another embodiment of the invention, the inhibitory sequence that is located upstream from the specific target/cleavage site and that is described in section 7 comprises an initiation codon, and the exogenous RNA of interest further comprises a stop codon downstream from the initiation codon, such that the stop codon and the initiation codon are in the same reading frame. In addition, the exogenous RNA ofinterest may further comprise an intron downstream from the stop codon, such that the exogenous RNA of interest is a target for nonsense-mediated decay (NMD) that degrades the exogenous RNA of interest. For example, see FIG. 24D.

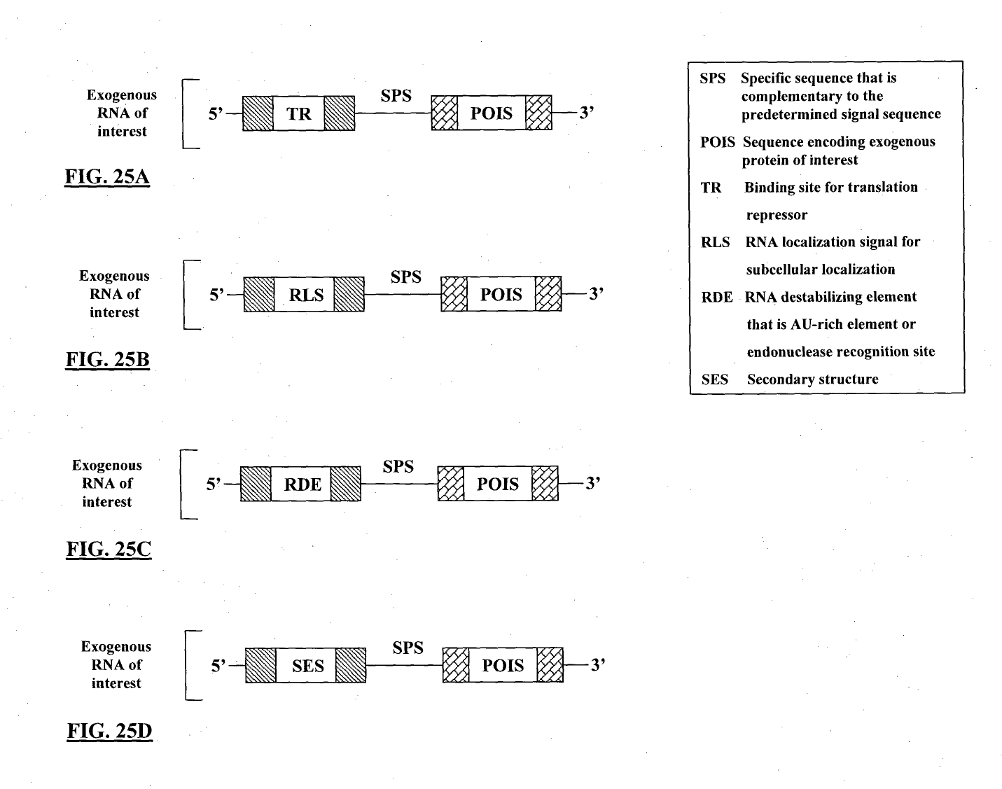

In another embodiment of the invention, the inhibitory sequence that is located upstream from the specific target/cleavage site and that is described in section 7 comprises a sequence that is capable of binding to a translation repressor protein, such that the translation repressor protein is an endogenous translation repressor protein or is encoded from the composition and such that the translation repressor protein, directly or indirectly, reduces the efficiency of translation of the protein of interest within the exogenous RNA of interest [26]. The sequence that is capable of binding to a translation repressor protein includes, for example, but is not limited to a sequence that binds the smaug repressor protein (5′-UGGAGCAGAGGCUCUGGCAGCUUUUGCAGCG-3′) (SEQ ID NO. 28) [27]. For example, see FIG. 25A.

In other embodiments of the invention, the inhibitory sequence that is located upstream from the specific target/cleavage site and that is described in section 7 comprises an RNA localization signal for subcellular localization (including cotranslational import) or an endogenous miRNA binding site, such that the subcellular localization of the exogenous RNA of interest of the invention inhibits the translation of the protein of interest and decreases the exogenous RNA of interest half-life. The RNA localization signal may be, for example, but is not limited to RNA localization signal for: myelinating periphery, mitochondria, myelin compartment, leading edge of the lamella or Perinuclear cytoplasm [24]. For example, the RNA localization signal for myelinating periphery is 5′-GCCAAGGAGCCAGAGAGCAUG-3′ (SEQ ID NO. 29) or 5′-GCCAAGGAGCC-3′ (SEQ ID NO. 30) [29]. For example, see FIG. 25B.

In another embodiment of the invention, the inhibitory sequence that is located upstream from the specific target/cleavage site and that is described in section 7 comprises an RNA destabilizing element that stimulates degradation of the exogenous RNA of interest, such that the RNA destabilizing element is an AU-rich element (ARE) or an endonuclease recognition site. The AU-rich element may be, for example, but is not limited to AU-rich elements that are at least about 35 nucleotides long. The AU-rich element may be, for example, but is not limited to: 5′-AUUUA-3′ (SEQ ID NO. 31), 5′-UUAUUUA(U/A)(U/A)-3′ (SEQ ID NO. 32) or 5′-AUUU-3′ (SEQ ID NO. 33) [28]. For example, see FIG. 25C.

In another embodiment of the invention, the inhibitory sequence that is located upstream from the specific target/cleavage site and that is described in section 7 comprises a sequence that is capable of forming a secondary structure that reduces the efficiency of translation of the downstream exogenous protein of interest. In one embodiment of the invention, and without wishing to bound to theory or mechanism, the free energy of folding of the secondary structure that described in the former embodiment may be lower than −30 kcal/mol (for example, −50 kcal/mol, −80 kcal/mol) and thus the secondary structure is sufficient to block scanning ribosomes to reach the start codon of the downstream protein of interest. For example, see FIG. 25D.

In further embodiment of the invention, the inhibitory sequence that is located upstream from the specific target/cleavage site and that is described in section 7 comprises a sequence immediately upstream from the specific target/cleavage site that is capable of binding to the nucleotide sequence that is located immediately downstream from the specific target/cleavage site for the formation of a secondary structure, such that the secondary structure, directly or indirectly, reduces the efficiency of translation of the downstream exogenous protein of interest.

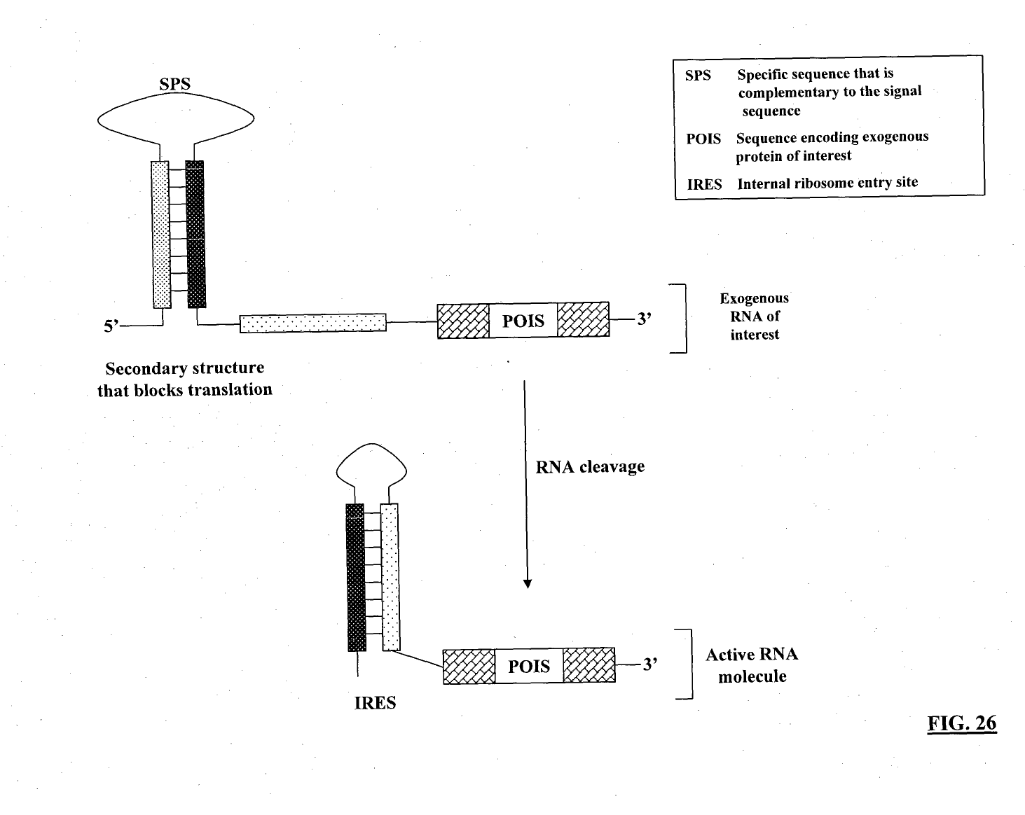

In some embodiments, the free energy of the folding of the secondary structure that is described in the former embodiment may be lower than −30 kcal/mol (for example, −50 kcal/mol and −80 kcal/mol) and thus this secondary structure is sufficient to block scanning ribosomes to reach the start codon of the protein of interest. In another embodiment, the specific target/cleavage site is located within the single stranded region or within the loop region in the secondary structure that is described in the former embodiment, such that the single stranded region or the loop region includes, but is not limited to region that is at least about 15 nucleotides long. In another embodiment, the exogenous RNA of interest, described in the former embodiment comprises an internal ribosome entry site (IRES) sequence downstream from the specific target/cleavage site and upstream from the sequence encoding protein of interest, such that the IRES sequence is more functional within the cleaved exogenous RNA of interest than within the intact exogenous RNA of interest. In other embodiment, at least part of the IRES sequence is located within the nucleotide sequence that is located immediately downstream from the specific target/cleavage site. For example, see FIG. 26.

In some embodiments, the IRES sequence includes, for example, but is not limited to a picornavirus IRES, a foot-and-mouth disease virus IRES, an encephalomyocarditis virus IRES, a hepatitis A virus IRES, a hepatitis C virus IRES, a human rhinovirus IRES, a poliovirus IRES, a swine vesicular disease virus IRES, a turnip mosaic potyvirus IRES, a human fibroblast growth factor 2 mRNA IRES, a pestivirus IRES, a Leishmania RNA virus IRES, a Moloney murine leukemia virus IRES a human rhinovirus 14 IRES, anaphthovirus IRES, a human immunoglobulin heavy chain binding protein mRNA IRES, a Drosophila Antennapedia mRNA IRES, a human fibroblast growth factor 2 mRNA IRES, a hepatitis G virus IRES, a tobamovirus IRES, a vascular endothelial growth factor mRNA IRES, a Coxsackie B group virus IRES, a c-myc protooncogene mRNA IRES, a human MYT2 mRNA IRES, a human parechovirus type 1 virus IRES, a human parechovirus type 2 virus IRES, a eukaryotic initiation factor 4GI mRNA IRES, a Plautia stali intestine virus IRES, a Theiler's murine encephalomyelitis virus IRES, a bovine enterovirus IRES, a connexin 43 mRNA IRES, a homeodomain protein Gtx mRNA IRES, an AML1 transcription factor mRNA IRES, an NF-kappa B repressing factor mRNA IRES, an X-linked inhibitor of apoptosis mRNA IRES, a cricket paralysis virus RNA IRES, a p58(PITSLRE) protein kinase mRNA IRES, an ornithine decarboxylase mRNA IRES, a connexin-32 mRNA IRES, a bovine viral diarrhea virus IRES, an insulin-like growth factor I receptor mRNA IRES, a human immunodeficiency virus type 1 gag gene IRES, a classical swine fever virus IRES, a Kaposi's sarcoma-associated herpes virus IRES, a short IRES selected from a library of random oligonucleotides, a Jembrana disease virus IRES, an apoptotic protease-activating factor 1 mRNA IRES, a Rhopalosiphum padi virus IRES, a cationic amino acid transporter mRNA IRES, a human insulin-like growth factor II leader 2 mRNA IRES, a giardiavirus IRES, a Smad5 mRNA IRES, a porcine teschovirus-1 talfan IRES, a Drosophila Hairless mRNA IRES, an hSNM1 mRNA IRES, a Cbfa1/Runx2 mRNA IRES, an Epstein-Barr virus IRES, a hibiscus chlorotic ringspot virus IRES, a rat pituitary vasopressin V1b receptor mRNA IRES and/or a human hsp70 mRNA IRES.

7.1.2. Additional Structures that May Increase the Efficiency of Translation of the Exogenous RNA of Interest that is Cleaved at the Specific Target/Cleavage Site at the 5′ End

This section describes further embodiments of additional structures of the composition of the invention that are described in embodiments in section 7, such that the additional structures may increase the efficiency of translation of the cleaved exogenous RNA of interest, wherein the cleaved exogenous RNA of interest is cleaved at the specific target/cleavage site at the 5′ end.

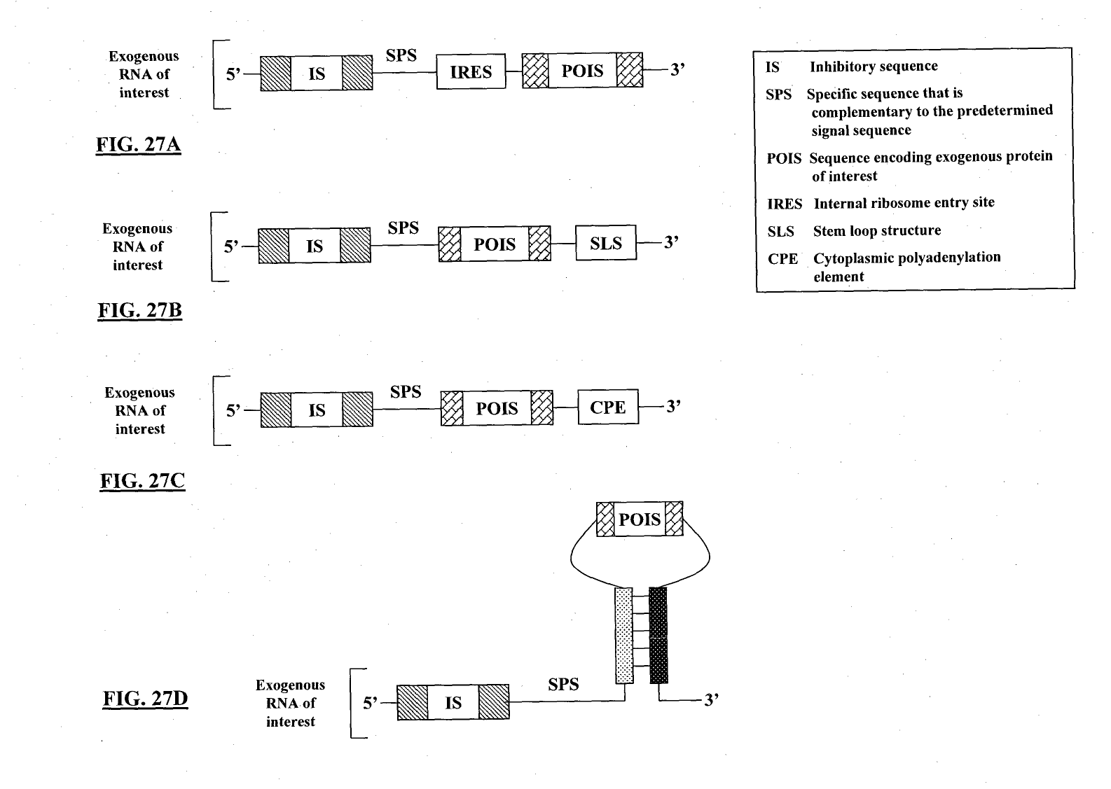

In some embodiments, the exogenous RNA of interest described in section 7 may comprise, for example, a sequence that comprises a unique internal ribosome entry site (IRES) sequence immediately upstream from the sequence encoding the exogenous protein of interest, such that the unique IRES sequence increases the efficiency of translation of the protein of interest in the cleaved exogenous RNA of interest. For example, see FIG. 27A.

In another embodiment of the invention, the exogenous RNA of interest that is described in section 7 may comprise a unique nucleotide sequence immediately downstream from the sequence encoding the protein of interest, such that the unique nucleotide sequence comprises a unique stem loop structure and such that the unique stem loop structure, directly or indirectly, increases the efficiency of translation of the protein of interest and the cleaved exogenous RNA of interest half-life. The unique stem loop structure may be, for example, but is not limited to the conserved stem loop structure of the human histone gene 3′-UTR or a functional derivative thereof. The conserved stem loop structure of the human histone gene 3′-UTR is 5′-GGCUCUUUUCAGAGCC-3′ (SEQ ID NO. 34). For example, see FIG. 27B.

In another embodiment of the invention, the exogenous RNA of interest that is described in the specific embodiment in section 7 may comprise a unique nucleotide sequence immediately downstream from the sequence encoding the protein of interest, such that the unique nucleotide sequence comprises a cytoplasmic polyadenylation element that, directly or indirectly, increases the efficiency of translation of the protein of interest and the half-life of the cleaved exogenous RNA of interest. The cytoplasmic polyadenylation element may be, for example, but is not limited to 5′-UUUUAU-3′(SEQ ID NO. 35 5′-UUUUUAU-3′(SEQ ID NO. 36), 5′-UUUUAAU-3′(SEQ ID NO. 37), 5′-UUUUUUAUU-3′(SEQ ID NO. 38), 5′-UUUUAUU-3′(SEQ ID NO. 39) or 5′-UUUUUAUAAAG-3′ (SEQ ID NO. 40) [25]. In some embodiments, the composition of the invention may also include, for example, a polynucleotide sequence that encodes a human cytoplasmic polyadenylation element binding protein (hCPEB), and/or a homologue thereof, for expressing hCPEB in any cell. For example, see FIG. 27C.

In another embodiment of the invention, the exogenous. RNA of interest that is described in section 7 comprises a unique nucleotide sequence that is located downstream from the specific target/cleavage site and upstream from the sequence encoding the exogenous protein of interest, such that the unique nucleotide sequence is capable of binding to a sequence that is located downstream from the sequence encoding protein of interest. Without wishing to be bound to theory or mechanism, in such embodiment, the cleaved exogenous RNA of interest may create a circular structure that increases the efficiency of translation of the protein of interest in the cleaved exogenous RNA of interest. For example, see FIG. 27D.

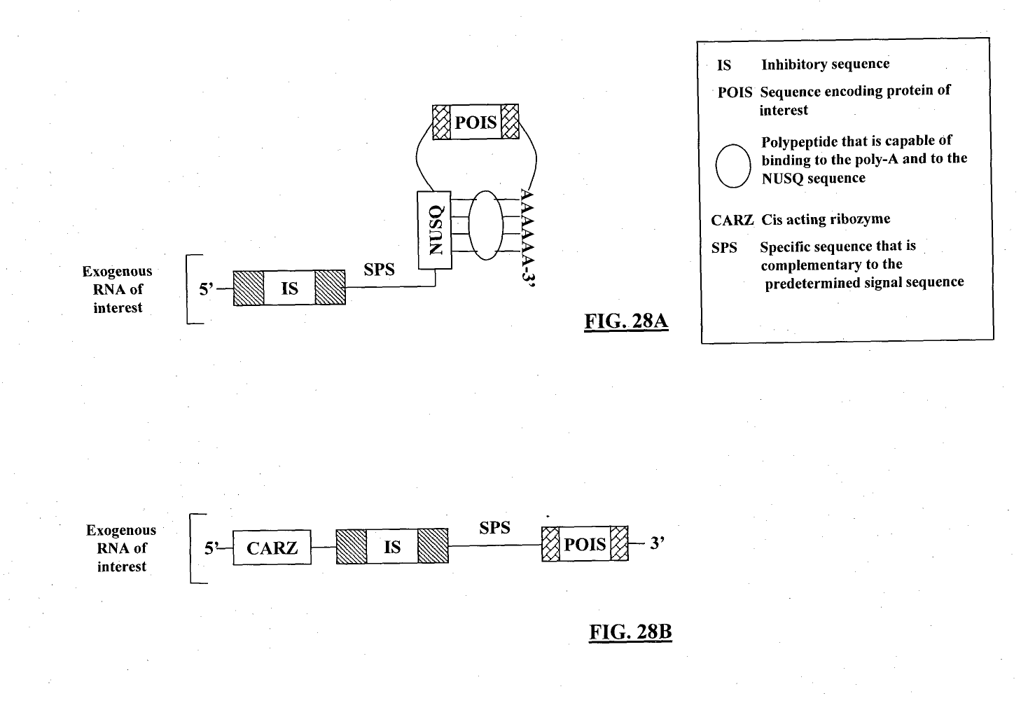

In another embodiment of the invention, the exogenous RNA of interest that is described in section 7 comprises a unique nucleotide sequence that is located downstream from the specific target/cleavage site and upstream from the sequence encoding protein of interest. The unique nucleotide sequence may be capable of binding to a unique polypeptide that is, directly or indirectly, capable of binding to the poly(A) tail in the cleaved exogenous RNA of interest, and the unique polypeptide may be encoded from the composition of the invention. Without wishing to bound to theory or mechanism, in such embodiment the unique polypeptide and the cleaved exogenous RNA of interest may create a circular structure that increases the efficiency of translation of the protein of interest in the cleaved exogenous RNA of interest. For example, see FIG. 28A.

7.1.3. Additional Structures that May Reduce the Efficiency of Translation of the Intact Exogenous RNA of Interest

This section describes further embodiments of additional structures of the composition of the invention, described in section 7, such that these additional structures may reduce the efficiency of translation of the exogenous RNA of interest of the invention before it is cleaved (that is, an intact exogenous RNA of interest).

In some embodiments, the composition that is described in section 7 may further comprise a particular cleaving component(s) that is capable of effecting the cleavage, directly or indirectly, of the exogenous RNA of interest of the invention at a position that is located upstream from the inhibitory sequence, wherein the inhibitory sequence is located upstream from the specific target/cleavage site. In some embodiments, the particular cleaving component(s) may comprise:
(a) a particular nucleic acid sequence that is located within the exogenous RNA of interest, such that the particular nucleic acid sequence may be, for example, but not limited to: endonuclease recognition site, endogenous miRNA binding site, cis acting ribozyme and/or miRNA sequence; or
(b) a particular inhibitory RNA that is encoded from the composition of the invention, such that the particular inhibitory RNA may be, for example, but is not limited to: microRNA (miRNA), lariat-form RNA, short-hairpin RNA (shRNA), siRNA expression domain, antisense RNA, double-stranded RNA (dsRNA), small-interfering RNA (siRNA) and/or ribozyme.

Without wishing to bound to theory or mechanism, in such embodiment the particular cleaving component(s) may remove the CAP structure from the intact exogenous RNA of interest of the invention for reducing the efficiency of translation of the protein of interest in the intact exogenous RNA of interest. For example, see FIG. 28B.

In some embodiments, a vpg recognition sequence may be introduced, such that upon cleave, the 5′ cleaved end contains a vpg recognition sequence. To the vpg recognition sequence a VPG protein may bind, thereby replacing the CAP. The vpg protein may be encoded by the composition of the invention or by the first ORF of the inhibitory sequence.

According to some embodiments, and without wishing to bound to theory or mechanism, the use of cis acting ribozyme may be advantageous because the exogenous RNA of interest that comprises it may be cleaved by itself [22]. The cis acting ribozyme may be, for example, but is not limited to cis-acting hammerhead ribozymes: snorbozyme [22] or N117 [23].

7.2. Structure of the Exogenous RNA of Interest Having its Inhibitory Sequence Located Downstream from the Specific Target/Cleavage Site
7.2.1. Structure of the Inhibitory Sequence that is Located Downstream from the Specific Target/Cleavage Site

The inhibitory sequence in the exogenous RNA of interest that is described in embodiments of section 7 can be located upstream or downstream from the specific target/cleavage site. In some embodiments, the inhibitory sequence may be located downstream from the specific target/cleavage site in the exogenous RNA of interest. For example, see FIG. 22B.

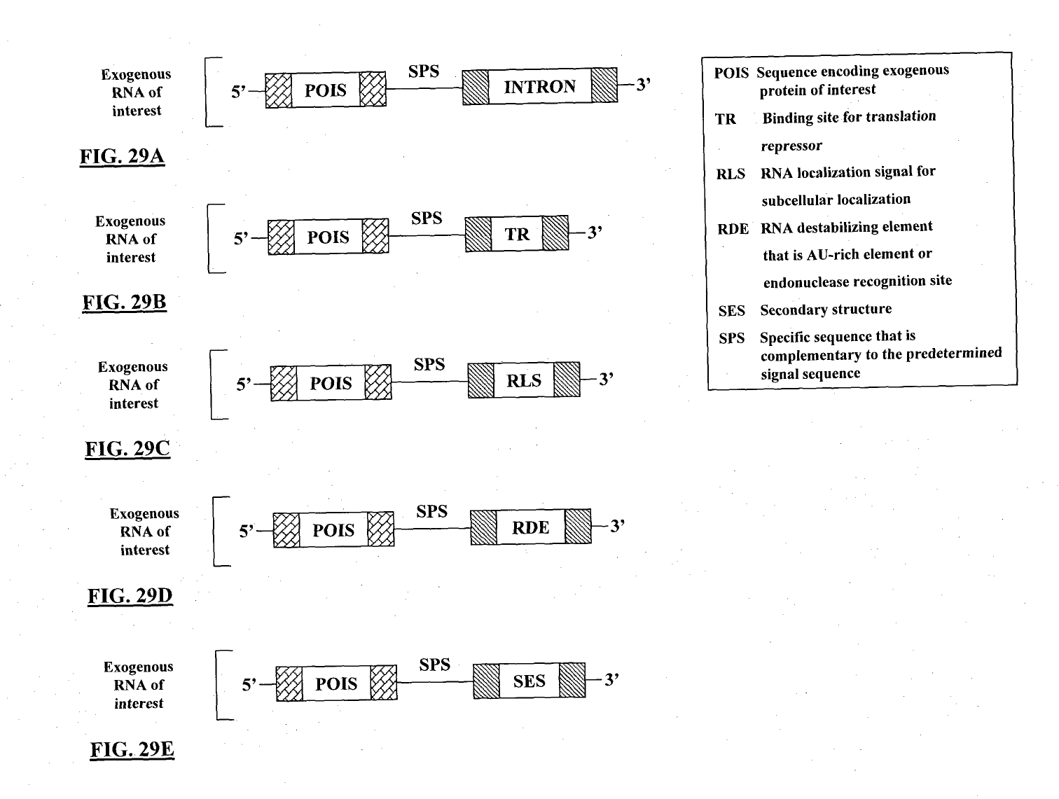

In another embodiment of the invention, the inhibitory sequence that is located downstream from the specific target/cleavage site that is described in section 7 may be, for example, but is not limited to an intron. Such that the exogenous RNA of interest is a target for nonsense-mediated decay (NMD) that degrades the exogenous RNA of interest [31]. For example, see FIG. 29A.

In other embodiment of the invention, the inhibitory sequence that is located downstream from the specific target/cleavage site and that is described in section 7 includes a sequence that is capable of binding to a translation repressor protein, such that the translation repressor protein is an endogenous translation repressor protein or is encoded from the composition and such that the translation repressor protein, directly or indirectly, reduces the efficiency of translation of the protein of interest within the exogenous RNA of interest [26]. The sequence that is capable of binding to a translation repressor protein may be, for example, the binding sequence of smaug repressor protein (5′-UGGAGCAGAGGCUCUGGCAGCUUUUGCAGCG-3′) (SEQ ID NO. 28) [27]. For example, see FIG. 29B.

In another embodiment of the invention, the inhibitory sequence that is located downstream from the specific target/cleavage site and that is described in section 7 comprises an RNA localization signal for subcellular localization (including cotranslational import) or an endogenous miRNA binding site, such that the subcellular localization of the exogenous RNA of interest of the invention inhibits the translation of the exogenous protein of interest and decreases the exogenous RNA of interest half-life. The RNA localization signal may be, for example, but is not limited to RNA localization signal for: myelinating periphery, mitochondria, myelin compartment, leading edge of the lamella and/or Perinuclear cytoplasm [24]. The RNA localization signal may be, for example, RNA localization signal for myelinating periphery 5′-GCCAAGGAGCCAGAGAGCAUG-3′ (SEQ ID NO. 29) or 5′-GCCAAGGAGCC-3′ (SEQ ID. NO. 30) [29]. For example, see FIG. 29C.

In another embodiment of the invention, the inhibitory sequence that is located downstream from the specific target/cleavage site and that is described in section 7 may be, for example, an RNA destabilizing element that stimulates degradation of the exogenous RNA of interest, such that the RNA destabilizing element is an AU-rich element (ARE) or an endonuclease recognition site. The AU-rich element may be, for example, AU-rich elements that are at least about 35 nucleotides long. The AU-rich element may be, for example, 5′-AUUUA-3′ (SEQ ID NO. 31), 5′-UUAUUUA(U/A)(U/A)-3′ (SEQ ID NO. 32) or 5′-AUUU-3′ (SEQ ID NO. 33) [28]. For example, see FIG. 29D.

In another embodiment of the invention, without wishing to be bound to theory or mechanism, the inhibitory sequence that is located downstream from the specific target/cleavage site and that is described in section 7 comprises a sequence that is capable of forming a secondary structure that reduces the efficiency of translation of the upstream protein of interest. For example, see FIG. 29E.

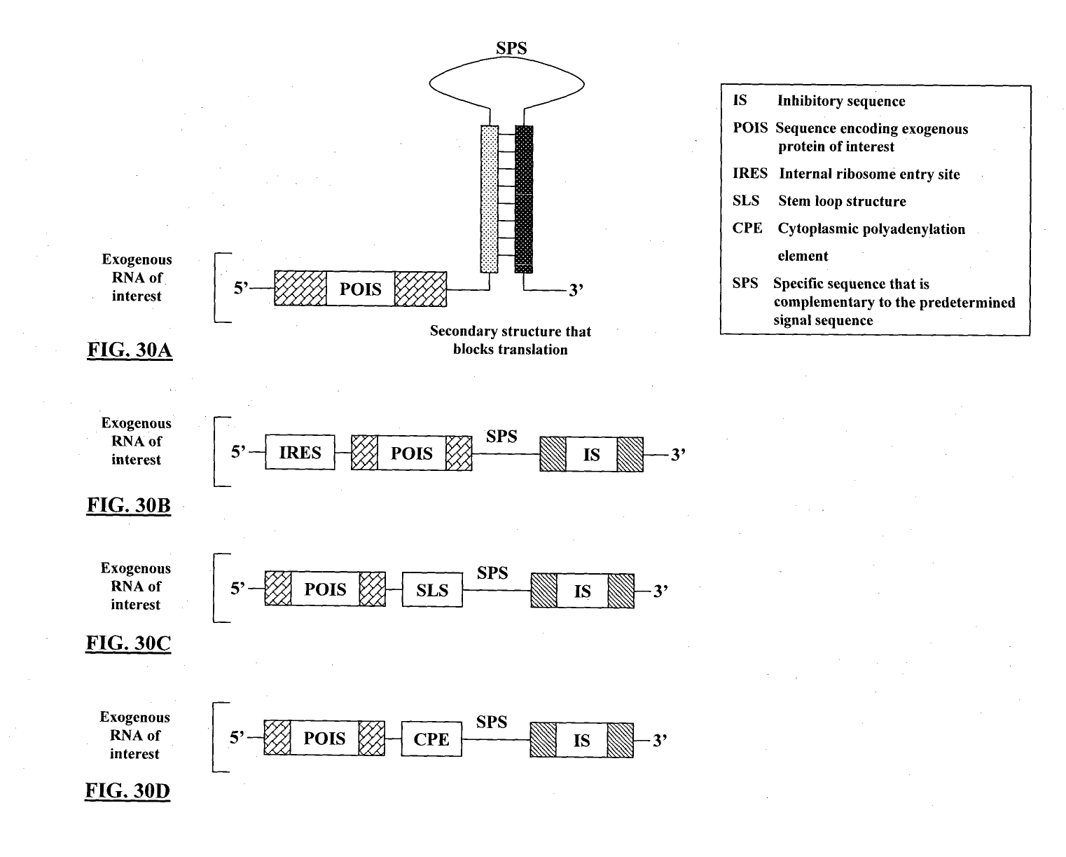

In another embodiment of the invention the inhibitory sequence that is located downstream from the specific target/cleavage site and that is described in section 7 comprises a sequence immediately downstream from the specific target/cleavage site that is capable of binding to the nucleotide sequence that is located immediately upstream from the specific target/cleavage site for the formation of a secondary structure, such that the secondary structure, directly or indirectly, reduces the efficiency of translation of the upstream protein of interest. In some embodiments, the free energy of the folding of the secondary structure that is described in the former embodiment may be lower than −30 kcal/mol (for example, −50 kcal/mol, −80 kcal/mol) and hence this secondary structure may be sufficient to block scanning ribosomes from reaching the stop codon of the protein of interest. In another embodiment, the specific target/cleavage site is located within the single stranded region or within the loop region in the secondary structure that is described in the former embodiment, such that the single stranded region or the loop region include, for example, but is not limited to a region that is at least about 15 nucleotides long. For example, see FIG. 30A.

7.2.2. Additional Structures that May Increase the Efficiency of Translation of the Exogenous RNA of Interest which is Cleaved at the Specific Target/Cleavage Site at the 3′ End

This section describes further embodiments of additional structures of the composition of the invention described in various embodiments of section 7, such that these additional structures may increase the efficiency of translation of the cleaved exogenous RNA of interest, wherein the cleaved exogenous RNA of interest is cleaved at the specific target/cleavage site at the 3′ end.

According to some embodiments, the exogenous RNA of interest of the invention that is described in section 7 may comprise a sequence that comprises a unique internal ribosome entry site (IRES) sequence immediately upstream from the sequence encoding protein of interest, such that the unique IRES sequence may increase the efficiency of translation of the protein of interest in the cleaved exogenous RNA of interest. For example, see FIG. 30B.

In another embodiment of the invention, the exogenous RNA of interest that is described in section 7 may comprise a unique nucleotide sequence immediately downstream from the sequence encoding protein of interest, such that the unique nucleotide sequence comprises a unique stem loop structure and such that the unique stem loop structure, directly or indirectly, increases the efficiency of translation of the protein of interest and the cleaved exogenous RNA of interest half-life. The unique stem loop structure may include such structures as, but not limited to: a conserved stem loop structure of the human histone gene 3′-UTR or a functional derivative thereof. For example, the conserved stem loop structure of the human histone gene 3′-UTR is 5′-GGCUCUUUUCAGAGCC-3′ (SEQ ID NO. 34). For example, see FIG. 30C.

In another embodiment, the exogenous RNA of interest that is described in section 7 may comprise a unique nucleotide sequence immediately downstream from the sequence encoding protein of interest, such that the unique nucleotide sequence comprises a cytoplasmic polyadenylation element that, directly or indirectly, may increase the efficiency of translation of the protein of interest and the cleaved exogenous RNA of interest half-life. The cytoplasmic polyadenylation element may be selected from such elements as, but not limited to: 5′-UUUUAU-3′(SEQ ID NO. 35), 5′-UUUUUAU-3′(SEQ ID NO. 36), UUUUAAU-3′(SEQ ID NO. 37), 5′-UUUUUUAUU-3′(SEQ ID NO. 38), 5′-UUUUAUU-3′(SEQ ID NO. 39) or 5′-UUUUUAUAAAG-3′ (SEQ ID NO. 40) [25]. The composition of the invention may also comprise, for example, a polynucleotide sequence that encodes a human cytoplasmic polyadenylation element binding protein (hCPEB), or a homologue thereof for expressing hCPEB in any cell. For example, see FIG. 30D.

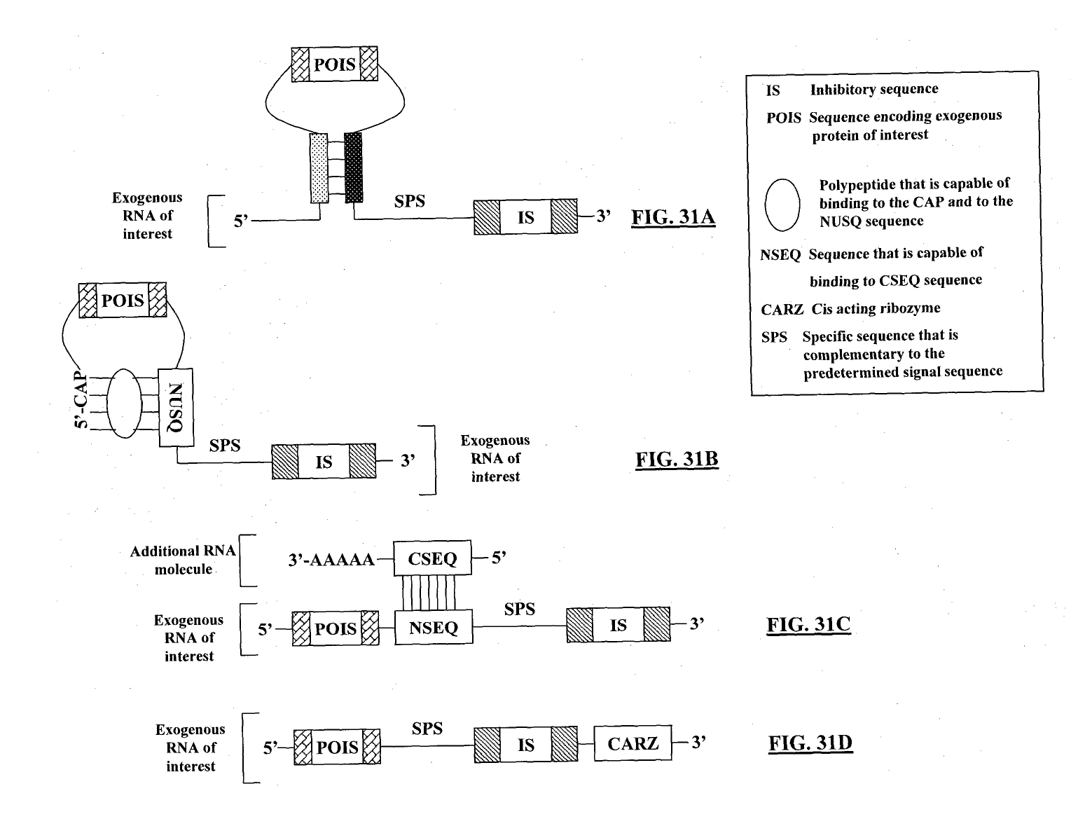

In additional embodiment of the invention, the exogenous RNA of interest that is described in section 7 may comprise a unique nucleotide sequence that is located upstream from the specific target/cleavage site and downstream from the sequence encoding protein of interest, such that the unique nucleotide sequence is capable of binding to a sequence that is located upstream from the sequence encoding protein of interest. In such embodiment and without wishing to be bound to theory or mechanism, the cleaved exogenous RNA of interest may create a circular structure that may increase the efficiency of translation of the protein of interest in the cleaved exogenous RNA of interest. For example, see FIG. 31A.

In another embodiment of the invention, the exogenous RNA of interest that is described in section 7 may comprise a unique nucleotide sequence that is located upstream from the specific target/cleavage site and downstream from the sequence encoding protein of interest, the unique nucleotide sequence may be capable of binding to a unique polypeptide that is, directly or indirectly, capable of binding to the CAP structure in the cleaved exogenous RNA of interest, wherein the unique polypeptide is encoded from the composition of the invention. In this embodiment and without wishing to be bound to theory or mechanism, the unique polypeptide and the cleaved exogenous RNA of interest may create a circular structure that may increase the efficiency of translation of the protein of interest in the cleaved exogenous RNA of interest. For example, see FIG. 31B.

In another embodiment, the composition of the invention that is described in section 7 may comprise an additional polynucleotide sequence, which may encode an additional RNA molecule that comprises at the 3′ end a nucleotide sequence that is capable of binding to a sequence that is located upstream from the specific target/cleavage site and downstream from the sequence encoding protein of interest, such that the expression of the additional polynucleotide sequence is driven by polymerase II based promoter. In such embodiment and without wishing to be bound to theory or mechanism, the additional RNA molecule is capable of binding to the cleaved exogenous RNA of interest and provide him poly-A which may increase the efficiency of translation of the exogenous protein of interest in the cleaved exogenous RNA of interest. For example, see FIG. 31C.

7.2.3. Additional Structures that May Reduce the Efficiency of Translation of the Intact Exogenous RNA of Interest

This section describes further embodiments of additional structures of the composition of the invention that is described in embodiments of section 7, such that these additional structures reduce the efficiency of translation of the exogenous RNA of interest of the invention before it is cleaved.

In some embodiments of the invention, the composition that is described in section 7 may comprise a particular cleaving component(s) that is capable of effecting the cleavage, directly or indirectly, of the exogenous RNA of interest of embodiments of the invention at a position that is located downstream from the inhibitory sequence, wherein the inhibitory sequence is located downstream from the specific target/cleavage site. The particular cleaving component(s) is:
(a) a particular nucleic acid sequence that is located within the exogenous RNA of interest, such that the particular nucleic acid sequence may be, for example: endonuclease recognition site, endogenous miRNA binding site, cis acting ribozyme, miRNA sequence and the like; or
(b) a particular inhibitory RNA that is encoded from the composition of the invention, such that the particular inhibitory RNA may be, for example, microRNA (miRNA), lariat-form RNA, short-hairpin RNA (shRNA), siRNA expression domain, antisense RNA, double-stranded RNA (dsRNA), small-interfering RNA (siRNA) or ribozyme.

In this embodiment and without wishing to be bound to theory or mechanism, the particular cleaving component(s) may remove the poly-A from the intact exogenous RNA of interest of the invention and may thus reduce the efficiency of translation of the protein of interest in the intact exogenous RNA of interest. For example, see FIG. 31D.

In some embodiments, and without wishing to be bound to theory or mechanism, the use of cis acting ribozyme may be advantageous because the exogenous RNA of interest that comprises it may be cleaved by itself [22]. The cis acting ribozyme may be, for example, cis-acting hammerhead ribozymes: snorbozyme [22] or N117 [23].

### 8. DETAILED DESCRIPTION OF EXEMPLARY EMBODIMENTS OF THE INVENTION

According to some embodiments, as detailed below, an exogenous protein of interest may be expressed in response to the presence of an endogenous signal RNA in a cell without the involvement of Risc (RNA-induced silencing complex) mechanism.

According to some specific embodiments, there is provided a composition for expressing an exogenous protein of interest in response to the presence of endogenous signal RNA in a cell, the exogenous protein of interest is encoded from the composition, the endogenous signal RNA is an RNA molecule which comprises a predetermined signal sequence, the predetermined signal sequence is a predetermined sequence that is at least 18 nucleotides in length and the composition may comprise one or more polynucleotide molecule(s), such as, for example, DNA molecules, that comprise:
(a) one or more polynucleotide sequence(s) encoding a functional RNA that is capable of effecting the cleavage, directly or indirectly, of the endogenous signal RNA at a predetermined cleavage site, such that the predetermined cleavage site is the 3′ end of the predetermined signal sequence; and
(b) a polynucleotide sequence encoding an exogenous RNA of interest molecule, which is an RNA molecule that is consisting essentially of:
   (1) a first sequence which is of sufficient complementarity to an edge sequence to hybridize therewith, the edge sequence is located 0-5 nucleotides upstream from the predetermined cleavage site and extends upstream in the endogenous signal RNA, such that the first sequence comprises one or more initiation codon(s), such that each of the initiation codons is consisting essentially of 5′-AUG-3′; and
   (2) a second sequence upstream from the first sequence, such that the second sequence is a random sequence that is 0-5 nucleotides in length; and
   (3) a third sequence downstream from the first sequence, such that the third sequence is 0-7000 nucleotides in length; and

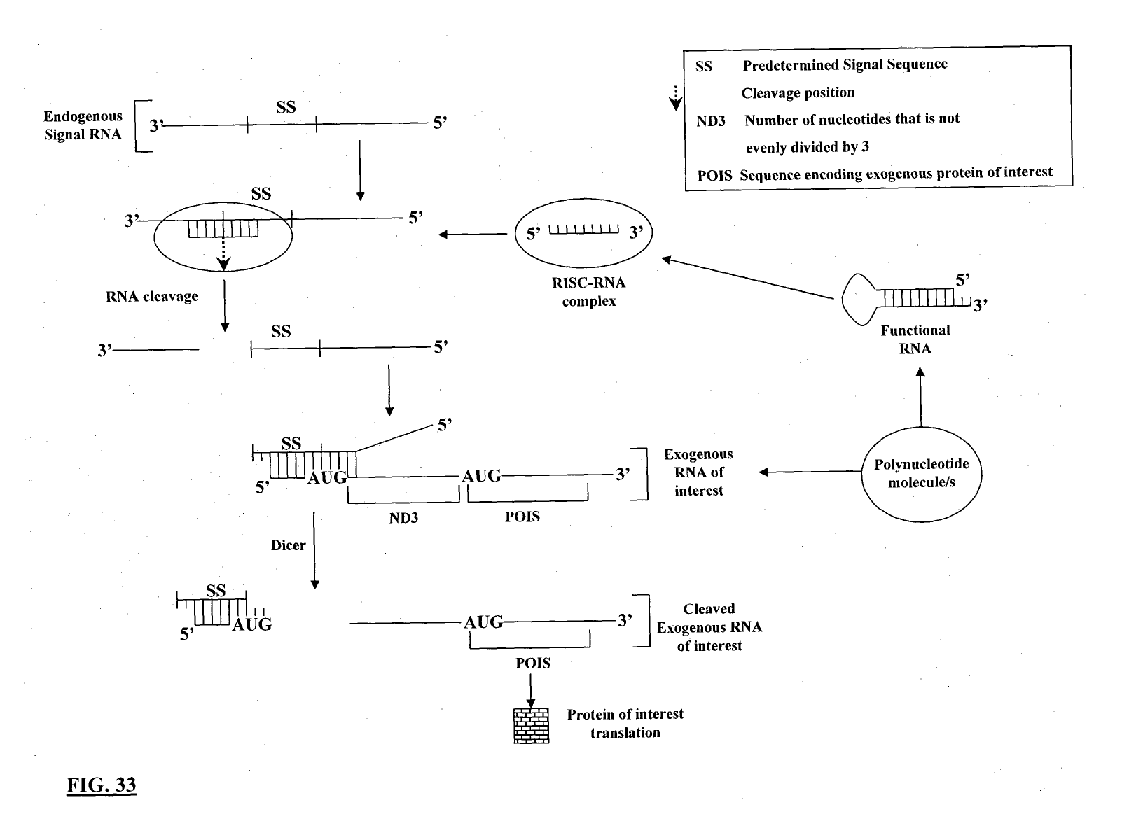

such that the exogenous RNA of interest molecule comprises a sequence encoding exogenous protein of interest at least 21 nucleotides downstream from the 5′ end of the exogenous RNA of interest molecule; and such that, following introduction of the composition into a cell comprising the endogenous signal RNA, the functional RNA effects the cleavage, directly or indirectly, of the endogenous signal RNA at the 3′ end of the predetermined signal sequence. Thereby, the exogenous RNA of interest molecule is hybridized to the edge sequence at the cleaved endogenous signal RNA and directs the predetermined signal sequence to Dicer processing that may cleave the exogenous RNA of interest molecule, such that each of the initiation codon(s) is detached from the sequence encoding the exogenous protein of interest and the exogenous protein of interest is capable of being expressed. For example, see FIG. 33.

In some embodiments of the invention, the edge sequence is 25-30 nucleotides in length and is located 2 nucleotides upstream from the predetermined cleavage site and extends upstream in the endogenous signal RNA and such that the second sequence is 0 nucleotides in length, such as shown, for example in FIG. 33.

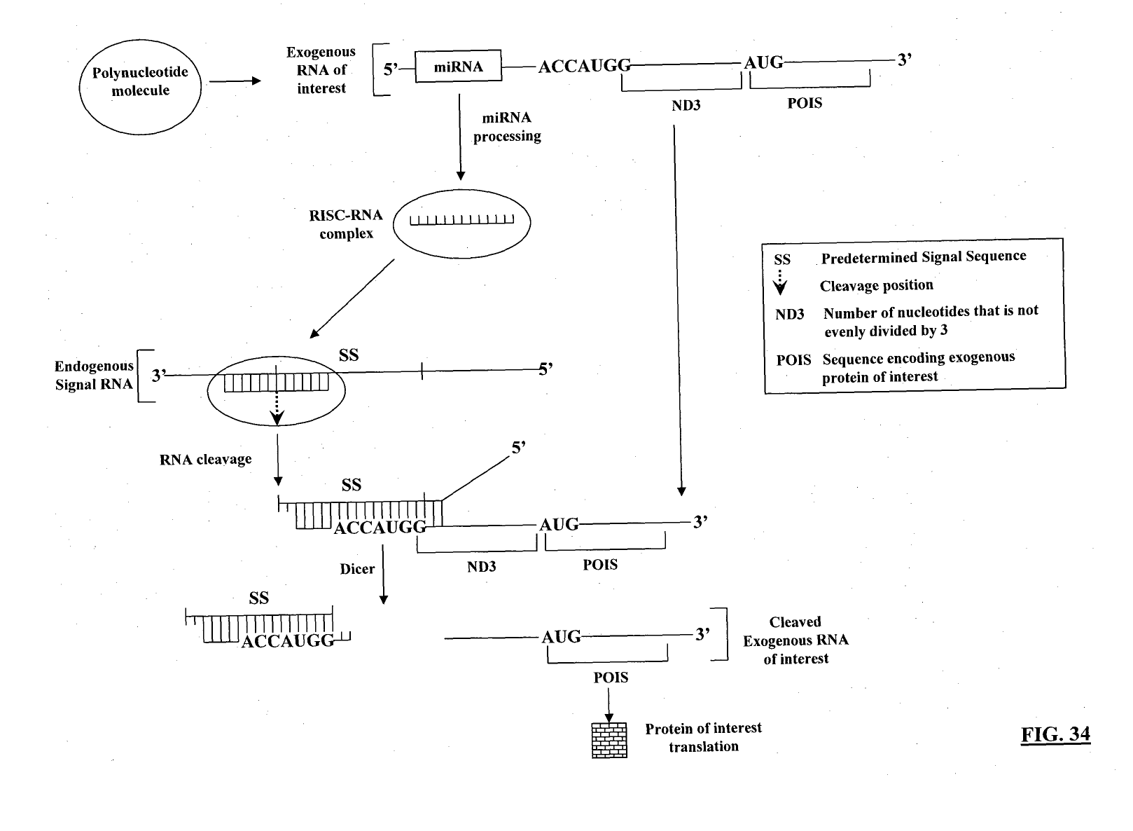

In another embodiment of the invention, at least one of the initiation codon(s) is located within a Kozak consensus sequence, such as, for example, Kozak consensus sequence 5′-ACCAUGG-3′ (SEQ ID NO. 25), demonstrated for example in FIG. 34.

In additional embodiment of the invention, each of the initiation codon(s) is located 0-21 nucleotides downstream from the 5′ end of the exogenous RNA of interest molecule, such that each of the initiation codon(s) and the sequence encoding the exogenous protein of interest are not in the same reading frame. In further embodiment of the invention, at least one of the initiation codon(s) described above is located within a Kozak consensus sequence such as, for example, Kozak consensus sequence 5′-ACCAUGG-3′ (SEQ ID NO. 25).

In some embodiments, the functional RNA may be, for example, but is not limited to: microRNA (miRNA), lariat-form RNA, short-hairpin RNA (shRNA), siRNA expression domain, antisense RNA, double-stranded RNA (dsRNA), small-interfering RNA (siRNA), ribozyme, or combinations thereof.

In some exemplary embodiments, the functional RNA may be, for example, a microRNA (miRNA), such that the miRNA and the exogenous RNA of interest molecule are capable of being located on the same or different RNA molecules. In some embodiments, the miRNA may be located upstream from the second sequence at the exogenous RNA of interest molecule, as demonstrated, for example, in FIG. 34.

According to some embodiments, and without wishing to be bound to theory or mechanism, the previous embodiment may be advantageous since in such embodiment, the CAP structure may be removed from the exogenous RNA of interest molecule and since in this embodiment the composition encodes for only one RNA molecule.

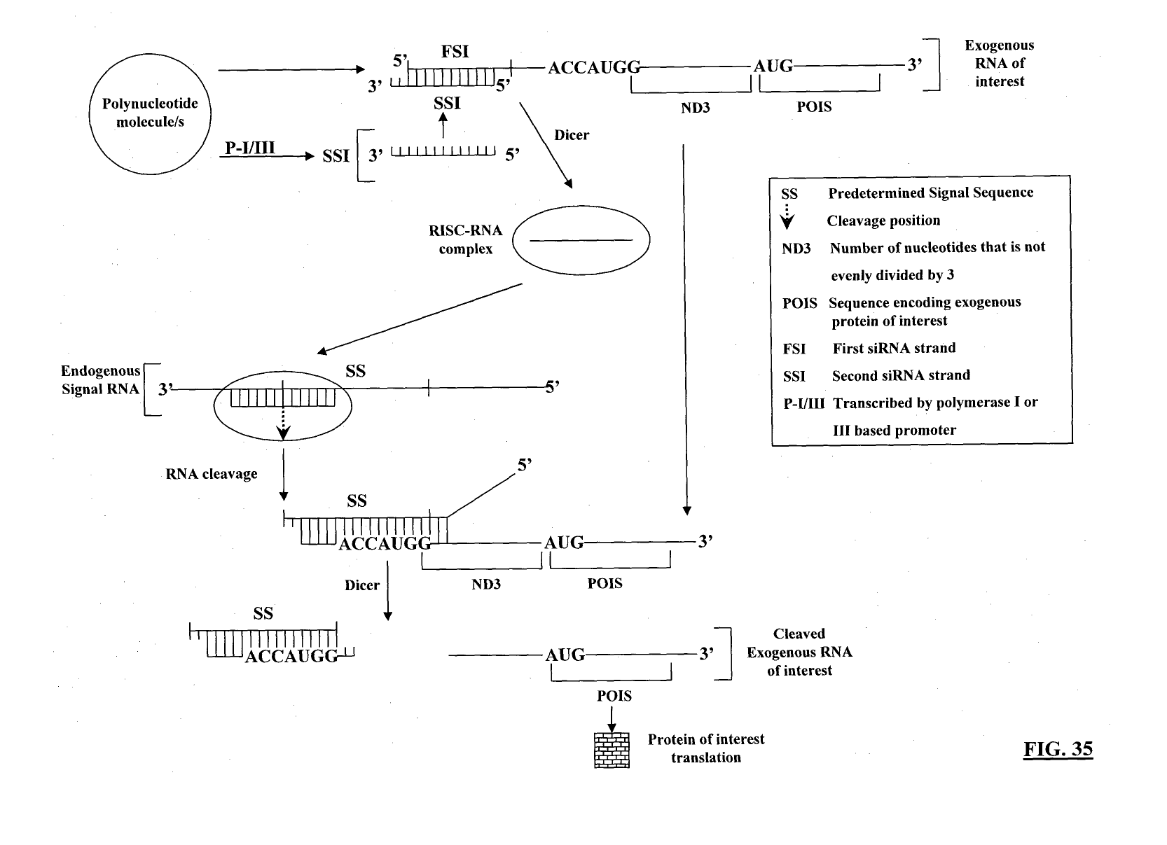

In another exemplary embodiment, the functional RNA may be, for example, a small-interfering RNA (siRNA), such that one RNA strand of the siRNA is located at the 5′ end of the exogenous RNA of interest molecule and the other strand of the siRNA is transcribed from the composition by, for example, polymerase I or polymerase III based promoter(s) and such that following introduction of the composition into a cell, both of the siRNA strands are hybridized and detached from the exogenous RNA of interest molecule, for example, by Dicer. This is demonstrated, for example, in FIG. 35.

According to some embodiments, and without wishing to be bound to theory or mechanism, the previous embodiment may be advantageous since in such embodiment the functional RNA and the exogenous RNA of interest molecule are located in the same RNA duplex, thus the exogenous RNA of interest molecule brings the functional RNA into proximity with the predetermined signal sequence of the endogenous signal RNA and by this may also bring also the components of the RNA interference pathway (such as, for example, Dicer) into proximity with the predetermined signal sequence. Another advantage includes the removal of the CAP structure from the exogenous RNA of interest molecule by Dicer.

In another embodiment of the invention, the exogenous RNA of interest molecule may further comprise a nucleotide sequence located upstream from the sequence encoding the protein of interest and downstream from each of the initiation codons, such that this nucleotide sequence is of sufficient complementarity to the predetermined signal sequence or to the sequence that is located at the 5′ end of the exogenous RNA of interest molecule, and is able to direct target-specific RNA interference. For example, the Risc processing that follows the Dicer processing can be used for activating more exogenous RNA of interest molecule molecules.

In another embodiment of the invention, the composition further comprises one or more polynucleotide sequence(s) encoding a functional nucleic acid that is capable of effecting the cleavage, directly or indirectly, of the exogenous RNA of interest molecule upstream from the second sequence, such that the functional nucleic acid is:
(a) a specific nucleic acid sequence that is located within the exogenous RNA of interest molecule, such that the specific nucleic acid sequence is: endonuclease recognition site, endogenous miRNA binding site, cis acting ribozyme or miRNA sequence; or
(b) an inhibitory RNA that is encoded from a DNA molecule(s), such that the inhibitory RNA is: microRNA (miRNA), lariat-form RNA, short-hairpin RNA (shRNA), siRNA expression domain, antisense RNA, double-stranded RNA (dsRNA), small-interfering RNA (siRNA) or ribozyme.

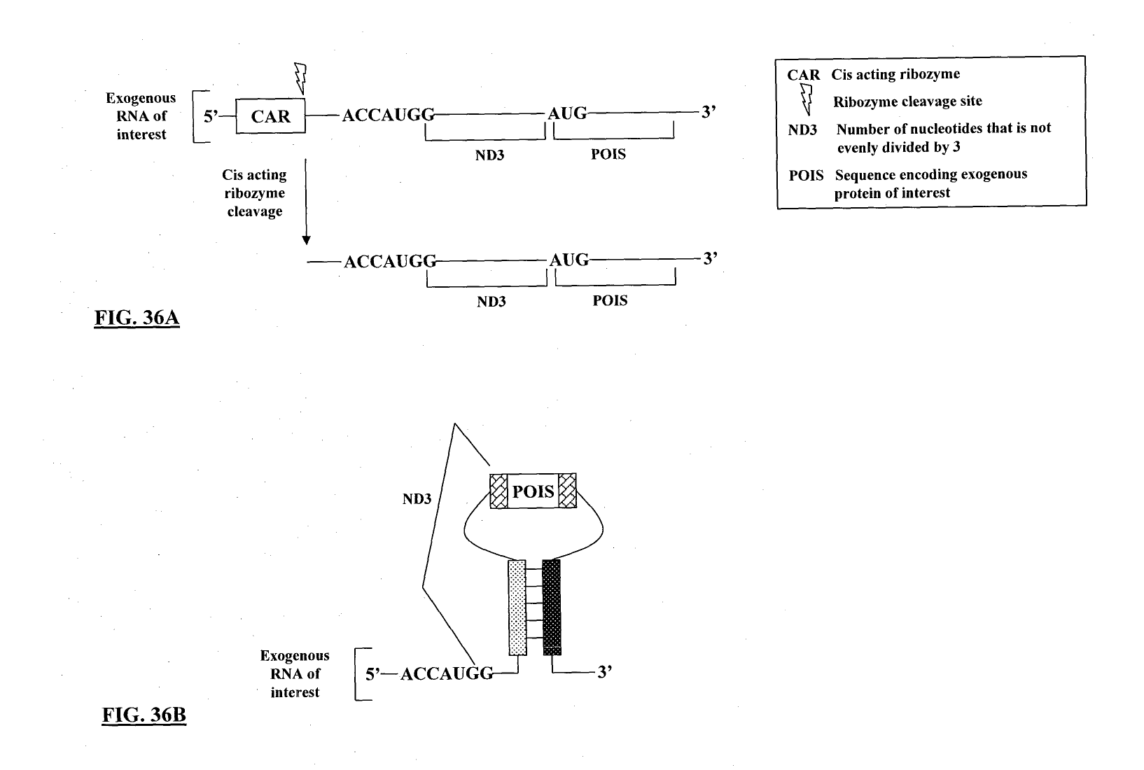

In this embodiment, the functional nucleic acid may remove the CAP structure from the intact exogenous RNA of interest for reducing the efficiency of translation of the exogenous protein of interest from the non-cleaved (intact) exogenous RNA of interest. For example, see FIG. 36A.

In another embodiment, the third sequence described above, includes a nucleotide sequence upstream from the sequence encoding protein of interest, such that the nucleotide sequence is capable of binding to a sequence that is located downstream from the sequence encoding protein of interest. In this embodiment the cleaved exogenous RNA of interest molecule creates a circular structure that may increase the efficiency of translation of the protein of interest in the cleaved exogenous RNA of interest molecule. For example, see FIG. 36B.

In another embodiment of the invention, the polynucleotide molecule(s) that is described above together further comprise a polynucleotide sequence encoding an additional functional RNA that is capable of effecting the cleavage, directly or indirectly, of the endogenous signal RNA at an additional cleavage site, such that the additional cleavage site is located 0-1000 nucleotides upstream from the 5′ end of the predetermined signal sequence. For example, see FIG. 21B. In another embodiment, the additional cleavage site that described in the former embodiment is located 0-5 nucleotides upstream from the 5′ end of the predetermined signal sequence. For example, see FIG. 21B.

### 9. DESCRIPTION OF ADDITIONAL EMBODIMENTS OF THE INVENTION

This section describes additional embodiments of the invention that are directed to the cleavage of the exogenous RNA of interest in response to the presence of an endogenous signal RNA in a cell, without the cleaving the endogenous signal RNA. Such embodiments may be useful, for example, for endogenous signal RNA of a viral origin.

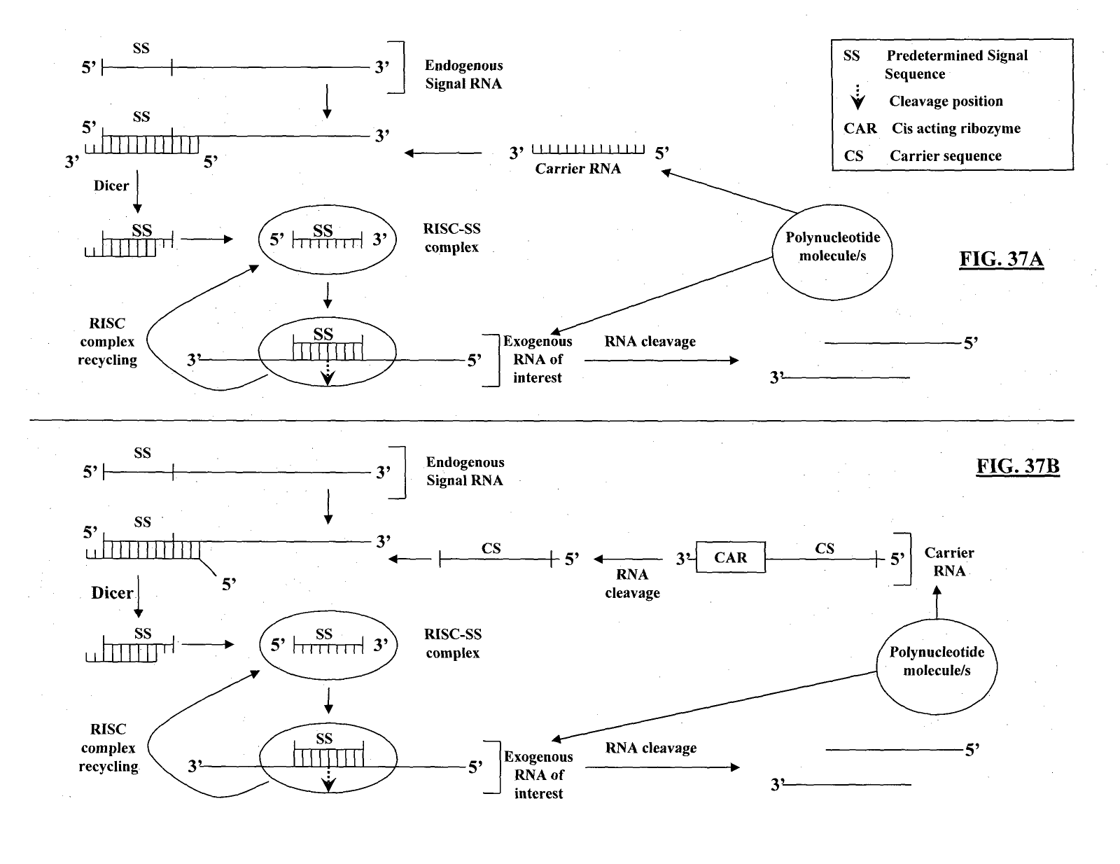

According to some embodiments, there is provided a composition for cleaving an exogenous RNA of interest in response to the presence of an endogenous signal RNA in a cell, the exogenous RNA of interest is encoded from the composition, the endogenous signal RNA is an RNA molecule which comprises a predetermined signal sequence at the 5′ end, the predetermined signal sequence is a predetermined sequence of from 18 to 25 nucleotides in length, the composition comprises one or more polynucleotides molecules (such as, for example, DNA and/or RNA molecules), that comprise:
(a) a polynucleotide sequence encoding the exogenous RNA of interest, such that the exogenous RNA of interest is an RNA sequence that comprises a specific sequence which is of sufficient complementarity to the predetermined signal sequence to direct, for example, target-specific RNA interference;
(b) a polynucleotide sequence encoding a carrier RNA, such that expression of the polynucleotide sequence the carrier RAN sequence is driven by a promoter selected from the group consisting of: polymerase I based promoter and polymerase III based promoter, such that the carrier RNA is an RNA molecule that is at least about 18 nucleotides in length and is consisting essentially of:
   (1) a first sequence of from 14 to 31 nucleotides in length which is of sufficient complementarity to an edge sequence to hybridize therewith, the edge sequence is 14-31 nucleotides in length and is located 0-5 nucleotides downstream from the 5′ end of the endogenous signal RNA and extends downstream in the endogenous signal RNA;
   (2) a second sequence downstream from the first sequence, such that the second sequence is a random sequence that is 0-5 nucleotides in length; and
   (3) a third sequence upstream from the first sequence, such that the third sequence is 0-7000 nucleotides in length; and

whereby, following introduction of the composition into a cell comprising the endogenous signal RNA, the carrier RNA is hybridized to the edge sequence and directs the processing of the predetermined signal sequence, and then the processed predetermined signal sequence may direct the cleavage of the exogenous RNA of interest at a specific target (cleavage) site that is located within the specific sequence. For example, see FIG. 37A.

In additional embodiments, there is provided a composition for cleaving exogenous RNA of interest in response to the presence of an endogenous signal RNA in a cell, the exogenous RNA of interest is encoded from the composition, the endogenous signal RNA is an RNA molecule which comprises a predetermined signal sequence at the 5′ end, the signal sequence is a predetermined sequence of from 18 to 25 nucleotides in length, the composition comprises one or more polynucleotide molecules (such as, for example, DNA molecules and/or RNA molecule), the polynucleotide molecules together comprise:
(a) a polynucleotide sequence encoding the exogenous RNA of interest, such that the exogenous RNA of interest is an RNA sequence that comprises a specific sequence which is of sufficient complementarity to the predetermined signal sequence to direct cleavage, for example, by target-specific RNA interference;
(b) a polynucleotide sequence encoding an RNA sequence that comprises a carrier sequence that is at least about 18 nucleotides in length, the carrier sequence consisting essentially of:
   (1) a first sequence of from 14 to 31 nucleotides in length which is of sufficient complementarity to an edge sequence to hybridize therewith, the edge sequence is 14-31 nucleotides in length and is located 0-5 nucleotides downstream from the 5′ end of the endogenous signal RNA and extends downstream in the endogenous signal RNA;
   (2) a second sequence downstream from the first sequence, such that the second sequence is a random sequence that is 0-5 nucleotides in length; and
   (3) a third sequence upstream from the first sequence, such that the third sequence is 0-7000 nucleotides in length; and
(c) one or more polynucleotide sequence(s) encoding a functional nucleic acid that is capable of effecting the cleavage, directly or indirectly, of the carrier RNA sequence at a carrier cleavage site, such that the carrier cleavage site is the 3′ end of the carrier sequence;

whereby, following introduction of the composition into a cell comprising the endogenous signal RNA, the functional nucleic acid effects the cleavage, directly or indirectly, of the carrier RNA sequence at the 3′ end of the carrier sequence and then the cleaved carrier sequence is hybridized to the edge sequence and directs the processing of the predetermined signal sequence and then the processed predetermined signal sequence directs the cleavage of the exogenous RNA of interest at a specific cleavage/target site that is located within the specific sequence. For example, see FIG. 37B.

In some embodiments of the invention, the edge sequence, described above is 23-29 nucleotides in length and may be located from the 5′ end of the endogenous signal RNA to about 23-29 nucleotides downstream, such that the second sequence may be 2 nucleotides in length and such that the third sequence may be 0 nucleotides in length. For example, see FIG. 37A, 37B.

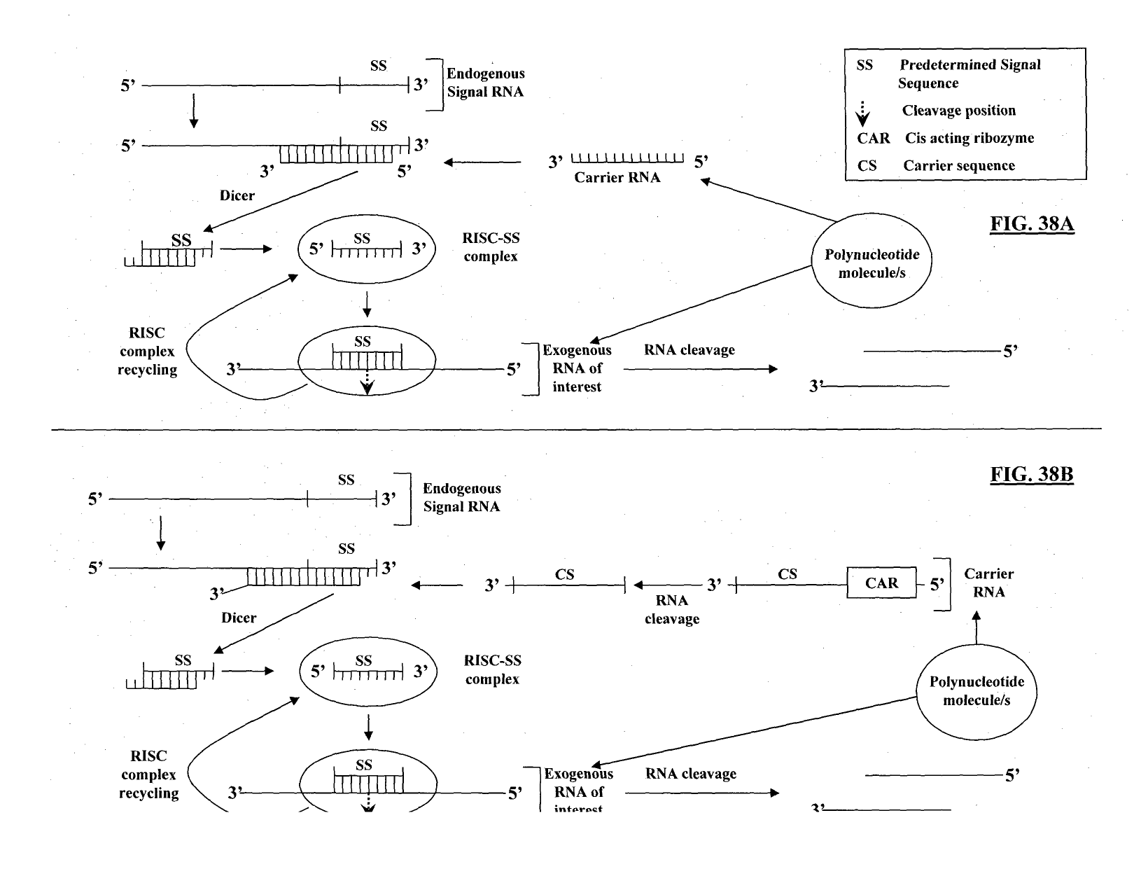

In an additional embodiment, there is provided a composition for cleaving exogenous RNA of interest in response to the presence of an endogenous signal RNA in a cell, the exogenous RNA of interest is encoded from the composition, the endogenous signal RNA is an RNA molecule which comprises a predetermined signal sequence at the 3′ end, the predetermined signal sequence is a random sequence of from 18 to 25 nucleotides in length, the composition comprises one or more polynucleotide molecules (such as, for example, DNA or RNA molecules), the polynucleotide molecules together comprise:
(a) a polynucleotide sequence encoding the exogenous RNA of interest, such that the exogenous RNA of interest is an RNA sequence that comprises a specific sequence which is of sufficient complementarity to the predetermined signal sequence to direct cleavage, for example, by target-specific RNA interference;
(b) a polynucleotide sequence encoding a carrier RNA, such that expression of the carrier RNA is driven by a promoter selected from the group consisting of: polymerase I based promoter and polymerase III based promoter, the carrier RNA is an RNA molecule that is at least about 18 nucleotides in length and is consisting essentially of:
   (1) a first sequence of from 14 to 31 nucleotides in length which is of sufficient complementarity to an edge sequence to hybridize therewith, the edge sequence is 14-31 nucleotides in length and is located 0-5 nucleotides upstream from the 3′ end of the endogenous signal RNA and extends upstream in the endogenous signal RNA;
   (2) a second sequence upstream from the first sequence, such that the second sequence is a random sequence that is 0-5 nucleotides in length; and
(3) a third sequence downstream from the first sequence, such that the third sequence is 0-7000 nucleotides in length; and

whereby, following introduction of the composition into a cell comprising the endogenous signal RNA, the carrier RNA is hybridized to the edge sequence and directs the processing of the predetermined signal sequence and then the processed predetermined signal sequence directs the cleavage of the exogenous RNA of interest at a specific cleavage/target site that is located within the specific sequence. For example, see FIG. 38A.

According to further embodiments, there is provided a composition for cleaving exogenous RNA of interest in response to the presence of an endogenous signal RNA in a cell, the exogenous RNA of interest is encoded from the composition, the endogenous signal RNA is an RNA molecule which comprises a predetermined signal sequence at the 3′ end, the predetermined signal sequence is a random/predetermined sequence of from 18 to 25 nucleotides in length, the composition comprises one or more polynucleotide molecules (such as, for example, DNA molecules and/or RNA molecules), the polynucleotide molecules together comprise:
(a) a polynucleotide sequence encoding the exogenous RNA of interest, such that the exogenous RNA of interest is an RNA sequence that comprises a specific sequence which is of sufficient complementarity to the predetermined signal sequence to direct cleavage, for example, by target-specific RNA interference;
(b) a polynucleotide sequence encoding an RNA sequence that comprises a carrier sequence that is at least about 18 nucleotides in length, the carrier sequence consisting essentially of:
   (1) a first sequence of from 14 to 31 nucleotides in length which is of sufficient complementarity to an edge sequence to hybridize therewith, the edge sequence is 14-31 nucleotides in length and is located 0-5 nucleotides upstream from the 3′ end of the endogenous signal RNA and extends upstream in the endogenous signal RNA;
   (2) a second sequence upstream from the first sequence, such that the second sequence is a random sequence that is 0-5 nucleotides in length; and
   (3) a third sequence downstream from the first sequence, such that the third sequence is 0-7000 nucleotides in length; and
(c) one or more polynucleotide sequence(s) encoding a functional nucleic acid that is capable of effecting the cleavage, directly or indirectly, of the carrier RNA sequence at a carrier cleavage site, such that the carrier cleavage site is the 5′ end of the carrier sequence;

whereby, following introduction of the composition into a cell comprising the endogenous signal RNA, the functional nucleic acid effects the cleavage, directly or indirectly, of the carrier RNA sequence at the 5′ end of the carrier sequence and then the cleaved carrier sequence is hybridized to the edge sequence and directs the processing of the predetermined signal sequence and then the processed predetermined signal sequence directs the cleavage of the exogenous RNA of interest at a specific cleavage/target site that is located within the specific sequence. For example, see FIG. 38B.

According to some exemplary embodiments, the edge sequence described above is about 25-30 nucleotides in length and may be located 2 nucleotides upstream from the 3′ end of the endogenous signal RNA and extends upstream in the endogenous signal RNA, such that the second sequence may be 0 nucleotides in length and such that the third sequence is 0 nucleotides in length. For example, see FIG. 38A, 38B.

According to some embodiments, the functional nucleic acid described above is:
(a) a specific nucleic acid sequence that is located within the carrier RNA sequence and such that the specific nucleic acid sequence is, for example, endonuclease recognition site, endogenous miRNA binding site, cis acting ribozyme, a miRNA sequence, and the like, or combinations thereof; or
(b) an inhibitory RNA that is encoded from the polynucleotide molecule(s), such that the inhibitory RNA is, for example, microRNA (miRNA), lariat-form RNA, short-hairpin RNA (shRNA), siRNA expression domain, antisense RNA, double-stranded RNA (dsRNA), small-interfering RNA (siRNA), ribozyme, and the like, or combinations thereof. For example, see FIG. 37B; 38B.

According to some embodiments, the exogenous RNA of interest described above is located at the third sequence.

According to further embodiments, the exogenous RNA of interest described above may further comprise:
(a) a sequence encoding an exogenous protein of interest; and
(b) an inhibitory sequence that is capable of inhibiting the expression of the exogenous protein of interest;

such that the specific target/cleavage site is located between the inhibitory sequence and the sequence encoding the protein of interest, such that, following introduction of the composition into a cell comprising the endogenous signal RNA, the exogenous RNA of interest is transcribed and cleaved at the specific target/cleavage site so that the inhibitory sequence is detached from the sequence encoding the protein of interest and the protein of interest is capable of being expressed.

In another embodiment, the inhibitory sequence described above, may be located upstream from the specific target/cleavage site, such that the inhibitory sequence comprises a plurality of initiation codons, such that each of the initiation codons and the sequence encoding the exogenous protein of interest are not in the same reading frame, such that each of the initiation codons is consisting essentially of 5′-AUG-3′, such that at least one of the initiation codons is located within a Kozak sequence.

### 10. ADDITIONAL EMBODIMENTS OF THE INVENTION

This section defines and describes further embodiments of the composition of the invention that are described in any of the previous embodiments in any of the previous sections.

The endogenous signal RNA may be, for example, but is not limited to: viral RNA, cellular RNA, such as, for example, mRNA, and the like, that comprises the predetermined signal sequence. The predetermined signal sequence may be, for example, signal sequence that is unique to neoplastic cells, signal sequence that is from viral origin, and the like, or combinations thereof. In some embodiments, the predetermined signal sequence does not comprise any other type of an endogenous RNA molecule (such as, for example, miRNA, shRNA, ribozyme, stRNA, and the like), that is able to direct or effect cleavage of an RNA molecule within the cell.

According to some embodiments, the cell that may be used in embodiments of the invention may be any type of cell from any origin, such as, for example, but not limited to: mammalian cell, avian cell, plant cell, human cell, animal cell, and the like. The cell may be a cultured cell (primary cell or a cell line), or any cell that is present in an organism or a plant.

According to some embodiments, and without wishing to be bound to theory or mechanism, the duplex that is formed when the carrier RNA/sequence is hybridized to the cleaved endogenous signal RNA, such as described, for example in section 1, may be a substrate for a Dicer.

In some embodiments, the edge sequence that is described in embodiments in section 1 is 23-28 nucleotides in length and is located from the predetermined cleavage site to about 23-28 nucleotides downstream, such that the second sequence is 2 nucleotides in length and such that the third sequence is 0 nucleotides in length.

In another embodiment, the edge sequence that is described in embodiments in section 1 is 25-30 nucleotides in length and is located 2 nucleotides upstream from the predetermined cleavage site and extends upstream in the endogenous signal RNA, such that the second sequence is 0 nucleotides in length and such that the third sequence is 0 nucleotides in length.

In additional embodiments, the carrier RNA or the carrier sequence that is described in section 1 or 9 above, may be designed such that the duplex that is formed when the predetermined signal sequence is cleaved, for example, by Dicer, is thermodynamically weaker at the 5′ end of the predetermined signal sequence than at the 3′ end of the predetermined signal sequence. Such that the strand that is loaded into Risc is the strand that comprises the predetermined signal sequence.

The term “sufficient complementarity” may include, but is not limited to: being capable of binding, or at least partially complementary. In some embodiments, the term sufficient complementarity is in the range of about 30-100%. For example, in some embodiments, the term sufficient complementarity is at least about 30% complementarity. For example, in some embodiments, the term sufficient complementarity is at least about 50% complementarity. For example, in some embodiments, the term sufficient complementarity is at least about 70% complementarity. For example, in some embodiments, the term sufficient complementarity is at least about 90% complementarity. For example, in some embodiments, the term sufficient complementarity is about 100% complementarity.

In one embodiment of the invention, the expression of the carrier RNA polynucleotide sequence that is described in section 1 may be driven by polymerase I based promoter or polymerase III based promoter. In some embodiments, the expression of the carrier RNA polynucleotide sequence described in section 1 may be driven by a promoter that may be, but is not limited to: RNA polymerase III 5S promoter, U6 promoter, adenovirus VA1 promoter, Vault promoter, H1 promoter, telomerase RNA or tRNA gene promoter or a functional derivative thereof.

The exogenous protein of interest that is described in any of sections 7, 8 or 9 may be any type of protein or peptide. In some embodiments, the exogenous protein of interest may be, for example, but is not limited to: alpha toxin, saporin, maize RIP, barley RIP, wheat RIP, corn RIP, rye RIP, flax RIP, Shiga toxin, Shiga-like RIP, momordin, pokeweed antiviral protein, gelonin, Pseudomonas exotoxin, Pseudomonas exotoxin A or modified forms thereof. In some embodiments, the exogenous protein of interest may be, for example, but is not limited to: Ricin A chain, Abrin A chain, Diphtheria toxin fragment A or modified forms thereof. The exogenous protein of interest may be, for example, but is not limited to, an enzyme (such as, for example, Luciferase), a fluorescent protein, a structural protein, and the like.

In some embodiment of the invention, the exogenous protein of interest may be a toxin that may also affect neighboring cells. This toxin may be, for example, but is not limited to: the complete form of: Ricin, Abrin, Diphtheria toxin or modified forms thereof. In another embodiment of the invention, the exogenous protein of interest may be an enzyme that its product can kill also the neighboring cells. Such as enzyme may be, for example, but is not limited to: HSV1 thymidine kinase, such that the composition of the invention further comprises the prodrug—ganciclovir; or Escherichia coli cytosine deaminase, such that the composition of the invention further comprises the prodrug-5-fluorocytosine (5-FC).

In another embodiment of the invention, the exogenous RNA of interest or the intermediate RNA that is described in any of sections 7, 8 or 9 is encoded from a viral vector and the exogenous protein of interest is a product of gene that is necessary for the viral vector reproduction, such that the viral vector reproduces in response to the presence of the endogenous signal RNA in a cell and kills the cell during the process of reproduction. This viral vector may also be, for example, but is not limited to a gene that is capable of stopping the viral vector reproduction when a specific molecule is present in the cell (for example, TetR-VP16/Doxycycline). Hence, when the viral vector is presumed to accumulate enough mutations for reproduction in cells that do not comprise the endogenous signal RNA, the specific molecule can be administered for stopping all the viral vectors reproduction in the body and then after the degradation of most of the viral vectors in the body cells new viral vectors can be administered again. This viral vector may also comprise, a gene that is capable of killing the cell when a specific prodrug is present (e.g. thymidine kinase/ganciclovir), such that when the viral vector is presumed to accumulate enough mutations for reproduction in cells that do not comprise the endogenous signal RNA the specific prodrug can be administered for killing all the viral vectors in the body and then new viral vectors can be administered again.

In another embodiment, the RNA molecule(s) that are encoded from the compositions of the invention are encoded from a viral vector that is capable of being reproduced in a way that may kill the cell during the process of reproduction. Such that the predetermined signal sequence is not present in, for example, cancer cells, and is present in most of the healthy or nonmetastatic tumourigenic cells of the body of a specific patient and such that the exogenous protein of interest that is described in any of sections 7, 8 or 9 is a toxin that may be, for example, but is not limited to: Ricin A chain, Abrin A chain, Diphtheria toxin fragment A or modified forms thereof. Such that when the viral vector enters a healthy or nonmetastatic tumourigenic cell it may kill the cell and when the viral vector enters a cancer cell it kills the cancer cell during the process of the viral vector reproduction, thus the major concentration of the viral vector is present in the cancer area of the body. This viral vector may also comprise, for example, a gene that is capable of stopping the viral vector reproduction when a specific molecule is present in the cell (e.g. TetR-VP 16/Doxycycline). Such that when the viral vector is presumed to get enough mutations for reproduction in cells that comprise the endogenous signal RNA the specific molecule can be administered for stopping all the viral vectors reproduction in the body and then after the degradation of most of the viral vectors in the body cells new viral vectors can be administered again. This viral vector may also comprise, for example, a gene that is capable of killing the cell when a specific prodrug is present (e.g. thymidine kinase/ganciclovir), such that when the viral vector is presumed to get enough mutations for reproduction in cells that comprise the endogenous signal RNA the specific prodrug can be administered for killing all the viral vectors in the body and then new viral vectors can be administered again.

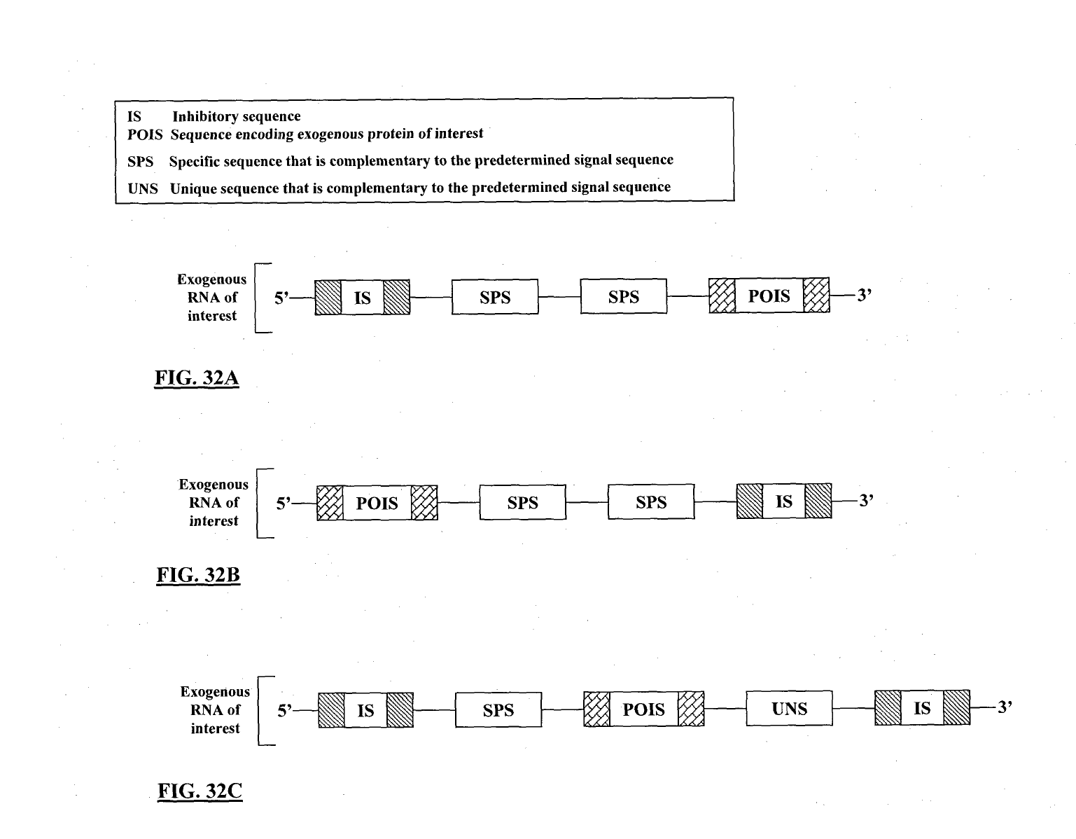

In another embodiment of the invention, the specific sequence that is located within the exogenous RNA of interest that is described in section 1 or 9 is a plurality of specific sequences and the specific target/cleavage site is a plurality of specific target/cleavage sites. Such that for the exogenous RNA of interest that is described in section 7, wherein said “upstream from the specific cleavage site” also includes upstream from all the cleavage sites and wherein said “downstream from the cleavage site” also includes downstream from all the specific cleavage sites. For example, see FIG. 32A, 32B.

In another embodiment, the specific sequence that is located within the exogenous RNA of interest that is described in section 1 or 9 is one or more specific sequence(s) and the specific target/cleavage site is one or more specific target/cleavage site(s) and the exogenous RNA of interest further comprises: a sequence encoding exogenous protein of interest downstream from the specific target/cleavage site(s), one or more unique sequence(s), such that each of the unique sequence(s) is of sufficient complementarity to the predetermined signal sequence to direct target-specific RNA interference, such that each of the unique sequence(s) is located downstream from the sequence encoding the exogenous protein of interest and 2 inhibitory sequences one at the 5′ end of the exogenous RNA of interest and other at the 3′ end of the exogenous RNA of interest, such that each of the inhibitory sequences is capable of inhibiting the expression of the exogenous protein of interest. Such that when the endogenous signal RNA is present in a cell, the two inhibitory sequences are detached from the sequence encoding the exogenous protein of interest and the exogenous protein of interest is capable of being expressed. For example, see FIG. 32C.

In another embodiment of the invention, the polynucleotide molecule(s) (such as DNA molecules and/or RNA molecules) of the composition that are described in any of sections 1, 8 or 9 may further comprise a polynucleotide sequence encoding Dicer, or a homologue thereof.

In another embodiment of the invention, the polynucleotide molecule(s) of the composition that are described in section 1 or 9 together further comprise a polynucleotide sequence encoding one or more RISC components, or a homologue thereof.

In another embodiment of the invention, the polynucleotide molecule(s) of the composition that are described in any of sections 1, 8 or 9 may further comprise a polynucleotide sequence encoding one or more RNA molecules that are capable of unwinding the secondary structure of the endogenous signal RNA at the predetermined signal sequence.

In another embodiment of the invention, the polynucleotide molecule(s) of the composition that are described in any of sections 1, 8 or 9 may further comprise a polynucleotide sequence encoding a special functional RNA that is capable of inhibiting the expression, directly or indirectly, of an endogenous exonuclease. The special functional RNA may be, for example, but is not limited to: microRNA (miRNA), lariat-form RNA, short-hairpin RNA (shRNA), siRNA expression domain, antisense RNA, double-stranded RNA (dsRNA), small-interfering RNA (siRNA) or ribozyme.

The inhibitory sequence that is described in any of the embodiments above may be a sequence or a part of a sequence such that when it is detached from the sequence encoding the exogenous protein of interest, the exogenous protein of interest is capable of being expressed, and when it is not detached from the sequence encoding the exogenous protein of interest, it is capable of inhibiting the expression of the exogenous protein of interest, when it is within its specific context in the exogenous RNA of interest. Such that the inhibitory sequence may also be, only a part of any of the inhibitory sequences that described above within its specific context. For example, instead of an inhibitory sequence that is an out of reading frame 5′-AUG-3′, the inhibitory sequence may also be only the A or the 5′-AU-3′ part in the context of -UG-3′ or -G-3′ respectively (in other words, the exogenous RNA of interest comprises an out of reading frame 5′-AUG-3′ at the 5′ end, however the sequence that will be detached is only the 5′-AU-3′ part).

In another embodiment, the carrier RNA that is described in section 1 can also be 14-18 nucleotides long.

In another embodiment, the first sequence and the edge sequence that are described in any of sections 1, 8 or 9 can also be 29-200 nucleotides long, as long as the duplex that is formed when they are hybridized does not activate the PKR in the cell.

In additional embodiment, the cells that are described in any of the previous embodiments in any of the previous sections to which the composition of the invention is inserted/introduced, may further be, cells extract or in vitro mixture that comprises cellular proteins (such as, for example, Dicer, Risc).

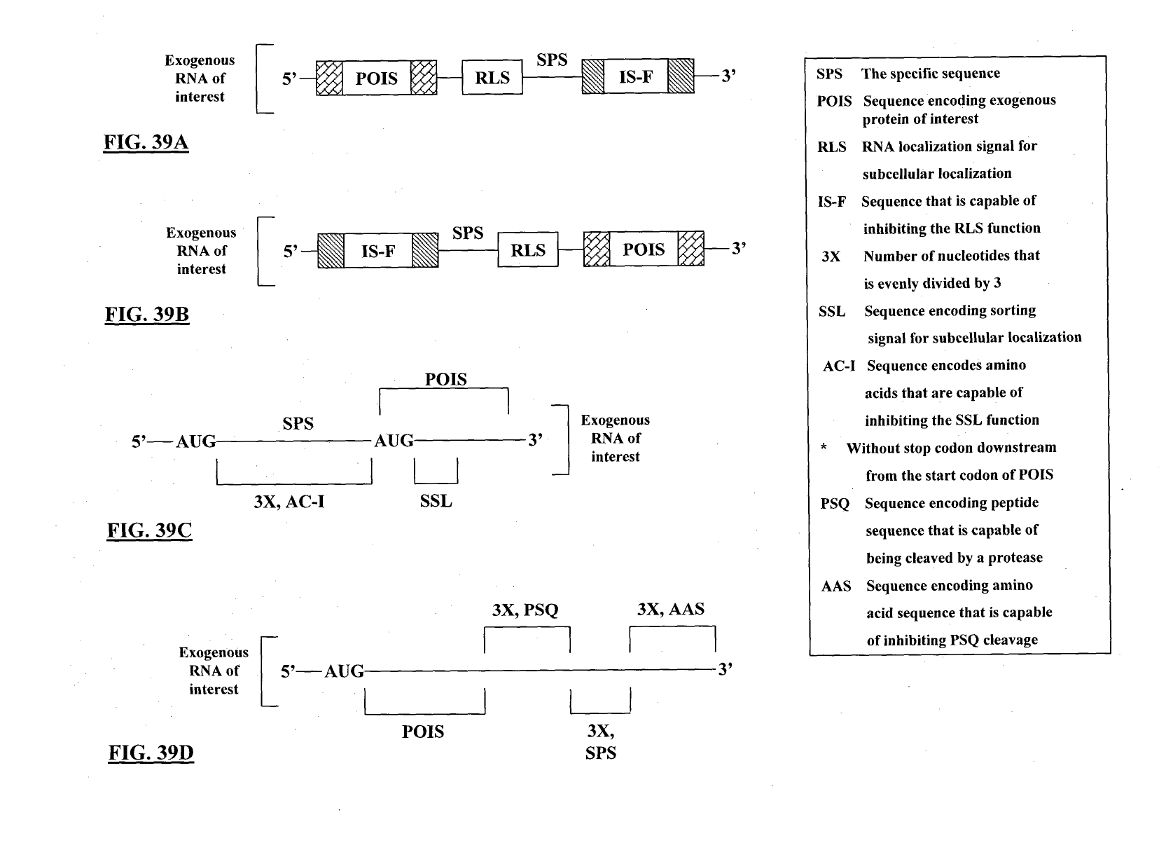

In another embodiment of the invention, the exogenous RNA of interest that is described in section 7 may further comprise an RNA localization signal for subcellular localization (including cotranslational import) between the specific target/cleavage site and the sequence encoding for the exogenous protein of interest, such that the inhibitory sequence is capable of inhibiting the function of the RNA localization signal for subcellular localization such that the subcellular localization of the exogenous RNA of interest is necessary for the proper expression of the protein of interest. For example, see FIG. 39A, 39B.

In another embodiment of the invention, the inhibitory sequence that is described in section 7 comprises an initiation codon upstream from the specific target/cleavage site, such that the initiation codon is consisting essentially of 5′-AUG-3′, such that the inhibitory sequence further comprises a nucleotide sequence encoding an amino acid sequence immediately downstream from the initiation codon, such that the nucleotide sequence and the sequence encoding the exogenous protein of interest are in the same reading frame, such that the amino acid sequence is capable of inhibiting the function of the sorting signal for subcellular localization of the exogenous protein of interest and such that the subcellular localization of the exogenous protein of interest is necessary for its proper expression. For example, see FIG. 39C.

In another embodiment of the invention, the exogenous RNA of interest that is described in section 7 does not comprise a stop codon downstream from the start codon of the sequence encoding the exogenous protein of interest, such that the inhibitory sequence is located downstream from the sequence encoding the exogenous protein of interest within the exogenous RNA of interest, such that the inhibitory sequence and the sequence encoding the exogenous protein of interest are in the same reading frame, such that the inhibitory sequence encodes an amino acid sequence that is selected from the group consisting of:
(a) an amino acid sequence that is capable of inhibiting the function/activity of the exogenous protein of interest;
(b) an amino acid sequence that is a sorting signal for subcellular localization;
(c) an amino acid sequence that is a protein degradation signal;
(d) an amino acid sequence that is capable of inhibiting the function of the sorting signal for subcellular localization of the protein of interest; and
(e) an amino acid sequence that is capable of inhibiting the cleavage of a peptide sequence that is encoded by a nucleotide sequence that is located between the specific target/cleavage site and the start codon of the sequence encoding the exogenous protein of interest, such that the nucleotide sequence and the sequence encoding the exogenous protein of interest are in the same reading frame and such that the peptide sequence is capable of being cleaved by a protease in a mammalian cell. It has been previously reported that in the human cell during translation of truncated mRNA without stop codon(s), the ribosome stalls at the terminal codon and the cognate tRNA molecule remains bound to the polypeptide chain and to the ribosome, however, it is possible for a peptidyl-tRNA species, in the midst of translation, to be processed by the endoplasmic reticulum signal peptidase [37]. For example, see FIG. 39D

### 11. PREPARATION OF THE COMPOSITION OF THE INVENTION

In one embodiment of the invention, the exogenous RNA of interest and the functional RNA, that are described in embodiments in section 1 are capable of being located on the same or different RNA molecules.

In another embodiment of the invention, the exogenous RNA of interest and/or the functional RNA, that are described in embodiments in section 1 are capable of being located within a third sequence.

In another embodiment of the invention, the exogenous RNA of interest, the functional RNA, the carrier RNA and the functional nucleic acid, that are described in embodiments in section 1 are capable of being located on one or more RNA molecules.

In some embodiments of the invention, the one or more polynucleotide molecule(s) described in any of the previous embodiments in any of the previous sections comprises one or more DNA molecule. In some embodiments, the one or more DNA molecule(s) are present in one or more DNA vectors (such as, for example, expression vectors), and/or viral vectors.

The polynucleotide molecule(s) (such as DNA molecules and/or RNA molecules) of the composition of the invention may be recombinantly engineered, by any of the methods known in the art, into a variety of host vector systems that may also provide for replication of the polynucleotide molecule(s) in large scale and which contain the necessary elements for directing the transcription of the RNA molecule(s) that are encoded from the composition of the invention. The use of such vectors to transfect target cells in the patient may result in the transcription of sufficient amounts of the RNA molecule(s) that are encoded from the composition of the invention. For example, a vector can be introduced in vivo such that it is taken up by a cell and directs the transcription of these RNA molecule(s). Such a vector can remain episomal or become chromosomally integrated, as long as it can be transcribed to produce the desired RNA molecule(s) that encoded from the composition of the invention. Such vectors can be constructed by recombinant DNA technology methods well known in the art or can be prepared by any method known in the art for the synthesis of DNA molecules.

The recombinant polynucleotide constructs (such as, for example, recombinant DNA constructs), that encode for the RNA molecule(s) which are encoded from the composition of the invention may be, for example, a plasmid, vector, viral construct, or others known in the art, used for replication and expression in the appropriate target cell (which may be, for example, mammalian cells). Expression of these RNA molecule(s) can be regulated by any promoter known in the art to act in the target cell (such as, for example, mammalian cells, which include, for example, human cells). Such promoters can be inducible or constitutive. Such promoters include, for example, but are not limited to: the SV40 early promoter region, the promoter contained in the 3′ long terminal repeat of Rous sarcoma virus, the herpes thymidine kinase promoter, the regulatory sequences of the metallothionein gene, the viral CMV promoter, the human chorionic gonadotropin-beta promoter, and the like. In some embodiments, the promoter may be an RNA Polymerase I promoter (i.e., a promoter that is recognized by RNA Pol. I), such as, for example, the promoter of ribosomal DNA (rDNA) gene. In such embodiments, the termination signal of the exogenous RNA of interest molecule may be a RNA Pol. I termination signal or a RNA polymerase II termination signal (such as, for example, a polyA signal). Any type of plasmid, cosmid, YAC or viral vector can be used to prepare the recombinant polynucleotide constructs which can be introduced directly into the target tissue/cell site. Alternatively, viral vectors can be used which selectively infect the desired target cell.

For the formation of transgenic organism that is resistant to viral infection, it is desirable that the vector that encodes for the RNA molecule(s) that are encoded from the composition of the invention will have a selectable marker. A number of selection systems can be used, including but not limited to selection for expression of the herpes simplex virus thymidine kinase, hypoxanthine-guanine phosphoribosyltransterase and adenine phosphoribosyl tranferase protein in tk-, hgprt- or aprt-deficient cells, respectively. Also, anti-metabolic resistance can be used as the basis of selection for dihydrofolate tranferase (dhfr), which confers resistance to methotrexate; xanthine-guanine phosphoribosyl transferase (gpt), which confers resistance to mycophenolic acid; neomycin (neo), which confers resistance to aminoglycoside G-418; and hygromycin B phosphotransferase (hygro) which confers resistance to hygromycin.

Vectors for use in the practice of the invention include any eukaryotic expression vectors. In some embodiments of the invention, the RNA molecule(s) that are encoded from the composition of the invention are encoded by a viral expression vector. The viral expression vector may be, for example, but is not limited to those belonging to a family of: Herpesviridae, Poxyiridae, Adenoviridae, Papillomaviridae, Parvoviridae, Hepadnoviridae, Retroviridae, Reoviridae, Filoviridae, Paramyxoviridae, Pneumoviridae, Rhabdoviridae, Orthomyxoviridae, Bunyaviridae, Hantaviridae, Picornaviridae, Caliciviridae, Togaviridae, Flaviviridae, Arenaviridae, Coronaviridae, or Hepaciviridae. The viral expression vector may also include, but is not limited to an adenoviral vector that its cellular tropism has been modified by the replacement of the adenovirus terminal knob domain of the fiber protein (HI loop), which is exposed at the fiber surface.

In another embodiment of the invention, the composition of the invention may comprise the RNA molecule(s) that is encoded from this composition, or derivatives or modified versions thereof, single-stranded or double-stranded. These RNA molecule(s) that are encoded from the composition of the invention may be, for example, but are not limited to deoxyribonucleotides, ribonucleosides, phosphodiester linkages, modified linkages or bases other than the five biologically occurring bases (adenine, guanine, thymine, cytosine and uracil).

The RNA molecule(s) that are encoded from the composition of the invention can be prepared by any method known in the art for the synthesis of RNA molecules. For example, these RNA molecule(s) may be chemically synthesized using commercially available reagents and synthesizers by methods that are well known in the art. Alternatively, these RNA molecule(s) can be generated by in vitro and in vivo transcription of DNA sequences that encoding these RNA molecule(s). Such DNA sequences can be incorporated into a wide variety of vectors which incorporate suitable RNA polymerase promoters such as the T7 or SP6 polymerase promoters. These RNA molecule(s) may be produced in high yield via in vitro transcription using plasmids such as SPS65. In addition, RNA amplification methods such as Q-beta amplification can be utilized to produce these RNA molecule(s).

The polynucleotide molecules, such as, the DNA molecule(s) and/or the RNA molecule(s), and/or the RNA molecules encoded by the composition can be modified at the base moiety, sugar moiety, or phosphate backbone, for example, in order to improve stability of the molecule, hybridization, transport into the cell, etc. In addition, modifications can be made to reduce susceptibility to nuclease degradation. The polynucleotide molecule(s) of the composition of the invention and/or the RNA molecules encoded by the composition may include any other appended groups such as, for example, peptides (for example, for targeting host cell receptors in vivo), or agents facilitating transport across the cell membrane or the blood-brain barrier, hybridization-triggered cleavage agents or intercalating agents. Various other well known modifications can be introduced as a means of increasing intracellular stability and half-life. Possible modifications include, but are not limited to, the addition of flanking sequences of ribo- or deoxy-nucleotides to the 5′ and/or 3′ ends of the molecule. In some circumstances where increased stability is desired, nucleic acids having modified intenucleoside linkages such as 2′-0-methylation may be preferred. Nucleic acids containing modified intenucleoside linkages may be synthesized using reagents and methods that are well known in the art.

The polynucleotide molecule(s) of the composition and/or the RNA molecule(s) that encoded from the composition of the invention may be purified by any suitable means, as are well known in the art (for example, reverse phase chromatography or gel electrophoresis).

Cells that produce viral vectors that together encode the RNA molecule(s) that are encoded from the composition of the invention, can also be used for transplantation in a patient for continuous treatment. These cells can further carry a specific gene that can kill them if a specific molecule is introduced to the patient's circulating system (for example: HSV1 Thymidine kinase/Ganciclovir).

In one embodiment, each one of the RNA molecule(s) that are encoded from the composition of the invention can be an RNA molecule or a reproducing RNA molecule. Such that the reproducing RNA molecule is an RNA molecule that comprises a sequence that is complementary to any of these RNA molecule(s) such that the reproducing RNA molecule is capable of being replicated in the cell for the formation of any of these RNA molecule(s).

In another embodiment, each of the RNA molecule(s) that are encoded from the composition of the invention can be prepared from various types, including, but are not limited to: synthetic RNA, synthetic RNA with modified bases, RNA that is produced by in vitro transcription, DNA molecule that encodes the RNA molecule, vector or viral vector that encodes the RNA molecule or DNA with modified bases that encodes the RNA molecule. For example, the functional RNA can be a synthetic siRNA while the exogenous RNA of interest can be encoded from a viral vector and while the Carrier RNA can be encoded from a plasmid.

### 12. USES AND ADMINISTRATION OF THE COMPOSITION OF THE INVENTION

The composition of the present invention may be used in various applications including, but not limited to: regulation of gene expression, targeted cell death, treatment, and/or prevention of various diseases and health related conditions (such as, for example, proliferative disorders (for example, cancer), infectious diseases and the like), diagnosis of various health related conditions, formation of transgenic organisms, suicide gene therapy, and the like. In one exemplary embodiment, the composition of the present invention can be used to activate toxic gene in cells that comprise viral RNA, in order to kill these cells. In another exemplary embodiment, the composition of the present invention can be used to activate toxic gene in cells that include an endogenous mRNA which comprises a predetermined signal sequence that is unique to cancer cells, for the targeted and specific killing of these cells.

According to some embodiments, there is thus provided a method for killing a specific cell/cell population, wherein the cell population comprises an endogenous signal RNA, comprising a predetermined signal sequence, which is unique and specific for these cells; the method includes introducing the cells with the composition of the invention, wherein the composition comprises one or more polynucleotides for directing the specific cleavage of an exogenous RNA of interest at a specific target site that is located within a specific sequence, which is of sufficient complementarity to hybridize with the predetermined signal sequence, wherein the cleavage of the exogenous RNA of interest leads to the expression of an exogenous protein of interest, capable of killing the cells.

According to some embodiments, the exogenous protein of interest may be selected from, but not limited to: any type of protein that can damage the cell function and as a result lead to the death of the cell. The protein may be selected from such types of proteins as, but not limited to: toxins, cell growth inhibitors, modulators of cellular growth, inhibitors of cellular signaling pathways, modulators of cellular signaling pathways, modulators of cell permeability, modulators of cellular processes, and the like:

According to some embodiments, and without wishing to be bound to theory or mechanism, the composition and methods of the present invention may provide a specific and targeted “all or none” response in a cell. In other words, compositions and methods of the present invention are such that the exogenous RNA of interest is cleaved (and consequently, the protein of interest is expressed and activated) only in those target cells, which include a specific endogenous signal RNA, whereas cells that do not include the endogenous signal RNA will not be effected by the composition of the invention. The composition and methods of the present invention may thus provide enhanced safety and control, since no leakiness in the expression of the exogenous protein of interest is observed in cells which do not include the endogenous signal RNA, which comprises the predetermined signal sequence.

In further embodiments, the composition of the present invention can be used to activate reporter gene in the presence of viral RNA for the diagnosis of viral infection diseases. In another embodiment, the composition of the present invention may be used to stably transfect cells for the formation of transgenic organism that is resistant to viral infection. In another embodiment, the composition of the present invention can be used to stably transfect cells for the formation of transgenic organism that is able to activate reporter gene in the presence of viral RNA for the diagnosis of viral infection diseases. In yet another embodiment, the composition of the present invention can be used to monitor in real time the changes in RNA sequence in the cell.

Various delivery systems and methods are well known in the art, which can be used to transfer/introduce/transfect the composition of the invention into target cells. The delivery systems and methods include, for example, use of various transfecting agents, encapsulation in liposomes, microparticles, microcapsules, recombinant cells that are capable of expressing the composition, receptor-mediated endocytosis, construction of the composition of the invention as part of a viral vector or other vector, viral vectors that are capable of being reproduced without killing the cell during the process of reproduction and that comprise the composition of the invention, viral vectors that are not capable of reproduction and that comprise the composition of the invention, injection of cells that produce viral vectors that comprise the composition of the invention, injection of DNA, electroporation, calcium phosphate mediated transfection, and the like, or any other suitable delivery system known or to be developed in the future.

In some embodiments, the present invention also provides for pharmaceutical compositions comprising an effective amount of the composition of the invention, and a pharmaceutically acceptable carrier. The term “Pharmaceutically acceptable” means approved by a regulatory agency of the Federal or a state government or listed in the U.S. Pharmacopeia or other generally recognized pharmacopeia for use in animals, and more particularly in humans. The term “Carrier” in the phrase “Pharmaceutically acceptable carrier” refers to a diluent, adjuvant, excipient, or vehicle with which the therapeutic is administered.

In some embodiments, the pharmaceutical compositions of the invention may be administered locally to the target area in need of treatment. This may be achieved by, for example, and not limited to: local infusion during surgery, topical application, e.g. in conjunction with a wound dressing after surgery, by injection, by means of a catheter, by means of a suppository, or by means of an implant, said implant being of a porous, non-porous, or gelatinous material, including membranes, such as sialastic membranes, or fibers. The local administration may be also achieved by control release drug delivery systems, such as nanoparticles, matrices such as controlled-release polymers or hydrogels.

In some embodiments, the composition of the invention can be administered in amounts which are effective to produce the desired effect in the targeted cell. Effective dosages of the composition of the invention can be determined through procedures well known to these in the art which address such parameters as biological half-life, bioavailability and toxicity. The amount of the composition of the invention which is effective depend on the nature of the disease or disorder being treated, and may be determined by standard clinical techniques. In addition, in vitro assays may optionally be employed to help identify optimal dosage ranges. The administered means may also include, but are not limited to permanent or continuous injection of the composition of the invention to the patient blood stream.

According to some embodiments, the present invention also provides for pharmaceutical pack or kit comprising one or more containers filled with one or more of the ingredients of the pharmaceutical compositions of the invention optionally associated with such container(s) can be a notice in the form prescribed by a governmental agency regulating the manufacture, use or sale of pharmaceuticals or biological products, which notice reflects approval by the agency of manufacture, use or sale for human or animal administration.

### EXAMPLES

The following examples are offered by way of illustration and not by way of limitation and are examples of embodiments of the present invention.

### Example 1

### Use of the Composition of the Invention to Kill the Cancer Cells of a Specific Patient

According to the American Cancer Society, 7.6 million people died from cancer in the world during 2007.

In this example the composition of the invention is designed to specifically kill the cancer cells of a specific patient. The first step in the designing of the composition of the invention for a specific patient is to identify the predetermined signal sequence, which is a sequence of 18-25 nucleotides long of an endogenous RNA molecule that is present in the cancer cells of this specific patient, such that the predetermined signal sequence is not present in any endogenous RNA molecule in the healthy or nonmetastatic tumourigenic cells of the body of the specific patient. Therefore, the predetermined signal sequence is an RNA sequence of a gene that is mutated in the cancer cells. On average each tumor comprises mutations in 90 protein-coding genes [16] and each tumor initiated from a single founder cell [38], therefore there is a need to identify only one of them that it is transcribed into RNA molecule in the cancer cells.

Various methods can be used for the identification of this predetermined signal sequence; these methods include, but are not limited to DNA microarray, Tilling (Targeting Induced Local Lesions in Genomes) and large-scale sequencing of cancer genomes. Furthermore the identification of the predetermined signal sequence can utilize the Cancer Genome Atlas project that has been cataloguing all the genetic mutations responsible for cancer by their genes.

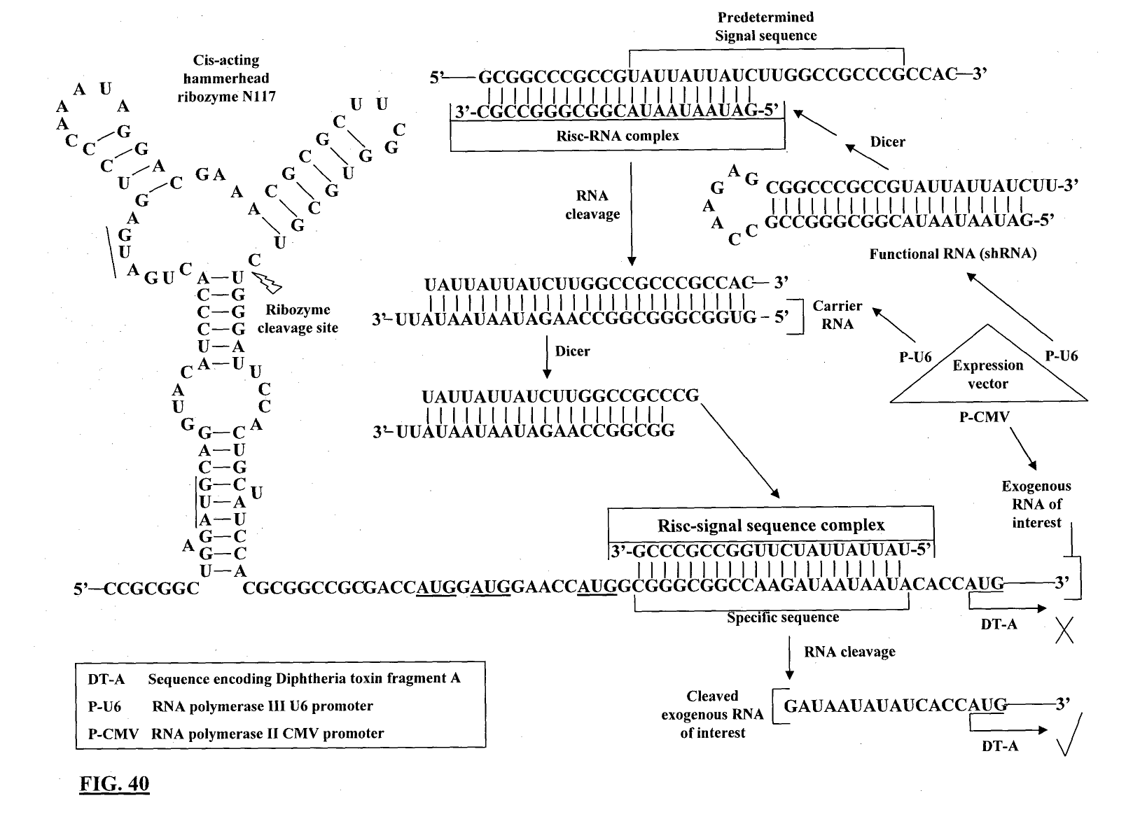

In this example, the predetermined signal sequence that is unique to the cancer cells of the specific patient is: 5′-UAUUAUUAUCUUGGCCGCCCG-3′ (SEQ ID NO. 41) and is located in an endogenous mRNA (SEQ ID NO. 42). Therefore, in this example the composition of the invention is designed to kill cells that comprise mRNA that comprises the sequence 5′-UAUUAUUAUCUUGGCCGCCCG-3′ (SEQ ID NO. 41). The functional RNA in this example (SEQ ID NO. 43) is shRNA that is designed to effect the cleavage of the 5′ end of the predetermined signal sequence. The sequence of the cleaved shRNA portion formed after processing by Dicer that hybridizes with the endogenous mRNA is set forth as SEQ ID NO. 44. The functional RNA is transcribed under the control of the very strong U6 promoter of RNA polymerase III. The G at the 5′ end and the UU at the 3′ end of the shRNA are necessary for the transcription by U6 promoter of RNA polymerase III. For example, see FIG. 40. It has been reported that in the cell the functional half-life of each of the two portions of a cleaved mRNA is reduced from the intact mRNA only by 2.6-1.7 fold [10]. It has also been reported that two portions of an mRNA that has been cleaved by RISC-RNA complex in a cell can be easily detected by Northern analysis [6].

The carrier RNA in this example is designed to be transcribed under the control of the very strong U6 promoter of RNA polymerase III and is designed to include the sequence: 3′-UUAUAAUAAUAGAACCGGCGGGCGGUG-5′ (SEQ ID NO. 45), the G at the 5′ end and the UU at the 3′ end of the carrier RNA are necessary for the transcription by U6 promoter of RNA polymerase III. In the cell, the carrier RNA is hybridized to the cleaved mRNA portion that includes the predetermined signal sequence and after processing by Dicer (SEQ ID NO. 46), the duplex that is formed is thermodynamically weaker at the 5′ end of the predetermined signal sequence, thus the predetermined signal sequence is be the strand that is loaded into Risc [3]. For example, see FIG. 40. The sequence of the cleaved carrier RNA portion formed after processing by Dicer is set forth as SEQ ID NO. 47.

It has been reported that in the cell, two RNA transcripts of about 23 nucleotides in length that have a complementary region of about 19 nucleotides in length at the 5′ end are hybridized with each other and are capable of directing target specific RNA interference [7]. It has also been reported that a dsRNA of 52 nucleotides long that further comprises 20 nucleotides long ssRNA at one of the 3′ ends is a substrate for a Dicer at the blunt end [8]. Furthermore it has also been reported that in mammal Risc is coupled to Dicer [9].

The specific sequence in the exogenous RNA of interest of this example is designed to comprise the sequence: 5′-CGGGCGGCCAAGAUAAUAAUA-3′ (SEQ ID NO. 48) that is 100% complementary to the predetermined signal sequence. For example, see FIG. 40. The exogenous RNA of interest is also designed to comprise a sequence encoding Diphtheria toxin fragment A (DT-A) downstream from the specific sequence. The exogenous RNA of interest is designed to be transcribed under the control of the strong viral CMV promoter. For example, see FIG. 40.

It has been reported that a single molecule of Diphtheria toxin fragment A introduced into a cell can kill the cell [14] and in mammal cells, the removal of a cap reduces translation of mRNA by 35-50 fold and reduces the functional mRNA half-life only by 1.7-fold [10].

The exogenous RNA of interest is also designed to comprise an inhibitory sequence upstream from the specific sequence, the inhibitory sequence comprises 3 initiation codons that 2 of them are located within the human Kozak consensus sequence: 5′-ACCAUGG-3′ (SEQ ID NO. 25) and each one of them is not in the same reading frame with the start codon of DT-A. For example, see FIG. 40.

The exogenous RNA of interest is also designed to comprise the very efficient cis-acting hammerhead ribozyme—N117 [23] at the 5′ end for reducing the efficiency of translation of the exogenous RNA of interest of the invention before it is cleaved. The cis-acting hammerhead ribozyme—N117 also comprises 2 initiation codons however each one of them is not in the same reading frame with the start codon of DT-A. For example, see FIG. 40. The entire sequence of the exogenous RNA of this example is set forth as SEQ ID NO. 49.

In this example, the functional RNA, the carrier RNA and the exogenous RNA of interest are transcribed by a viral vector. For example, see FIG. 40.

Such that, in the cell, the viral vector transcribes: the functional RNA, the carrier RNA and the exogenous RNA of interest. The cis acting ribozyme N117 in the exogenous RNA of interest removes the CAP structure from the 5′ end for reducing any translation by the exogenous RNA of interest and the out of reading frame initiation codons prevent translation of DT-A. The functional RNA (shRNA) effects the cleavage of the 5′ end of the predetermined signal sequence. The carrier RNA is hybridized to the cleaved mRNA portion that comprises the predetermined signal sequence and the predetermined signal sequence is processed by Dicer and loaded into Risc. The Risc-signal sequence complex cleaves the exogenous RNA of interest at the specific sequence and the out of reading frame initiation codons are detached, so that DT-A is expressed at least one time, which enough to cause cell death. For example, see FIG. 40. The sequence of the cleaved exogenous RNA of this example is set forth as SEQ ID NO. 50.

### Example 2

### Use of the Composition of the Invention to Kill EBV-Associated Gastric Carcinomas Cancer Cells, Nasopharyngeal Carcinoma Cancer Cells, Burkitt's Lymphoma Cancer Cells and Hodgkin's Lymphoma Cancer Cells

Epstein-Barr virus (EBV) is a ubiquitous human gammaherpesvirus that establishes life-long latent infections in B lymphocytes following the primary infection. EBV infects the majority of the population worldwide and has been implicated in the pathogenesis of several human malignancies including Burkitt's and Hodgkin's lymphomas, gastric carcinoma and nasopharyngeal carcinoma (NPC) [32]. EBV infection is mainly characterized by the expression of latent genes including EBNA1, LMP1, LMP2 and EBER [32]. LMP1 (latent membrane protein 1) was the first EBV latent gene found to be able to transform cell lines and alter the phenotype of cells due to its oncogenic potential [32]. In human epithelial cells, LMP1 alters many functional properties that are involved in tumor progression and invasions [32].

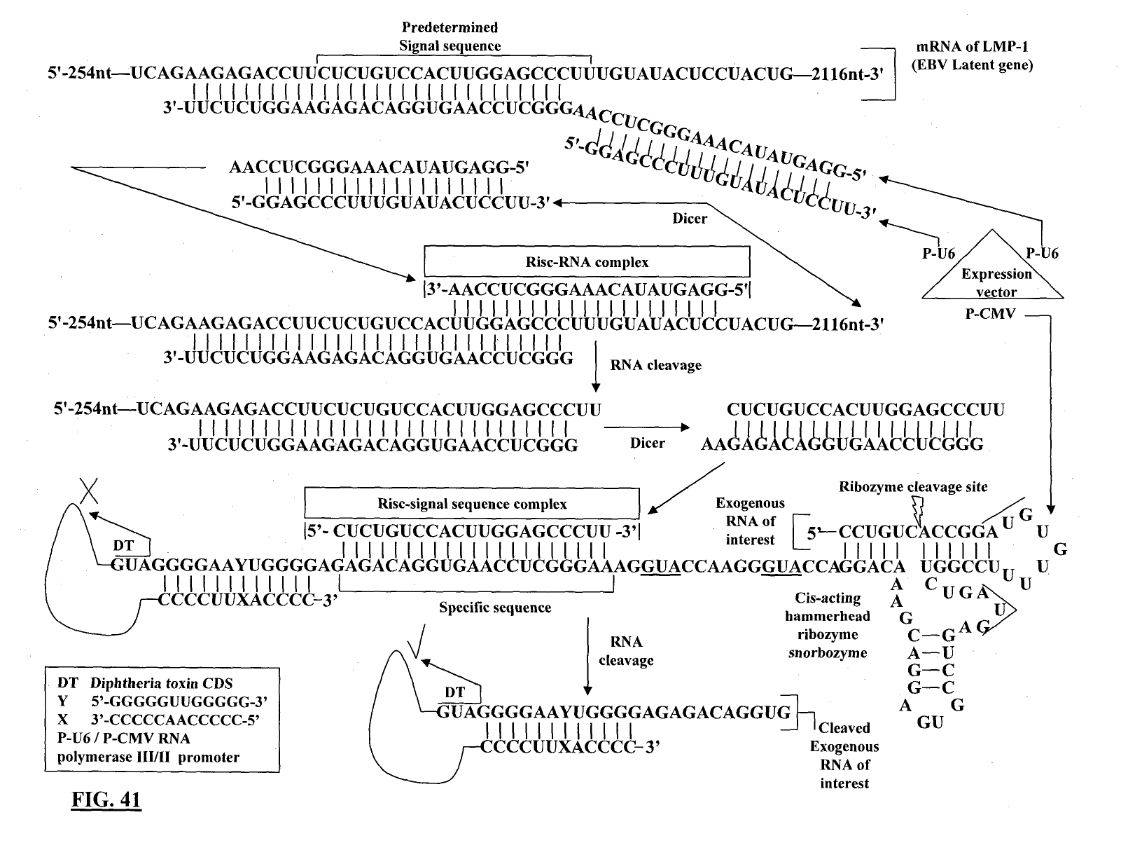

In this example the composition of the invention is designed to kill cancer cells of Burkitt's lymphomas, Hodgkin's lymphomas, gastric carcinoma and nasopharyngeal carcinoma, which are latently infected with EBV, by using the LMP1 mRNA as the endogenous signal RNA and by using the sequence: 5′-CUCUGUCCACUUGGAGCCCUU-3′ (SEQ ID NO. 51—nucleotides 269-289 of LMP1 mRNA) as the predetermined signal sequence. For example, see FIG. 41. Nucleotides 255-304 of LMP1 mRNA are also shown in the figure and set forth as SEQ ID NO. 52. This predetermined signal sequence is chosen because it is located in a region that does not have RNA secondary structure and because it is located in a region that has been shown to be a good target for siRNA [33]. Furthermore, this predetermined signal sequence is also chosen because its cleavage creates a relatively short RNA molecule of 289 nucleotides long.

In this example, the carrier sequence and the functional RNA are located in the same RNA duplex that is hybridized in the cell, such that the double strand region is located at the 5′ end of the carrier sequence and such that when the double strand region is processed by Dicer, the carrier sequence is detached from the RNA duplex and the siRNA duplex that is formed is the functional RNA and is capable of effecting the cleavage of the mRNA of LMP-1 at the 3′ end of the predetermined signal sequence. The 2 strands of the RNA duplex are: 3′-UUCUCUGGAAGAGACAGGUGAACCUCGGGAACCUCGGGAAACAUAUGAGG-5′(SEQ ID NO. 53) and 5′-GGAGCCCUUUGUAUACUCCUU-3′ (SEQ ID NO. 54). The 2 strands of the RNA duplex are transcribed under the control of the very strong U6 promoter of RNA polymerase III, thus their 5′ end is G and their 3′ end is UU. For example, see FIG. 41. The sequence of the cleaved strand (after Dicer processing) capable of hybridizing to the mRNA of LMP-1 and affecting its cleavage at the 3′ end of the predetermined signal sequence is set forth as SEQ ID NO. 55.

When the mRNA of LMP-1 is cleaved at the 3′ end of the predetermined signal sequence, the carrier sequence (SEQ ID NO. 56) directs the predetermined signal sequence to Dicer processing and the duplex that is formed is thermodynamically weaker at the 5′ end of the predetermined signal sequence, thus the predetermined signal sequence will be the strand that will be loaded into Risc [3]. For example, see FIG. 41. The sequence of the second strand, namely the cleaved carrier sequence after processing by Dicer is set forth as SEQ ID NO. 57.

The specific sequence in the exogenous RNA of interest of the example is designed to comprise the sequence: 3′-GAGACAGGUGAACCUCGGGAA-5′ (SEQ ID NO. 58) that is 100% complementary to the predetermined signal sequence. The exogenous RNA of interest is also designed to comprise a sequence encoding Diphtheria toxin (DT) downstream from the specific sequence. The exogenous RNA of interest is designed to be transcribed under the control of the strong viral CMV promoter. The exogenous RNA of interest is also designed to comprise an inhibitory sequence upstream from the specific sequence. The inhibitory sequence comprises 2 initiation codons that are located within the human Kozak consensus sequence: 5′-ACCAUGG-3′ (SEQ ID NO. 25) and each one of them is not in the same reading frame with the start codon of DT. The exogenous RNA of interest is also designed to comprise the very efficient cis-acting hammerhead ribozyme-snorbozyme [22] at the 5′ end for reducing the efficiency of translation of the exogenous RNA of interest of the invention before it is cleaved. The cis-acting hammerhead ribozyme-snorbozyme also comprises 2 initiation codons however each one of them is not in the same reading frame with the start codon of DT. The exogenous RNA of interest is also designed to comprise a nucleotide sequence of 23 nucleotides downstream from the specific sequence and upstream from the sequence encoding DT, such that the nucleotide sequence is capable of binding to a sequence of 23 nucleotides that is located downstream from the sequence encoding DT, such that the exogenous RNA of interest forms a circular structure that increases the efficiency of translation of DT particularly when the exogenous RNA of interest is cleaved. For example, see FIG. 41. The entire sequence of the exogenous RNA of this example is set forth as SEQ ID NO. 59.

In this example, the two strands of the RNA duplex and the exogenous RNA of interest are transcribed by a viral vector (see FIG. 41). Such that, in the cell, the viral vector transcribes: the two strands of the RNA duplex and the exogenous RNA of interest. The cis acting ribozyme, snorbozyme, in the exogenous RNA of interest removes the CAP structure from the 5′ end for reducing any translation by the exogenous RNA of interest and the out of reading frame initiation codons prevent translation of DT. The two strands of the RNA duplex are hybridized with each other and with the predetermined signal sequence at the LMP-1 mRNA, the double strand region of the RNA duplex is cleaved by Dicer and forms the functional RNA that is siRNA and the carrier sequence. The siRNA cleaves the predetermined signal sequence at the 3′ end and the carrier sequence directs the cleaved predetermined signal sequence to Dicer processing. The processed predetermined signal sequence is loaded into Risc and then the Risc-signal sequence complex cleaves the exogenous RNA of interest at the specific sequence and the out of reading frame initiation codons are detached, so that DT is capable of being expressed. The sequence of the cleaved exogenous RNA of this example is set forth as SEQ ID NO. 60. The RNA portion that comprises the sequence encoding DT forms a circular structure that increases DT translation for killing the cancer cell and the neighboring cells. For example, see FIG. 41.

In this example, the functional RNA and the carrier sequence are located in the same RNA duplex, thus the carrier sequence may bring the functional RNA into proximity with the predetermined signal sequence and by this may also bring the components of the RNA interference pathway (e.g. Dicer and Risc) into proximity with the predetermined signal sequence.

### Example 3

### Use of the Composition of the Invention to Kill HIV-1 Infected Cells

According to the World Health Organization in 2006 there were about 39.5 million people with HIV worldwide. According to current estimates of the Joint United Nations Program on HIV and AIDS, HIV is set to infect 90 million people in Africa. HIV (Human immunodeficiency virus) can lead to the acquired immunodeficiency syndrome (AIDS). Two species of HIV infect humans: HIV-1 and HIV-2. HIV-1 is more virulent, relatively easily transmitted, and is the cause of the majority of HIV infections globally. HIV-2 is less transmittable than HIV-1 and is largely confined to West Africa.

Many viruses, including HIV, exhibit a dormant or latent phase, during which little or no protein synthesis is conducted. The viral infection is essentially invisible to the immune system during such phases. Current antiviral treatment regimens are largely ineffective at eliminating cellular reservoirs of latent viruses [15].

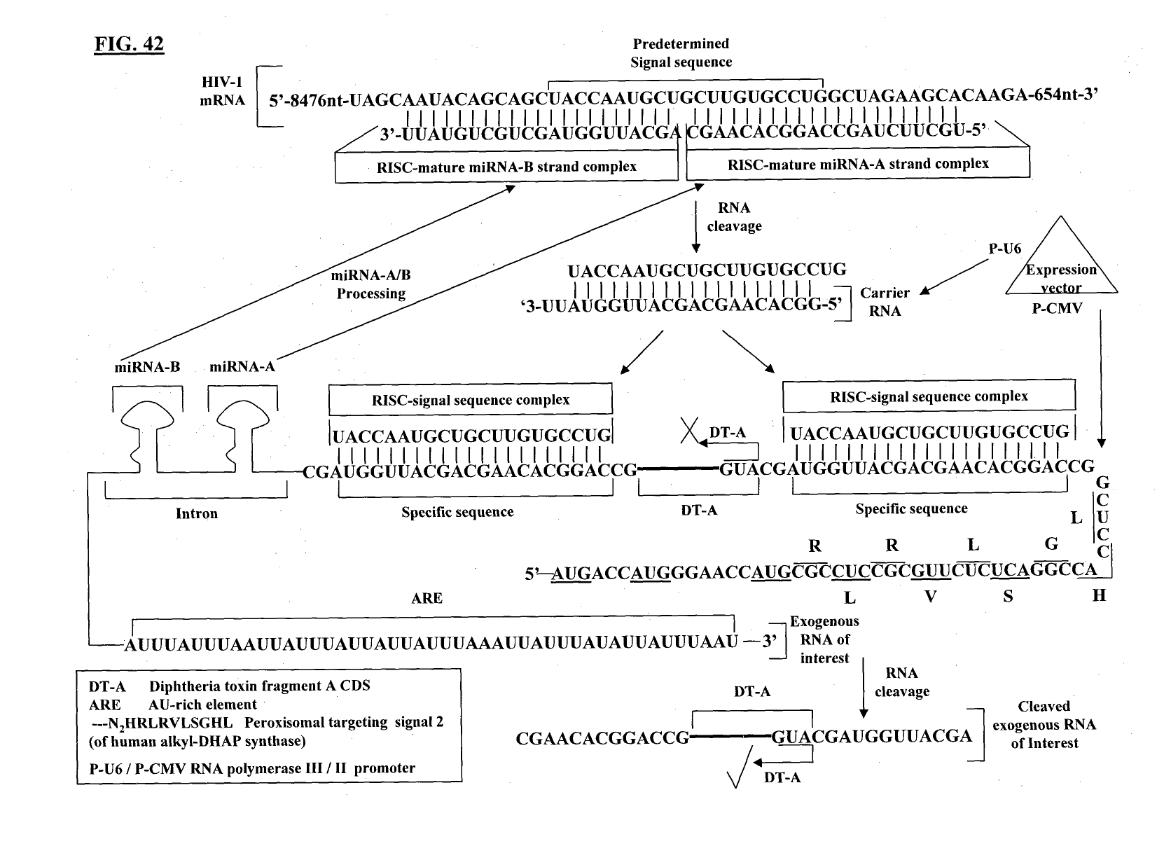

In this example, the composition of the invention is designed to kill HIV-1 infected cells by using the HIV-1 mRNA as the endogenous signal RNA and by using the sequence: 5′-UACCAAUGCUGCUUGUGCCUG-3′ (SEQ ID NO. 61—nucleotides 8492-8512 of HIV-1 mRNA) as the predetermined signal sequence. For example, see FIG. 42. Nucleotides 8477-8527 of HIV-1 mRNA are also shown in the figure and set forth as SEQ ID NO. 62. This predetermined signal sequence is chosen because it is located in a region that does not include an RNA secondary structure and because it is located in a region that has been shown to be a good target for siRNA [34].

The exogenous RNA of interest of this example is transcribed under the control of the strong viral CMV promoter and is designed to comprise 2 specific sequences, such that each one of them is: 3′-AUGGUUACGACGAACACGGAC-5′ (SEQ ID NO. 63) that is 100% complementary to the predetermined signal sequence. The exogenous RNA of interest is also designed to comprise a sequence encoding Diphtheria toxin fragment A (DT-A) between the 2 specific sequences. In mammal cells single molecule of Diphtheria toxin fragment A introduced into a cell can kill the cell [14]. The exogenous RNA of interest is also designed to comprise two inhibitory sequences one at the 5′ end and other at the 3′ end. The inhibitory sequence that is located at the 5′ end of the exogenous RNA of interest is designed to include 3 initiation codons, such that one of them is located within the human Kozak consensus sequence: 5′-ACCAUGG-3′ (SEQ ID NO. 25), such that each one of them is not in the same reading frame with the start codon of DT-A and such that all the 3 initiation codons are in the same reading frame. The inhibitory sequence that is located at the 5′ end of the exogenous RNA of interest also comprises a nucleotide sequence downstream from the 3 initiation codons and upstream from the 2 specific sequences, such that the nucleotide sequence is in the same reading frame with the 3 initiation codons and such that the nucleotide sequence encodes for a sorting signal for the subcellular localization that is the Peroxisomal targeting signal 2 of the human alkyl dihydroxyacetonephosphate synthase (H₂N - - - RLRVLSGHL—SEQ ID NO. 27) [30]. In mammal cells proteins that bear a sorting signal for the subcellular localization can be localized to the subcellular localization while they are being translated with their mRNA. For example, see FIG. 42.

The inhibitory sequence that is located at the 3′ end of the exogenous RNA of interest is designed to include an intron downstream from the 2 specific sequences, such that the exogenous RNA of interest is a target for nonsense-mediated decay (NMD) that degrades RNA molecule that comprises an intron downstream from the coding sequence [31]. The intron comprises 2 artificial microRNAs that are designed to affect the cleavage of the predetermined signal sequence at the 5′ end and at the 3′ end (SEQ ID NOs. 64 and 65, respectively) [35]. The inhibitory sequence that is located at the 3′ end of the exogenous RNA of interest also comprises an AU-rich element (ARE) at the 3′ end that stimulates degradation of the exogenous RNA of interest. The AU-rich elements is 47 nucleotides long and it comprises the sequences: 5′-AUUUA-3′ (SEQ ID NO. 31) and 5′-UUAUUUA(U/A)(U/A)-3′(SEQ ID NO. 32) [28]. For example, see FIG. 42. The entire sequence of the exogenous RNA of this example is composed of SEQ ID NO. 66, SEQ ID NO. 113 and an intron comprising the two artificial microRNAs described above in between. The carrier RNA in this example, is designed to be transcribed under the control of the very strong U6 promoter of RNA polymerase III and is designed to include the sequence: 3′-UUAUGGUUACGACGAACACGG-5′ (SEQ ID NO. 67), the G at the 5′ end and the UU at the 3′ end of the carrier RNA are necessary for the transcription by U6 promoter of RNA polymerase III. In the cell the carrier RNA of the invention may be hybridized to the cleaved predetermined signal sequence and the duplex that is formed is thermodynamically weaker at the 5′ end of the predetermined signal sequence, thus the predetermined signal sequence is the strand that is loaded into Risc [3]. For example, see FIG. 42.

In this example, the carrier RNA and the exogenous RNA of interest are transcribed by a viral vector. Such that, in the cell, the viral vector transcribe: the carrier RNA and the exogenous RNA of interest. The out of reading frame initiation codons prevent translation of DT-A, the Peroxisomal targeting signal 2 sends the erroneous protein and the exogenous RNA of interest to the peroxisome. The intron targets the exogenous RNA of interest to degradation by the nonsense-mediated decay (NMD) and the AU-rich element also stimulates degradation of the exogenous RNA of interest. In the presence of the HIV-1 mRNA in the cell the two artificial microRNAs cleave the predetermined signal sequence at the 5′ end and at the 3′ end and the carrier RNA is hybridized to the cleaved predetermined signal sequence, and the signal sequence may be loaded into Risc. Then the Risc-signal sequence complex may cleave the exogenous RNA of interest at the two specific sequences and all the inhibitory sequences are detached, so that DT-A is expressed at least one time, which enough to cause cell death. For example, see FIG. 42. The sequence of the cleaved exogenous RNA of this example is set forth as SEQ ID NO. 68.

In this example, the predetermined signal sequence is cleaved from both of its ends and thus with the carrier RNA it is a better substrate for Dicer or Risc.

The viral vector may also encode transcriptional factors that are capable of enhancing the transcription of HIV-1 mRNA in HIV-1 infected cell (e.g. NF-κB). The viral vector may also encode genes that are capable of preventing new HIV-1 particles production (e.g. Rev, which prevents HIV-1 mRNA splicing).

### Example 4

### Use of the Composition of the Invention to Kill Hsv-1 Infected Cells

Many viruses, including HSV-1 (herpes simplex virus-1) exhibit a dormant or latent phase, during which no protein synthesis is conducted. The viral infection is essentially invisible to the immune system during such phases. Current antiviral treatment regimens are largely ineffective at eliminating cellular reservoirs of latent viruses [15]. The latency-associated transcript (LAT) of herpes simplex virus-1 (HSV-1) is the only viral gene that is expressed during latent infection in neurons. LAT inhibits apoptosis and maintains latency by promoting the survival of infected neurons. No protein product has been attributed to the LAT gene.

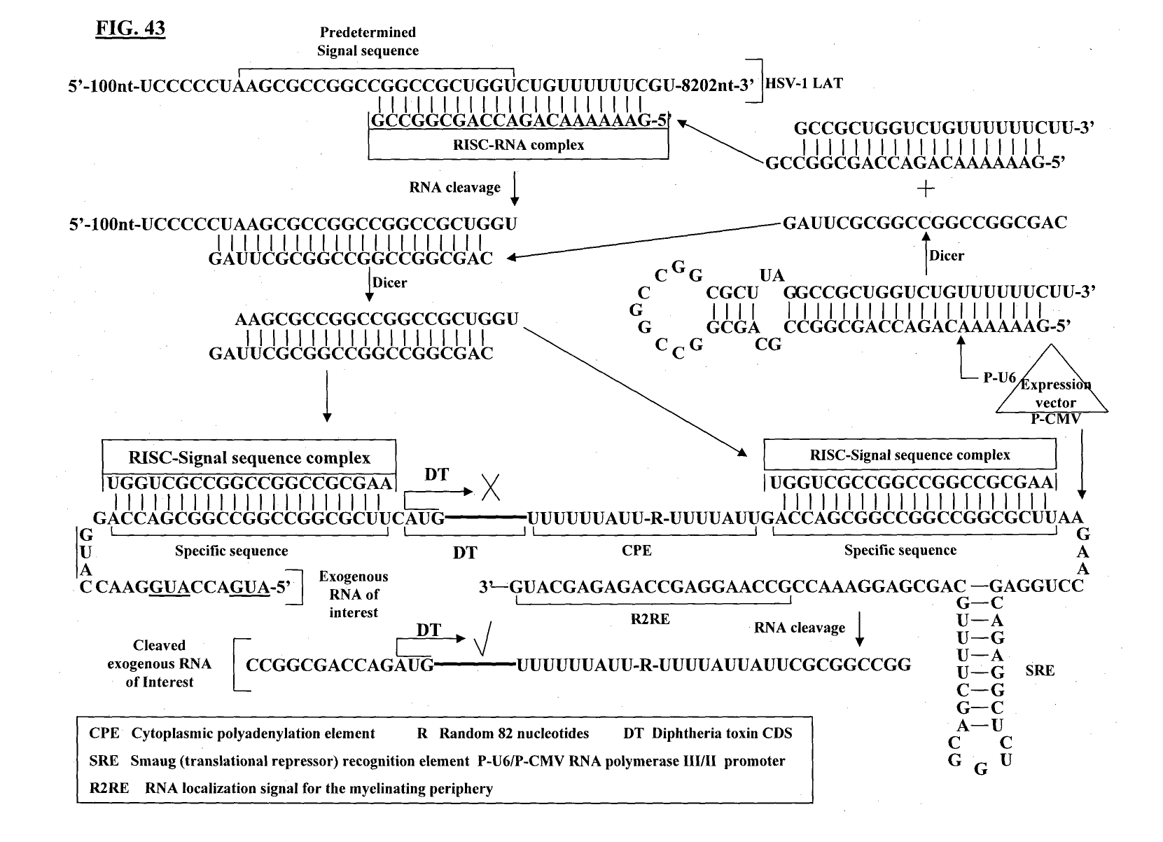

In this example, the composition of the invention is designed to kill HSV-1 infected cells by using the latency-associated transcript (LAT) as the endogenous signal RNA and by using the sequence: 5′-AAGCGCCGGCCGGCCGCUGGU-3′ (SEQ ID NO. 69—nucleotides 108-128 of the latency-associated transcript—LAT of HSV-1) as the predetermined signal sequence. For example, see FIG. 43. Nucleotides 101-140 of HSV-1 LAT mRNA are also shown in the figure and set forth as SEQ ID NO. 70. This predetermined signal sequence is chosen because its cleavage creates a relatively short RNA molecule of 128 nucleotides long. For example, see FIG. 43.

In this example, the carrier sequence and the functional RNA are located in the same stem loop structure (SEQ ID NO. 71) that is transcribed by the RNA polymerase III U6 promoter. Such that when the stem loop structure is processed by Dicer, the carrier sequence (SEQ ID NO. 72) is detached from the stem loop structure and the siRNA duplex that is formed is the functional RNA, which is capable of effecting the cleavage of LAT at the 3′ end of the predetermined signal sequence. The sequences of the strands of the siRNA duplex that is formed are set forth as SEQ ID NOs. 73 and 74. The sequence of the cleaved LAT portion that comprises the predetermined signal sequence is set forth as SEQ ID NO. 75. The G at the 5′ end and the UU at the 3′ end of the stem loop structure are necessary for the transcription by U6 promoter of RNA polymerase III.

In the cell, the carrier sequence is hybridized to the cleaved LAT portion that comprises the predetermined signal sequence and after processing by Dicer, the duplex that is formed is thermodynamically weaker at the 5′ end of the predetermined signal sequence, thus the predetermined signal sequence will be the strand that will be loaded into Risc [3]. For example, see FIG. 43.

The exogenous RNA of interest of this example is transcribed under the control of the strong viral CMV promoter and is designed to comprise 2 specific sequences, such that each one of them is: 5′-ACCAGCGGCCGGCCGGCGCUU-3′ (SEQ ID NO. 76) that is 100% complementary to the predetermined signal sequence. The exogenous RNA of interest is also designed to comprise a sequence encoding Diphtheria toxin (DT) between the 2 specific sequences. The exogenous RNA of interest is also designed to comprise 2 inhibitory sequences one at the 5′ end and other at the 3′ end of the exogenous RNA of interest. The inhibitory sequence that is located at the 5′ end of the exogenous RNA of interest is designed to include 3 initiation codons, such that 2 of them are located within the human Kozak consensus sequence: 5′-ACCAUGG-3′ (SEQ ID NO. 25), such that each one of them is not in the same reading frame with the start codon of DT. The inhibitory sequence that is located at the 3′ end of the exogenous RNA of interest is designed to comprise the translational repressor smaug recognition elements (SRE): 5′-UGGAGCAGAGGCUCUGGCAGCUUUUGCAGCG-3′ (SEQ ID NO. 28) downstream from the 2 specific sequences. For example, see FIG. 43. Smaug 1 is encoded in human chromosome 14 and is capable of repressing translation of SRE-containing messengers [26, 27]. Murine Smaug 1 is expressed in the brain and is abundant in synaptoneurosomes, a subcellular region where translation is tightly regulated by synaptic stimulation [26]. The inhibitory sequence that is located at the 3′ end of the exogenous RNA of interest also comprises an RNA localization signal for myelinating periphery (A2RE—Nuclear Ribonucleoprotein A2 Response Element): 5′-GCCAAGGAGCCAGAGAGCAUG-3′ (SEQ ID NO. 29) at the 3′ end [29]. For example, see FIG. 43. A2RE is a cis-acting sequence that is located at the 3′-untranslated region of MBP (Myelin basic protein) mRNA and is sufficient and necessary for MBP mRNA transport to the myelinating periphery of oligodendrocytes [29]. The hnRNP (Heterogeneous Nuclear Ribonucleoprotein) A2 binds the A2RE and mediates transport of MBP [29].

The exogenous RNA of interest of this example also comprises a cytoplasmic polyadenylation element (CPE) immediately downstream from the sequence encoding DT. The CPE comprises the sequence 5′-UUUUUUAUU-3′ (SEQ ID NO. 38) immediately downstream from the sequence encoding DT and the sequence 5′-UUUUAUU-3′ (SEQ ID NO. 39) 91 nucleotides downstream from the sequence encoding DT [25]. For example, see FIG. 43. In mammals, CPEB (cytoplasmic polyadenylation element binding protein) is present in the dendritic layer of the hippocampus (the portion of the brain that is responsible for long-term memory) [36]. In the synapto-dendritic compartment of mammalian hippocampal neurons, CPEB appears to stimulate the translation of α-CaMKII mRNA, which comprises CPE, by polyadenylation-induced translation [36]. The entire sequence of the exogenous RNA of this example is set forth as SEQ ID NO. 77.

In this example the exogenous RNA of interest and the stem loop structure are transcribed by a viral vector. Such that after the transcription of the exogenous RNA of interest and the stem loop structure, the out of reading frame initiation codons prevent translation of DT, the Smaug1 (translational repressor) binds to the smaug recognition elements (SRE) and inhibits DT translation and the hnRNP A2 binds the A2RE and mediates the transport of the RNA molecule to the myelinating periphery. Correspondingly the stem loop structure is processed by Dicer such that the carrier sequence is detached from the stem loop structure and the siRNA duplex that is formed is the functional RNA, and then the functional RNA effects the cleavage of LAT at the 3′ end of the predetermined signal sequence. Then the carrier sequence is hybridized to the LAT portion that comprises the predetermined signal sequence and the predetermined signal sequence is processed by Dicer and loaded into Risc. Then, the Risc-signal sequence complex cleaves the exogenous RNA of interest at the 2 specific sequences and all the inhibitory sequences are detached, so that the CPEB (cytoplasmic polyadenylation element binding protein) binds to the CPE and stimulates the extension of the poly-A tail in the cleaved exogenous RNA of interest, such that DT is capable of being expressed and kills the cell and the neighboring cells. For example, see FIG. 43. The sequence of the cleaved exogenous RNA of this example is set forth as SEQ ID NO. 78.

In this example, the functional RNA and the carrier sequence are located in the same RNA molecule, which requires less transcriptional units. The major advantage of this proximity of the functional RNA and the carrier sequence is that they are created in the same place in the cell and in the same time and also at a constant ratio.

### Example 5

### Use of the Composition of the Invention to Kill Cancer Cells of a Specific Patient

In this example the composition of the invention is designed to kill the cancer cells of a specific patient.

As described in Example 1 above, the first step in the designing of the composition of the invention for a specific patient is to identify the predetermined signal sequence, which is a sequence of 18-25 nucleotides long of an RNA molecule that present in the cancer cells of this specific patient, such that the predetermined signal sequence is not present in any RNA molecule in the healthy or nonmetastatic tumourigenic cells of the body of this specific patient. Therefore, the predetermined signal sequence is an RNA sequence of a gene that is mutated in the cancer cells. On average each tumor comprises mutations in 90 protein-coding genes [16] and each tumor initiated from a single founder cell [38], therefore there is a need to identify only one of them that it is transcribed into an RNA molecule in the cancer cells. Various methods can be used for the identification of this predetermined signal sequence; these methods include, but are not limited to DNA microarray, Tilling (Targeting Induced Local Lesions in Genomes) and large-scale sequencing of cancer genomes. Furthermore the identification of the predetermined signal sequence can utilize the Cancer Genome Atlas project that has been cataloguing all the genetic mutations responsible for cancer by their genes.

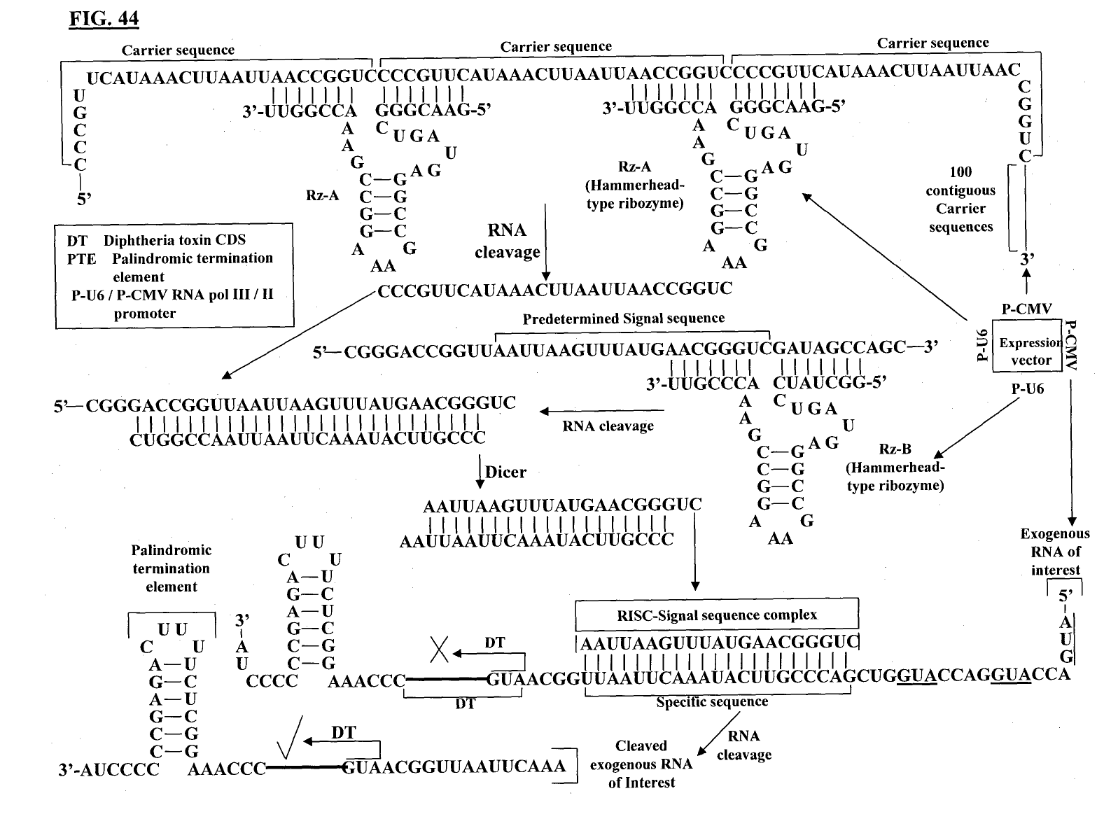

In this example, the predetermined signal sequence that is unique to the cancer cells of the specific patient is: 5′-AAUUAAGUUUAUGAACGGGUC-3′ (SEQ ID NO. 79) and is located in an endogenous mRNA. Therefore, in this example the composition of the invention is designed to kill cells that comprise endogenous mRNA (as the endogenous signal RNA) that comprises the sequence 5′-AAUUAAGUUUAUGAACGGGUC-3′ (SEQ ID NO. 79). An exemplary endogenous mRNA comprising said predetermined signal sequence is shown in FIG. 44 and set forth as SEQ ID NO. 80.

The functional RNA in this example is Rz-B, a hammerhead-type ribozyme (SEQ ID NO. 81) [21] that is designed to effect the cleavage of the 3′ end of the predetermined signal sequence. The sequence of the exemplary endogenous mRNA comprising the predetermined signal sequence after cleavage is set forth as SEQ ID NO. 82. The hammerhead-type ribozyme Rz-B is transcribed under the control of the very strong U6 promoter of RNA polymerase III. The G at the 5′ end and the UU at the 3′ end of the hammerhead-type ribozyme Rz-B are necessary for the transcription by U6 promoter of RNA polymerase III. For example, see FIG. 44. It has been reported that in the cell the functional half-life of each of the two portions of a cleaved mRNA is reduced from the intact mRNA only by 2.6-1.7 fold [10]. It has also been reported that two portions of an mRNA that has been cleaved by RISC-RNA complex in a cell can be easily detected by Northern analysis [6].

The carrier sequence of this example is: 5′-CCCGUUCAUAAACUUAAUUAACCGGUC-3′ (SEQ ID NO. 83) and 103 contiguous carrier sequences are located in an RNA sequence that is transcribed under the control of the strong viral CMV promoter. Such that Rz-A, a hammerhead-type ribozyme (SEQ ID NO. 84) [21], is designed to effect the cleavage of the 3′ end of the carrier sequence that is located at the 5′ end of the RNA sequence. The hammerhead-type ribozyme Rz-A is transcribed under the control of the very strong U6 promoter of RNA polymerase III. The G at the 5′ end and the UU at the 3′ end of the hammerhead-type ribozyme Rz-A are necessary for the transcription by U6 promoter of RNA polymerase. III. For example, see FIG. 44.

In the cell, the hammerhead-type ribozyme Rz-A detaches up to 101 perfect carrier sequences from 1 RNA sequence. The detached carrier sequence is hybridized to the cleaved mRNA portion that comprises the predetermined signal sequence and after Dicer processing the duplex that is formed is thermodynamically weaker at the 5′ end of the predetermined signal sequence, thus the predetermined signal sequence will be the strand that will be loaded into Risc [3]. For example, see FIG. 44. The sequence of the second strand of the duplex that is formed, namely the cleaved carrier sequence after processing by Dicer, is set forth as SEQ ID NO. 85.

The specific sequence in the exogenous RNA of interest of the example is designed to comprise the sequence: 3′-UUAAUUCAAAUACUUGCCCAG-5′ (SEQ ID NO. 86) that is 100% complementary to the predetermined signal sequence. The exogenous RNA of interest is also designed to comprise a sequence encoding Diphtheria toxin (DT) downstream from the specific sequence. The exogenous RNA of interest is designed to be transcribed under the control of the strong viral CMV promoter. The exogenous RNA of interest is also designed to comprise an inhibitory sequence upstream from the specific sequence. The inhibitory sequence comprises 3 initiation codons that 2 of them are located within the human Kozak consensus sequence: 5′-ACCAUGG-3′ (SEQ ID NO. 25) and each one of them is not in the same reading frame with the start codon of DT. The exogenous RNA of interest of the invention also comprises the palindromic termination element (PTE) from the human HIST1H2AC(H2ac) gene 3′UTR (5′-GGCUCUUUUCAGAGCC-3′ —SEQ ID NO. 34)) downstream from the sequence encoding DT. For example, see FIG. 44. The PTE plays an important role in mRNA processing and stability [11]. Transcripts from HIST1H2AC gene lack poly(A) tails and are still stable, due to the PTE. The entire sequence of the exogenous RNA of this example is set forth as SEQ ID NO. 87.

In this example the exogenous RNA of interest, the hammerhead-type ribozyme Rz-B/Rz-A and the RNA sequence that comprising 103 carrier sequences are transcribed by a viral vector. Such that in the cell, the viral vector transcribes: the exogenous RNA of interest, the hammerhead-type ribozyme Rz-B/Rz-A and the RNA sequence that comprising 103 carrier sequences. The out of reading frame initiation codons prevent translation of DT. The hammerhead-type ribozyme Rz-B cleaves the 3′ end of the predetermined signal sequence. The hammerhead-type ribozyme Rz-A detaches up to 101 perfect carrier sequences from 1 RNA sequence. The detached carrier sequence is hybridized to the cleaved mRNA portion that comprises the predetermined signal sequence and the predetermined signal sequence is processed by Dicer and loaded into Risc. The Risc-signal sequence complex cleaves the exogenous RNA of interest at the specific sequence and the out of reading frame initiation codons are detached and the palindromic termination element stabilizes the cleaved exogenous RNA of interest and protects it from degradation, so that the DT is capable of being expressed and kills the cell and the neighboring cells population. For example, see FIG. 44. The sequence of the cleaved exogenous RNA of this example is set forth as SEQ ID NO. 88.

### Example 6

### Specific Cellular Expression of an Exogenous Protein of Interest Encoded by an Exogenous RNA of Interest

General protocol for experiments described herein below:
The day before transfection, about 120,000 of T293 cells per well were seeded in 24 well plate, at the day of transfection each well was co-transfected with:
1. Renila/luciferase plasmid—170 ng of plasmid expressing Renilla luciferase gene & firefly luciferase gene (plasmid E11, Psv40-INTRON-MCS-RLuc - - - Phsvtk-Fluc, SEQ ID NO: 22 or plasmid E65, Psv40-INTRON-Tsp-TD1-TLacZ-RLuc-PTS-60ATG - - - Phsvtk-FLuc, SEQ ID NO. 23).
2. Tested plasmid=30 ng of tested plasmid (as detailed below).
3. siRNA+ or siRNA−=10 pmole of siRNA double stranded molecule that can induce cleavage (siRNA+) or does not induce cleavage (siRNA−) of the mRNA encoded by the tested plasmid. (detailed below).
The transfection was performed using lipofectamine 2000 transfection reagent (Invitrogen) according to manufacturer protocol. 48 hrs post transfection the Renilla luciferase gene expression was measured using the dual luciferase reported assay kit (Promega) and luminometer (glomax 20/20 promega), and the relative light units (RLU) were determined.
The tested plasmid may be any type of the following plasmids:
Negative control=Plasmid that does not encode for a diphtheria toxin (DTA);
Positive control=Plasmid that constitutively encodes for diphtheria toxin (DTA);
Test plasmid=plasmid of the composition of the invention, i.e. plasmid comprising target sites for siRNA+ between an inhibitory sequence and a downstream sequence encoding for diphtheria toxin (DTA). For the test plasmid, when the co-transfected siRNA+ cleaves the inhibitory sequence of the test plasmid, the diphtheria toxin is capable of being expressed and kills the cells in which it is expressed, thereby—reducing Renilla expression and overall measurement of RLU.
The tested plasmid was tested with 2 different siRNAs+ and with 2 different siRNAs−, separately, and each in triplicate.
The results are calculated as follows:
Fold of Activation=Average of measured RLU (Relative light unit) in the presence of each of the 2 siRNA− with the test plasmid (6 wells) divided by the average of RLU using one of the siRNA+ with the test plasmid (3 wells).
Fold of leakage=Average of RLU using all the siRNAs−/+ with the negative control plasmid divided by the Average of RLU using each of the 2 siRNA− with the test plasmid.
siRNA+/−RLU=Average of measured RLU in the presence of one co-transfected siRNA+ or the presence of two co-transfected siRNA−, independently.

The plasmids were constructed using common and known methods practiced in the art of molecular biology. The backbone vectors for the constructed plasmids described herein below are: psiCHECK™-2 Vectors (promega, Cat. No. C8021) or pcmv6-A-GFP (OriGene, Cat. No. PS100026). The appended name of each plasmid indicates sequences which are comprised within the plasmid sequence, as further detailed below, with respect to the test plasmids.

siRNA Sequences:
1. RL Duplex (Dharmacon, Cat. No. P-002070-01-20) (SEQ ID NO. 65 (sense strand) and SEQ ID 66 (anti sense strand)).
2. GFPDuplex II (Dharmacon, Cat. No. P-002048-02-20), (SEQ ID NO. 67 (sense strand) and SEQ ID NO. 68 (anti sense strand)).
3. siRNA—Control (Sigma, Cat. No., VC30002 000010), (SEQ ID NO. 69 (sense strand) and SEQ ID NO. 70, (anti sense strand)).
4. Anti βGal siRNA-1 ((target site: Tlacz (SEQ ID NO. 71), Dharmacon, Cat. No. P-002070-01-20) (SEQ ID NO. 72 (sense strand) and SEQ ID NO. 73 (antisense strand)).
5. Luciferase GL3 Duplex ((target site: Tfluc (SEQ ID NO. 74), Dharmacon, Cat. No. D-001400-01-20), (SEQ ID NO. 75 (sense strand) and SEQ ID NO. 76 (antisense strand)).
6. GFPDuplex I ((target site: TD1, (SEQ ID NO. 77), Dharmacon, Cat. No. P-002048-01-20), (SEQ ID NO. 78 (sense strand) and SEQ ID NO. 79 (antisense strand)).
7. TCTL (target site: TCTL (SEQ ID NO. 80), Dharmacon, SEQ ID NO. 81 (sense strand) and SEQ ID NO. 82 (anti sense strand)).

In each experiment, the siRNA that has target site in the test plasmid is used as siRNA+, and the other siRNAs that do not have a corresponding target site in the tested plasmid was used as siRNA−.

### Negative Control Plasmids:

1. E34 (SEQ ID NO. 10)—Pcmv-4ORF̂-TD1-Tfluc - - - Psv40-TGFP.
2. E71 (SEQ ID. NO. 17)—Psv40-INTRON-4ORF̂- - - Phsvtk-Fluc.
3. E38-3CARz-4S&L. The insert of E38 (SEQ ID. NO. 19) was ligated into a PMK shuttle vector (GeneArt) at pacI and XhoI restriction sites.

### Positive Control Plasmids:

1. E28 (SEQ ID. NO. 11)—Pcmv-Tfluc-TD1-cDTAWT - - - Psv40-TGFP.

### 2. E20 (SEQ ID. NO. 12)—Pcmv-nsDTA - - - Psv40-TGFP

### 3. E70 (SEQ ID. NO. 13)—Psv40-INTRON-cDTAWT - - - Phsvtk-Fluc

### 4. E3 (SEQ ID. NO. 14)—Pcmv-KDTA - - - Psv40-TGFP

5. E89 (SEQ ID. NO. 15)—Pcmv - - - DT̂A - - - Psv40-TGFP
6. E110 (SEQ ID. NO. 16)—Pcmv-D5̂TA - - - Psv40-TGFP

### 7. E4 (SEQ ID. NO. 18)—Pcmv-KDTA - - - Psv40-Hygro

### 8. E11) (SEQ ID. NO. 20)—Pef1-DTA24- - - ZEO::GFP-Pcmv

9. E143 (SEQ ID. NO. 21)—3PolyA-Prp119-cDTAWT - - - Phsvtk-Fluc

### Test Plasmids

1. E80 (SEQ ID. NO. 1)—Pcmv-4ORF̂-TD1-Tfluc-S-cDTAWT - - - Psv40-TGFP (pCMV promoter (nts. 420-938 of SEQ ID NO. 1); 4ORF̂=Inhibitory sequence composed of: 9 TISU sequences and 57 kozak sequences, with 57, 57, 36, 36, 21, 21, 21, and 21 nt between adjacent ATG codons, in 4 consecutive ORFs (nt 1027-3547 of SEQ ID NO. 1). The first ORF (nt. 1031-1651 of SEQ ID NO. 1) is 621 nt & is translated from TISU (nt. 1027-1038 of SEQ ID NO. 1), and the next 3ORF̂(nt. 1662-2996, nt. 2306-2941 and nt 2951-3547 of SEQ ID NO. 1) are translated from Kozak sequence, The last ORF (nt 2951-3547 of SEQ ID NO. 1) stops before the coding sequence of the wild type DTA (cDTAwt=wt DTA coding sequence, without promoter/splicing/termination/polyA sites and with kozak sequence (nt 3568-4155 of SEQ ID NO. 1); followed by TGFP coding sequence under the control of the SV40 promoter)). The plasmid further comprises target sites TD1 (SEQ ID NO. 77) and Tfluc (SEQ ID NO. 74).
2. E54 (SEQ ID. NO. 2)—Pcmv-4CARZ-PTS-60ATĜ-3ORF̂-TD1-Tfluc-incDTAWT - - - Psv40-TGFP (pCMV promoter (nucleotides (nt.) 420-938 of SEQ ID NO. 2); 4CAR=4 Cis Acting Ribozyme (nt. 1013-1373 of SEQ ID NO. 2); PTS=Peroxisomal targeting signal (nt. 1420-1500 of SEQ ID NO. 2); 60ATĜ=61 ATG, 46 in Kozak sequence with 53 nt between almost every 2 ATG (nt. 1534-4554 of SEQ ID NO. 2) and with stop codons inside the DTA coding sequence (nt. 6745-7332 of SEQ ID NO. 2); TGFP coding sequence (nt. 8452-9143 of SEQ ID NO. 2) under the control of the psv40 promoter (nt. 8092-8399 of SEQ ID. NO. 2)). The plasmid further comprises target sites TD1 (SEQ ID NO. 77) and Tfluc (SEQ ID NO. 74).
3. E113 (SEQ ID. NO. 3)—Pcmv-4ORF̂-TD1-Tfluc-PK-D5̂TA - - - Psv40-TGFP (pCMV promoter (nts. 420-938 of SEQ ID NO. 3); 4ORF̂(nt. 1027-3547 of SEQ ID NO. 3); PK=pseudoknot—stem and loop, such that the 6 nt of the loop are hybridized to the start codon of DTA (nt 3561-3611 of SEQ ID No. 3); 5̂=5 human introns (nts. 3712-3801, 3856-3960, 4066-4173, 4380-4519 and 4617-4783 of SEQ ID NO. 3) that are located within the coding sequence of the DTA (nts. 3609-3806 of SEQ ID NO. 3) and contain T-rich sequences for terminating RNA Polymerase 1 and/or 3 transcription, the introns are embedded in cDTAwt coding sequence; TGFP coding sequence (nts 5906-6597 of SEQ ID NO. 3) under the control of the psv40 promoter (nts. 5546-5853 of SEQ ID NO. 3)). The plasmid further comprises target sites TD1 (SEQ ID NO. 77) and Tfluc (SEQ ID NO. 74).
4. E91 (SEQ ID. NO. 4)—Pcmv-4ORF̂-TD1-Tfluc-DT̂A - - - Psv40-TGFP (pCMV promoter (nts. 420-938 of SEQ ID NO. 4), 4ORF̂ (nt. 1027-3507 of SEQ ID NO. 4); DT̂A=kozak DTA with an intron from Human Collagen 16A1 gene and without promoter/splicing/polyA signal (nt. 3520-4444 of SEQ ID NO. 4); TGFP coding sequence (nt. 5544-6235 of SEQ ID NO. 4) under the control of pSV40 promoter (nt. 5184-5491) The plasmid further comprises target sites TD1 (SEQ ID NO. 77) and Tfluc (SEQ ID NO. 74).
5. E112 (SEQ ID. NO. 5)—Pcmv-4ORF̂-2xTLacZinINTRON-8X[TCTL+TD1]-PK-D5̂TA - - - Psv40-TGFP (pCMV promoter (nts. 420-938 of SEQ ID NO. 5), 4ORF̂ (nt. 1027-3436 of SEQ ID NO. 5); 2xTLacZinINTRON=2 target of TLacZ in the intron of the commercial plasmid pSELECT-GFPzeo-LacZ (nt. 3438-3638 of SEQ ID NO. 5); 8X[TCTL+TD1] (nt. 3647-4052 of SEQ ID NO. 5); PK=pseudoknot—stem and loop, such that the Ent of the loop are hybridized to the start codon of DTA (nt 4059-4109 of SEQ ID No. 5); 5̂=5 human introns (nts. 4210-4299, 4354-4458, 4564-4671, 4878-5017 and 5115-5281 of SEQ ID NO. 5) that are located within the coding sequence of the DTA (nt. 4107-5304 of SEQ ID NO. 5) and contain T-rich sequences for terminating RNA Polymerase 1 and/or 3 transcription, the introns are embedded in a cDTAwt coding sequence; TGFP coding sequence (nt 6404-7095 of SEQ ID NO. 5) under the control of the psv40 promoter (nts. 6044-6351 of SEQ ID NO. 5)). The plasmid further comprises 8 copies of target sites TD1 (SEQ ID NO. 77), TCTL (SEQ ID NO. 80) and 2 copies of TLacZ (SEQ ID NO. 71).
6. E87 (SEQ ID. NO. 6)—Pcmv-4ORF̂-TD1-3TLacZ-Tctl-BGlob-25G-XRN1S&L-DT̂A - - - Psv40-TGFP (pCMV promoter (nts. 420-938 of SEQ ID NO. 6); 4ORF̂ (nt. 1027-3430 of SEQ ID NO. 6); BGlob=beta globin 5′ truncated end that is capped (nt. 3577-3655 of SEQ ID NO 6). 25G=a stretch of 25 consecutive G nucleotides (nt. 3660-3684 of SEQ ID NO. 6) that can block/interfere with XRN exoribonuclease enzyme; XRN1S&L=stem and loop structure of the yellow fever virus 3′UTR that can block XRN1 exoribonuclease (nt. 3687-3767 of SEQ ID. NO. 6). DT̂A=kozak DTA with an intron from Human Collagen 16A1 gene and without promoter/splicing/polyA signal (nt. 3787-4711 of SEQ ID NO. 6); TGFP coding sequence (nt 6404-7095 of SEQ ID NO. 6) under the control of the psv40 promoter (nts. 5811-6502 of SEQ ID NO. 6)). The plasmid further comprises TD1 (SEQ ID NO. 77), 3 copies of TLacz (SEQ ID NO. 71) and TCTL target sites (SEQ ID NO. 80).
7. E123 (SEQ ID. NO. 7)—Psv40-INTRON-4ORF̂-3X[TD1-TLacZ]-4PTE-SV40intron-HBB-DTA - - - Phsvtk-Fluc (pSV40 promoter (nt. 7-419 of SEQ ID NO. 7), 4ORF̂=9 TISU sequences and 57 kozak sequences, with 57, 57, 36, 36, 21, 21, 21, and 21 nt between adjacent ATG codons, in 4 consecutive ORFs (nt 722-2387 of SEQ ID NO. 7); 4PTE=4 kinds of the stem and loop structures of the Palindromic termination element (nt. 3318-3473 of SEQ ID NO. 7). SV40intron ═SV40 small t antigen intron (nt. 3505-3596 of SEQ ID NO. 7); HBB=hemoglobin beta mRNA without ATG and including its first intron (nt. 3627-4406 of SEQ ID NO. 7); cDTAwt coding sequence (nt. 4431-5014 of SEQ ID NO. 7); HSKVK promoter (nt. 5106-5858 of SEQ ID NO. 7) and firefly luciferase coding sequence (nt. 5894-7546 of SEQ ID. NO. 7). The plasmid further comprises 3 copies of TD1 (SEQ ID NO. 77) and TLacz target sites (SEQ ID NO. 71).
8. E30 (SEQ ID. NO. 8)—Pcmv-4ORF̂-TD1-Tfluc-incDTAWT - - - Psv40-TGFP (pCMV promoter (nts. 420-938 of SEQ ID NO. 8); 4ORF̂=9 TISU sequences and 57 kozak sequences, with 57, 57, 36, 36, 21, 21, 21, and 21 nt between adjacent ATG codons, in 4 consecutive ORFs (nt 1027-3547 of SEQ ID NO. 8). The first ORF (nt. 1031-1651 of SEQ ID NO. 8) is translated from TISU (nt. 1027-1038 of SEQ ID NO. 8), and the next 3ORF̂(nt. 1662-2996, nt. 2306-2941 and nt 2951-3547 of SEQ ID NO. 8) are translated from Kozak sequence, The last ORF (nt 2951-3516 of SEQ ID NO. 8) stops inside the coding sequence of the wild type DTA (cDTAwt=wt DTA coding region, without promoter/splicing/termination/polyA sites and with kozak sequence (nt 3568-4155 of SEQ ID NO. 8); followed by TGFP coding sequence under the control of the SV40 promoter)). The plasmid further comprises target sites TD1 (SEQ ID NO. 77) and Tfluc (SEQ ID NO. 74).
9. E142 (SEQ ID. NO. 9)-3PolyA-Prp119-4ORF̂-TD1-Tfluc-S-cDTAWT - - - Phsvtk-Fluc. 3PolyA=HSV poly A, SV40 poly A, synthetic poly A (nt. 60-247 of SEQ ID NO. 9); Prp119=promoter of RPL19 (ribosomal protein L19) taken with its first intron (nt. 248-1941 of SEQ ID NO. 9); 4ORF̂=9 TISU sequences and 57 kozak sequences, with 57, 57, 36, 36, 21, 21, 21, and 21 nt between adjacent ATG codons, in 4 consecutive ORFs (nt 1.948- - - 4366 of SEQ ID NO. 9); coding sequence of the wild type DTA (nt. 4457-5044 of SEQ ID NO. 9); HSKVK promoter (nt. 5136-5888 of SEQ ID NO. 9) and firefly luciferase coding sequence (nt. 5924-7576 of SEQ ID. NO. 9). The plasmid further comprises target sites TD1 (SEQ ID NO. 77) and Tfluc (SEQ ID NO. 74).

### Results:

The results are presented in following tables 1-5 and 6A-C. The results show the RLU measured in cells transfected with the indicated plasmids and siRNA molecules under various experimental conditions. The siRNA+ molecules used are the siRNA molecules that can bind their corresponding target sequence(s) within the tested plasmid.

TABLE 1

| | | | RLU in the | RLU in the |
|---|---|---|---|---|
| | Fold of | Fold of | presence of | presence of |
| Tested plasm id | Activation | leakage | siRNA+ | siRNA− |
| E34 (SEQ ID NO. 10) - Pcmv-4ORF\hat{ }-TD1-Tfluc--- | | | | 93M |
| Psv40-TGFP | | | | |
| E28 (SEQ ID NO. 11) - Pcmv-Tfluc-TD1- | | | | 35K |
| cDTAWT---Psv40-TGFP | | | | |
| E20 (SEQ ID NO. 12) - Pcmv-nsDTA---Psv40- | | | | 52K |
| TGFP | | | | |
| E70 (SEQ ID NO. 13) - Psv40-INTRON-cDTAWT--- | | | | 249K |
| Phsvtk-Fluc | | | | |
| E54 (SEQ ID. NO. 2) - Pcmv-4CARZ-PTS- | 4 | 5.1 | 4.4M | 18M |
| 60ATG\hat{ }-3ORF\hat{ }-TD1-Tfluc-incDTAWT---Psv40- | | | | |
| TGFP | | | | |

TABLE 2

| | | | RLU in the | RLU in the |
|---|---|---|---|---|
| | Fold of | Fold of | presence of | presence of |
| Tested plasmid | Activation | leakage | siRNA+ | siRNA− |
| E34 (SEQ ID NO. 10)- Pcmv-4ORF\hat{ }-TD1-Tfluc--- | | | | 33M |
| Psv40-TGFP | | | | |
| E28 (SEQ ID NO. 11) - Pcmv-Tfluc-TD1- | | | | 33K |
| cDTAWT---Psv40-TGFP | | | | |
| E3 (SEQ ID NO. 14) - Pcmv-KDTA---Psv40-TGFP | | | | 45K |
| E89 (SEQ ID NO. 15) - Pcmv---DT\hat{ }A---Psv40- | | | | 16K |
| TGFP | | | | |
| E110 (SEQ ID NO. 16) - Pcmv-D5\hat{ }TA---Psv40- | | | | 21K |
| TGFP | | | | |
| E113 (SEQ ID. NO. 3) - Pcmv-4ORF\hat{ }-TD1-Tfluc- | 6 | 15 | 367K | 2.2M |
| PK-D5\hat{ }TA---Psv40-TGFP | | | | |
| E80 (SEQ ID. NO. 1) - Pcmv-4ORF\hat{ }-TD1-Tfluc-S- | 5.2 | 15 | 427K | 2.2M |
| cDTAWT---Psv40-TGFP | | | | |
| E91 (SEQ ID. NO. 4) - Pcmv-4ORF\hat{ }-TD1-Tfluc- | 4.73 | 15 | 467K | 2.2M |
| DT\hat{ }A---Psv40 TGFP | | | | |
| E112 (SEQ ID. NO. 5) - Pcmv-4ORF\hat{ }- | 4.25 | 18.3 | 425K | 1.8M |
| 2xTLacZinINTRON-8X[TCTL + TD1]-PK-D5\hat{ }TA--- | | | | |
| Psv40-TGFP | | | | |
| E87 (SEQ ID. NO. 6) - Pcmv-4ORF\hat{ }-TD1- | 4.15 | 22 | 364K | 1.5M |
| 3TLacZ-Tctl-BGlob-25G-XRN1S&L-DT\hat{ }A--- | | | | |
| Psv40-TGFP | | | | |

TABLE 3

| | | | RLU in the | RLU in the |
|---|---|---|---|---|
| | Fold of | Fold of | presence of | presence of |
| Tested plasmid | Activation | leakage | siRNA+ | siRNA− |
| E71 (SEQ ID NO. 17) - Psv40-INTRON-4ORF\hat{ }--- | | | | 22.5M |
| Phsvtk-Fluc | | | | |
| E70 (SEQ ID NO. 3) - Psv40-INTRON-cDTAWT--- | | | | 819K |
| Phsvtk-Fluc | | | | |
| E123 (SEQ ID. NO. 7) - Psv40-INTRON-4ORF\hat{ }- | 3.37 | 1.8 | 3.7M | 12.5M |
| 3X[TD1-TLacZ]-4PTE-SV40intron-HBB-DTA--- | | | | |
| Phsvtk-Fluc | | | | |

TABLE 4

| | | | RLU in the | RLU in the |
|---|---|---|---|---|
| | Fold of | Fold of | presence of | presence of |
| Tested plasmid | Activation | leakage | siRNA+ | siRNA− |
| E34 (SEQ ID NO. 10) - Pcmv-4ORF\hat{ }-TD1-Tfluc--- | | | | 35M |
| Psv40-TGFP | | | | |
| E3 (SEQ ID NO. 14) - Pcmv-KDTA---Psv40-TGFP | | | | 47K |
| E4 (SEQ ID NO. 18) - Pcmv-KDTA---Psv40-Hygro | | | | 54K |
| E30 (SEQ ID. NO. 8) - Pcmv-4ORF\hat{ }-TD1-Tfluc- | 2.96 | 10.9 | 1.1M | 3.2M |
| incDTAWT---Psv40-TGFP | | | | |

TABLE 5

| | | | RLU in the | RLU in the |
|---|---|---|---|---|
| | Fold of | Fold of | presence of | presence of |
| Tested plasmid | Activation | leakage | siRNA+ | siRNA− |
| E38 (SEQ ID NO. 19) - 3CARz-4S&L | | | | 137M |
| E10 (SEQ ID NO. 20) - Pefl-DTA24---ZEO::GFP- | | | | 55K |
| Pcmv | | | | |
| E143 (SEQ ID NO. 21) - 3PolyA-Prpl19-cDTAWT--- | | | | 132K |
| Phsvtk-Fluc | | | | |
| E142 (SEQ ID. NO. 9) - 3PolyA-Prpl19-4ORF\hat{ }- | 2.53 | 5.9 | 9.1M | 23M |
| TD1-Tfluc-S-cDTAWT---Phsvtk-Fluc | | | | |

TABLE 6A

| | | Experiment number | | | | | | |
|---|---|---|---|---|---|---|---|---|
| | | 1 | 2 | 3 | 4 | 5 | 6 | 7 |
| Number of | #293cells | 135K | 180K | 150K | 120K | 150K | 120K | 90K |
| 293HEK cells | | | | | | | | |
| per well (24 | | | | | | | | |
| well plate) | | | | | | | | |
| Hours post | hrPT | 5 hr | 9 hr | 48 hr | 48 hr | 48 hr | 48 hr | 48 hr |
| transfection | | | | | | | | |
| co-transfection | REN | E11[170] | E11[170] | E11[170] | E11[170] | E11[170] | E11[170] | E11[170] |
| of Renilla | | | | | | | | |
| expressing | | | | | | | | |
| plasmid [ng] | | | | | | | | |
| co-transfection | siRNA | [10] | [10] | [10] | [10] | [10] | [10] | [10] |
| of siRNA+ or | | | | | | | | |
| siRNA−: [pico | | | | | | | | |
| mole] | | | | | | | | |
| co-transfection | ↓/RLU | [30] | [30] | [30] | [30] | [30] | [30] | [30] |
| of one of the | | | | | | | | |
| test plasmids | | | | | | | | |
| below [ng]: / | | | | | | | | |
| Results shown | | | | | | | | |
| below for each | | | | | | | | |
| plasmid are | | | | | | | | |
| RLU measured | | | | | | | | |
| under the | | | | | | | | |
| indicated | | | | | | | | |
| experimental | | | | | | | | |
| condition | | | | | | | | |
| Co transfection | E28 | 8.38K | 37.89K | 81.5K | 33K | 30.6K | 9.8K | 7.59K |
| of a Plasmid | (SEQ ID | | | | | | | |
| comprising the | NO. 11) | | | | | | | |
| sequence: | | | | | | | | |
| Pcmv-Tfluc- | | | | | | | | |
| TD1- | | | | | | | | |
| cDTAWT--- | | | | | | | | |
| Psv40-TGFP. | | | | | | | | |
| Co transfection | E34 | 161K | 8.8M | 83M | 33M | 40M | 23M | 11M |
| of Plasmid | (SEQ ID | | | | | | | |
| comprising the | NO. 10) | | | | | | | |
| sequence: | | | | | | | | |
| Pcmv-4ORF\hat{ }- | | | | | | | | |
| TD1-Tfluc--- | | | | | | | | |
| Psv40-TGFP | | | | | | | | |
| Co transfection | E80 | 110K | 4.15M | 7.17M | 2.2M | 4.33M | 2.3M | 1.1M |
| of Plasmid | (SEQ ID. | | | | | | | |
| comprising the | NO. 1) | | | | | | | |
| sequence: | | | | | | | | |
| Pcmv-4ORF\hat{ }- | | | | | | | | |
| TD1-Tfluc-S- | | | | | | | | |
| cDTAWT--- | | | | | | | | |
| Psv40-TGFP + | | | | | | | | |
| co-transfected | | | | | | | | |
| with siRNA− | | | | | | | | |
| Co transfection | E80 | 33K* | 1.35M* | 3M* | 427K* | 1.65M* | 800K | 354K |
| of Plasmid | (SEQ ID. | | | | | | | |
| comprising the | NO. 1) | | | | | | | |
| sequence | | | | | | | | |
| Pcmv-4ORF\hat{ }- | | | | | | | | |
| TD1-Tfluc-S- | | | | | | | | |
| cDTAWT--- | | | | | | | | |
| Psv40-TGFP | | | | | | | | |
| co-transfected | | | | | | | | |
| with siRNA+ | | | | | | | | |
| Fold of | si−/si+ | 3.33 | 3 | 2.4 | 5.1 | 2.6 | 2.87 | 3.1 |
| activation = | | | | | | | | |
| RLU measured | | | | | | | | |
| in the presence | | | | | | | | |
| of siRNA− | | | | | | | | |
| divided by RLU | | | | | | | | |
| measured in the | | | | | | | | |
| presence of | | | | | | | | |
| siRNA+ | | | | | | | | |
| Fold of | E34/ | 1.46 | 2.12 | 11.57 | 15 | 9.23 | 10 | 10 |
| leakiness = E34 | E80- | | | | | | | |
| (SEQ ID NO./ | | | | | | | | |
| E80-{smaller | | | | | | | | |
| than 1 = 0 | | | | | | | | |
| leakage} | | | | | | | | |

TABLE 6B

| | | Experiment number | | | | | | |
|---|---|---|---|---|---|---|---|---|
| | | 8 | 9 | 10 | 11 | 12 | 13 | 14 |
| Number of | #293cells | 100K | 120K | 120K | 100K | 100K | 100K | 125K |
| 293HEK cells | | | | | | | | |
| per well (24 well | | | | | | | | |
| plate) | | | | | | | | |
| Hours post | hrPT | 72 hr | 48 hr | 48 hr | 48 hr | 48 hr | 48 hr | 48 hr |
| transfection | | | | | | | | |
| co-transfection | REN | E11[195] | E65[15] | E11[170] | E11[170] | E11[140] | E11[110] | E11[170] |
| of Renilla | | | | | | | | |
| expressing | | | | | | | | |
| plasmid [ng] | | | | | | | | |
| co-transfection | siRNA | [10] | [10] | [5.5] | [10] | [10] | [10] | [10] |
| of siRNA+ or | | | | | | | | |
| siRNA−: [pico | | | | | | | | |
| mole] | | | | | | | | |
| co-transfection | ↓/RLU | [5] | [30]** | [30] | [30] | [60] | [90] | [30] |
| of one of the test | | | | | | | | |
| plasmids below | | | | | | | | |
| [ng]: /results | | | | | | | | |
| shown are RLU | | | | | | | | |
| under the | | | | | | | | |
| indicated | | | | | | | | |
| experimental | | | | | | | | |
| condition | | | | | | | | |
| Co transfection | E28 | 128K | 2.43K | | | | | |
| of Plasmid | (SEQ ID | | | | | | | |
| comprising the | NO. 11) | | | | | | | |
| sequence: Pcmv- | | | | | | | | |
| Tfluc-TD1- | | | | | | | | |
| cDTAWT--- | | | | | | | | |
| Psv40-TGFP | | | | | | | | |
| Co transfection | E34 | 117M | 1.1M | 97M | | | | |
| of Plasmid | (SEQ ID | | | | | | | |
| comprising the | NO. 10) | | | | | | | |
| sequence: Pcmv- | | | | | | | | |
| 4ORF\hat{ }-TD1- | | | | | | | | |
| Tfluc---Psv40- | | | | | | | | |
| TGFP | | | | | | | | |
| Co transfection | E80 | 14M | 65K | 10.3M | 4.9M | 2.4M | 1.4M | 7.2M |
| of Plasmid | (SEQ ID. | | | | | | | |
| comprising the | NO. 1) | | | | | | | |
| sequence: Pcmv- | | | | | | | | |
| 4ORF\hat{ }-TD1- | | | | | | | | |
| Tfluc-S- | | | | | | | | |
| cDTAWT--- | | | | | | | | |
| Psv40-TGFP + | | | | | | | | |
| co-transfected | | | | | | | | |
| with siRNA− | | | | | | | | |
| Co transfection | E80 | 2.69M* | 18K* | 2.7M* | 1.2M* | 586K* | 347K* | 2.1M* |
| of Plasmid | (SEQ ID. | | | | | | | |
| comprising the | NO. 1) | | | | | | | |
| sequence Pcmv- | | | | | | | | |
| 4ORF\hat{ }-TD1- | | | | | | | | |
| Tfluc-S- | | | | | | | | |
| cDTAWT--- | | | | | | | | |
| Psv40-TGFP co- | | | | | | | | |
| transfected with | | | | | | | | |
| siRNA+ | | | | | | | | |
| Fold of | si−/si+ | 5.2 | 3.6 | 3.8 | 4 | 4.1 | 4 | 3.4 |
| activation = | | | | | | | | |
| RLU measured | | | | | | | | |
| in the presence | | | | | | | | |
| of siRNA− | | | | | | | | |
| divided by RLU | | | | | | | | |
| measured in the | | | | | | | | |
| presence of | | | | | | | | |
| siRNA+ | | | | | | | | |
| Fold of leakiness = | E34/ | 8.35 | 16.92 | 9.41 | | | | |
| E34 (SEQ ID | E80- | | | | | | | |
| NO./E80- | | | | | | | | |
| {smaller than 1 = | | | | | | | | |
| 0 leakage} | | | | | | | | |

TABLE 6C

| | | Experiment number | | | | | | |
|---|---|---|---|---|---|---|---|---|
| | | 15 | 16 | 17 | 18 | 19 | 20 | 21 |
| Number of | #293cells | 125K | 125K | 100K | 100K | 100K | 100K | 200K |
| 293HEK cells | | | | | | | | |
| per well (24 well | | | | | | | | |
| plate) | | | | | | | | |
| Hours post | hrPT | 48 hr | 48 hr | 72 hr | 72 hr | 72 hr | 72 hr | 24 hr |
| transfection | | | | | | | | |
| co-transfection | REN | E11[140] | E11[110] | E11[150] | E11[150] | E11[750] | E11[750] | E11[170] |
| of Renilla | | | | | | | | |
| expressing | | | | | | | | |
| plasmid [ng] | | | | | | | | |
| co-transfection | siRNA | [10] | [10] | [10] | [15] | [10] | [15] | [10] |
| of siRNA+ or | | | | | | | | |
| siRNA−: [pico | | | | | | | | |
| mole] | | | | | | | | |
| co-transfection | ↓/RLU | [60] | [90] | [50] | [50] | [50] | [50] | [30] |
| of one of the test | | | | | | | | |
| plasmids below | | | | | | | | |
| [ng]: /results | | | | | | | | |
| shown are RLU | | | | | | | | |
| under the | | | | | | | | |
| indicated | | | | | | | | |
| experimental | | | | | | | | |
| condition | | | | | | | | |
| Co transfection | E28 | | | | | | | 97K |
| of Plasmid | (SEQ ID | | | | | | | |
| comprising the | NO. 11) | | | | | | | |
| sequence: Pcmv- | | | | | | | | |
| Tfluc-TD1- | | | | | | | | |
| cDTAWT--- | | | | | | | | |
| Psv40-TGFP | | | | | | | | |
| Co transfection | E34 | | | | | | | 10.7M |
| of Plasmid | (SEQ ID | | | | | | | |
| comprising the | NO. 10) | | | | | | | |
| sequence: Pcmv- | | | | | | | | |
| 4ORF\hat{ }-TD1- | | | | | | | | |
| Tfluc---Psv40- | | | | | | | | |
| TGFP | | | | | | | | |
| Co transfection | E80 | 3.16M | 1.76M | 3.67M | 4.3M | 13.3M | 13.3M | 4.2M |
| of Plasmid | (SEQ ID. | | | | | | | |
| comprising the | NO. 1) | | | | | | | |
| sequence: Pcmv- | | | | | | | | |
| 4ORF\hat{ }-TD1- | | | | | | | | |
| Tfluc-S- | | | | | | | | |
| cDTAWT--- | | | | | | | | |
| Psv40-TGFP + | | | | | | | | |
| co-transfected | | | | | | | | |
| with siRNA− | | | | | | | | |
| Co transfection | E80 | 950K* | 573K* | 1.4M* | 1.4M* | 5.8M* | 6.1M* | 2.1M* |
| of Plasmid | (SEQ ID. | | | | | | | |
| comprising the | NO. 1) | | | | | | | |
| sequence Pcmv- | | | | | | | | |
| 4ORF\hat{ }-TD1- | | | | | | | | |
| Tfluc-S- | | | | | | | | |
| cDTAWT--- | | | | | | | | |
| Psv40-TGFP co- | | | | | | | | |
| transfected with | | | | | | | | |
| siRNA+ | | | | | | | | |
| Fold of | si−/si+ | 3.32 | 3 | 2.6 | 3 | 2.3 | 2.18 | 2 |
| activation = | | | | | | | | |
| RLU measured | | | | | | | | |
| in the presence | | | | | | | | |
| of siRNA− | | | | | | | | |
| divided by RLU | | | | | | | | |
| measured in the | | | | | | | | |
| presence of | | | | | | | | |
| siRNA+ | | | | | | | | |
| Fold of leakiness = | E34/ | | | | | | | 2.54 |
| E34 (SEQ ID | E80- | | | | | | | |
| NO./E80- | | | | | | | | |
| {smaller than 1 = | | | | | | | | |
| 0 leakage} | | | | | | | | |

With respect to Table 6A-6C:
*= Indicate that the 2 siRNA+ show significant activation;
**= co-transfected also with 155 ng of plasmid E38 (SEQ ID NO. 19).

The results presented above in Tables 1-5 and 6A-6C clearly show that in the presence of an siRNA molecule(s) capable of inducing cleavage of the exogenous RNA of interest, the exogenous protein of interest (DTA) is expressed which, in turn results in increased cell death. The increased cell death results in reduced overall RLU measurements in the well, since less cells are expressing/producing the luciferase gene. The results demonstrate that indeed, only in cells which comprise a specific siRNA, the exogenous protein of interest (DTA in this example) is expressed, since only in these cells, cleavage of the exogenous RNA of interest at the cleavage site is induced, thereby allowing expression of the exogenous protein of interest in the cells.

### REFERENCES

1. T. Dalmay, 2008, MicroRNAs and cancer, Journal of Internal Medicine 263, 4: 366-375.
2. Y Zeng, 2006, Principles of micro-RNA production and maturation, Oncogene 25: 6156-6162.
3. BM Engels and G Hutvagner, 2006, Principles and effects of microRNA-mediated post-transcriptional gene regulation, Oncogene 25: 6163-6169.
4. Benjamin Haley & Phillip D Zamore, 2004, Kinetic analysis of the RNAi enzyme complex, Nature Structural & Molecular Biology 11: 599-606.
5. William C S Cho, 2007, OncomiRs: the discovery and progress of microRNAs in cancers. Molecular Cancer 6: 60.
6. Yan Zeng, Rui Yi and Bryan R. Cullen, 2003, MicroRNAs and small interfering RNAs can inhibit mRNA expression by similar mechanisms. Proc Natl Acad Sci USA 100, 17: 9779-9784.
7. Wadhwa R, Kaul S C, Miyagishi M, Taira K, 2004, Know-how of RNA interference and its applications in research and therapy. Mutat Res 567, 1: 71-84.
8. Sayda M. Elbashir, Winfried Lendeckel and Thomas Tuschl, 2001, RNA interference is mediated by 21- and 22-nucleotide RNAs. Genes & Dev 15, 2: 188-200.
9. Richard I. Gregory, Thimmaiah P Chendrimada, Neil Cooch, Ramin Shiekhattar, 2005, Human RISC Couples MicroRNA Biogenesis and Posttranscriptional Gene Silencing. Cell 123: 631-640.
10. Gallie D R, 1991, The cap and poly(A) tail function synergistically to regulate mRNA translational efficiency. Genes Dev 5, 11: 2108-16.
11. Entrez Gene: HIST1H2AC histone cluster 1, H2ac.

### 12. Lord M J, Jolliffe N A, Marsden C J, et al, 2003, Ricin Mechanisms of Cytotoxicity, Toxicol Rev 22, 1: 53-64.

13. Jeen-Kuan Chen, Chih-Hung Hung, Yen-Chywan Liaw and Jung-Yaw Lin, 1997, Identification of amino acid residues of Abrin-a A chain is essential for catalysis and reassociation with Abrin-a B chain by site-directed mutagenesis, Protein Engineering 10: 827-833.
14. Yamaizumi, M, Mekada, E, Uchida, T. and Okada Y, 1978, One molecule of Diphtheria toxin fragment A introduced into a cell can kill the cell, cell 15, 1: 245-50.
15. Weinberg M. S and Morris K. V, 2006, Are viral-encoded microRNAs mediating latent HIV-1 infection?, DNA Cell Biol 25: 223-231.

### 16. Velculescu V E et al, 2006, The Consensus Coding Sequences of Human Breast and Colorectal Cancers, Science 314, 5797: 268-274.

### 17. Intronn, Inc.

18. M. Puttarajul, Sharon F. Jamison, S. Gary Mansfield, Mariano A. Garcia-Blanco and Lloyd G. Mitchell, 1999, Spliceosome-mediated RNA trans-splicing as a tool for gene therapy. Nature Biotechnology 17: 246-252.
19. Song M S, Lee S W, 2006, Cancer-selective induction of cytotoxicity by tissue-specific expression of targeted trans-splicing ribozyme, FEBS Lett 580, 21: 5033-43.
20. Yaakov Benenson, Binyamin Gil, Uri Ben-Dor, Rivka Adar & Ehud Shapiro, 2004, An autonomous molecular computer for logical control of gene expression, Nature 429, 6990: 423-9.
21. Sano M, Kato Y, Taira K, Functional gene-discovery systems based on libraries of hammerhead and hairpin ribozymes and short hairpin RNAs, 2005, Mol Biosyst. 1:27-35.
22. Maurille J. Fournier et al, 1999, A small nucleolar RNA: ribozyme hybrid cleaves a nucleolar RNA target in vivo with near-perfect efficiency, PNAS 96, 12: 6609-6614.
23. Laising Yen, Jennifer Svendsen, Jeng-Shin Lee, John T. Gray, Maxime Magnier, Takashi Baba, Robert J. D'Amato & Richard C. Mulligan, 2004, Exogenous control of mammalian gene expression through modulation of RNA self-cleavage, Nature 431, 471-476.
24. Chabanon Herve and Ian Mickleburgh, 2004, Zipcodes and postage stamps: mRNA localisation signals and their trans-acting binding proteins, Briefings in Functional Genomics and Proteomics 3:240-256.
25. Wu L, Wells D, Tay J, Mendis D, Abbott M A, Barnitt A, Quinlan E, Heynen A, Fallon J R, Richter J D, 1998, CPEB-mediated cytoplasmic polyadenylation and the regulation of experience-dependent translation of alpha-CaMKII mRNA at synapses, Neuron 21, 5: 936-8.
26. Maria V. Baez and Graciela L. Boccaccio, Career investigator of the Consejo Nacional de Investigaciones Cientificas y Tecnologicas, 2005, Mammalian Smaug Is a Translational Repressor That Forms Cytoplasmic Foci Similar to Stress Granules, J. Biol. Chem 280, 52: 43131-43140.
27. C A Smibert, J E Wilson, K Kerr, and P M Macdonald, 1996, smaug protein represses translation of unlocalized nanos mRNA in the Drosophila embryo, GENES & DEVELOPMENT 10:2600-2609.
28. Carine Barreau, Luc Paillard and H. Beverley Osborne, 2006, AU-rich elements and associated factors: are there unifying principles?, Nucleic Acids Research 33, 22: 7138-7150.
29. Trent P. Munro, Rebecca J. Magee, Grahame J. Kidd, John H. Carson, Elisa Barbarese, Lisa M. Smith, and Ross Smith, 1999, Mutational Analysis of a Heterogeneous Nuclear Ribonucleoprotein A2 Response Element for RNA Trafficking, J Biol Chem, 274, 48: 34389-34395.
30. Suresh subramani, 1998, Components Involved in Peroxisome Import, Biogenesis, Proliferation, Turnover, and Movement, PHYSIOLOGICAL REVIEWS 78: 171-188.
31. Isken O, Maquat L E, 2007, Quality control of eukaryotic mRNA: safeguarding cells from abnormal mRNA function, Genes Dev 21, 15:1833-56.
32. Zheng H, Li L L, Hu D S, Deng X Y, Cao Y, 2007, Role of Epstein-Barr virus encoded latent membrane protein 1 in the carcinogenesis of nasopharyngeal carcinoma, Cell Mol Immunol, 3: 185-96.
33. Mei Y P, Zhou J M, Wang Y, Huang H, Deng R, Feng G K, Zeng Y X, Zhu X F, 2007, Silencing of LMP1 induces cell cycle arrest and enhances chemosensitivity through inhibition of AKT signaling pathway in EBV-positive nasopharyngeal carcinoma cells, Cell Cycle 6, 11: 1379-85.
34. Jacque J M, Triques K, Stevenson M, 2002, Modulation of HIV-1 replication by RNA interference, Nature 418, 6896: 435-8.
35. Zeng Y, Wagner E J, Cullen B R, 2002, Both natural and designed micro RNAs can inhibit the expression of cognate mRNAs when expressed in human cells, Mol Cell 6: 1327-33.
36. Joel D. Richter, 2001, Think globally, translate locally: What mitotic spindles and neuronal synapses have in common, Proc Natl Acad Sci USA 98, 13: 7069-7071.
37. Michael S. Wollenberg and Sanford M. Simon, 2004, Signal Sequence Cleavage of Peptidyl-tRNA Prior to Release from the Ribosome and Translocon, J. Biol. Chem. 279: 24919-24922.

### 38. Dan Frumkin, Adam Wasserstrom, Shalev Itzkovitz, Tomer Stern, Alon Harmelin, Raya Eilam, Gideon Rechavi and Ehud Shapiro, 2008, Cell Lineage Analysis of a Mouse Tumor, Cancer Research 68: 5924-5931.

39. Elfakess R, Dikstein R. (2008). A translation initiation element specific to mRNAs with very short 5′UTR that also regulates transcription. PloS One. 2008 Aug. 28; 3(8):e3094.

1-69. (canceled)

70. A composition comprising one or more polynucleotides for directing specific cleavage of an exogenous RNA of interest at a specific target site, the cleavage taking place only in the presence of an endogenous signal RNA in a cell, the endogenous signal RNA being an RNA molecule which comprises a signal sequence, the signal sequence being any predetermined sequence of from 18 to 25 nucleotides in length,
whereby introduction of said composition into a cell comprising said endogenous signal RNA, directs the cleavage of said exogenous RNA of interest at the specific target site that is located within a specific sequence, which is of sufficient complementarity to hybridize with the predetermined signal sequence.

71. The composition of claim 70, wherein said one or more polynucleotides comprises:
a first polynucleotide sequence encoding said exogenous RNA of interest;
a second polynucleotide sequence encoding a functional RNA capable of mediating the cleavage of the endogenous signal RNA at a predetermined cleavage site; and
a third polynucleotide sequence encoding a carrier RNA.

72. The composition of claim 71, wherein said carrier RNA is:
an RNA molecule that is at least about 18 nucleotides in length and is consisting essentially of:
(1) a first sequence of from 14 to 31 nucleotides in length which is of sufficient complementarity to an edge sequence to hybridize therewith, said edge sequence is 14-31 nucleotides in length and is located 0-5 nucleotides downstream from said predetermined cleavage site and extends downstream in said endogenous signal RNA;
(2) a second sequence downstream from said first sequence, wherein said second sequence is a random sequence that is 0-5 nucleotides in length;
(3) a third sequence upstream from said first sequence, wherein said third sequence is 0-7000 nucleotides in length; and wherein said predetermined cleavage site is the 5′ end of said predetermined signal sequence; or
wherein said carrier RNA comprises an RNA molecule that is at least about 18 nucleotides in length and is consisting essentially of:
(1) a first sequence of from 14 to 31 nucleotides in length which is of sufficient complementarity to an edge sequence to hybridize therewith, said edge sequence is 14-31 nucleotides in length and is located 0-5 nucleotides upstream from said predetermined cleavage site and extends upstream in said endogenous signal RNA;
(2) a second sequence upstream from the first sequence, wherein said second sequence is a random sequence that is 0-5 nucleotides in length;
(3) a third sequence downstream from the first sequence, wherein said third sequence is 0-7000 nucleotides in length; and wherein said predetermined cleavage site is the 3′ end of said predetermined signal sequence; or
wherein said carrier RNA is processed from a polynucleotide sequence comprising a carrier sequence that is at least about 18 nucleotides in length, said carrier sequence consisting essentially of:
(1) a first sequence of from 14 to 31 nucleotides in length which is of sufficient complementarity to an edge sequence to hybridize therewith, said edge sequence is 14-31 nucleotides in length and is located 0-5 nucleotides downstream from said predetermined cleavage site and extends downstream in said endogenous signal RNA;
(2) a second sequence downstream from said first sequence, wherein said second sequence is a random sequence that is 0-5 nucleotides in length; and
(3) a third sequence upstream from said first sequence, wherein said third sequence is 0-7000 nucleotides in length; wherein said polynucleotide sequence is cleaved within the cell at a carrier cleavage site that is a 3′ end of said carrier sequence; wherein the cleavage at the carrier cleavage site is effected by a functional nucleic acid which is encoded by a fourth polynucleotide sequence of the composition; and wherein the predetermined cleavage site is the 5′ end of said predetermined signal sequence; or
wherein said carrier RNA is processed from a polynucleotide sequence comprising a carrier sequence that is at least about 18 nucleotides in length, said carrier sequence consisting essentially of:
(1) a first sequence of from 14 to 31 nucleotides in length which is of sufficient complementarity to an edge sequence to hybridize therewith, said edge sequence is 14-31 nucleotides in length and is located 0-5 nucleotides upstream from said predetermined cleavage site and extends upstream in said endogenous signal RNA;
(2) a second sequence upstream from the first sequence, wherein said second sequence is a random sequence that is 0-5 nucleotides in length; and
(3) a third sequence downstream from said first sequence, wherein said third sequence is 0-7000 nucleotides in length; wherein said polynucleotide sequence is cleaved within the cell at a carrier cleavage site that is 5′ end of said carrier sequence; wherein the cleavage at the carrier cleavage site is effected by a functional nucleic acid which is encoded by a fourth polynucleotide sequence of the composition; and wherein said predetermined cleavage site is the 3′ end of said predetermined signal sequence.

73. The composition of claim 72, wherein said edge sequence is 23-28 nucleotides in length and is located from the predetermined cleavage site to about 23-28 nucleotides downstream, wherein said second sequence is 2 nucleotides in length and wherein said third sequence is 0 nucleotides in length; or
wherein said edge sequence is 25-30 nucleotides in length and is located 2 nucleotides upstream from the predetermined cleavage site and extends upstream in said endogenous signal RNA, wherein said second sequence is 0 nucleotides in length and wherein said third sequence is 0 nucleotides in length.

74. The composition of claim 70, wherein said endogenous signal RNA is a cellular mRNA, viral RNA, or both.

75. The composition of claim 70, wherein said predetermined signal sequence is unique to neoplastic cells, viral infected cells, or both.

76. The composition of claim 71, wherein said functional RNA is selected from the group consisting of: microRNA (miRNA), lariat-form RNA, short-hairpin RNA (shRNA), siRNA expression domain, antisense RNA, double-stranded RNA (dsRNA), small-interfering RNA (siRNA) and ribozyme.

77. The composition of claim 70, wherein said exogenous RNA of interest further comprises:
(a) a sequence encoding an exogenous protein of interest; and
(b) an inhibitory sequence that is capable of inhibiting the expression of the exogenous protein of interest;
wherein said specific target site is located between the inhibitory sequence and the sequence encoding an exogenous protein of interest, whereby following introduction of said composition into a cell comprising the endogenous signal RNA, said exogenous RNA of interest is transcribed and cleaved at said specific target site whereby said inhibitory sequence is detached from said sequence encoding the exogenous protein of interest and the exogenous protein of interest is capable of being expressed.

78. The composition of claim 77, wherein said exogenous protein of interest is selected from the group consisting of: Ricin, Ricin A chain, Abrin, Abrin A chain, Diphtheria toxin A chain, alpha toxin, saporin, maize RIP, barley RIP, wheat RIP, corn RIP, rye RIP, flax RIP, Shiga toxin, Shiga-like RIP, momordin, thymidine kinase, pokeweed antiviral protein, gelonin, Pseudomonas exotoxin, Pseudomonas exotoxin A, Escherichia coli cytosine deaminase, and modified forms thereof.

79. The composition of claim 77, wherein said inhibitory sequence comprises a plurality of initiation codons, wherein each of said initiation codons and the sequence encoding the exogenous protein of interest are not in the same reading frame; or
wherein said exogenous RNA of interest further comprises a stop codon located between said initiation codon and the start codon of said sequence encoding the exogenous protein of interest, wherein the stop codon and the initiation codon are in the same reading frame; or
wherein said inhibitory sequence further comprises a nucleotide sequence downstream from the initiation codon, wherein said nucleotide sequence and said initiation codon are in the same reading frame, and wherein the nucleotide sequence encodes a sorting signal for subcellular localization, the subcellular localization is selected from mitochondria, nucleus, endosome, lysosome, peroxisome and endoplastic reticulum (ER).

80. The composition of claim 79, wherein said inhibitory sequence further comprises a nucleotide sequence downstream from the initiation codon, wherein said nucleotide sequence and said initiation codon are in the same reading frame; and wherein said nucleotide sequence encodes a protein degradation signal; and/or
wherein said inhibitory sequence further comprises a nucleotide sequence downstream from the initiation codon, wherein said nucleotide sequence and said initiation codon are in the same reading frame; wherein said nucleotide sequence and said sequence encoding the exogenous protein of interest are in the same reading frame, wherein said nucleotide sequence encodes an amino acid sequence, whereby when the amino acid sequence is fused to the exogenous protein of interest the biological function of the exogenous protein of interest is inhibited.

81. The composition of claim 79, wherein said exogenous RNA of interest further comprises a stop codon downstream from said initiation codon, wherein said stop codon and said initiation codon are in the same reading frame and wherein said exogenous RNA of interest further comprises an intron downstream from the stop codon, whereby the exogenous RNA of interest is a target for nonsense-mediated decay (NMD).

82. The composition of claim 77, wherein said composition further comprises an additional polynucleotide sequence that encodes an additional RNA molecule, said additional RNA molecule comprises at the 3′ end a nucleotide sequence that is capable of binding to a sequence that is located upstream of said specific target site and downstream from the sequence encoding the exogenous protein of interest, wherein said additional RNA molecule, increases the efficiency of translation of said exogenous protein of interest in the cleaved exogenous RNA of interest.

83. The composition of claim 77, wherein said composition further comprises an additional polynucleotide sequence that encodes a cleaving component that is capable of effecting the cleavage of said exogenous RNA of interest at a position that is located upstream from the inhibitory sequence, wherein said cleaving component(s) is selected from the group consisting of:
(a) a nucleic acid sequence that is located within said exogenous RNA of interest, wherein said nucleic acid sequence is selected from the group consisting of: endonuclease recognition site, endogenous miRNA binding site, cis acting ribozyme and miRNA sequence, wherein said nucleic acid sequence, reduces the efficiency of translation of said exogenous protein of interest in the exogenous RNA of interest; and
(b) an inhibitory RNA, wherein said inhibitory RNA is selected from the group consisting of: microRNA (miRNA), lariat-form RNA, short-hairpin RNA (shRNA), siRNA expression domain, antisense RNA, double-stranded RNA (dsRNA), small-interfering RNA (siRNA) and ribozyme, wherein said inhibitory RNA, reduces the efficiency of translation of said exogenous protein of interest in said exogenous RNA of interest.

84. The composition of claim 70, wherein said one or more polynucleotides are integrated into the cell genome.

85. A method of treating cancer in a subject in need thereof, the method comprising administering the composition of claim 77 to said subject, whereby the cancer cells of said subject comprises the specific endogenous signal RNA in a cell, thereby treating cancer in said subject.

86. A composition comprising one or more polynucleotides for directing specific expression of an exogenous protein of interest in a cell, wherein the exogenous protein of interest is expressed only in the presence of an endogenous signal RNA in a cell, the endogenous signal RNA being an RNA molecule which comprises a signal sequence, the signal sequence being any predetermined sequence of from 18 to 25 nucleotides in length,
whereby introduction of said composition into a cell comprising said endogenous signal RNA, directs the cleavage of an exogenous RNA of interest at a specific target site that is located within a specific sequence, which is of sufficient complementarity to hybridize with the predetermined signal sequence, wherein only after the cleavage of said exogenous RNA of interest in the cell, the exogenous protein of interest, which is encoded by said cleaved exogenous RNA of interest is capable of being expressed in the cell.

87. The composition of claim 86, wherein said one or more polynucleotides comprises:
a first polynucleotide sequence encoding said exogenous RNA of interest;
a second polynucleotide sequence encoding a functional RNA capable of mediating the cleavage of the endogenous signal RNA at a predetermined cleavage site; and
a third polynucleotide sequence encoding a carrier RNA.

88. The composition of claim 87, wherein said carrier RNA is:
an RNA molecule that is at least about 18 nucleotides in length and is consisting essentially of:
(1) a first sequence of from 14 to 31 nucleotides in length which is of sufficient complementarity to an edge sequence to hybridize therewith, said edge sequence is 14-31 nucleotides in length and is located 0-5 nucleotides downstream from said predetermined cleavage site and extends downstream in said endogenous signal RNA;
(2) a second sequence downstream from said first sequence, wherein said second sequence is a random sequence that is 0-5 nucleotides in length; and
(3) a third sequence upstream from said first sequence, wherein said third sequence is 0-7000 nucleotides in length; or
wherein said carrier RNA comprises an RNA molecule that is at least about 18 nucleotides in length and is consisting essentially of:
(1) a first sequence of from 14 to 31 nucleotides in length which is of sufficient complementarity to an edge sequence to hybridize therewith, said edge sequence is 14-31 nucleotides in length and is located 0-5 nucleotides upstream from said predetermined cleavage site and extends upstream in said endogenous signal RNA;
(2) a second sequence upstream from the first sequence, wherein said second sequence is a random sequence that is 0-5 nucleotides in length; and
(3) a third sequence downstream from the first sequence, wherein said third sequence is 0-7000 nucleotides in length; or
wherein said carrier RNA is processed from a polynucleotide sequence comprising a carrier sequence that is at least about 18 nucleotides in length, said carrier sequence consisting essentially of:
(1) a first sequence of from 14 to 31 nucleotides in length which is of sufficient complementarity to an edge sequence to hybridize therewith, said edge sequence is 14-31 nucleotides in length and is located 0-5 nucleotides downstream from said predetermined cleavage site and extends downstream in said endogenous signal RNA;
(2) a second sequence downstream from said first sequence, wherein said second sequence is a random sequence that is 0-5 nucleotides in length; and
(3) a third sequence upstream from said first sequence, wherein said third sequence is 0-7000 nucleotides in length; wherein said polynucleotide sequence is cleaved within the cell at a carrier cleavage site that is a 3′ end of said carrier sequence; and wherein the cleavage at the carrier cleavage site is effected by a functional nucleic acid which is encoded by a fourth polynucleotide sequence of the composition; or
wherein said carrier RNA is processed from a polynucleotide sequence comprising a carrier sequence that is at least about 18 nucleotides in length, said carrier sequence consisting essentially of:
(1) a first sequence of from 14 to 31 nucleotides in length which is of sufficient complementarity to an edge sequence to hybridize therewith, said edge sequence is 14-31 nucleotides in length and is located 0-5 nucleotides upstream from said predetermined cleavage site and extends upstream in said endogenous signal RNA;
(2) a second sequence upstream from the first sequence, wherein said second sequence is a random sequence that is 0-5 nucleotides in length; and
(3) a third sequence downstream from said first sequence, wherein said third sequence is 0-7000 nucleotides in length; wherein said polynucleotide sequence is cleaved within the cell at a carrier cleavage site that is 5′ end of said carrier sequence; and
wherein the cleavage at the carrier cleavage site is effected by a functional nucleic acid which is encoded by a fourth polynucleotide sequence of the composition.

89. The composition of claim 86, wherein said exogenous RNA of interest further comprises:
a) a sequence encoding the exogenous protein of interest; and
b) an inhibitory sequence that is capable of inhibiting the expression of the exogenous protein of interest;
wherein said specific target site is located between the inhibitory sequence and the sequence encoding the exogenous protein of interest, whereby, following introduction of said composition into a cell comprising the endogenous signal RNA, said exogenous RNA of interest is transcribed and cleaved at said specific target site whereby the inhibitory sequence is detached from said sequence encoding the exogenous protein of interest and the exogenous protein of interest is capable of being expressed.

90. The composition of claim 86, wherein said endogenous signal RNA is a cellular mRNA, viral RNA, or both; and
wherein said predetermined signal sequence is unique to neoplastic cells, viral infected cells, or both.

91. The composition of claim 86, wherein said exogenous protein of interest is selected from the group consisting of: Ricin, Ricin A chain, Abrin, Abrin A chain, Diphtheria toxin A chain, alpha toxin, saporin, maize RIP, barley RIP, wheat RIP, corn RIP, rye RIP, flax RIP, Shiga toxin, Shiga-like RIP, momordin, thymidine kinase, pokeweed antiviral protein, gelonin, Pseudomonas exotoxin, Pseudomonas exotoxin A, Escherichia coli cytosine deaminase and modified forms thereof.

92. The composition of claim 86, wherein said functional RNA is selected from the group consisting of: microRNA (miRNA), lariat-form RNA, short-hairpin RNA (shRNA), siRNA expression domain, antisense RNA, double-stranded RNA (dsRNA), small-interfering RNA (siRNA) and ribozyme.

93. The composition of claim 86, wherein said one or more polynucleotides are integrated into the cell genome.

94. A method of treating cancer in a subject in need thereof, the method comprising administering the pharmaceutical composition of claim 86 to said subject, whereby cancer cells of said subject comprises the specific endogenous signal RNA in a cell, thereby treating cancer in said subject.

95. A method for killing a specific cell population which comprises an endogenous signal RNA, the method comprises: introducing the cell population with a composition comprising one or more polynucleotides for directing specific cleavage of an exogenous RNA of interest at a specific target site that is located within a specific sequence, which is of sufficient complementarity to hybridize with the endogenous signal RNA, the endogenous signal RNA being an RNA molecule which comprises a signal sequence, the signal sequence being any predetermined sequence of from 18 to 25 nucleotides in length; and
wherein the cleavage of the exogenous RNA of interest in the cell population, allows the expression of an exogenous protein of interest, capable of killing the cell population.

96. The method of claim 95 wherein said one or more polynucleotides comprises:
a first polynucleotide sequence encoding said exogenous RNA of interest;
a second polynucleotide sequence encoding a functional RNA capable of mediating the cleavage of the endogenous signal RNA at a predetermined cleavage site; and
a third polynucleotide sequence encoding a carrier RNA.

97. The method of claim 96, wherein said endogenous signal RNA is a cellular mRNA, viral RNA, or both.

98. The method of claim 96, wherein said exogenous protein of interest is selected from the group consisting of: Ricin, Ricin A chain, Abrin, Abrin A chain, Diphtheria toxin A chain, alpha toxin, saporin, maize RIP, barley RIP, wheat RIP, corn RIP, rye RIP, flax RIP, Shiga toxin, Shiga-like RIP, momordin, thymidine kinase, pokeweed antiviral protein, gelonin, Pseudomonas exotoxin, Pseudomonas exotoxin A, Escherichia coli cytosine deaminase and modified forms thereof.

99. The method claim 96, wherein said cell population is a neoplastic cell population.

100. The method of claim 96, wherein said cell population is present in an organism.
